(19) United States
(12) Patent Application Publication
(10) Pub. No.: US 2026/0248809 A1
(43) Pub. Date: Aug. 27, 2026

(54) ESTRADIOL PRODRUGS AND METHODS OF USE THEREOF

(71) Applicants: UNIVERSITY OF MARYLAND, BALTIMORE, Baltimore, MD (US); THE UNITED STATES GOVERNMENT as represented by THE DEPARTMENT OF VETERANS AFFAIRS, Washington, DC (US); The United States of America, as represented by The Secretary, Department of Health and Human Servic, Bethesda, MD (US)

(72) Inventors: Todd Gould, Elkridge, MD (US); Istvan Merchenthaler, Street, MD (US); Polymnia Georgiou, Shorewood, WI (US); Patrick Joseph Morris, Laurel, MD (US)

(21) Appl. No.: 18/726,051

(22) PCT Filed: Jan. 6, 2023
(86) PCT No.: PCT/US2023/060233

§ 371 Date: Jul. 1, 2024

Related U.S. Application Data
(60) Provisional application No. 63/297,655, filed on Jan. 7, 2022.

(51) Int. Cl. A61K 31/565; A61K 31/58; A61P 25/24; C07J 1/00; C07J 41/00; C07J 43/00

(52) U.S. Cl. CPC A61K 31/565; A61K 31/58; A61P 25/24; C07J 1/0074; C07J 41/0088; C07J 43/003

(57) ABSTRACT

Estradiol compounds and methods of using the same for treating diseases and conditions including depressive disorder, an anxiety disorder, post-traumatic stress disorder (PTSD), drug addiction, schizophrenia, Alzheimer's dementia, Parkinson's disease, stroke, traumatic brain injury (TBI), amyotrophic lateral sclerosis (ALS), complex regional pain syndrome (CRPS), chronic pain, neuropathic pain, anhedonia, fatigue, andropause-induced symptoms, and orchiectomy-induced symptoms are provided.

### CROSS REFERENCE TO RELATED APPLICATIONS

The present application claims priority to U.S. Provisional Patent Application No. 63/297,655, filed Jan. 7, 2022, the contents of which are incorporated herein by reference in their entireties.

### STATEMENT AS TO FEDERALLY SPONSORED RESEARCH

This invention was made with government support under VA Grant Numbers BX004062 and BX003631 awarded by the United States Department of Veterans Affairs. The government has certain rights in the invention.

### FIELD

The disclosure relates generally to estradiol compounds, modifications and derivatives thereof, and methods of using the same for treating diseases and conditions including a depressive disorder, an anxiety disorder, post-traumatic stress disorder (PTSD), drug addiction, schizophrenia, Alzheimer's dementia, Parkinson's disease, stroke, traumatic brain injury (TBI), amyotrophic lateral sclerosis (ALS), complex regional pain syndrome (CRPS), chronic pain, neuropathic pain, anhedonia, fatigue, andropause-induced symptoms, and orchiectomy-induced symptoms.

### BACKGROUND

Estradiol and molecules with similar properties toward estrogen receptors are used to treat a number of neuropsychiatric conditions including depression and schizophrenia. Unfortunately, administration of estrogen and certain estrogen analogs have off-target consequences in several other organs including the uterus and breasts, which can result in cancer and other undesirable effects. In addition, excess estrogen in male patients is undesirable for a number of reasons including gynecomastia.

There is a need for methods and compositions to easily administer estrogen and have it selectively concentrated in the brain while minimizing off-target consequences in other organs. The present disclosure fulfills this need and provides additional advantages set forth herein.

### SUMMARY

In one aspect, the disclosure provides a compound of formula (I), or a pharmaceutically acceptable salt, solvate, hydrate, cocrystal, or prodrug thereof:

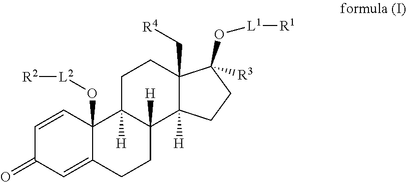

wherein in formula (I):
L¹ and L² are each a linker comprising independently one or more of a bond, —C(O)—, —O—, —S—, —NR^{a}—, —CR^{a}₂—, —C(O)O—, —C(O)S—, —C(O)NR^{a}—, —C(O)NR^{a}SO₂—, disubstituted alkyl, disubstituted heteroalkyl, disubstituted alkenyl, disubstituted alkynyl, disubstituted cycloalkyl, disubstituted heterocycloalkyl, disubstituted aryl, disubstituted arylalkyl, disubstituted heteroaryl, and disubstituted heteroarylalkyl;
R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl;
R¹ is selected from optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl;
R² is selected from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl;
R³ is selected from hydrogen and optionally substituted alkynyl; and
R⁴ is selected from hydrogen and optionally substituted alkyl.

In some embodiments, L¹ comprises one or more of a bond, disubstituted alkyl, —C(O)—, —C(O)O—, and —(CH₂)ₚO— wherein p is an integer from 1 to 5. In some embodiments, the compound of formula (I) is a compound of formula (10), formula (11), or formula (12), or a pharmaceutically acceptable salt, solvate, hydrate, cocrystal, or prodrug thereof. In some embodiments, R¹ is selected from optionally substituted alkyl, optionally substituted aryl, optionally substituted heterocycloalkyl, and optionally substituted heteroaryl. In some embodiments, R¹ is selected from methyl, ethyl, i-propyl, -n-butyl, n-pentyl, n-hexyl, n-heptyl, n-octyl, -i-butyl, -t-butyl, —C₉H₁₉, —C₁₀H₂₁, —C₁₁H₂₃,

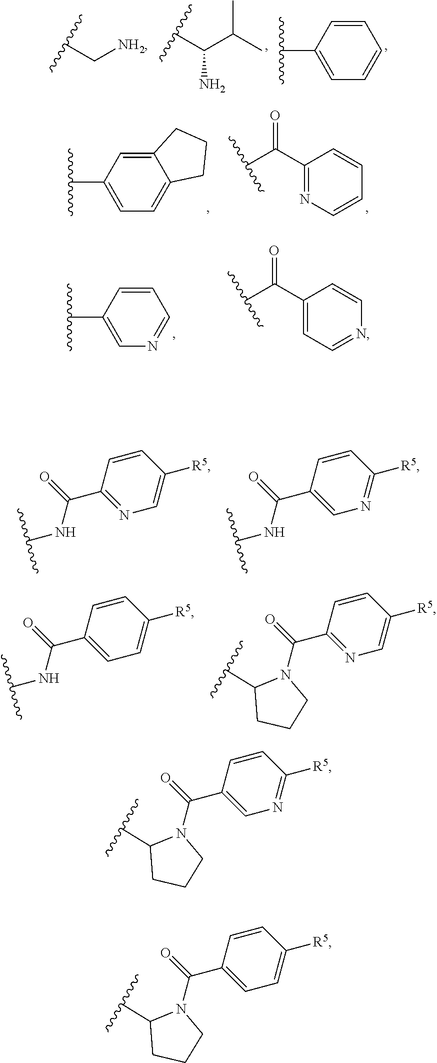

wherein R⁵ is selected from hydrogen, optionally substituted alkyl, —C(O)R⁶, —C(O)N(R⁶)₂, —S(O)R⁶, —S(O)₂R⁶, —S(O)N(R⁶)₂, —S(O)₂N(R⁶)₂,

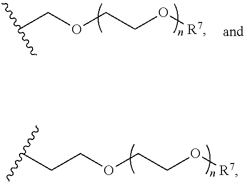

optionally R⁵ is selected from hydrogen and —S(O)₂NH₂, optionally R⁵ is —S(O)₂NH₂, wherein R⁶ is selected from hydrogen and optionally substituted alkyl; n is an integer from 1 to 5, and R⁷ is optionally substituted alkyl, optionally R⁷ is methyl. In some embodiments, R¹-L¹- is selected from

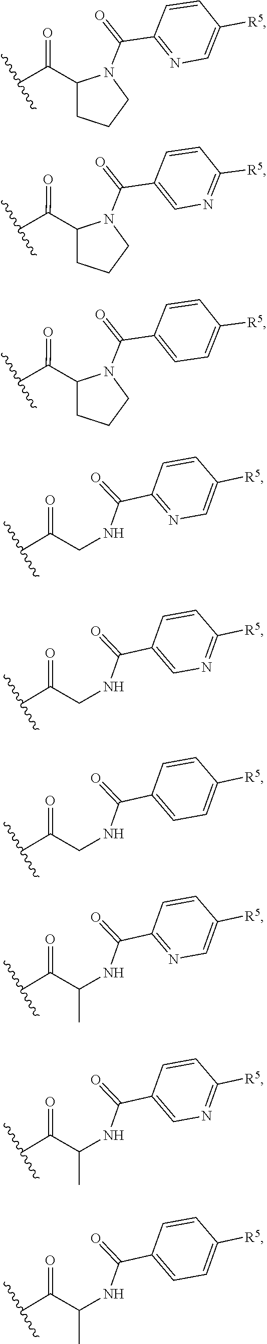

wherein R⁵ is selected from hydrogen, optionally substituted alkyl, —C(O)R⁶, —C(O)N(R⁶)₂, —S(O)R⁶, —S(O)₂R⁶, —S(O)N(R⁶)₂, —S(O)₂N(R⁶)₂,

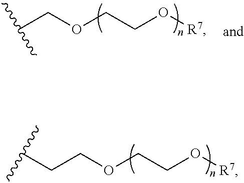

optionally R⁵ is selected from hydrogen and —S(O)₂NH₂, optionally R⁵ is —S(O)₂NH₂, wherein R⁶ is selected from hydrogen and optionally substituted alkyl; n is an integer from 1 to 5, and R⁷ is optionally substituted alkyl, optionally R⁷ is methyl. In some embodiments, R¹-L¹- is selected from

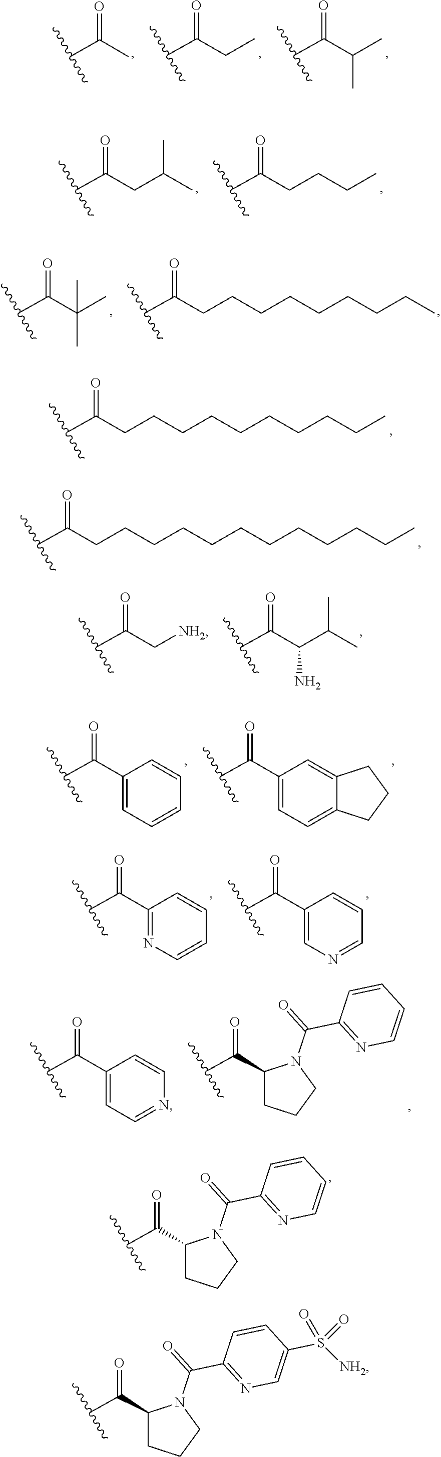

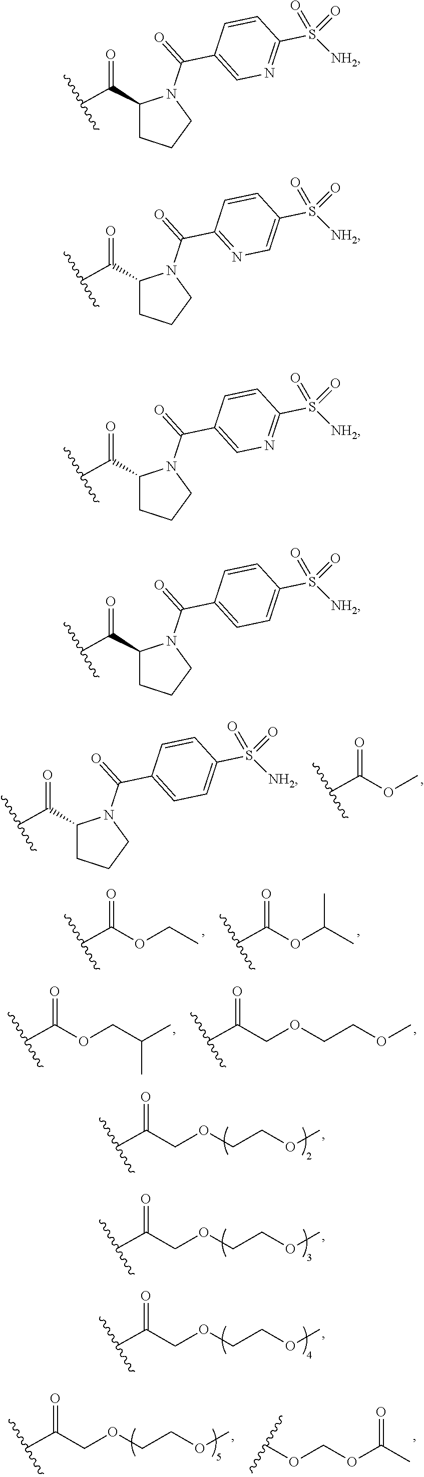

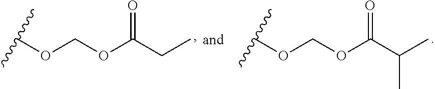

In some embodiments, L² comprises one or more of a bond, —C(O)—, —C(O)O—, and —(CH₂)ₚO— wherein p is an integer from 1 to 5. In some embodiments, R² is selected from hydrogen, optionally substituted alkyl, optionally substituted aryl, optionally substituted heterocycloalkyl, and optionally substituted heteroaryl. In some embodiments, R² is selected from methyl, ethyl, i-propyl,

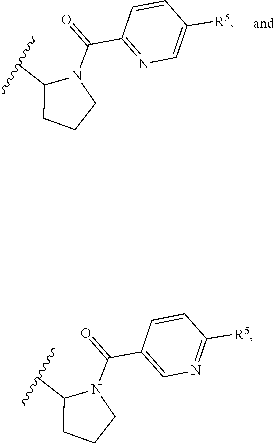

wherein R⁵ is selected from hydrogen, optionally substituted alkyl, —C(O)R⁶, —C(O)N(R⁶)₂, —S(O)R⁶, —S(O)₂R⁶, —S(O)N(R⁶)₂, —S(O)₂N(R⁶)₂,

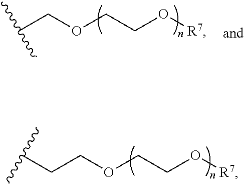

optionally R⁵ is selected from hydrogen and —S(O)₂NH₂, optionally R⁵ is —S(O)₂NH₂, wherein R⁶ is selected from hydrogen and optionally substituted alkyl; n is an integer from 1 to 5, and R⁷ is optionally substituted alkyl, optionally R⁷ is methyl. In some embodiments, R³ is selected from hydrogen and

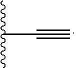 [SMILES: *C#C]

In some embodiments, R⁴ is selected from hydrogen and methyl.

In some embodiments, the compound of formula (I) is a compound of any one of formulas 1001-1256, or a pharmaceutically acceptable salt, solvate, hydrate, cocrystal, or prodrug thereof.

In another aspect, the disclosure provides a pharmaceutical composition comprising one or more compounds according to any one of formula (I), formula (10), formula (11), formula (12), formulas 1001-1256, or a pharmaceutically acceptable salt, solvate, hydrate, cocrystal, or prodrug thereof, and a pharmaceutically acceptable carrier.

In another aspect, the disclosure provides a pharmaceutical composition for treating or preventing a disease or condition alleviated by activating and/or enhancing estrogen receptor-β (ERβ)-activity, the pharmaceutical composition comprising one or more compounds according to any one of formula (I), formula (10), formula (11), formula (12), formulas 1001-1256, or a pharmaceutically acceptable salt thereof, and a physiologically compatible carrier. In some embodiments, the disease or condition is selected from a depressive disorder, an anxiety disorder, post-traumatic stress disorder (PTSD), drug addiction, schizophrenia, Alzheimer's dementia, Parkinson's disease, stroke, traumatic brain injury (TBI), amyotrophic lateral sclerosis (ALS), complex regional pain syndrome (CRPS), chronic pain, neuropathic pain, anhedonia, fatigue, andropause-induced symptoms, and orchiectomy-induced symptoms.

In another aspect, the disclosure provides a pharmaceutical composition for treating a disease or condition selected from a depressive disorder, an anxiety disorder, post-traumatic stress disorder (PTSD), drug addiction, schizophrenia, Alzheimer's dementia, Parkinson's disease, stroke, traumatic brain injury (TBI), amyotrophic lateral sclerosis (ALS), complex regional pain syndrome (CRPS), chronic pain, neuropathic pain, anhedonia, fatigue, andropause-induced symptoms, and orchiectomy-induced symptoms, the pharmaceutical composition comprising one or more compounds according to any one of formula (I), formula (10), formula (11), formula (12), formulas 1001-1240, or a pharmaceutically acceptable salt, solvate, hydrate, cocrystal, or prodrug thereof, and a pharmaceutically acceptable carrier. In some embodiments, the depressive disorder is selected from major depressive disorder, bipolar disorder, postpartum depression, drug withdrawal induced depression, treatment refractory depression, persistent depressive disorder (dysthymia), perinatal depression, seasonal affective disorder, seasonal depression, premenstrual dysphoric disorder, psychotic depression, suicidal ideation, suicidal actions, disruptive mood dysregulation disorder, premenstrual dysphoric disorder, substance/medication-induced depressive disorder, depressive disorder due to another medical condition, other specified depressive disorder, and an unspecified depressive disorder. In some embodiments, the anxiety disorder is selected from general anxiety disorder, obsessive-compulsive disorder (OCD), separation anxiety disorder, selective mutism, specific phobia, social anxiety disorder (social phobia), panic disorder, panic attack (specifier), agoraphobia, substance/medication-induced anxiety disorder, anxiety disorder due to another medical, other specified anxiety disorder, anhedonia, and unspecified anxiety disorder. In some embodiments, the drug addiction is selected from nicotine addiction, alcohol addiction, cannabis addiction, cocaine addiction, and opioid addiction.

In some embodiments, the pharmaceutical composition is formulated for oral administration.

In another aspect, the disclosure provides a method of treating or preventing a disease or condition alleviated by activating and/or enhancing estrogen receptor-β (ERβ)-activity in a patient in need thereof, the method comprising administering a therapeutically effective amount of one or more compounds according to any one of formula (I), formula (10), formula (11), formula (12), formulas 1001-1256, or a pharmaceutically acceptable salt, solvate, hydrate, cocrystal, or prodrug thereof. In some embodiments, the disease or condition is selected from a depressive disorder, an anxiety disorder, post-traumatic stress disorder (PTSD), drug addiction, schizophrenia, Alzheimer's dementia, Parkinson's disease, stroke, traumatic brain injury (TBI), amyotrophic lateral sclerosis (ALS), complex regional pain syndrome (CRPS), chronic pain, neuropathic pain, anhedonia, fatigue, andropause-induced symptoms, and orchiectomy-induced symptoms.

In another aspect, the disclosure provides a method of treating or preventing a disease or condition selected from a depressive disorder, an anxiety disorder, post-traumatic stress disorder (PTSD), drug addiction, schizophrenia, Alzheimer's dementia, Parkinson's disease, stroke, traumatic brain injury (TBI), amyotrophic lateral sclerosis (ALS), complex regional pain syndrome (CRPS), chronic pain, neuropathic pain, anhedonia, fatigue, andropause-induced symptoms, and orchiectomy-induced symptoms in a patient in need thereof, the method comprising administering a therapeutically effective amount of one or more compounds according to any one of formula (I), formula (10), formula (11), formula (12), formulas 1001-1240, or a pharmaceutically acceptable salt, solvate, hydrate, cocrystal, or prodrug thereof. In some embodiments, the depressive disorder is selected from major depressive disorder, bipolar disorder, postpartum depression, drug withdrawal induced depression, treatment refractory depression, persistent depressive disorder (dysthymia), perinatal depression, seasonal affective disorder, seasonal depression, premenstrual dysphoric disorder, psychotic depression, suicidal ideation, suicidal actions, disruptive mood dysregulation disorder, premenstrual dysphoric disorder, substance/medication-induced depressive disorder, depressive disorder due to another medical condition, other specified depressive disorder, and an unspecified depressive disorder. In some embodiments, the anxiety disorder is selected from general anxiety disorder, obsessive-compulsive disorder (OCD), separation anxiety disorder, selective mutism, specific phobia, social anxiety disorder (social phobia), panic disorder, panic attack (specifier), agoraphobia, substance/medication-induced anxiety disorder, anxiety disorder due to another medical, other specified anxiety disorder, anhedonia, and unspecified anxiety disorder. In some embodiments, the drug addiction is selected from nicotine addiction, alcohol addiction, cannabis addiction, cocaine addiction, and opioid addiction. In some embodiments, the patient is male.

### BRIEF DESCRIPTION OF THE FIGURES

FIGS. 1A-1N illustrate experimental data demonstrating that estrogen receptor beta (ERβ) underlies male stress susceptibility. FIG. 1A illustrates a timeline and schematics of the subthreshold social defeat stress (SSDS) and behavioral paradigms. Male estrogen receptor alpha knockout mice demonstrated social interaction deficits (FIG. 1B) but not anhedonia following SSDS (FIG. 1C). Male ERβ knockout mice demonstrated social interaction deficits (FIG. 1D) and anhedonia (FIG. 1E) following SSDS. Orchiectomized (ORX) mice, characterized by the absence of testosterone and consequently estradiol (E2), developed social interaction deficits (FIG. 1F) and anhedonia (FIG. 1G) following SSDS. FIGS. 1H-1J illustrate experimental data showing that a single s.c. administration of E2 was effective in preventing social interaction deficits and anhedonia when administered 45 min prior to SSDS, while an administration of E2 5 min after SSDS reversed only the observed anhedonia. FIG. 1K illustrates experimental data showing that rAAV-DIO-tdTomato injected in the nucleus accumbens (NAc) of ERβ-cre mice reveals a strong ERβ projection from the basolateral amygdala (BLA). FIGS. 1L-1N illustrate experimental data showing that whole-cell patch clamp measurement of optically-induced postsynaptic currents (oPSC) in the NAc of ERβ-cre mice that received an injection of ChR2-DIO-eYFP in the BLA. oPSCs were blocked with bath applied NBQX and APV, indicating that the ERβ-expressing BLA-NAc neurons are glutamatergic. Data shown are the mean S.E.M. * p<0.05; **p<0.01; ***p<0.001.

FIGS. 2A-2J illustrate experimental data demonstrating that estrogen receptor beta (ERβ)-expressing basolateral amygdala (BLA) neurons projecting to the nucleus accumbens (NAc) are involved in stress-induced social deficits. FIG. 2A illustrates a scheme showing that AAV-DIO-tdTomato was injected in the NAc of male ERβ-cre mice and retrogradely transported to cell bodies localized in the BLA. Orchiectomized (ORX) mice received estradiol (E2; s.c.) 45 min prior to subthreshold social defeat stress (SSDS). FIG. 2B illustrates representative immunofluorescence images from the BLA showing the co-localization of c-Fos and tdTomato in mice who received either E2 or vehicle (sesame oil) administration. FIG. 2C illustrates experimental data showing that increased activation of ERβ-expressing BLA to NAc cells in the anterior (A), medial (M), and posterior (P) BLA was observed in mice that received E2 prior to SSDS. FIG. 2D illustrates a schematic of the ChR2-DIO-eYFP injection in the BLA, optic fiber implantation in the NAc. FIG. 2E illustrates representative images from the injection and fiber implantation site. FIGS. 2F-2G illustrate experimental data showing that blue light stimulation (470 nm; 20 Hz) of ERβ-expressing BLA to NAc terminals induced a real-time place preference, shown by the increased time spent in the stimulation-paired compartment. FIG. 2H illustrates a schematic of the ChR2-DIO-eYFP injection in the BLA and optic fiber implantation in the NAc. FIGS. 2I-2J illustrate experimental data showing that blue light stimulation (470 nm; 4 Hz) of ERβ-expressing BLA to NAc terminals for 45 min prior to stress in ORX mice prevented social interaction deficits (FIG. 2I) but not anhedonia (FIG. 2J).

FIGS. 3A-3H illustrate experimental data demonstrating that testosterone does not directly mediate stress susceptibility in male mice. FIG. 3A shows a timeline, and the schematic of the stress and behavioral paradigms for which WT male C57BL/6J were assessed. FIG. 3B illustrates a representative heatmap traces during the social interaction test. FIGS. 3C-3D illustrate experimental data demonstrating that chronic administration of testosterone reversed the orchiectomy and SSDS-induced social interaction deficits (FIG. 3C) and anhedonia (FIG. 3D). FIGS. 3C-3D illustrate experimental data demonstrating that in intact mice, blockade of the androgen receptor with chronic flutamide did not induce any deficits in social interaction (FIG. 3E) and (FIG. 3F) anhedonia. FIGS. 3G-3H illustrate experimental data demonstrating that chronic administration of the aromatase inhibitor letrozole in minipumps to gonadally intact mice induced SSDS susceptibility as shown with social interaction deficits (FIG. 3G) and anhedonia (FIG. 3H). Data shown are the mean S.E.M. *p<0.05; **p<0.01; ***p<0.001.

FIGS. 4A-4F illustrate experimental data demonstrating that estradiol (E2) mediates stress susceptibility in male mice. FIGS. 4A-4B illustrate experimental data showing that dose-response following chronic administration of estradiol (E2) reversed the orchiectomy (ORX) and SSDS-induced social interaction deficits (FIG. 4A) and anhedonia (FIG. 4B). FIG. 4C illustrates the conversion of a brain selective E2 prodrug DHED to E2. FIGS. 4D-4E illustrate experimental data showing that chronic administration of DHED with minipumps prevented the SSDS/orchiectomy-induced (D) social interaction deficits (FIG. 4D) and anhedonia (FIG. 4E).

FIG. 4F illustrates a schematic representation and summary of the suggested mechanism for stress susceptibility in male mice.

FIGS. 5A-5B illustrate experimental data demonstrating the effects of E2 (FIG. 5A) and DHED (FIG. 5B) treatments on seminal vesicles weight.

FIGS. 6A-6B illustrate experimental data demonstrating DHED administered by IV and PO routes measured in plasma (FIG. 6A) and in brain (FIG. 6B).

FIGS. 7A-7B illustrate experimental data demonstrating the effects of inescapable foot shock stress on social interaction (FIG. 7A) and female urine preference behaviors (FIG. 7B).

FIG. 8 illustrates experimental data demonstrating that the exposure to CSDS did not modify fear conditioning (Day 1, Block 1), but did robustly reduce extinction.

FIG. 9 illustrates a reaction sequence the preparation of DHED prodrugs and conversion to estradiol E2.

FIG. 10 illustrates a non-limiting example of the preparation of testosterone prodrugs. Testosterone-decanoate and -EC586 are testosterone prodrugs that substantially enhance the oral bioavailability of testosterone.

FIGS. 11A-11B illustrate non-limiting examples of prodrug compounds of the disclosure.

FIG. 12 illustrates non-limiting examples of prodrug compounds of the disclosure.

FIGS. 13A-13N illustrate experimental data demonstrating behavioral characterization of estrogen receptor beta (ERβ) and alpha (ERα) knockout mice. FIGS. 13A-13E illustrate experimental data demonstrating that male ERα knockout mice did not demonstrate any baseline maladaptive behaviors in open-field (FIG. 13A), novel-object recognition (FIG. 13B), sucrose preference (FIG. 13C), elevated plus-maze (FIG. 13D), and forced-swim tests (FIG. 13E). FIGS. 13F-13J illustrate experimental data demonstrating that male ERβ knockout mice did not demonstrate any baseline maladaptive behaviors in the open-field (FIG. 13F), novel-object recognition (FIG. 13G), sucrose preference (FIG. 13H), elevated plus-maze (FIG. 13I), and forced-swim tests (FIG. 13J). FIG. 13K illustrates experimental data demonstrating the timeline of inescapable footshock stress in male ERβ knockout mice. FIGS. 13L-13M illustrate experimental data demonstrating that following footshock stress, male ERβ knockout mice developed social interaction deficits (FIG. 13L) and anhedonia (FIG. 13M). FIG. 13N illustrates experimental data demonstrating the timeline of the acute estradiol (E2) behavioral experiment. Data shown are the mean S.E.M. *p<0.05, Abbreviations: estrogen receptor alpha—ERα; estrogen receptor beta-ERβ-oPSCs; social interaction—SI; subthreshold social defeat stress—SSDS, ventral—V.

FIGS. 14A-14N illustrate experimental data demonstrating that estrogen receptor beta (ERβ) underlies male stress susceptibility. FIG. 14A illustrates a timeline and schematics of the subthreshold social defeat stress (SSDS) and behavioral paradigms. FIGS. 14B-14C illustrate experimental data demonstrating that male estrogen receptor alpha knockout mice demonstrated social interaction deficits (FIG. 14B), but not anhedonia (FIG. 14C) following SSDS. FIGS. 13D-13E illustrate experimental data demonstrating that male ERβ knockout mice demonstrated social interaction deficits (FIG. 14D) and anhedonia (FIG. 14E) following SSDS. FIGS. 14F-14G illustrate experimental data demonstrating orchiectomized (ORX) mice, characterized by the absence of testosterone and consequently estradiol (E2), developed social interaction deficits (FIG. 14F) and anhedonia (FIG. 14G) following SSDS. FIGS. 14H-14J illustrate experimental data demonstrating that a single s.c. administration of E2 was effective in preventing social interaction deficits and anhedonia when administered 45 min prior to SSDS, while an administration of E2 5 min after SSDS reversed only the observed anhedonia. FIG. 14K illustrates rAAV-DIO-tdTomato injected in the nucleus accumbens (NAc) of ERβ-cre mice reveals a strong ERβ projection from the basolateral amygdala (BLA). FIGS. 14L-14N illustrate whole-cell patch clamp measurement of optically-induced postsynaptic currents (oPSC) in the NAc of ERβ-cre mice that received an injection of ChR2-DIO-eYFP in the BLA. oPSCs were blocked with bath applied NBQX and APV, indicating that the ERβ-expressing BLA-NAc neurons are glutamatergic. Data shown are the mean S.E.M. *p<0.05; **p<0.01; ***p<0.001. Abbreviations: artificial cerebrospinal fluid—αCSF; (2R)-amino-5-phosphonovaleric acid—APV; basolateral amygdala—BLA; dorsal—D; 17β-Estradiol—E2; estrogen receptor alpha—ERα; estrogen receptor beta—ERβ; female urine—FU; female urine sniffing test—FUST; lateral—L; medial—M; male urine—MU; nucleus accumbens—NAc; 2,3-Dioxo-6-nitro-1,2,3,4-tetrahydrobenzofquinoxaline-7-sulfonamide—NBQX; orchiectomy—ORX; optical postsynaptic currents—oPSCs; social interaction—SI; subthreshold social defeat stress—SSDS, ventral—V.

FIGS. 15A-15B illustrate experiment data demonstrating the confirmation of cre expression in ERβ expressing cells in male ERβ-cre mice. FIG. 15A illustrates representative images and FIG. 15B illustrates quantification of RNAscope for Esr2 and cre in DAPI-labelled cells from the NAc and the BLA in male heterozygous ERβ mice. To confirm predominant cre expression on ERβ cells, brains from male heterozygous ERβ mice were fresh frozen and processed for RNAscope. Analysis for the expression of cre on Esr2⁺ cells was performed. Abbreviations: basolateral amygdala—BLA; nucleus accumbens—NAc

FIGS. 16A-16C illustrate experiment data demonstrating assessment of ERβ-expressing basolateral amygdala (BLA) to Nucleus accumbens (NAc) circuit. FIG. 16A illustrates representative images and FIGS. 16B-16C illustrate quantification of RNAscope for Esr2 (transcript coding for ERβ) and retrograde tracer positive cells. Male wildtype mice received a conjugated cholera toxin (tracer) in the NAc. Following 2 weeks, the brains were fresh frozen and processed for Esr2 expression and colocalization with tracer⁺ cells. Abbreviations: basolateral amygdala—BLA; nucleus accumbens—NAc.

FIGS. 17A-17J illustrate experiment data demonstrating that estrogen receptor beta (ERβ)—expressing basolateral amygdala (BLA) neurons projecting to the nucleus accumbens (NAc) are involved in stress-induced social deficits. FIG. 17A illustrates rAAV-DIO-tdTomato was injected in the NAc of male ERβ-cre mice and retrogradely transported to cell bodies localized in the BLA. Orchiectomized (ORX) mice received estradiol (E2; s.c.) 45 min prior to subthreshold social defeat stress (SSDS). FIG. 17B illustrates representative immunofluorescence images from the BLA showing the co-localization of c-Fos and tdTomato in mice who received either E2 or vehicle (sesame oil) administration. FIG. 17C illustrates experimental data demonstrating increased activation of ERβ-expressing BLA to NAc cells in the anterior (A), medial (M), and posterior (P) BLA was observed in mice that received E2 prior to SSDS. FIG. 17D illustrates a schematic of the ChR2-DIO-eYFP injection in the BLA, optic fiber implantation in the NAc, and FIG. 17E illustrates representative images from the injection and fiber implantation site. FIGS. 17F-17G illustrate experiment data demonstrating that blue light stimulation (470 nm; 20 Hz) of ERβ-expressing BLA to NAc terminals induced a real-time place preference, shown by the increased time spent in the stimulation-paired compartment. FIG. 17H illustrates a schematic of the ChR2-DIO-eYFP injection in the BLA and optic fiber implantation in the NAc. FIGS. 17I-17J illustrate experiment data demonstrating that blue light stimulation (470 nm; 4 Hz) of ERβ-expressing BLA to NAc terminals for 45 min prior to stress in ORX mice prevented social interaction (FIG. 17I) deficits but not anhedonia (FIG. 17J). Abbreviations: Anterior—A; basolateral amygdala—BLA; channelrhodopsin-2—ChR2; 17β-Estradiol—E2; estrogen receptor beta—ERβ, immunohistochemistry—IHC; middle—M; nucleus accumbens—NAc; orchiectomy—ORX; posterior—P; subthreshold social defeat stress—SSDS.

FIGS. 18A-18C illustrate experiment data demonstrating that analysis on the cFos-measured activation of the prefrontal, cingulate and insular cortex ERβ projections to the nucleus accumbens (NAc) by estradiol.

FIGS. 19A-19B illustrate experiment data demonstrating that the inclusion of data from female mice showing that activation of the ERβ BLA to NAc circuit does not induce RTPP than male mice.

FIGS. 20A-20K illustrate experiment data demonstrating that estrogen receptor beta (ERβ)—expressing basolateral amygdala (BLA) neurons projecting to the nucleus accumbens (NAc) decreased terminal activity is involved in stress-induced social deficits. FIG. 20A illustrates a schematic of the GCaMP6s-FLEX injection in the BLA, optic fiber implantation in the NAc, and FIG. 20B illustrates representative images from the injection and implantation sites. FIG. 20C illustrates representative traces from the fiber photometry recordings of ERβ-expressing BLA to NAc terminals in which each horizontal arrow represents an interaction bout. FIG. 20D illustrates a heatmap representation of the changes in z-score fluorescence when gonadally intact and ORX mice were interacting with a stranger or an empty cage. FIGS. 20E-20G illustrate experiment data demonstrating that increased calcium transients were observed in ERβ-expressing BLA to NAc terminals while gonadally-intact mice were interacting with the stranger mouse. In contrast, no difference was observed in calcium activity during social interaction in ORX mice. FIG. 20H illustrates experimental data demonstrating that increased calcium activity was also observed in anticipation of social interaction only in gonadally intact mice. FIG. 20I illustrates experimental data demonstrating that decreased in peak width calcium activity was observed during social interaction in ORX mice. FIGS. 20J-20K illustrate experiment data demonstrating that calcium activity in gonadally-intact mice during interaction with the stranger mouse was associated with higher interaction preference compared with ORX mice. Data shown are the mean±S.E.M. *p<0.05; **p<0.01; ***p<0.001. Abbreviations: anticipation—Ant; basolateral amygdala—BLA; baseline—BS; estrogen receptor beta—ERβ, interaction—Int; nucleus accumbens—NAc; orchiectomy—ORX; subthreshold social defeat stress-SSDS.

FIGS. 21A-21C illustrate experiment data demonstrating correlations of calcium activity recorded in the nucleus accumbens terminals of the basolateral amygdala ERβ projection neurons and social interaction. Pearson's correlation coefficients analysis demonstrated that there is a statistical trend between social interaction times from gonadally intact animals and z-score fluorescence (FIG. 21A), a significant correlation between interaction with the stranger mouse and z-score fluorescence (FIG. 21B) and a significant correlation between peak width changes and social interaction score changes. *p<0.05; ***p<0.001. Abbreviations: social interaction, SI.

FIGS. 22A-22L illustrate experiment data demonstrating behavioral characterization of orchiectomized (ORX) male mice. FIGS. 22A-22E illustrate experiment data demonstrating ORX mice did not demonstrate any baseline maladaptive behaviors in open-field (FIG. 22A), novel-object recognition (FIG. 22B), sucrose preference (FIG. 22C), elevated plus-maze (FIG. 22D), and forced-swim tests (FIG. 22E). FIG. 22F illustrates experimental data demonstrating that following acute stress ORX mice underwent a battery of tests. FIGS. 22G-22H illustrate experiment data demonstrating ORX mice did not show sucrose preference deficits (FIG. 22G); however, they demonstrated deficits in light/dark box (FIG. 22H). FIG. 22I illustrates experimental data demonstrating that no difference was observed in the elevated-plus maze. FIGS. 22J-22L illustrates experimental data demonstrating that decreased time spent in center in the open field test (FIG. 22J) and deficits in novel object recognition were observed (FIGS. 22K-22L). FIG. 22L illustrates experimental data demonstrating that no differences were found in the Forced-sim test. *p<0.05; **p<0.01.

FIGS. 23A-23H illustrate experimental data demonstrating that testosterone does not directly mediate stress susceptibility in male mice. FIG. 23A illustrates a timeline, and the schematic of the stress and behavioral paradigms for which WT male C57Bl/6J were assessed. FIG. 23B illustrates representative heatmap traces during the social interaction test. FIGS. 23C-23D illustrate experimental data demonstrating that chronic administration of testosterone reversed the orchiectomy and SSDS-induced social interaction deficits (FIG. 23C) and anhedonia (FIG. 23D). FIGS. 23E-23F illustrate experimental data demonstrating that in intact mice, blockade of the androgen receptor with chronic flutamide did not induce any deficits in (E) social interaction and (F) anhedonia. Chronic administration of the aromatase inhibitor letrozole in minipumps to gonadally intact mice induced SSDS susceptibility as shown with (G) social interaction deficits and (H) anhedonia. Data shown are the mean S.E.M. *p<0.05; **p<0.01; ***p<0.001. Abbreviations: female urine sniffing test—FUST; letrozole—LTZ; orchiectomy—ORX; social interaction—SI; subthreshold social defeat stress—SSDS; testosterone—T; veh—vehicle

FIGS. 24A-24F illustrate experimental data demonstrating that estradiol (E2) mediates stress susceptibility in male mice. FIGS. 24A-24B illustrate experimental data demonstrating that dose-response following chronic administration of estradiol (E2) reversed the orchiectomy (ORX) and SSDS-induced social interaction deficits (FIG. 24A) and anhedonia (FIG. 24B). FIG. 24C illustrates a scheme of the conversion of a brain selective E2 prodrug DHED to E2. FIGS. 24D-24E illustrate experimental data demonstrating that chronic administration of DHED with minipumps prevented the SSDS/orchiectomy-induced social interaction deficits (FIG. 24D) and anhedonia (FIG. 24E). FIG. 24F illustrates a schematic representation and summary of the suggested mechanism for stress susceptibility in male mice. Data shown are the mean S.E.M. * p<0.05; **p<0.01; ***p<0.001. basolateral amygdala—BLA; (17β)-10,17-Dihydroxy-estra-1,4-dien-3-one—DHED; 17β-Estradiol—E2; estrogen receptor beta—ERβ; orchiectomy—ORX; nucleus accumbens—NAc.

FIGS. 25A-25I illustrate experimental data demonstrating determination of the effectiveness of testosterone, flutamide and letrozole treatments. FIGS. 25A-25B illustrate experimental data demonstrating that mice that received chronic testosterone replaced therapy showed increased body weight (FIG. 25A) and decreased testis weight (FIG. 25B). FIG. 25C illustrates experimental data demonstrating that seminal vesicles weight was decreased in orchiectomized (ORX) mice and was reversed with the chronic testosterone replacement therapy. FIGS. 25D-25E illustrate experimental data demonstrating that mice that chronically received the androgen receptor antagonist flutamide did not manifest any differences in body weight (FIG. 25D) or testis weight (FIG. 25E). FIG. 25F illustrates experimental data demonstrating that chronic treatment with flutamide resulted in a decrease in seminal vesicle weight. FIGS. 25G-25I illustrate experimental data demonstrating that mice that chronically received the aromatase inhibitor, letrozole, showed increased body weight (FIG. 25G), no difference in testes weight (FIG. 25H), and increased seminal vesicle weight (FIG. 25I). Data shown are the mean S.E.M. *p<0.05; **p<0.01; ***p<0.001. Abbreviations: orchiectomized—ORX; testosterone, T; vehicle, veh.

FIGS. 26A-26D illustrate experimental data demonstrating effects of estradiol (E2) and DHED treatments on body and seminal vesicles weight changes. FIGS. 26A-26B illustrate experimental data demonstrating that mice that received estradiol (E2) treatment manifested increased body weight (FIG. 26A) and seminal vesicles weight (FIG. 26B). FIGS. 26C-26D illustrate experimental data demonstrating that mice that received DHED treatment had no difference in their body weight (FIG. 26C) and seminal vesicles weight (FIG. 26D) compared to control mice. Abbreviations: (17β)-10,17-Dihydroxy-estra-1,4-dien-3-one, DHED; 17β-Estradiol, E2; vehicle, veh.

FIGS. 27A-27H illustrate experimental data demonstrating effects of hormonal treatments on aggressiveness behaviors. No difference in the number of attacks was observed in ERβ^{-/-} (FIG. 27A), ERα^{-/-} (FIG. 27B), acute E2 (FIG. 27C), chronic testosterone (FIG. 27D), flutamide (FIG. 27E), letrozole (FIG. 27F), chronic E2 (FIG. 27G), and DHED treatment (FIG. 27H) versus controls. Abbreviations: (17β)-10,17-Dihydroxy-estra-1,4-dien-3-one, DHED; 17β-Estradiol, E2; estrogen receptor Alpha, ERα; estrogen receptor beta, ERβ; testosterone, T; vehicle, veh.

FIGS. 28A-28E illustrate experimental data demonstrating experiments showing % FU preference for ERα (FIGS. 28A-28B) and ERβ (FIGS. 28C-28D) with control or SSDS, and Sham or ORX with SSDS (FIG. 28E).

FIGS. 29A-29B illustrate the pharmacokinetic assessment of the DHED-acetate prodrug. The concentration levels of the DHED-acetate prodrug (FIG. 29A) and DHED (FIG. 29B) were measured in mice (n=3) after IV and PO administration of 5 mg/kg solution dose of DHED-acetate prodrug.

### DETAILED DESCRIPTION

Unless defined otherwise, all technical and scientific terms used herein have the same meaning as is commonly understood by one of skill in the art to which this disclosure belongs. All patents and publications referred to herein are incorporated by reference in their entireties.

### Definitions

As used herein, the terms “administer,” “administration” or “administering” refer to (1) providing, giving, dosing, and/or prescribing by either a health practitioner or his authorized agent or under his or her direction according to the disclosure; and/or (2) putting into, taking or consuming by the mammal, according to the disclosure.

The terms “co-administration,” “co-administering,” “administered in combination with,” “administering in combination with,” “simultaneous,” and “concurrent,” as used herein, encompass administration of two or more active pharmaceutical ingredients to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The terms “active pharmaceutical ingredient” and “drug” include, but are not limited to, the compounds described herein and, more specifically, compounds of any of formula (I), formula (10), formula (11), formula (12), formulas 1001-1256, and their features and limitations as described herein.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., increased sensitivity to apoptosis). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “QD,” “qd,” or “q.d.” mean quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

The term “pharmaceutically acceptable salt” refers to salts derived from a variety of organic and inorganic counter ions known in the art. Pharmaceutically acceptable acid addition salts can be formed with inorganic acids and organic acids. Preferred inorganic acids from which salts can be derived include, for example, hydrochloric acid, hydrobromic acid, sulfuric acid, nitric acid and phosphoric acid. Preferred organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve hydrogen transfer between the cocrystal and the drug, and instead involves intermolecular interactions, such as hydrogen bonding, aromatic ring stacking, or dispersive forces, between the cocrystal former and the drug in the crystal structure.

“Pharmaceutically acceptable carrier” or “pharmaceutically acceptable excipient” is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs disclosed herein, can also be incorporated into the described compositions and methods.

As used herein, the terms “treat,” “treatment,” and/or “treating” may refer to the management of a disease, disorder, or pathological condition, or symptom thereof with the intent to cure, ameliorate, stabilize, and/or control the disease, disorder, pathological condition or symptom thereof. Regarding control of the disease, disorder, or pathological condition more specifically, “control” may include the absence of condition progression, as assessed by the response to the methods recited herein, where such response may be complete (e.g., placing the disease in remission) or partial (e.g., lessening or ameliorating any symptoms associated with the condition).

As used herein, the terms “modulate” and “modulation” refer to a change in biological activity for a biological molecule (e.g., a protein, gene, peptide, antibody, and the like), where such change may relate to an increase in biological activity (e.g., increased activity, agonism, activation, expression, upregulation, and/or increased expression) or decrease in biological activity (e.g., decreased activity, antagonism, suppression, deactivation, downregulation, and/or decreased expression) for the biological molecule.

As used herein, the term “prodrug” refers to a derivative of a compound described herein, the pharmacologic action of which results from the conversion by chemical or metabolic processes in vivo to the active compound. Prodrugs include compounds wherein an amino acid residue, or a polypeptide chain of two or more (e.g., two, three or four) amino acid residues is covalently joined through an amide or ester bond to a free amino, hydroxyl or carboxylic acid group of a compound of any of formula (I), formula (10), formula (11), formula (12), formulas 1001-1256. The amino acid residues include but are not limited to the 20 naturally occurring amino acids commonly designated by one or three letter symbols but also include, for example, 4-hydroxyproline, hydroxylysine, desmosine, isodesmosine, 3-methylhistidine, beta-alanine, gamma-aminobutyric acid, citrulline, homocysteine, homoserine, ornithine and methionine sulfone.

Additional types of prodrugs are also encompassed. For instance, free carboxyl groups can be derivatized as amides or alkyl esters (e.g., methyl esters and acetoxy methyl esters). Prodrug esters as employed herein includes esters and carbonates formed by reacting one or more hydroxyls of compounds of the method of the disclosure with alkyl, alkoxy, or aryl substituted acylating agents employing procedures known to those skilled in the art to generate acetates, pivalates, methylcarbonates, benzoates and the like. As further examples, free hydroxyl groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxyl and amino groups are also included, as are carbonate prodrugs, sulfonate prodrugs, sulfonate esters and sulfate esters of hydroxyl groups. Free amines can also be derivatized to amides, sulfonamides or phosphonamides. All of the stated prodrug moieties may incorporate groups including but not limited to ether, amine and carboxylic acid functionalities. Moreover, any compound that can be converted in vivo to provide the bioactive agent (e.g., a compound of any of formula (I), formula (10), formula (11), formula (12), formulas 1001-1256) is a prodrug within the scope of the disclosure. Various forms of prodrugs are well known in the art. A comprehensive description of pro drugs and prodrug derivatives are described in: (a) The Practice of Medicinal Chemistry, Camille G. Wermuth et al., (Academic Press, 1996); (b) Design of Prodrugs, edited by H. Bundgaard, (Elsevier, 1985); (c) A Textbook of Drug Design and Development, P. Krogsgaard-Larson and H. Bundgaard, eds., (Harwood Academic Publishers, 1991). In general, prodrugs may be designed to improve the penetration of a drug across biological membranes in order to obtain improved drug absorption, to prolong duration of action of a drug (slow release of the parent drug from a prodrug, decreased first-pass metabolism of the drug), to target the drug action (e.g. organ or tumor-targeting, lymphocyte targeting), to modify or improve aqueous solubility of a drug (e.g., i.v. preparations and eyedrops), to improve topical drug delivery (e.g. dermal and ocular drug delivery), to improve the chemical/enzymatic stability of a drug, or to decrease off-target drug effects, and more generally in order to improve the therapeutic efficacy of the compounds utilized in the disclosure.

Unless otherwise stated, the chemical structures depicted herein are intended to include compounds which differ only in the presence of one or more isotopically enriched atoms. For example, compounds where one or more hydrogen atoms is replaced by deuterium or tritium, or wherein one or more carbon atoms is replaced by ¹³C- or ¹⁴C-enriched carbons, are within the scope of this disclosure.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, preferably from 0% to 10%, more preferably from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

“Depressive symptoms” include low mood, diminished interest in activities, psychomotor slowing or agitation, changes in appetite, poor concentration or indecisiveness, excessive guilt or feelings of worthlessness, suicidal ideations, and/or suicidal actions may occur in the context of depressive disorders, bipolar disorders, mood disorders due to a general medical condition, substance-induced mood disorders, other unspecified mood disorders, and also may be present in association with a range of other psychiatric disorders, including but not limited to psychotic disorders, cognitive disorders, eating disorders, anxiety disorders and personality disorders. The longitudinal course of the disorder, the history, and type of symptoms, and etiologic factors help distinguish the various forms of mood disorders from each other.

“Depression symptoms rating scale” refers to any one of a number of standardized questionnaires, clinical instruments, or symptom inventories utilized to measure symptoms and symptom severity in depression. Such rating scales are often used in clinical studies to define treatment outcomes, based on changes from the study's entry point(s) to endpoint(s). Such depression symptoms rating scales include, but are not limited to, The Quick Inventory of Depressive-Symptomatology Self-Report (QIDS-SR₁₆), the 17-Item Hamilton Rating Scale of Depression (HRSD₁₇), the 30-Item Inventory of Depressive Symptomatology (IDS-C₃₀), or The Montgomery-Asperg Depression Rating Scale (MADRS). Such ratings scales may involve patient self-report or be clinician rated. A 50% or greater reduction in a depression ratings scale score over the course of a clinical trial (starting point to endpoint) is typically considered a favorable response for most depression symptoms rating scales. “Remission” in clinical studies of depression often refers to achieving at, or below, a particular numerical rating score on a depression symptoms rating scale (for instance, less than or equal to 7 on the HRSD₁₇; or less than or equal to 5 on the QIDS-SR₁₆; or less than or equal to 10 on the MADRS).

“Anxiety symptom rating scale” refers to any one of a number of standardized questionnaires, clinical instruments, or symptom inventories utilized to measure symptoms and symptom severity in anxiety. Such rating scales are often used in clinical studies to define treatment outcomes, based on changes from the study's entry point(s) to endpoint(s). Such anxiety symptoms rating scales include, but are not limited to, State-Trait Anxiety Inventory (STAI), the Hamilton Anxiety Rating Scale (HAM-A), the Beck Anxiety Inventory (BAT), and the Hospital Anxiety and Depression Scale-Anxiety (HADS-A). Such ratings scales may involve patient self-report or be clinician rated. A 50% or greater reduction in a depression or anxiety ratings scale score over the course of a clinical trial (starting point to endpoint) is typically considered a favorable response for most depression and anxiety symptoms rating scales. “Remission” in clinical studies of depression often refers to achieving at, or below, a particular numerical rating score on a depression symptoms rating scale (for instance, less than or equal to 39 on the STAI; or less than or equal to 9 on the BAI; or less than or equal to 7 on the HADS-A).

“Alkyl” refers to a straight or branched hydrocarbon chain radical consisting solely of carbon and hydrogen atoms, containing no unsaturation, having from one to ten carbon atoms (e.g., (C₁₋₁₀)alkyl or C₁₋₁₀ alkyl). Whenever it appears herein, a numerical range such as “1 to 10” refers to each integer in the given range—e.g., “1 to 10 carbon atoms” means that the alkyl group may consist of 1 carbon atom, 2 carbon atoms, 3 carbon atoms, etc., up to and including 10 carbon atoms, although the definition is also intended to cover the occurrence of the term “alkyl” where no numerical range is specifically designated. Typical alkyl groups include, but are in no way limited to, methyl, ethyl, propyl, isopropyl, n-butyl, isobutyl, sec-butyl isobutyl, tertiary butyl, pentyl, isopentyl, neopentyl, hexyl, septyl, octyl, nonyl and decyl. The alkyl moiety may be attached to the rest of the molecule by a single bond, such as for example, methyl (Me), ethyl (Et), n-propyl (Pr), 1-methylethyl (isopropyl), n-butyl, n-pentyl, 1,1-dimethylethyl (t-butyl) and 3-methylhexyl. Unless stated otherwise specifically in the specification, an alkyl group is optionally substituted by one or more of substituents which are independently heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂ where each R^{a} is independently hydrogen, fluoroalkyl, carbocyclyl, carbocyclylalkyl, aryl, aralkyl, heterocycloalkyl, heterocycloalkylalkyl, heteroaryl or heteroarylalkyl.

“Alkylaryl” refers to an -(alkyl)aryl radical where aryl and alkyl are as disclosed herein and which are optionally substituted by one or more of the substituents described as suitable substituents for aryl and alkyl respectively.

“Alkylhetaryl” refers to an -(alkyl)hetaryl radical where hetaryl and alkyl are as disclosed herein and which are optionally substituted by one or more of the substituents described as suitable substituents for aryl and alkyl respectively.

“Alkylheterocycloalkyl” refers to an -(alkyl) heterocyclyl radical where alkyl and heterocycloalkyl are as disclosed herein and which are optionally substituted by one or more of the substituents described as suitable substituents for heterocycloalkyl and alkyl respectively.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (i.e., (C₂₋₁₀)alkenyl or C₂₋₁₀ alkenyl). Whenever it appears herein, a numerical range such as “2 to 10” refers to each integer in the given range—e.g., “2 to 10 carbon atoms” means that the alkenyl group may consist of 2 carbon atoms, 3 carbon atoms, etc., up to and including 10 carbon atoms. The alkenyl moiety may be attached to the rest of the molecule by a single bond, such as for example, ethenyl (i.e., vinyl), prop-1-enyl (i.e., allyl), but-1-enyl, pent-1-enyl and penta-1,4-dienyl. Unless stated otherwise specifically in the specification, an alkenyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkenyl-cycloalkyl” refers to an -(alkenyl)cycloalkyl radical where alkenyl and cycloalkyl are as disclosed herein and which are optionally substituted by one or more of the substituents described as suitable substituents for alkenyl and cycloalkyl respectively.

“Alkynyl” refers to a straight or branched hydrocarbon chain radical group consisting solely of carbon and hydrogen atoms, containing at least one triple bond, having from two to ten carbon atoms (i.e., (C₂₋₁₀)alkynyl or C₂₋₁₀ alkynyl). Whenever it appears herein, a numerical range such as “2 to 10” refers to each integer in the given range—e.g., “2 to 10 carbon atoms” means that the alkynyl group may consist of 2 carbon atoms, 3 carbon atoms, etc., up to and including 10 carbon atoms. The alkynyl may be attached to the rest of the molecule by a single bond, for example, ethynyl, propynyl, butynyl, pentynyl and hexynyl. Unless stated otherwise specifically in the specification, an alkynyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkynyl-cycloalkyl” refers to an -(alkynyl)cycloalkyl radical where alkynyl and cycloalkyl are as disclosed herein and which are optionally substituted by one or more of the substituents described as suitable substituents for alkynyl and cycloalkyl respectively.

“Carboxaldehyde” refers to a —(C═O)H radical.

“Carboxyl” refers to a —(C═O)OH radical.

“Cyano” refers to a —CN radical.

“Cycloalkyl” refers to a monocyclic or polycyclic radical that contains only carbon and hydrogen, and may be saturated, or partially unsaturated. Cycloalkyl groups include groups having from 3 to 10 ring atoms (i.e. (C₃₋₁₀)cycloalkyl or C₃₋₁₀ cycloalkyl). Whenever it appears herein, a numerical range such as “3 to 10” refers to each integer in the given range—e.g., “3 to 10 carbon atoms” means that the cycloalkyl group may consist of 3 carbon atoms, etc., up to and including 10 carbon atoms. Illustrative examples of cycloalkyl groups include, but are not limited to the following moieties: cyclopropyl, cyclobutyl, cyclopentyl, cyclopentenyl, cyclohexyl, cyclohexenyl, cycloheptyl, cyclooctyl, cyclononyl, cyclodecyl, norbornyl, and the like. Unless stated otherwise specifically in the specification, a cycloalkyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Cycloalkyl-alkenyl” refers to a -(cycloalkyl)alkenyl radical where cycloalkyl and alkenyl are as disclosed herein and which are optionally substituted by one or more of the substituents described as suitable substituents for cycloalkyl and alkenyl, respectively.

“Cycloalkyl-heterocycloalkyl” refers to a -(cycloalkyl)heterocycloalkyl radical where cycloalkyl and heterocycloalkyl are as disclosed herein and which are optionally substituted by one or more of the substituents described as suitable substituents for cycloalkyl and heterocycloalkyl, respectively.

“Cycloalkyl-heteroaryl” refers to a -(cycloalkyl)heteroaryl radical where cycloalkyl and heteroaryl are as disclosed herein and which are optionally substituted by one or more of the substituents described as suitable substituents for cycloalkyl and heteroaryl, respectively.

The term “alkoxy” refers to the group —O-alkyl, including from 1 to 8 carbon atoms of a straight, branched, cyclic configuration and combinations thereof attached to the parent structure through an oxygen. Examples include, but are not limited to, methoxy, ethoxy, propoxy, isopropoxy, cyclopropyloxy and cyclohexyloxy. “Lower alkoxy” refers to alkoxy groups containing one to six carbons.

The term “substituted alkoxy” refers to alkoxy wherein the alkyl constituent is substituted (i.e., —O-(substituted alkyl)). Unless stated otherwise specifically in the specification, the alkyl moiety of an alkoxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “alkoxycarbonyl” refers to a group of the formula (alkoxy)(C═O)— attached through the carbonyl carbon wherein the alkoxy group has the indicated number of carbon atoms. Thus a (C₁₋₆)alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group.

The term “substituted alkoxycarbonyl” refers to the group (substituted alkyl)-O—C(O)— wherein the group is attached to the parent structure through the carbonyl functionality. Unless stated otherwise specifically in the specification, the alkyl moiety of an alkoxycarbonyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyl” refers to the groups (alkyl)-C(O)—, (aryl)-C(O)—, (heteroaryl)-C(O)—, (heteroalkyl)-C(O)—and (heterocycloalkyl)-C(O)—, wherein the group is attached to the parent structure through the carbonyl functionality. If the R radical is heteroaryl or heterocycloalkyl, the hetero ring or chain atoms contribute to the total number of chain or ring atoms. Unless stated otherwise specifically in the specification, the alkyl, aryl or heteroaryl moiety of the acyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyloxy” refers to a R(C═O)O— radical wherein R is alkyl, aryl, heteroaryl, heteroalkyl or heterocycloalkyl, which are as described herein. If the R radical is heteroaryl or heterocycloalkyl, the hetero ring or chain atoms contribute to the total number of chain or ring atoms. Unless stated otherwise specifically in the specification, the R of an acyloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acylsulfonamide” refers a —S(O)₂—N(R^{a})—C(═O)— radical, where R^{a} is hydrogen, alkyl, fluoroalkyl, carbocyclyl, carbocyclylalkyl, aryl, aralkyl, heterocycloalkyl, heterocycloalkylalkyl, heteroaryl or heteroarylalkyl. Unless stated otherwise specifically in the specification, an acylsulfonamide group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a —N(R^{a})₂ group has two R^{a} substituents other than hydrogen, they can be combined with the nitrogen atom to form a 4-, 5-, 6- or 7-membered ring. For example, —N(R^{a})₂ is intended to include, but is not limited to, 1-pyrrolidinyl and 4-morpholinyl. Unless stated otherwise specifically in the specification, an amino group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “substituted amino” also refers to N-oxides of the groups —NHR^{a}, and NR^{a}R^{a} each as described above. N-oxides can be prepared by treatment of the corresponding amino group with, for example, hydrogen peroxide or m-chloroperoxybenzoic acid.

“Amide” or “amido” refers to a chemical moiety with formula —C(O)N(R)₂ or —NHC(O)R, where R is selected from the group consisting of hydrogen, alkyl, cycloalkyl, aryl, heteroaryl (bonded through a ring carbon) and heteroalicyclic (bonded through a ring carbon), each of which moiety may itself be optionally substituted. The R₂ of —N(R)₂ of the amide may optionally be taken together with the nitrogen to which it is attached to form a 4-, 5-, 6- or 7-membered ring. Unless stated otherwise specifically in the specification, an amido group is optionally substituted independently by one or more of the substituents as described herein for alkyl, cycloalkyl, aryl, heteroaryl, or heterocycloalkyl. An amide may be an amino acid or a peptide molecule attached to a compound disclosed herein, thereby forming a prodrug. The procedures and specific groups to make such amides are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety.

“Aromatic” or “aryl” or “Ar” refers to an aromatic radical with six to ten ring atoms (e.g., C₆-C₁₀ aromatic or C₆-C₁₀ aryl) which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “aryloxy” refers to the group —O-aryl.

The term “substituted aryloxy” refers to aryloxy wherein the aryl substituent is substituted (i.e., —O-(substituted aryl)). Unless stated otherwise specifically in the specification, the aryl moiety of an aryloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Aralkyl” or “arylalkyl” refers to an (aryl)alkyl-radical where aryl and alkyl are as disclosed herein and which are optionally substituted by one or more of the substituents described as suitable substituents for aryl and alkyl respectively.

“Ester” refers to a chemical radical of formula —COOR, where R is selected from the group consisting of alkyl, cycloalkyl, aryl, heteroaryl (bonded through a ring carbon) and heteroalicyclic (bonded through a ring carbon). The procedures and specific groups to make esters are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety. Unless stated otherwise specifically in the specification, an ester group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Halo,” “halide,” or, alternatively, “halogen” is intended to mean fluoro, chloro, bromo or iodo. The terms “haloalkyl,” “haloalkenyl,” “haloalkynyl,” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroalkyl,” “heteroalkenyl,” and “heteroalkynyl” refer to optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range may be given—e.g., C₁-C₄ heteroalkyl which refers to the chain length in total, which in this example is 4 atoms long. A heteroalkyl group may be substituted with one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heteroalkylaryl” refers to an -(heteroalkyl)aryl radical where heteroalkyl and aryl are as disclosed herein and which are optionally substituted by one or more of the substituents described as suitable substituents for heteroalkyl and aryl, respectively.

“Heteroalkylheteroaryl” refers to an -(heteroalkyl)heteroaryl radical where heteroalkyl and heteroaryl are as disclosed herein and which are optionally substituted by one or more of the substituents described as suitable substituents for heteroalkyl and heteroaryl, respectively.

“Heteroalkylheterocycloalkyl” refers to an -(heteroalkyl)heterocycloalkyl radical where heteroalkyl and heterocycloalkyl are as disclosed herein and which are optionally substituted by one or more of the substituents described as suitable substituents for heteroalkyl and heterocycloalkyl, respectively.

“Heteroalkylcycloalkyl” refers to an -(heteroalkyl)cycloalkyl radical where heteroalkyl and cycloalkyl are as disclosed herein and which are optionally substituted by one or more of the substituents described as suitable substituents for heteroalkyl and cycloalkyl, respectively.

“Heteroaryl” or “heteroaromatic” or “HetAr” or “Het” refers to a 5- to 18-membered aromatic radical (e.g., C₅-C₁₃ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range—e.g., “5 to 18 ring atoms” means that the heteroaryl group may consist of 5 ring atoms, 6 ring atoms, etc., up to and including 18 ring atoms. Bivalent radicals derived from univalent heteroaryl radicals whose names end in “-yl” by removal of one hydrogen atom from the atom with the free valence are named by adding “-idene” to the name of the corresponding univalent radical—e.g., a pyridyl group with two points of attachment is a pyridylidene. A N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical are optionally oxidized. One or more nitrogen atoms, if present, are optionally quaternized. The heteroaryl may be attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl(benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e., thienyl). Unless stated otherwise specifically in the specification, a heteroaryl moiety is optionally substituted by one or more substituents which are independently: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

Substituted heteroaryl also includes ring systems substituted with one or more oxide (—O—) substituents, such as, for example, pyridinyl N-oxides.

“Heteroarylalkyl” refers to a moiety having an aryl moiety, as described herein, connected to an alkylene moiety, as described herein, wherein the connection to the remainder of the molecule is through the alkylene group.

“Heterocycloalkyl” refers to a stable 3- to 18-membered non-aromatic ring radical that comprises two to twelve carbon atoms and from one to six heteroatoms selected from nitrogen, oxygen and sulfur. Whenever it appears herein, a numerical range such as “3 to 18” refers to each integer in the given range—e.g., “3 to 18 ring atoms” means that the heterocycloalkyl group may consist of 3 ring atoms, 4 ring atoms, etc., up to and including 18 ring atoms. Unless stated otherwise specifically in the specification, the heterocycloalkyl radical is a monocyclic, bicyclic, tricyclic or tetracyclic ring system, which may include fused or bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocycloalkyl moiety is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

“Nitro” refers to the —NO₂ radical.

“Oxa” refers to the —O— radical.

“Oxo” refers to the ═O radical.

“Isomers” are different compounds that have the same molecular formula. “Stereoisomers” are isomers that differ only in the way the atoms are arranged in space—i.e., having a different stereochemical configuration. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon can be specified by either (R) or (S). Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R) or (S). The present chemical entities, pharmaceutical compositions and methods are meant to include all such possible isomers, including racemic mixtures, optically pure forms and intermediate mixtures. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)-isomer and 20% (R)-isomer, the enantiomeric purity of the compound with respect to the (S)-isomeric form is 80%. The enantiomeric purity of a compound can be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or Pirkle's reagents, or derivatization of a compounds using a chiral compound such as Mosher's acid followed by chromatography or nuclear magnetic resonance spectroscopy.

In some embodiments, the enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (HPLC) and the formation and crystallization of chiral salts; or preferred enantiomers can be prepared by asymmetric syntheses. See, for example, Jacques, et al., Enantiomers, Racemates and Resolutions, Wiley Interscience, New York (1981); E. L. Eliel, Stereochemistry of Carbon Compounds, McGraw-Hill, New York (1962); and E. L. Eliel and S. H. Wilen, Stereochemistry of Organic Compounds, Wiley-Interscience, New York (1994).

The terms “enantiomerically enriched” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as at least 75% by weight, or such as at least 80% by weight. In some embodiments, the enrichment can be significantly greater than 80% by weight, providing a “substantially enantiomerically enriched” or a “substantially non-racemic” preparation, which refers to preparations of compositions which have at least 85% by weight of one enantiomer relative to other enantiomer, such as at least 90% by weight, or such as at least 95% by weight. The terms “enantiomerically pure” or “substantially enantiomerically pure” refers to a composition that comprises at least 98% of a single enantiomer and less than 2% of the opposite enantiomer.

“Moiety” refers to a specific segment or functional group of a molecule. Chemical moieties are often recognized chemical entities embedded in or appended to a molecule.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

A “leaving group or atom” is any group or atom that will, under selected reaction conditions, cleave from the starting material, thus promoting reaction at a specified site. Examples of such groups, unless otherwise specified, include halogen atoms and mesyloxy, p-nitrobenzensulphonyloxy and tosyloxy groups.

“Protecting group” is intended to mean a group that selectively blocks one or more reactive sites in a multifunctional compound such that a chemical reaction can be carried out selectively on another unprotected reactive site and the group can then be readily removed or deprotected after the selective reaction is complete. A variety of protecting groups are disclosed, for example, in T. H. Greene and P. G. M. Wuts, Protective Groups in Organic Synthesis, Third Edition, John Wiley & Sons, New York (1999).

“Solvate” refers to a compound in physical association with one or more molecules of a pharmaceutically acceptable solvent.

“Substituted” means that the referenced group may have attached one or more additional groups, radicals or moieties individually and independently selected from, for example, acyl, alkyl, alkylaryl, cycloalkyl, aralkyl, aryl, carbohydrate, carbonate, heteroaryl, heterocycloalkyl, hydroxy, alkoxy, aryloxy, mercapto, alkylthio, arylthio, cyano, halo, carbonyl, ester, thiocarbonyl, isocyanato, thiocyanato, isothiocyanato, nitro, oxo, perhaloalkyl, perfluoroalkyl, phosphate, silyl, sulfinyl, sulfonyl, sulfonamidyl, sulfoxyl, sulfonate, urea, and amino, including mono- and di-substituted amino groups, and protected derivatives thereof. The substituents themselves may be substituted, for example, a cycloalkyl substituent may itself have a halide substituent at one or more of its ring carbons. The term “optionally substituted” means optional substitution with the specified groups, radicals or moieties.

“Sulfanyl” refers to groups that include —S-(optionally substituted alkyl), —S-(optionally substituted aryl), —S-(optionally substituted heteroaryl) and —S-(optionally substituted heterocycloalkyl).

“Sulfinyl” refers to groups that include —S(O)—H, —S(O)-(optionally substituted alkyl), —S(O)-(optionally substituted amino), —S(O)-(optionally substituted aryl), —S(O)-(optionally substituted heteroaryl) and —S(O)-(optionally substituted heterocycloalkyl).

“Sulfonyl” refers to groups that include —S(O₂)—H, —S(O₂)-(optionally substituted alkyl), —S(O₂)-(optionally substituted amino), —S(O₂)-(optionally substituted aryl), —S(O₂)-(optionally substituted heteroaryl), and —S(O₂)-(optionally substituted heterocycloalkyl).

“Sulfonamidyl” or “sulfonamido” refers to a —S(═O)₂—NRR radical, where each R is selected independently from the group consisting of hydrogen, alkyl, cycloalkyl, aryl, heteroaryl (bonded through a ring carbon) and heteroalicyclic (bonded through a ring carbon). The R groups in —NRR of the —S(═O)₂—NRR radical may be taken together with the nitrogen to which it is attached to form a 4-, 5-, 6- or 7-membered ring. A sulfonamido group is optionally substituted by one or more of the substituents described for alkyl, cycloalkyl, aryl, heteroaryl, respectively.

“Sulfoxyl” refers to a —S(═O)₂OH radical.

“Sulfonate” refers to a —S(═O)₂—OR radical, where R is selected from the group consisting of alkyl, cycloalkyl, aryl, heteroaryl (bonded through a ring carbon) and heteroalicyclic (bonded through a ring carbon). A sulfonate group is optionally substituted on R by one or more of the substituents described for alkyl, cycloalkyl, aryl, heteroaryl, respectively.

Compounds of the disclosure also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

For the avoidance of doubt, it is intended herein that particular features (for example integers, characteristics, values, uses, diseases, formulae, compounds or groups) described in conjunction with a particular aspect, embodiment or example of the disclosure are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The disclosure is not restricted to any details of any disclosed embodiments. The disclosure extends to any novel one, or novel combination, of the features disclosed in this specification (including any accompanying claims, abstract and drawings), or to any novel one, or any novel combination, of the steps of any method or process so disclosed.

Moreover, as used herein, the term “about” means that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the disclosure. All embodiments of the disclosure can, in the alternative, be more specifically defined by any of the transitional terms “comprising,” “consisting essentially of,” and “consisting of.”

### Estradiol Compounds

Estrogen deficiency in the human brain can cause several psychiatric diseases, including depression. Estradiol and molecules with similar properties toward estrogen receptors are used to treat a number of neuropsychiatric conditions including depression. Unfortunately, administration of estrogen and certain estrogen analogs have off-target consequences in several other organs including the uterus and breasts, which can result in cancer and other undesirable effects. In addition, excess estrogen in male patients is undesirable for a number of reasons including gynecomastia. Ideally, a method would be developed in order to easily administer estrogen and have it selectively concentrated in the brain. In such a system, the off target consequences in other organs would be minimized.

DHED is a 17β-estradiol precursor (prodrug) without having any effects at the estrogen receptors throughout the body. Upon exposure to a reductase localized within the brain, DHED converts to 17β-estradiol. In this manner, DHED represents a molecule which selectively focuses estradiol within the brain. Unfortunately, DHED is limited in its clinical utility due to a number of reasons, including a rapid half-life and suspected limited oral bioavailability. Modifications to the DHED structure could have pronounced effects in extending that half-life an increasing oral bioavailability resulting in an ideal drug candidate to treat estrogen-responsive neuropsychiatric disorders.

In one aspect, the present disclosure relates to estradiol prodrugs and analogs in the context of brain-selective compounds, designed to have good bioavailability and release estradiol or an active analog of estradiol within the central nervous system, without extensive systemic exposure. Currently estrogen therapy (ET) is primarily used to alleviate menopausal hot flushes, depression/anxiety, halt or improve cognitive and memory functions, prevent osteoporosis, and genitourinary atrophy. ET has also been used in men undergoing androgen deprivation therapy due to androgen-sensitive prostate cancer to treat hot flushes and depression. Although ET has efficacy in all of these conditions, it has peripheral side effects, including the stimulation of breast (in both men and women) and uterus, and occasionally blot clot formation and subsequent pulmonary embolism. Preclinical studies strongly support the motion that ET can successfully be used in men to treat depression/anxiety without the side effects of estrogen in the periphery.

Military personnel and veterans are disproportionately affected by neuropsychiatric conditions influenced adversely by stress such as depression and post-traumatic stress disorder, which are extremely debilitating and difficult to treat. While it is well known that estrogen signaling mediates stress susceptibility in females, it was recently discovered that estrogen signaling—via conversion from testosterone—also mediates stress susceptibility in males. To translate this discovery into improved care for stress-related disorders, the role of estrogen signaling in animal models of stress-induced neuropsychiatric disorders can be examined, allowing for identification of a pharmacological approach to target estrogen receptors selectively in the brain, thus conferring efficacy for stress-related disorders in both men and women without peripheral side effects. The knowledge generated from these studies can support the development of new treatments, offering substantial benefit to military personnel and Veterans who urgently need improved care.

Stress is a major risk factor for the development of various neuropsychiatric conditions, including depression, anxiety, and post-traumatic stress disorder (PTSD), which disproportionately affect military personnel and Veterans. It has been discovered that brain conversion of 17β-estradiol (E2) from circulating testosterone promotes a maladaptive response to stress in male mice. It has been found that the absence of E2 in brain, but not testosterone per se, underlies this susceptibility. Prior data provides evidence for the effectiveness of E2 as a novel antidepressant and has established a circuit-based mechanism through which stress interacts with hypogonadism to mediate depressive-like behavior in males. These basic scientific discoveries can be translated into tangible benefits for military personnel and Veterans of both sexes who urgently need improved care for stress-related disorders. While E2 is not a viable treatment in human male populations due to its peripheral side effects, the E2 bioprecursor 10β,17β-dihydroxyestra-1,4-dien-3-one (DHED) overcomes this limitation by delivering E2 directly to the brain after conversion by NADPH-dependent reductase. Unfortunately, DHED has poor oral bioavailability, which limits its potential use as a novel therapeutic in patients. As described herein, the role of targeting E2 signaling can be useful as a treatment mechanism for stress-induced neuropsychiatric disorders, and further the discovery of a brain-selective and orally bioavailable prodrug of DHED based on the previously characterized structure of the molecule.

Although 17β-estradiol (E2) is commonly considered a “female hormone”, it is well distributed in the male brain via aromatization of testosterone. While the deleterious effects of fluctuating levels of E2 are well described in women with regard to stress-related disorders, including depression, its effects in men are not well understood. Although testosterone replacement therapy is often used for the treatment of stress-related disorders in men, particularly those with hypogonadism, treatment with testosterone can be associated with undesirable side effects. Additionally, the mechanisms underlying the therapeutic actions of testosterone may not be due to testosterone itself, but rather E2.

It has been discovered that testosterone, via aromatase-dependent conversion to E2 in the brain, is a primary mediator of maladaptive stress responses in male mice. The absence of E2 in the brain, but not testosterone per se, underlies male susceptibility to develop maladaptive behaviors following stress via changes in the activity of an estrogen receptor-β(ERβ)-mediated neural circuit. Activity of ERβ-expressing projections from the basolateral amygdala (BLA) to nucleus accumbens (NAc) is reduced in hypogonadal male mice following exposure to acute stress whereas activation of this circuit promotes stress resilience. These studies reveal that E2 acts on ERβ within the BLA-NAc pathway to regulate hormone deprivation-induced stress susceptibility at both the cellular and behavioral level. These findings provide translationally relevant and paradigm-shifting evidence for the effectiveness of E2 as an antidepressant approach in males, as well as its underlying circuit-based mechanism in hypogonadal individuals. However, E2 is not a viable treatment in human male populations due to its peripheral side effects, including gynecomastia and erectile dysfunction. As described herein, this science discovery can be used for the benefits of veterans in need of care for stress-related disorders. The brain-selective E2 bioprecursor 10β,17β-dihydroxyestra-1,4-dien-3-one (DHED) selectively increases E2 in the brain following conversion by an NADPH-dependent reductase, hence avoiding E2 peripheral effects. Prior data reveals that DHED has poor oral bioavailability, which restricts its potential use as a human therapeutic. In one aspect, the disclosure describes E2 signaling as a treatment mechanism for stress-induced neuropsychiatric disorders, and further the discovery of a brain-selective and orally bioavailable prodrug of DHED based on the previously characterized structure of the molecule.

The Veterans Administration has recognized that psychiatric disorders associated with stress, including depression, anxiety, and PTSD, are of increasing concern for veterans. While medications that increase synaptic monoamine levels are the most prescribed drug treatments for depression anxiety, and PTSD, many veterans treated with these medications do not show a therapeutic response. Even while undergoing treatment, many veterans still experience symptoms and have suicidal thoughts (or succumb to such thoughts) while waiting and hoping for the treatment to take effect. This high non-response rate emphasizes the need for improved approaches to treat these serious and life-threatening psychiatric manifestations that are the result of stress. Low testosterone is associated with increased mortality and is depression in veterans. Additionally, low testosterone is observed in non-psychiatric conditions that are prevalent in veterans such as diabetes, in addition to traumatic injuries and physiological ageing. veterans also experience greater, and unique stressors compared to other populations. Thus, there is the potential for a combination of stress and low testosterone to combine to increase risk of psychiatric disorders. While low testosterone has not been consistently associated with susceptibility to develop PTSD, augmenting downstream signaling could still be a useful form of treatment combined at specific time points with cognitive behavioral therapy.

17β-estradiol (E2) is a gonadal steroid hormone with actions in different tissues including the uterus, anterior pituitary, and skeletal muscles, and the central and peripheral nervous systems. In women, especially those with low or fluctuating hormones, estrogen replacement therapy has been shown efficacy for multiple neuropsychiatric conditions, though sometimes with undesirable estrogen-related peripheral side effects. For example, fluctuations in E2 levels are associated with an increased risk for development of mood disorders in women and there is extensive evidence regarding the role of this hormone in depression. Indeed, E2 administration to women exerts antidepressant effects. Although E2 is commonly considered to be a “female hormone”, it is well distributed in the male brain due to the conversion of testosterone (derived from the testis) to E2 through the actions of the aromatase enzyme. While testosterone itself has clear effects on behaviors and testosterone replacement therapy is often used for the treatment of stress-related disorders, including depression, particularly in hypogonadal men, the mechanisms underlying testosterone's effects may not be due to testosterone itself, but rather E2. However, the role of E2 in males has received limited attention due to the assumption that the demonstrated antidepressant effects of testosterone in males are through the actions of testosterone itself. Although testosterone replacement therapy is effective for the treatment of refractory-depression in men, potentially serious life-threatening side effects exist, such as polycythemia and cardiac dysfunction. The potential beneficial effects of E2 in males have been considered only to a limited degree due to undesirable peripheral effects, including gynecomastia and erectile dysfunction. Indeed, a drug treatment with peripheral effects of E2 would not be viable as a routine treatment option for men.

It has been discovered that in the brain E2, via aromatase-dependent conversion from testosterone, is a primary mediator of maladaptive stress response male mice. These data indicate a potential clinical use of E2 in males for the treatment of stress-mediated disorders, potentially both in individuals with low circulating levels of testosterone and in those with normal testosterone levels. The 10β,17β-dihydroxyestra-1,4-dien-3-one (DHED) molecule naturally occurs in the mammalian brain as a breakdown product of E2. When administered exogenously and following exposure to a brain-localized reductase enzyme, DHED is converted to E2 selectively in the brain. DHED itself has no effects on estrogen or other hormone receptors throughout the body, effectively eliminating any peripheral effects of E2. While previous studies have found that high doses of DHED administered orally to rodents results in E2-like efficacy in male and female models of hot flushes, DHED has limited human clinical utility as an orally administered drug due to its low oral bioavailability (see FIGS. 6A-6B).

17β-estradiol (E2) is a gonadal steroid hormone with actions in different tissues including the uterus, anterior pituitary, skeletal muscles, as well as the central and peripheral nervous systems. Although E2 is commonly considered as the “female hormone”, it is well distributed in the male brain as a consequence of testosterone's conversion to E2 by the actions of the aromatase enzyme. Fluctuations in E2 levels are associated with an increased risk for development of mood disorders in women and E2 administration enhances reward sensitivity in female rodents, suggesting a possible role of this hormone in reward-deficit associated disorders such as depression. Indeed, E2 administration to women exerts antidepressant effects. Similarly, testosterone has been shown to exert antidepressant effects in males; however, the role of E2 in males has received limited attention due to the assumption that the demonstrated antidepressant effects of testosterone in males are through the actions of testosterone itself.

The disclosure provides a compound of formula (I), or a pharmaceutically acceptable salt, solvate, hydrate, cocrystal, or prodrug thereof:

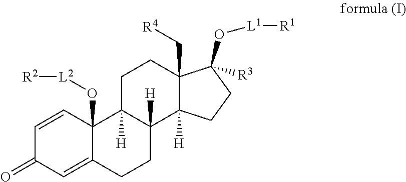

wherein in formula (I):
L¹ and L² are each a linker comprising independently one or more of a bond, —C(O)—, —O—, —S—, —NR^{a}—, —CR^{a}₂—, —C(O)O—, —C(O)S—, —C(O)NR^{a}—, —C(O)NR^{a}SO₂—, disubstituted alkyl, disubstituted heteroalkyl, disubstituted alkenyl, disubstituted alkynyl, disubstituted cycloalkyl, disubstituted heterocycloalkyl, disubstituted aryl, disubstituted arylalkyl, disubstituted heteroaryl, and disubstituted heteroarylalkyl;
R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl;
R¹ is selected from optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl;
R² is selected from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl;
R³ is selected from hydrogen and optionally substituted alkynyl; and
R⁴ is selected from hydrogen and optionally substituted alkyl.

In some embodiments, L¹ comprises one or more of a bond, disubstituted alkyl, —C(O)—, —C(O)O—, and —(CH₂)ₚO— wherein p is an integer from 1 to 5. In some embodiments, L¹ comprises

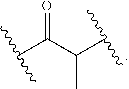 [SMILES: *C(=O)C(*)C]

In another aspect, the disclosure provides a compound of formula (10), formula (11), or formula (12), or a pharmaceutically acceptable salt, solvate, hydrate, cocrystal, or prodrug thereof:

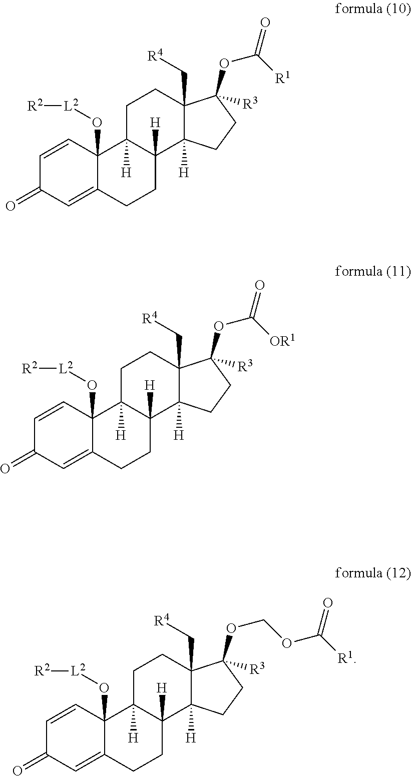

In some embodiments, R¹ is selected from optionally substituted alkyl, optionally substituted aryl, optionally substituted heterocycloalkyl, and optionally substituted heteroaryl. In some embodiments, R¹ is selected from methyl, ethyl, i-propyl, -n-butyl, n-pentyl, n-hexyl, n-heptyl, n-octyl, -i-butyl, -t-butyl, —C₉H₁₉, —C₁₀H₂₁, —C₁₁H₂₃,

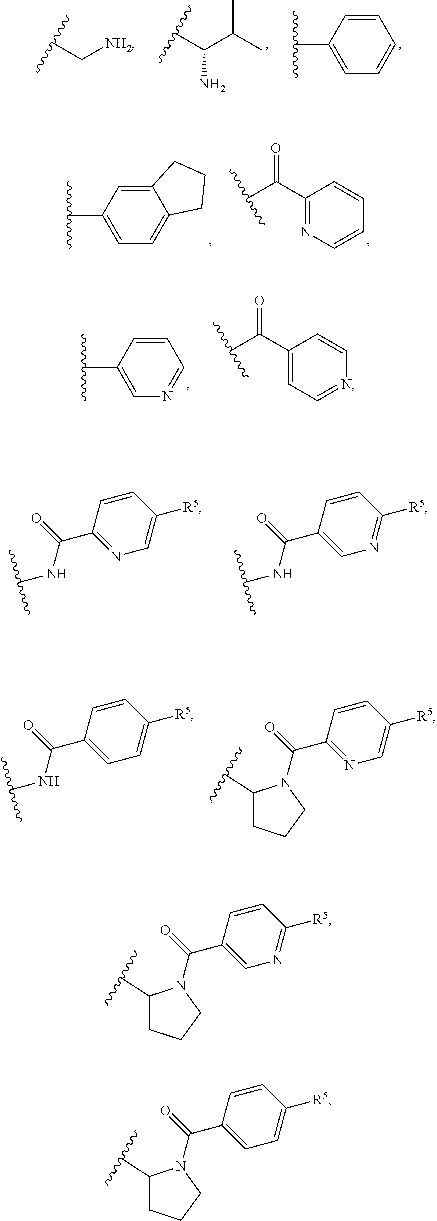

wherein R⁵ is selected from hydrogen, optionally substituted alkyl, —C(O)R⁶, —C(O)N(R⁶)₂, —S(O)R⁶, —S(O)₂R⁶, —S(O)N(R⁶)₂, and —S(O)₂N(R⁶)₂, wherein R⁶ is selected from hydrogen and optionally substituted alkyl, optionally R⁵ is selected from hydrogen and —S(O)₂NH₂;

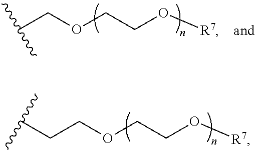

wherein n is an integer from 1 to 5, and R⁷ is optionally substituted alkyl, optionally R⁷ is methyl. In some embodiments, R⁵ is —S(O)₂NH₂

In some embodiments, R¹-L¹- is selected from

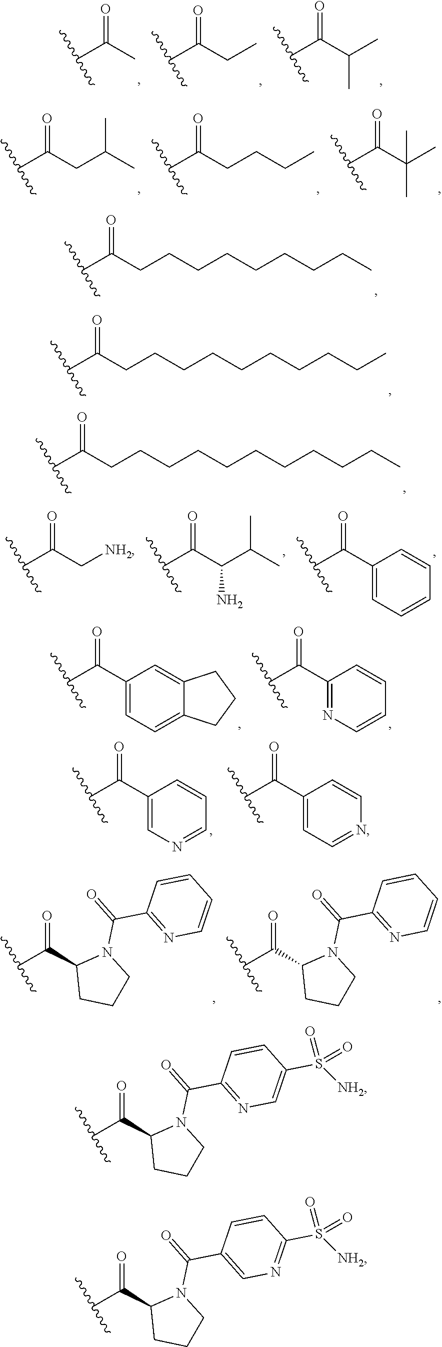

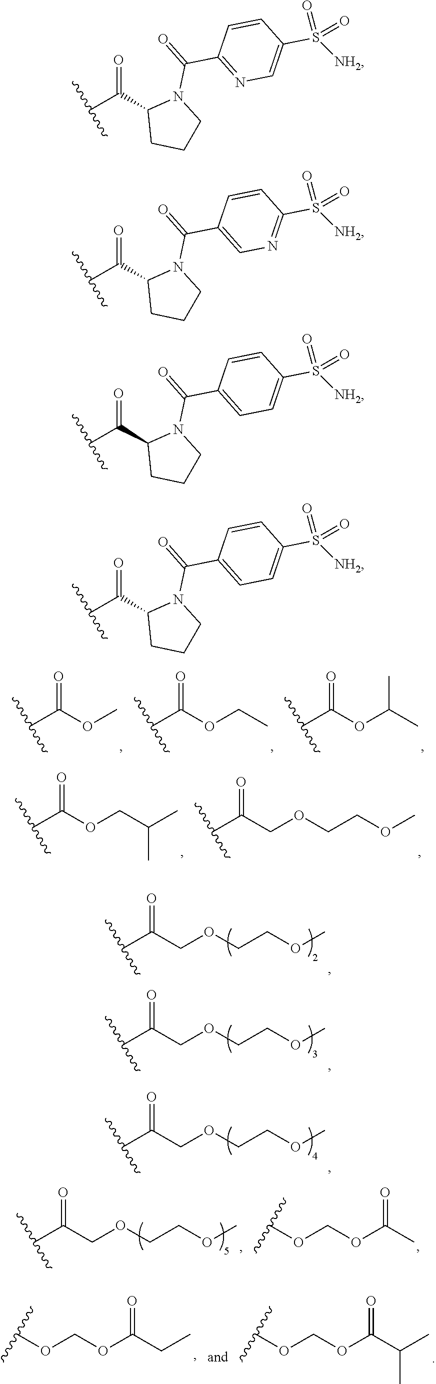

In some embodiments, R¹-L¹- is selected from

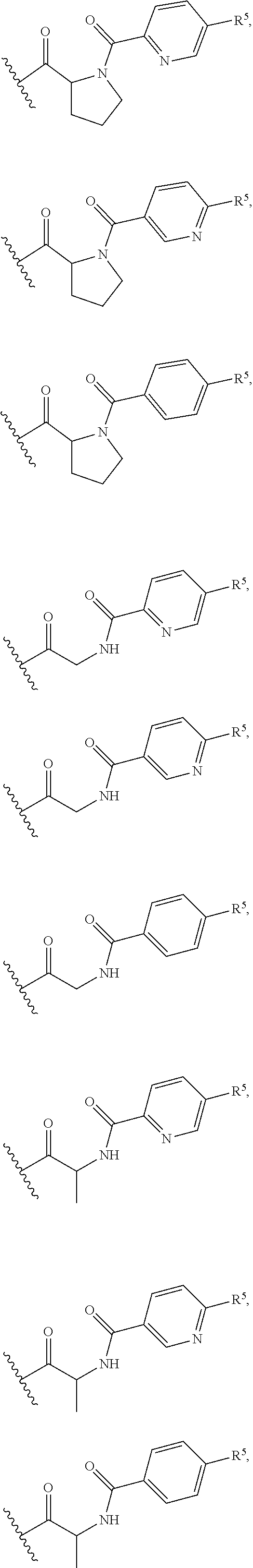

wherein R⁵ is selected from hydrogen,
optionally substituted alkyl, —C(O)R⁶, —C(O)N(R⁶)₂, —S(O)R⁶, —S(O)₂R⁶, —S(O)N(R⁶)₂, and —S(O)₂N(R⁶)₂, wherein R⁶ is selected from hydrogen and optionally substituted alkyl; optionally R⁵ is selected from hydrogen and —S(O)₂NH₂;

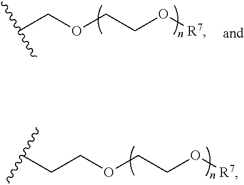

wherein n is an integer from 1 to 5, and R⁷ is optionally substituted alkyl, optionally R⁷ is methyl. In some embodiments, R⁵ is-S(O)₂NH₂.

In some embodiments, L² comprises one or more of a bond, —C(O)—, —C(O)O—, and —(CH₂)ₚO— wherein p is an integer from 1 to 5.

In some embodiments, R² is selected from hydrogen, optionally substituted alkyl, optionally substituted aryl, optionally substituted heterocycloalkyl, and optionally substituted heteroaryl. In some embodiments, R² is selected from methyl, ethyl, i-propyl,

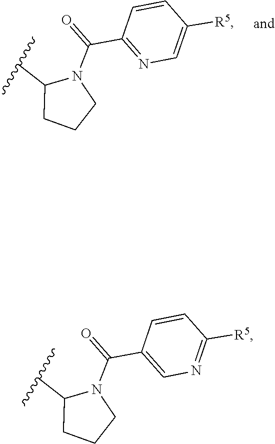

wherein R⁵ is selected from hydrogen, optionally substituted alkyl, —C(O)R⁶, —C(O)N(R⁶)₂, —S(O)R⁶, —S(O)₂R⁶, —S(O)N(R⁶)₂, and —S(O)₂N(R⁶)₂, and R⁶ is selected from hydrogen and optionally substituted alkyl, optionally R⁵ is selected from hydrogen and —S(O)₂NH₂;

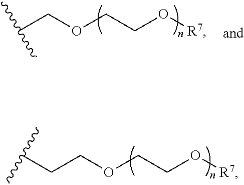

wherein n is an integer from 1 to 5, and R⁷ is optionally substituted alkyl, optionally R⁷ is methyl. In some embodiments, R⁵ is —S(O)₂NH₂.

In some embodiments, R²-L²- is selected from hydrogen and

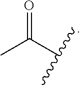 [SMILES: *C(C)=O]

In some embodiments, R³ is selected from hydrogen and

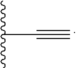 [SMILES: *C#C]

In some embodiments, R⁴ is selected from hydrogen and methyl.

In some embodiments, the compound of formula (I) is a compound of any one of formulas 1001-1256, or a pharmaceutically acceptable salt, solvate, hydrate, cocrystal, or prodrug thereof:

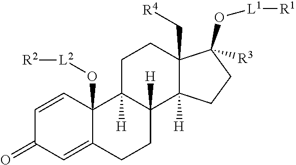

| Formula | | | | |
|---|---|---|---|---|
| No. | —L¹—R¹ | R²—L²— | R³ | R⁴ |
| 1001 | 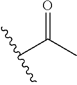 [SMILES: *C(C)=O] | H | H | H |
| 1002 | 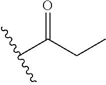 [SMILES: *C(=O)CC] | H | H | H |
| 1003 | 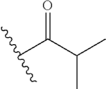 [SMILES: *C(=O)C(C)C] | H | H | H |
| 1004 | 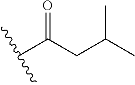 [SMILES: *C(=O)CC(C)C] | H | H | H |
| 1005 | 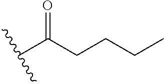 [SMILES: *C(=O)CCCC] | H | H | H |
| 1006 | 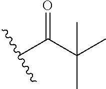 [SMILES: *C(=O)C(C)(C)C] | H | H | H |
| 1007 | 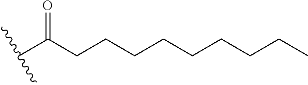 [SMILES: *C(=O)CCCCCCCCC] | H | H | H |
| 1008 | 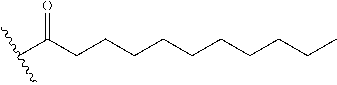 [SMILES: *C(=O)CCCCCCCCCC] | H | H | H |
| 1009 | 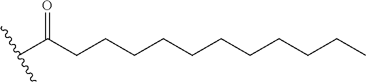 [SMILES: *C(=O)CCCCCCCCCCC] | H | H | H |
| 1010 | 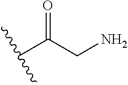 [SMILES: *C(=O)CN] | H | H | H |
| 1011 | 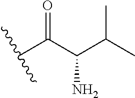 [SMILES: *C(=O)[C@@H](N)C(C)C] | H | H | H |
| 1012 | 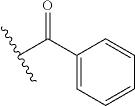 [SMILES: *C(=O)c1ccccc1] | H | H | H |
| 1013 | 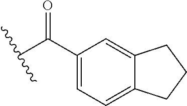 [SMILES: *C(=O)c1ccc2c(c1)CCC2] | H | H | H |
| 1014 | 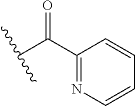 [SMILES: *C(=O)c1ccccn1] | H | H | H |
| 1015 | 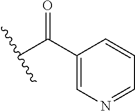 [SMILES: *C(=O)c1cccnc1] | H | H | H |
| 1016 | 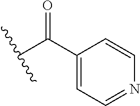 [SMILES: *C(=O)c1ccncc1] | H | H | H |
| 1017 | 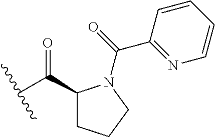 [SMILES: *C(=O)[C@@H]1CCCN1C(=O)c1ccccn1] | H | H | H |
| 1018 | 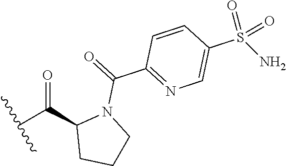 [SMILES: *C(=O)[C@@H]1CCCN1C(=O)c1ccc(S(N)(=O)=O)cn1] | H | H | H |
| 1019 | 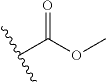 [SMILES: *C(=O)OC] | H | H | H |
| 1020 | 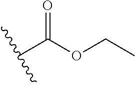 [SMILES: *C(=O)OCC] | H | H | H |
| 1021 | 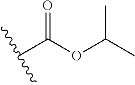 [SMILES: *C(=O)OC(C)C] | H | H | H |
| 1022 | 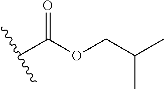 [SMILES: *C(=O)OCC(C)C] | H | H | H |
| 1023 | 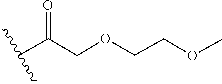 [SMILES: *C(=O)COCCOC] | H | H | H |
| 1024 | 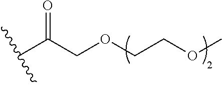 | H | H | H |
| 1025 | 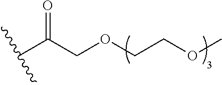 | H | H | H |
| 1026 | 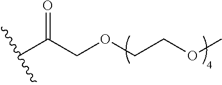 | H | H | H |
| 1027 | 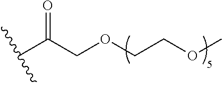 | H | H | H |
| 1028 | 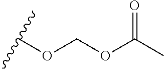 [SMILES: *OCOC(C)=O] | H | H | H |
| 1029 | 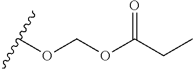 [SMILES: *OCOC(=O)CC] | H | H | H |
| 1030 | 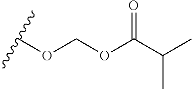 [SMILES: *OCOC(=O)C(C)C] | H | H | H |
| 1031 | 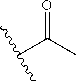 [SMILES: *C(C)=O] | 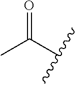 [SMILES: *C(C)=O] | H | H |
| 1032 | 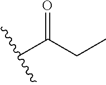 [SMILES: *C(=O)CC] | 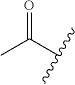 [SMILES: *C(C)=O] | H | H |
| 1033 | 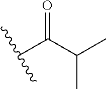 [SMILES: *C(=O)C(C)C] | 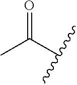 [SMILES: *C(C)=O] | H | H |
| 1034 | 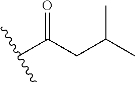 [SMILES: *C(=O)CC(C)C] | 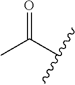 [SMILES: *C(C)=O] | H | H |
| 1035 | 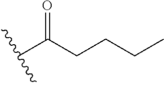 [SMILES: *C(=O)CCCC] | 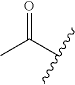 [SMILES: *C(C)=O] | H | H |
| 1036 | 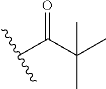 [SMILES: *C(=O)C(C)(C)C] | 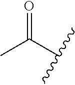 [SMILES: *C(C)=O] | H | H |
| 1037 | 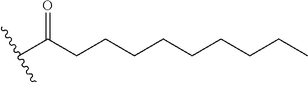 [SMILES: *C(=O)CCCCCCCCC] | 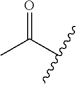 [SMILES: *C(C)=O] | H | H |
| 1038 | 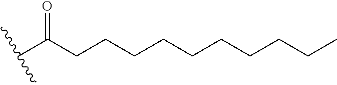 [SMILES: *C(=O)CCCCCCCCCC] | 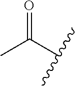 [SMILES: *C(C)=O] | H | H |
| 1039 | 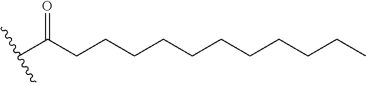 [SMILES: *C(=O)CCCCCCCCCCC] | 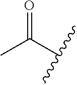 [SMILES: *C(C)=O] | H | H |
| 1040 | 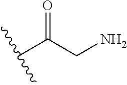 [SMILES: *C(=O)CN] | 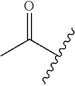 [SMILES: *C(C)=O] | H | H |
| 1041 | 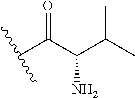 [SMILES: *C(=O)[C@@H](N)C(C)C] | 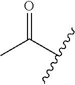 [SMILES: *C(C)=O] | H | H |
| 1042 | 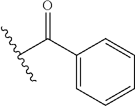 [SMILES: *C(=O)c1ccccc1] | 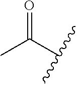 [SMILES: *C(C)=O] | H | H |
| 1043 | 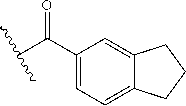 [SMILES: *C(=O)c1ccc2c(c1)CCC2] | 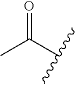 [SMILES: *C(C)=O] | H | H |
| 1044 | 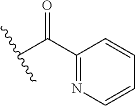 [SMILES: *C(=O)c1ccccn1] | 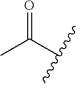 [SMILES: *C(C)=O] | H | H |
| 1045 | 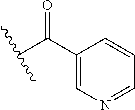 [SMILES: *C(=O)c1cccnc1] | 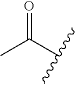 [SMILES: *C(C)=O] | H | H |
| 1046 | 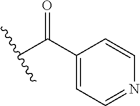 [SMILES: *C(=O)c1ccncc1] | 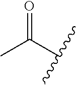 [SMILES: *C(C)=O] | H | H |
| 1047 | 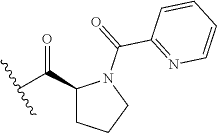 [SMILES: *C(=O)[C@@H]1CCCN1C(=O)c1ccccn1] | 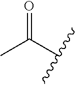 [SMILES: *C(C)=O] | H | H |
| 1048 | 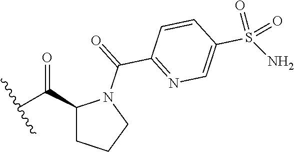 [SMILES: *C(=O)[C@@H]1CCCN1C(=O)c1ccc(S(N)(=O)=O)cn1] | 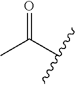 [SMILES: *C(C)=O] | H | H |
| 1049 | 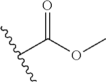 [SMILES: *C(=O)OC] | 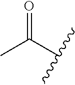 [SMILES: *C(C)=O] | H | H |
| 1050 | 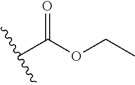 [SMILES: *C(=O)OCC] | 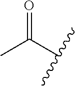 [SMILES: *C(C)=O] | H | H |
| 1051 | 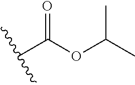 [SMILES: *C(=O)OC(C)C] | 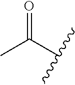 [SMILES: *C(C)=O] | H | H |
| 1052 | 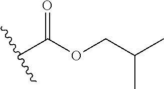 [SMILES: *C(=O)OCC(C)C] | 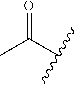 [SMILES: *C(C)=O] | H | H |
| 1053 | 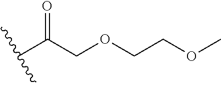 [SMILES: *C(=O)COCCOC] | 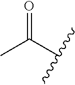 [SMILES: *C(C)=O] | H | H |
| 1054 | 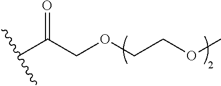 | 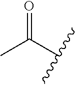 [SMILES: *C(C)=O] | H | H |
| 1055 | 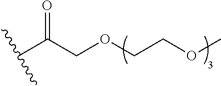 | 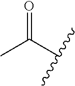 [SMILES: *C(C)=O] | H | H |
| 1056 | 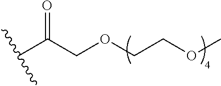 | 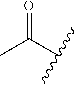 [SMILES: *C(C)=O] | H | H |
| 1057 | 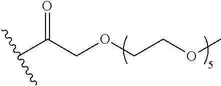 | 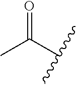 [SMILES: *C(C)=O] | H | H |
| 1058 | 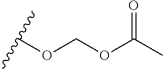 [SMILES: *OCOC(C)=O] | 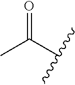 [SMILES: *C(C)=O] | H | H |
| 1059 | 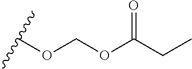 [SMILES: *OCOC(=O)CC] | 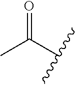 [SMILES: *C(C)=O] | H | H |
| 1060 | 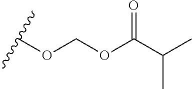 [SMILES: *OCOC(=O)C(C)C] | 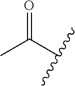 [SMILES: *C(C)=O] | H | H |
| 1061 | 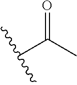 [SMILES: *C(C)=O] | H | 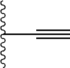 [SMILES: *C#C] | H |
| 1062 | 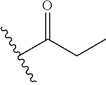 [SMILES: *C(=O)CC] | H | 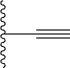 [SMILES: *C#C] | H |
| 1063 | 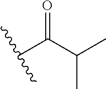 [SMILES: *C(=O)C(C)C] | H | 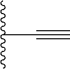 [SMILES: *C#C] | H |
| 1064 | 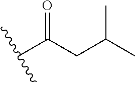 [SMILES: *C(=O)CC(C)C] | H | 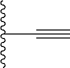 [SMILES: *C#C] | H |
| 1065 | 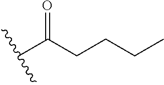 [SMILES: *C(=O)CCCC] | H | 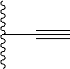 [SMILES: *C#C] | H |
| 1066 | 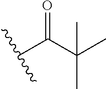 [SMILES: *C(=O)C(C)(C)C] | H | 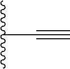 [SMILES: *C#C] | H |
| 1067 | 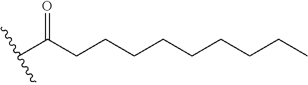 [SMILES: *C(=O)CCCCCCCCC] | H | 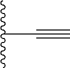 [SMILES: *C#C] | H |
| 1068 | 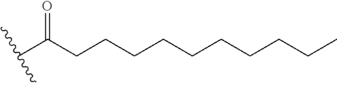 [SMILES: *C(=O)CCCCCCCCCC] | H | 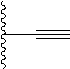 [SMILES: *C#C] | H |
| 1069 | 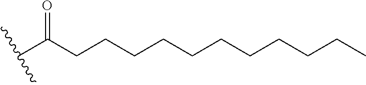 [SMILES: *C(=O)CCCCCCCCCCC] | H | 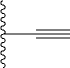 [SMILES: *C#C] | H |
| 1070 | 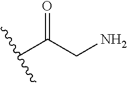 [SMILES: *C(=O)CN] | H | 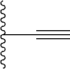 [SMILES: *C#C] | H |
| 1071 | 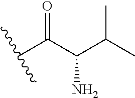 [SMILES: *C(=O)[C@@H](N)C(C)C] | H | 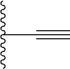 [SMILES: *C#C] | H |
| 1072 | 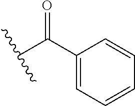 [SMILES: *C(=O)c1ccccc1] | H | 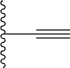 [SMILES: *C#C] | H |
| 1073 | 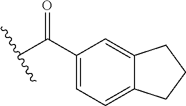 [SMILES: *C(=O)c1ccc2c(c1)CCC2] | H | 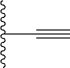 [SMILES: *C#C] | H |
| 1074 | 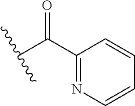 [SMILES: *C(=O)c1ccccn1] | H | 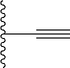 [SMILES: *C#C] | H |
| 1075 | 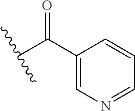 [SMILES: *C(=O)c1cccnc1] | H | 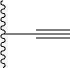 [SMILES: *C#C] | H |
| 1076 | 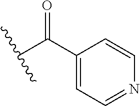 [SMILES: *C(=O)c1ccncc1] | H | 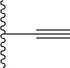 [SMILES: *C#C] | H |
| 1077 | 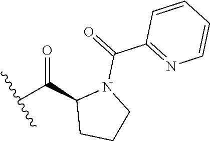 [SMILES: *C(=O)[C@@H]1CCCN1C(=O)c1ccccn1] | H | 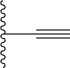 [SMILES: *C#C] | H |
| 1078 | 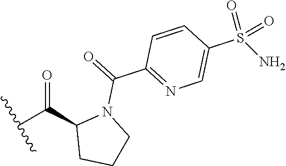 [SMILES: *C(=O)[C@@H]1CCCN1C(=O)c1ccc(S(N)(=O)=O)cn1] | H | 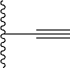 [SMILES: *C#C] | H |
| 1079 | 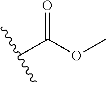 [SMILES: *C(=O)OC] | H | 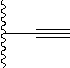 [SMILES: *C#C] | H |
| 1080 | 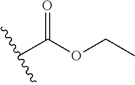 [SMILES: *C(=O)OCC] | H | 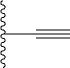 [SMILES: *C#C] | H |
| 1081 | 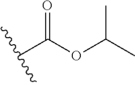 [SMILES: *C(=O)OC(C)C] | H | 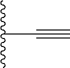 [SMILES: *C#C] | H |
| 1082 | 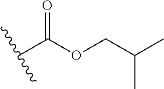 [SMILES: *C(=O)OCC(C)C] | H | 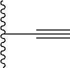 [SMILES: *C#C] | H |
| 1083 | 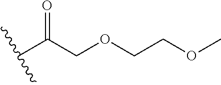 [SMILES: *C(=O)COCCOC] | H | 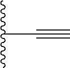 [SMILES: *C#C] | H |
| 1084 | 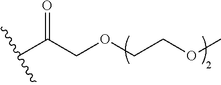 | H | 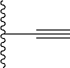 [SMILES: *C#C] | H |
| 1085 | 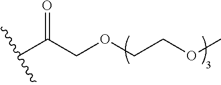 | H | 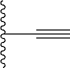 [SMILES: *C#C] | H |
| 1086 | 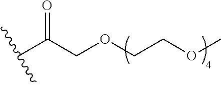 | H | 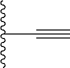 [SMILES: *C#C] | H |
| 1087 | 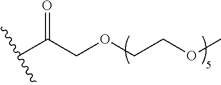 | H | 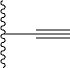 [SMILES: *C#C] | H |
| 1088 | 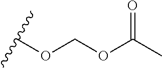 [SMILES: *OCOC(C)=O] | H | 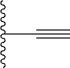 [SMILES: *C#C] | H |
| 1089 | 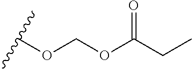 [SMILES: *OCOC(=O)CC] | H | 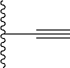 [SMILES: *C#C] | H |
| 1090 | 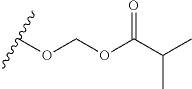 [SMILES: *OCOC(=O)C(C)C] | H | 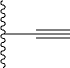 [SMILES: *C#C] | H |
| 1091 | 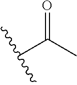 [SMILES: *C(C)=O] | 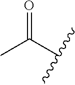 [SMILES: *C(C)=O] | 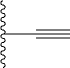 [SMILES: *C#C] | H |
| 1092 | 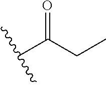 [SMILES: *C(=O)CC] | 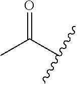 [SMILES: *C(C)=O] | 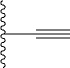 [SMILES: *C#C] | H |
| 1093 | 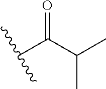 [SMILES: *C(=O)C(C)C] | 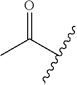 [SMILES: *C(C)=O] | 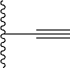 [SMILES: *C#C] | H |
| 1094 | 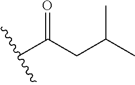 [SMILES: *C(=O)CC(C)C] | 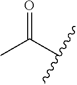 [SMILES: *C(C)=O] | 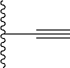 [SMILES: *C#C] | H |
| 1095 | 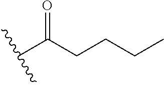 [SMILES: *C(=O)CCCC] | 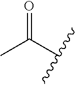 [SMILES: *C(C)=O] | 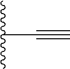 [SMILES: *C#C] | H |
| 1096 | 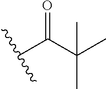 [SMILES: *C(=O)C(C)(C)C] | 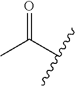 [SMILES: *C(C)=O] | 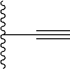 [SMILES: *C#C] | H |
| 1097 | 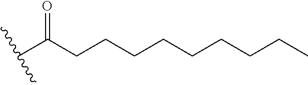 [SMILES: *C(=O)CCCCCCCCC] | 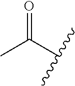 [SMILES: *C(C)=O] | 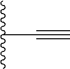 [SMILES: *C#C] | H |
| 1098 | 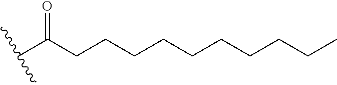 [SMILES: *C(=O)CCCCCCCCCC] | 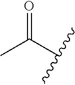 [SMILES: *C(C)=O] | 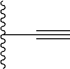 [SMILES: *C#C] | H |
| 1099 | 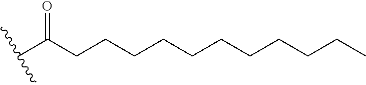 [SMILES: *C(=O)CCCCCCCCCCC] | 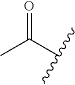 [SMILES: *C(C)=O] | 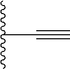 [SMILES: *C#C] | H |
| 1100 | 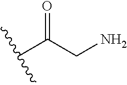 [SMILES: *C(=O)CN] | 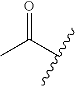 [SMILES: *C(C)=O] | 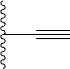 [SMILES: *C#C] | H |
| 1101 | 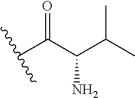 [SMILES: *C(=O)[C@@H](N)C(C)C] | 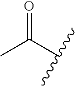 [SMILES: *C(C)=O] | 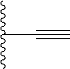 [SMILES: *C#C] | H |
| 1102 | 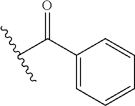 [SMILES: *C(=O)c1ccccc1] | 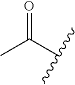 [SMILES: *C(C)=O] | 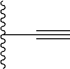 [SMILES: *C#C] | H |
| 1103 | 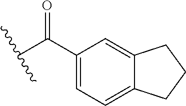 [SMILES: *C(=O)c1ccc2c(c1)CCC2] | 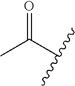 [SMILES: *C(C)=O] | 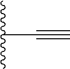 [SMILES: *C#C] | H |
| 1104 | 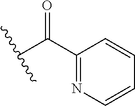 [SMILES: *C(=O)c1ccccn1] | 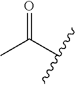 [SMILES: *C(C)=O] | 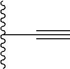 [SMILES: *C#C] | H |
| 1105 | 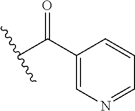 [SMILES: *C(=O)c1cccnc1] | 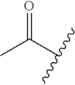 [SMILES: *C(C)=O] | 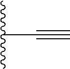 [SMILES: *C#C] | H |
| 1106 | 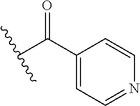 [SMILES: *C(=O)c1ccncc1] | 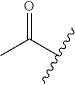 [SMILES: *C(C)=O] | 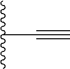 [SMILES: *C#C] | H |
| 1107 | 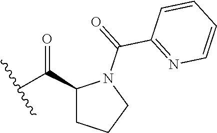 [SMILES: *C(=O)[C@@H]1CCCN1C(=O)c1ccccn1] | 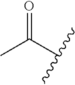 [SMILES: *C(C)=O] | 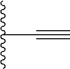 [SMILES: *C#C] | H |
| 1108 | 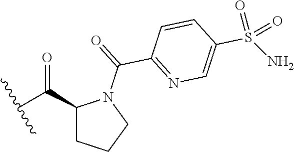 [SMILES: *C(=O)[C@@H]1CCCN1C(=O)c1ccc(S(N)(=O)=O)cn1] | 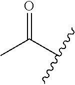 [SMILES: *C(C)=O] | 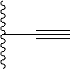 [SMILES: *C#C] | H |
| 1109 | 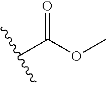 [SMILES: *C(=O)OC] | 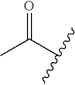 [SMILES: *C(C)=O] | 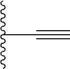 [SMILES: *C#C] | H |
| 1110 | 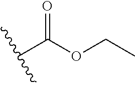 [SMILES: *C(=O)OCC] | 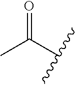 [SMILES: *C(C)=O] | 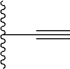 [SMILES: *C#C] | H |
| 1111 | 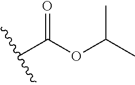 [SMILES: *C(=O)OC(C)C] | 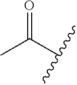 [SMILES: *C(C)=O] | 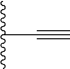 [SMILES: *C#C] | H |
| 1112 | 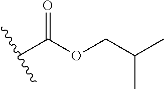 [SMILES: *C(=O)OCC(C)C] | 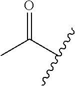 [SMILES: *C(C)=O] | 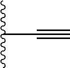 [SMILES: *C#C] | H |
| 1113 | 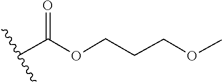 [SMILES: *C(=O)OCCCOC] | 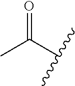 [SMILES: *C(C)=O] | 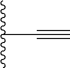 [SMILES: *C#C] | H |
| 1114 | 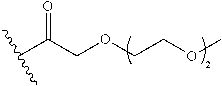 | 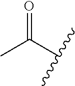 [SMILES: *C(C)=O] | 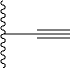 [SMILES: *C#C] | H |
| 1115 | 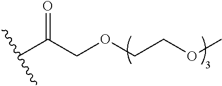 | 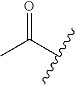 [SMILES: *C(C)=O] | 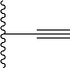 [SMILES: *C#C] | H |
| 1116 | 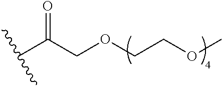 | 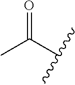 [SMILES: *C(C)=O] | 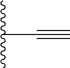 [SMILES: *C#C] | H |
| 1117 | 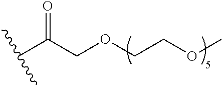 | 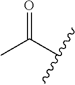 [SMILES: *C(C)=O] | 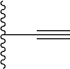 [SMILES: *C#C] | H |
| 1118 | 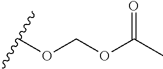 [SMILES: *OCOC(C)=O] | 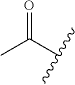 [SMILES: *C(C)=O] | 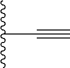 [SMILES: *C#C] | H |
| 1119 | 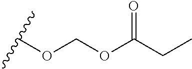 [SMILES: *OCOC(=O)CC] | 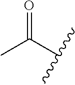 [SMILES: *C(C)=O] | 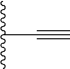 [SMILES: *C#C] | H |
| 1120 | 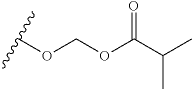 [SMILES: *OCOC(=O)C(C)C] | 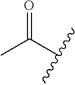 [SMILES: *C(C)=O] | 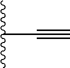 [SMILES: *C#C] | H |
| 1121 | 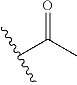 [SMILES: *C(C)=O] | H | H | Me |
| 1122 | 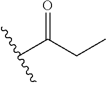 [SMILES: *C(=O)CC] | H | H | Me |
| 1123 | 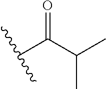 [SMILES: *C(=O)C(C)C] | H | H | Me |
| 1124 | 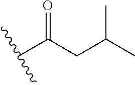 [SMILES: *C(=O)CC(C)C] | H | H | Me |
| 1125 | 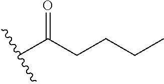 [SMILES: *C(=O)CCCC] | H | H | Me |
| 1126 | 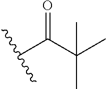 [SMILES: *C(=O)C(C)(C)C] | H | H | Me |
| 1127 | 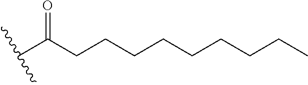 [SMILES: *C(=O)CCCCCCCCC] | H | H | Me |
| 1128 | 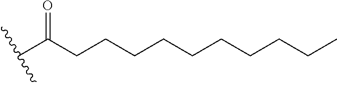 [SMILES: *C(=O)CCCCCCCCCC] | H | H | Me |
| 1129 | 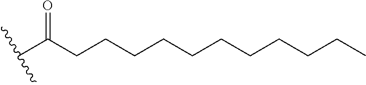 [SMILES: *C(=O)CCCCCCCCCCC] | H | H | Me |
| 1130 | 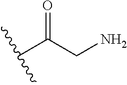 [SMILES: *C(=O)CN] | H | H | Me |
| 1131 | 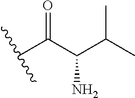 [SMILES: *C(=O)[C@@H](N)C(C)C] | H | H | Me |
| 1132 | 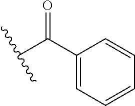 [SMILES: *C(=O)c1ccccc1] | H | H | Me |
| 1133 | 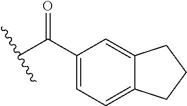 [SMILES: *C(=O)c1ccc2c(c1)CCC2] | H | H | Me |
| 1134 | 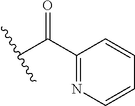 [SMILES: *C(=O)c1ccccn1] | H | H | Me |
| 1135 | 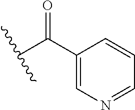 [SMILES: *C(=O)c1cccnc1] | H | H | Me |
| 1136 | 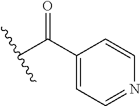 [SMILES: *C(=O)c1ccncc1] | H | H | Me |
| 1137 | 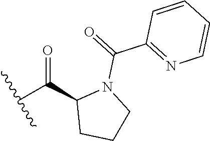 [SMILES: *C(=O)[C@@H]1CCCN1C(=O)c1ccccn1] | H | H | Me |
| 1138 | 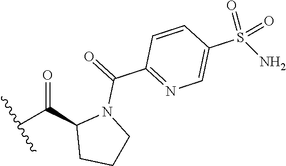 [SMILES: *C(=O)[C@@H]1CCCN1C(=O)c1ccc(S(N)(=O)=O)cn1] | H | H | Me |
| 1139 | 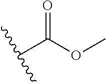 [SMILES: *C(=O)OC] | H | H | Me |
| 1140 | 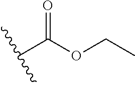 [SMILES: *C(=O)OCC] | H | H | Me |
| 1141 | 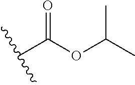 [SMILES: *C(=O)OC(C)C] | H | H | Me |
| 1142 | 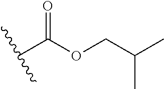 [SMILES: *C(=O)OCC(C)C] | H | H | Me |
| 1143 | 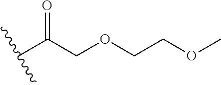 [SMILES: *C(=O)COCCOC] | H | H | Me |
| 1144 | 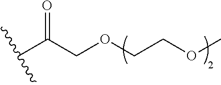 | H | H | Me |
| 1145 | 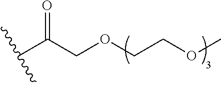 | H | H | Me |
| 1146 | 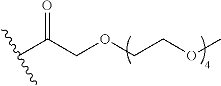 | H | H | Me |
| 1147 | 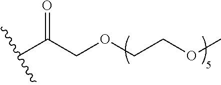 | H | H | Me |
| 1148 | 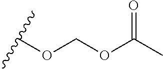 [SMILES: *OCOC(C)=O] | H | H | Me |
| 1149 | 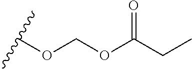 [SMILES: *OCOC(=O)CC] | H | H | Me |
| 1150 | 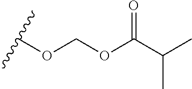 [SMILES: *OCOC(=O)C(C)C] | H | H | Me |
| 1151 | 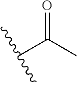 [SMILES: *C(C)=O] | 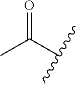 [SMILES: *C(C)=O] | H | Me |
| 1152 | 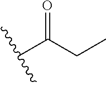 [SMILES: *C(=O)CC] | 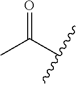 [SMILES: *C(C)=O] | H | Me |
| 1153 | 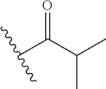 [SMILES: *C(=O)C(C)C] | 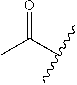 [SMILES: *C(C)=O] | H | Me |
| 1154 | 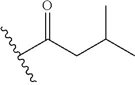 [SMILES: *C(=O)CC(C)C] | 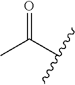 [SMILES: *C(C)=O] | H | Me |
| 1155 | 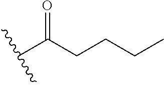 [SMILES: *C(=O)CCCC] | 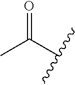 [SMILES: *C(C)=O] | H | Me |
| 1156 | 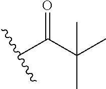 [SMILES: *C(=O)C(C)(C)C] | 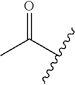 [SMILES: *C(C)=O] | H | Me |
| 1157 | 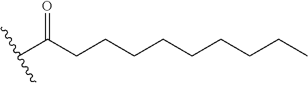 [SMILES: *C(=O)CCCCCCCCC] | 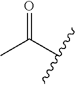 [SMILES: *C(C)=O] | H | Me |
| 1158 | 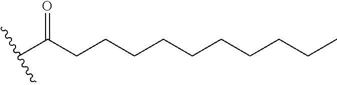 [SMILES: *C(=O)CCCCCCCCCC] | 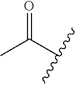 [SMILES: *C(C)=O] | H | Me |
| 1159 | 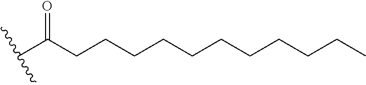 [SMILES: *C(=O)CCCCCCCCCCC] | 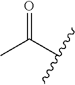 [SMILES: *C(C)=O] | H | Me |
| 1160 | 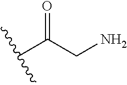 [SMILES: *C(=O)CN] | 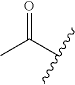 [SMILES: *C(C)=O] | H | Me |
| 1161 | 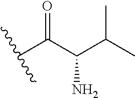 [SMILES: *C(=O)[C@@H](N)C(C)C] | 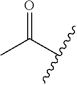 [SMILES: *C(C)=O] | H | Me |
| 1162 | 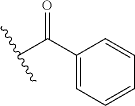 [SMILES: *C(=O)c1ccccc1] | 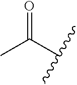 [SMILES: *C(C)=O] | H | Me |
| 1163 | 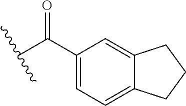 [SMILES: *C(=O)c1ccc2c(c1)CCC2] | 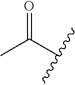 [SMILES: *C(C)=O] | H | Me |
| 1164 | 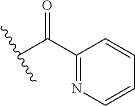 [SMILES: *C(=O)c1ccccn1] | 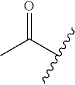 [SMILES: *C(C)=O] | H | Me |
| 1165 | 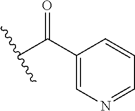 [SMILES: *C(=O)c1cccnc1] | 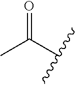 [SMILES: *C(C)=O] | H | Me |
| 1166 | 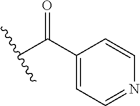 [SMILES: *C(=O)c1ccncc1] | 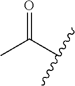 [SMILES: *C(C)=O] | H | Me |
| 1167 | 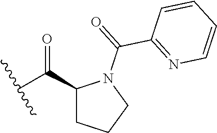 [SMILES: *C(=O)[C@@H]1CCCN1C(=O)c1ccccn1] | 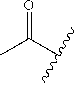 [SMILES: *C(C)=O] | H | Me |
| 1168 | 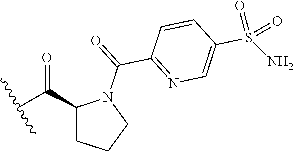 [SMILES: *C(=O)[C@@H]1CCCN1C(=O)c1ccc(S(N)(=O)=O)cn1] | 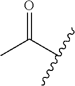 [SMILES: *C(C)=O] | H | Me |
| 1169 | 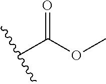 [SMILES: *C(=O)OC] | 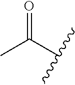 [SMILES: *C(C)=O] | H | Me |
| 1170 | 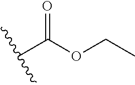 [SMILES: *C(=O)OCC] | 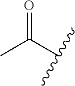 [SMILES: *C(C)=O] | H | Me |
| 1171 | 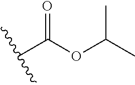 [SMILES: *C(=O)OC(C)C] | 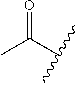 [SMILES: *C(C)=O] | H | Me |
| 1172 | 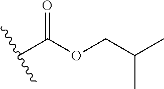 [SMILES: *C(=O)OCC(C)C] | 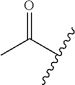 [SMILES: *C(C)=O] | H | Me |
| 1173 | 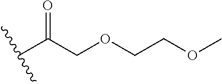 [SMILES: *C(=O)COCCOC] | 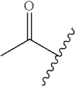 [SMILES: *C(C)=O] | H | Me |
| 1174 | 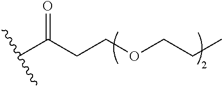 | 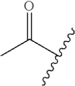 [SMILES: *C(C)=O] | H | Me |
| 1175 | 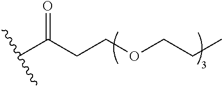 | 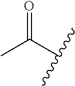 [SMILES: *C(C)=O] | H | Me |
| 1176 | 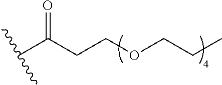 | 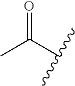 [SMILES: *C(C)=O] | H | Me |
| 1177 | 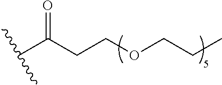 | 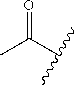 [SMILES: *C(C)=O] | H | Me |
| 1178 | 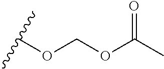 [SMILES: *OCOC(C)=O] | 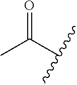 [SMILES: *C(C)=O] | H | Me |
| 1179 | 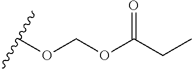 [SMILES: *OCOC(=O)CC] | 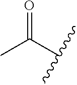 [SMILES: *C(C)=O] | H | Me |
| 1180 | 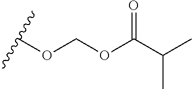 [SMILES: *OCOC(=O)C(C)C] | 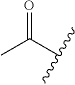 [SMILES: *C(C)=O] | H | Me |
| 1181 | 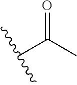 [SMILES: *C(C)=O] | H | 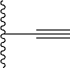 [SMILES: *C#C] | Me |
| 1182 | 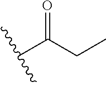 [SMILES: *C(=O)CC] | H | 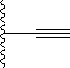 [SMILES: *C#C] | Me |
| 1183 | 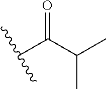 [SMILES: *C(=O)C(C)C] | H | 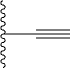 [SMILES: *C#C] | Me |
| 1184 | 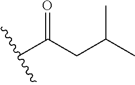 [SMILES: *C(=O)CC(C)C] | H | 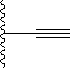 [SMILES: *C#C] | Me |
| 1185 | 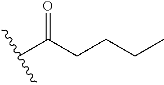 [SMILES: *C(=O)CCCC] | H | 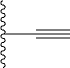 [SMILES: *C#C] | Me |
| 1186 | 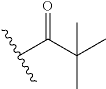 [SMILES: *C(=O)C(C)(C)C] | H | 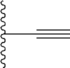 [SMILES: *C#C] | Me |
| 1187 | 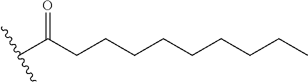 [SMILES: *C(=O)CCCCCCCCC] | H | 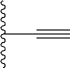 [SMILES: *C#C] | Me |
| 1188 | 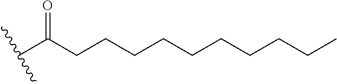 [SMILES: *C(=O)CCCCCCCCCC] | H | 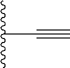 [SMILES: *C#C] | Me |
| 1189 | 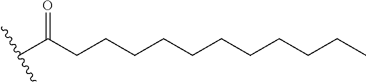 [SMILES: *C(=O)CCCCCCCCCCC] | H | 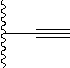 [SMILES: *C#C] | Me |
| 1190 | 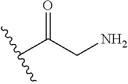 [SMILES: *C(=O)CN] | H | 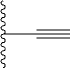 [SMILES: *C#C] | Me |
| 1191 | 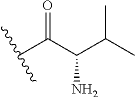 [SMILES: *C(=O)[C@@H](N)C(C)C] | H | 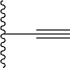 [SMILES: *C#C] | Me |
| 1192 | 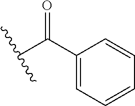 [SMILES: *C(=O)c1ccccc1] | H | 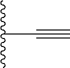 [SMILES: *C#C] | Me |
| 1193 | 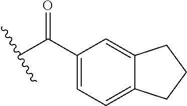 [SMILES: *C(=O)c1ccc2c(c1)CCC2] | H | 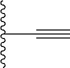 [SMILES: *C#C] | Me |
| 1194 | 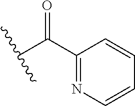 [SMILES: *C(=O)c1ccccn1] | H | 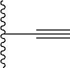 [SMILES: *C#C] | Me |
| 1195 | 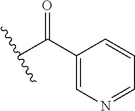 [SMILES: *C(=O)c1cccnc1] | H | 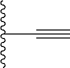 [SMILES: *C#C] | Me |
| 1196 | 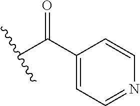 [SMILES: *C(=O)c1ccncc1] | H | 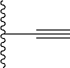 [SMILES: *C#C] | Me |
| 1197 | 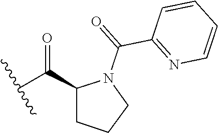 [SMILES: *C(=O)[C@@H]1CCCN1C(=O)c1ccccn1] | H | 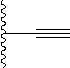 [SMILES: *C#C] | Me |
| 1198 | 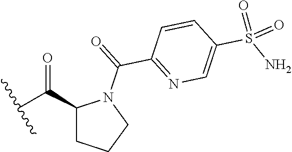 [SMILES: *C(=O)[C@@H]1CCCN1C(=O)c1ccc(S(N)(=O)=O)cn1] | H | 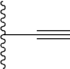 [SMILES: *C#C] | Me |
| 1199 | 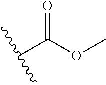 [SMILES: *C(=O)OC] | H | 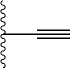 [SMILES: *C#C] | Me |
| 1200 | 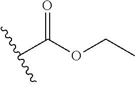 [SMILES: *C(=O)OCC] | H | 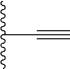 [SMILES: *C#C] | Me |
| 1201 | 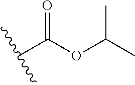 [SMILES: *C(=O)OC(C)C] | H | 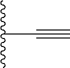 [SMILES: *C#C] | Me |
| 1202 | 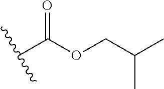 [SMILES: *C(=O)OCC(C)C] | H | 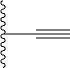 [SMILES: *C#C] | Me |
| 1203 | 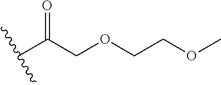 [SMILES: *C(=O)COCCOC] | H | 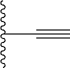 [SMILES: *C#C] | Me |
| 1204 | 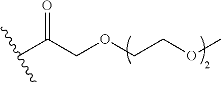 | H | 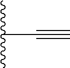 [SMILES: *C#C] | Me |
| 1205 | 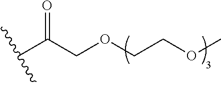 | H | 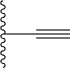 [SMILES: *C#C] | Me |
| 1206 | 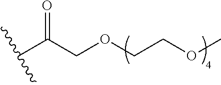 | H | 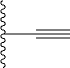 [SMILES: *C#C] | Me |
| 1207 | 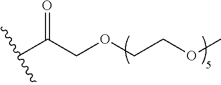 | H | 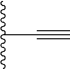 [SMILES: *C#C] | Me |
| 1208 | 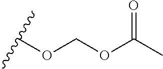 [SMILES: *OCOC(C)=O] | H | 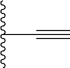 [SMILES: *C#C] | Me |
| 1209 | 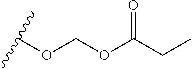 [SMILES: *OCOC(=O)CC] | H | 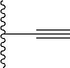 [SMILES: *C#C] | Me |
| 1210 | 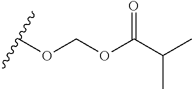 [SMILES: *OCOC(=O)C(C)C] | H | 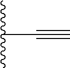 [SMILES: *C#C] | Me |
| 1211 | 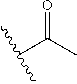 [SMILES: *C(C)=O] | 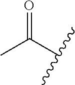 [SMILES: *C(C)=O] | 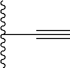 [SMILES: *C#C] | Me |
| 1212 | 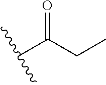 [SMILES: *C(=O)CC] | 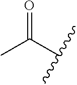 [SMILES: *C(C)=O] | 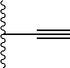 [SMILES: *C#C] | Me |
| 1213 | 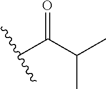 [SMILES: *C(=O)C(C)C] | 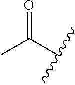 [SMILES: *C(C)=O] | 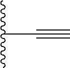 [SMILES: *C#C] | Me |
| 1214 | 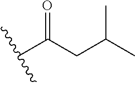 [SMILES: *C(=O)CC(C)C] | 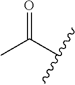 [SMILES: *C(C)=O] | 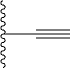 [SMILES: *C#C] | Me |
| 1215 | 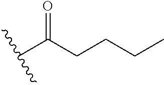 [SMILES: *C(=O)CCCC] | 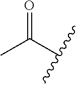 [SMILES: *C(C)=O] | 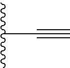 [SMILES: *C#C] | Me |
| 1216 | 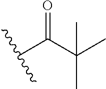 [SMILES: *C(=O)C(C)(C)C] | 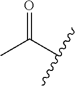 [SMILES: *C(C)=O] | 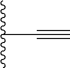 [SMILES: *C#C] | Me |
| 1217 | 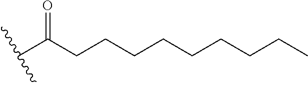 [SMILES: *C(=O)CCCCCCCCC] | 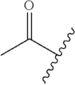 [SMILES: *C(C)=O] | 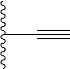 [SMILES: *C#C] | Me |
| 1218 | 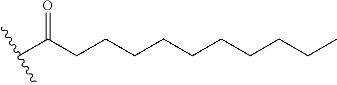 [SMILES: *C(=O)CCCCCCCCCC] | 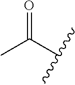 [SMILES: *C(C)=O] | 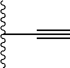 [SMILES: *C#C] | Me |
| 1219 | 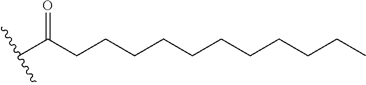 [SMILES: *C(=O)CCCCCCCCCCC] | 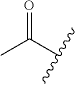 [SMILES: *C(C)=O] | 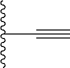 [SMILES: *C#C] | Me |
| 1220 | 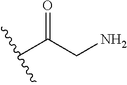 [SMILES: *C(=O)CN] | 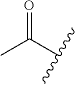 [SMILES: *C(C)=O] | 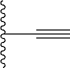 [SMILES: *C#C] | Me |
| 1221 | 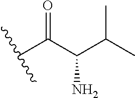 [SMILES: *C(=O)[C@@H](N)C(C)C] | 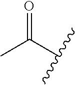 [SMILES: *C(C)=O] | 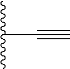 [SMILES: *C#C] | Me |
| 1222 | 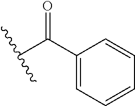 [SMILES: *C(=O)c1ccccc1] | 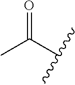 [SMILES: *C(C)=O] | 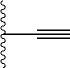 [SMILES: *C#C] | Me |
| 1223 | 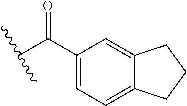 [SMILES: *C(=O)c1ccc2c(c1)CCC2] | 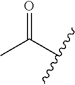 [SMILES: *C(C)=O] | 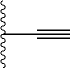 [SMILES: *C#C] | Me |
| 1224 | 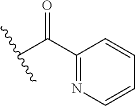 [SMILES: *C(=O)c1ccccn1] | 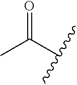 [SMILES: *C(C)=O] | 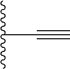 [SMILES: *C#C] | Me |
| 1225 | 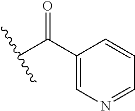 [SMILES: *C(=O)c1cccnc1] | 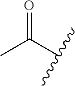 [SMILES: *C(C)=O] | 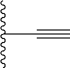 [SMILES: *C#C] | Me |
| 1226 | 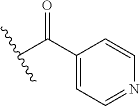 [SMILES: *C(=O)c1ccncc1] | 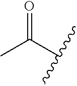 [SMILES: *C(C)=O] | 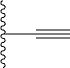 [SMILES: *C#C] | Me |
| 1227 | 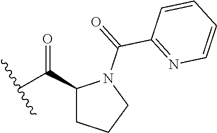 [SMILES: *C(=O)[C@@H]1CCCN1C(=O)c1ccccn1] | 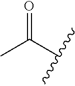 [SMILES: *C(C)=O] | 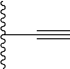 [SMILES: *C#C] | Me |
| 1228 | 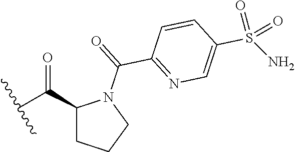 [SMILES: *C(=O)[C@@H]1CCCN1C(=O)c1ccc(S(N)(=O)=O)cn1] | 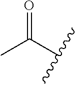 [SMILES: *C(C)=O] | 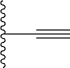 [SMILES: *C#C] | Me |
| 1229 | 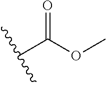 [SMILES: *C(=O)OC] | 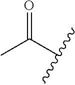 [SMILES: *C(C)=O] | 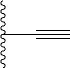 [SMILES: *C#C] | Me |
| 1230 | 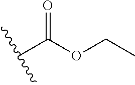 [SMILES: *C(=O)OCC] | 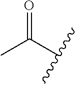 [SMILES: *C(C)=O] | 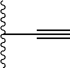 [SMILES: *C#C] | Me |
| 1231 | 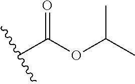 [SMILES: *C(=O)OC(C)C] | 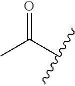 [SMILES: *C(C)=O] | 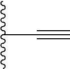 [SMILES: *C#C] | Me |
| 1232 | 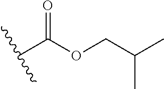 [SMILES: *C(=O)OCC(C)C] | 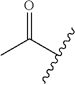 [SMILES: *C(C)=O] | 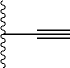 [SMILES: *C#C] | Me |
| 1233 | 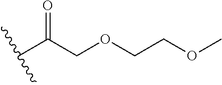 [SMILES: *C(=O)COCCOC] | 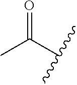 [SMILES: *C(C)=O] | 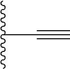 [SMILES: *C#C] | Me |
| 1234 | 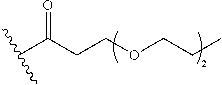 | 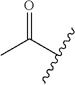 [SMILES: *C(C)=O] | 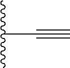 [SMILES: *C#C] | Me |
| 1235 | 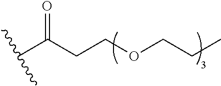 | 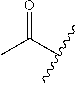 [SMILES: *C(C)=O] | 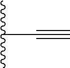 [SMILES: *C#C] | Me |
| 1236 | 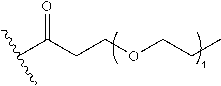 | 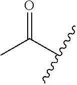 [SMILES: *C(C)=O] | 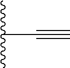 [SMILES: *C#C] | Me |
| 1237 | 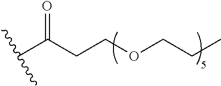 | 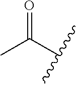 [SMILES: *C(C)=O] | 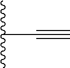 [SMILES: *C#C] | Me |
| 1238 | 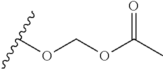 [SMILES: *OCOC(C)=O] | 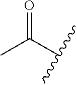 [SMILES: *C(C)=O] | 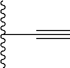 [SMILES: *C#C] | Me |
| 1239 | 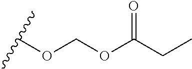 [SMILES: *OCOC(=O)CC] | 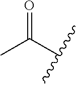 [SMILES: *C(C)=O] | 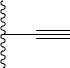 [SMILES: *C#C] | Me |
| 1240 | 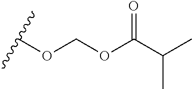 [SMILES: *OCOC(=O)C(C)C] | 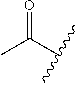 [SMILES: *C(C)=O] | 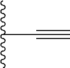 [SMILES: *C#C] | Me |
| 1241 | 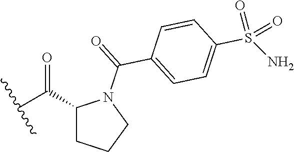 [SMILES: *C(=O)[C@H]1CCCN1C(=O)c1ccc(S(N)(=O)=O)cc1] | H | H | H |
| 1242 | 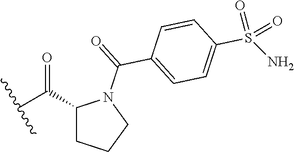 [SMILES: *C(=O)[C@H]1CCCN1C(=O)c1ccc(S(N)(=O)=O)cc1] | 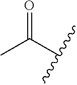 [SMILES: *C(C)=O] | H | H |
| 1243 | 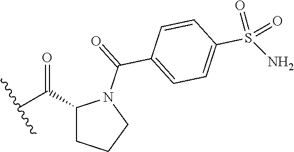 [SMILES: *C(=O)[C@H]1CCCN1C(=O)c1ccc(S(N)(=O)=O)cc1] | H | 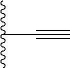 [SMILES: *C#C] | H |
| 1244 | 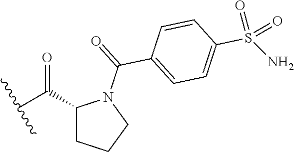 [SMILES: *C(=O)[C@H]1CCCN1C(=O)c1ccc(S(N)(=O)=O)cc1] | 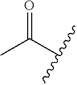 [SMILES: *C(C)=O] | 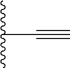 [SMILES: *C#C] | H |
| 1245 | 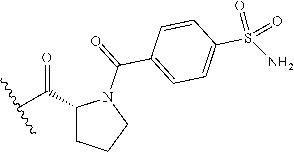 [SMILES: *C(=O)[C@H]1CCCN1C(=O)c1ccc(S(N)(=O)=O)cc1] | H | H | Me |
| 1246 | 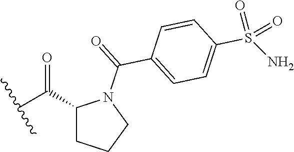 [SMILES: *C(=O)[C@H]1CCCN1C(=O)c1ccc(S(N)(=O)=O)cc1] | 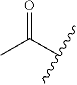 [SMILES: *C(C)=O] | H | Me |
| 1247 | 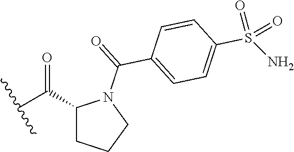 [SMILES: *C(=O)[C@H]1CCCN1C(=O)c1ccc(S(N)(=O)=O)cc1] | H | 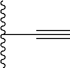 [SMILES: *C#C] | Me |
| 1248 | 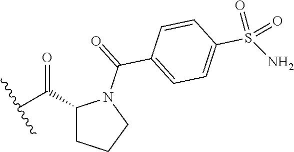 [SMILES: *C(=O)[C@H]1CCCN1C(=O)c1ccc(S(N)(=O)=O)cc1] | 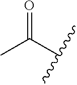 [SMILES: *C(C)=O] | 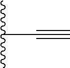 [SMILES: *C#C] | Me |
| 1249 | 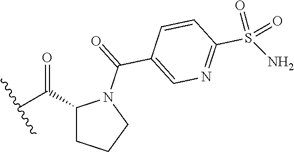 [SMILES: *C(=O)[C@H]1CCCN1C(=O)c1ccc(S(N)(=O)=O)nc1] | H | H | H |
| 1250 | 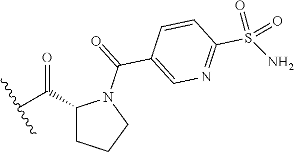 [SMILES: *C(=O)[C@H]1CCCN1C(=O)c1ccc(S(N)(=O)=O)nc1] | 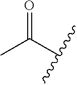 [SMILES: *C(C)=O] | H | H |
| 1251 | 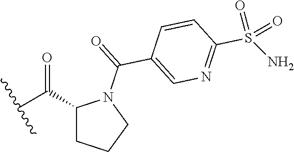 [SMILES: *C(=O)[C@H]1CCCN1C(=O)c1ccc(S(N)(=O)=O)nc1] | H | 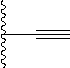 [SMILES: *C#C] | H |
| 1252 | 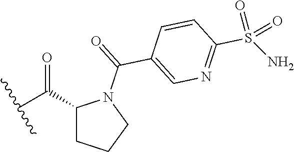 [SMILES: *C(=O)[C@H]1CCCN1C(=O)c1ccc(S(N)(=O)=O)nc1] | 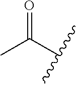 [SMILES: *C(C)=O] | 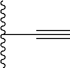 [SMILES: *C#C] | H |
| 1253 | 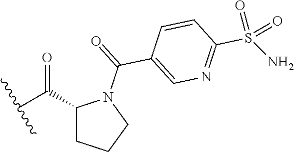 [SMILES: *C(=O)[C@H]1CCCN1C(=O)c1ccc(S(N)(=O)=O)nc1] | H | H | Me |
| 1254 | 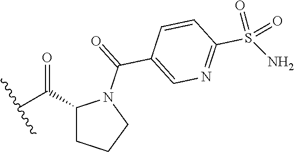 [SMILES: *C(=O)[C@H]1CCCN1C(=O)c1ccc(S(N)(=O)=O)nc1] | 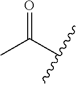 [SMILES: *C(C)=O] | H | Me |
| 1255 | 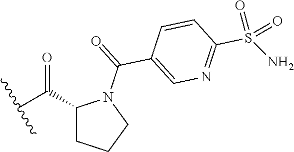 [SMILES: *C(=O)[C@H]1CCCN1C(=O)c1ccc(S(N)(=O)=O)nc1] | H | 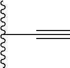 [SMILES: *C#C] | Me |
| 1256 | 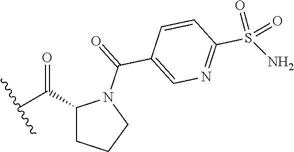 [SMILES: *C(=O)[C@H]1CCCN1C(=O)c1ccc(S(N)(=O)=O)nc1] | 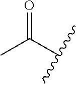 [SMILES: *C(C)=O] | 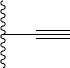 [SMILES: *C#C] | Me |

### Methods of Treatment

The compounds and compositions described herein can be used in methods for treating or preventing conditions and diseases, including but not limited to: a method of treating or preventing a disease or condition alleviated by activating and/or enhancing estrogen receptor-β (ERβ)-activity in a patient in need of said treatment, the method comprising administering to the patient a therapeutically effective amount of a compound of any of formula (I), formula (10), formula (11), formula (12), formulas 1001-1256, or pharmaceutically acceptable salts, solvates, hydrates, cocrystals, or prodrugs thereof; a method of treating or preventing a depressive disorder in a patient in need of said treatment, the method comprising administering to the patient a therapeutically effective amount of a compound of any of formula (I), formula (10), formula (11), formula (12), formulas 1001-1256, or pharmaceutically acceptable salts, solvates, hydrates, cocrystals, or prodrugs thereof; a method of treating or preventing an anxiety disorder in a patient in need of said treatment, the method comprising administering to the patient a therapeutically effective amount of a compound of any of formula (I), formula (10), formula (11), formula (12), formulas 1001-1256, or pharmaceutically acceptable salts, solvates, hydrates, cocrystals, or prodrugs thereof, a method of treating or preventing post-traumatic stress disorder (PTSD) in a patient in need of said treatment, the method comprising administering to the patient a therapeutically effective amount of a compound of any of formula (I), formula (10), formula (11), formula (12), formulas 1001-1256, or pharmaceutically acceptable salts, solvates, hydrates, cocrystals, or prodrugs thereof, a method of treating or preventing drug addiction in a patient in need of said treatment, the method comprising administering to the patient a therapeutically effective amount of a compound of any of formula (I), formula (10), formula (11), formula (12), formulas 1001-1256, or pharmaceutically acceptable salts, solvates, hydrates, cocrystals, or prodrugs thereof; a method of treating or preventing schizophrenia in a patient in need of said treatment, the method comprising administering to the patient a therapeutically effective amount of a compound of any of formula (I), formula (10), formula (11), formula (12), formulas 1001-1256, or pharmaceutically acceptable salts, solvates, hydrates, cocrystals, or prodrugs thereof; a method of treating or preventing Alzheimer's dementia in a patient in need of said treatment, the method comprising administering to the patient a therapeutically effective amount of a compound of any of formula (I), formula (10), formula (11), formula (12), formulas 1001-1256, or pharmaceutically acceptable salts, solvates, hydrates, cocrystals, or prodrugs thereof; a method of treating or preventing Parkinson's disease in a patient in need of said treatment, the method comprising administering to the patient a therapeutically effective amount of a compound of any of formula (I), formula (10), formula (11), formula (12), formulas 1001-1256, or pharmaceutically acceptable salts, solvates, hydrates, cocrystals, or prodrugs thereof; a method of treating or preventing stroke in a patient in need of said treatment, the method comprising administering to the patient a therapeutically effective amount of a compound of any of formula (I), formula (10), formula (11), formula (12), formulas 1001-1256, or pharmaceutically acceptable salts, solvates, hydrates, cocrystals, or prodrugs thereof, a method of treating or preventing traumatic brain injury (TBI) in a patient in need of said treatment, the method comprising administering to the patient a therapeutically effective amount of a compound of any of formula (I), formula (10), formula (11), formula (12), formulas 1001-1256, or pharmaceutically acceptable salts, solvates, hydrates, cocrystals, or prodrugs thereof; a method of treating or preventing amyotrophic lateral sclerosis (ALS) in a patient in need of said treatment, the method comprising administering to the patient a therapeutically effective amount of a compound of any of formula (I), formula (10), formula (11), formula (12), formulas 1001-1256, or pharmaceutically acceptable salts, solvates, hydrates, cocrystals, or prodrugs thereof; a method of treating or preventing complex regional pain syndrome (CRPS) in a patient in need of said treatment, the method comprising administering to the patient a therapeutically effective amount of a compound of any of formula (I), formula (10), formula (11), formula (12), formulas 1001-1256, or pharmaceutically acceptable salts, solvates, hydrates, cocrystals, or prodrugs thereof; a method of treating or preventing chronic pain in a patient in need of said treatment, the method comprising administering to the patient a therapeutically effective amount of a compound of any of formula (I), formula (10), formula (11), formula (12), formulas 1001-1256, or pharmaceutically acceptable salts, solvates, hydrates, cocrystals, or prodrugs thereof; a method of treating or preventing neuropathic pain in a patient in need of said treatment, the method comprising administering to the patient a therapeutically effective amount of a compound of any of formula (I), formula (10), formula (11), formula (12), formulas 1001-1256, or pharmaceutically acceptable salts, solvates, hydrates, cocrystals, or prodrugs thereof; a method of treating or preventing anhedonia in a patient in need of said treatment, the method comprising administering to the patient a therapeutically effective amount of a compound of any of formula (I), formula (10), formula (11), formula (12), formulas 1001-1256, or pharmaceutically acceptable salts, solvates, hydrates, cocrystals, or prodrugs thereof, a method of treating or preventing fatigue in a patient in need of said treatment, the method comprising administering to the patient a therapeutically effective amount of a compound of any of formula (I), formula (10), formula (11), formula (12), formulas 1001-1256, or pharmaceutically acceptable salts, solvates, hydrates, cocrystals, or prodrugs thereof, a method of treating or preventing fatigue in a patient in need of said treatment, the method comprising administering to the patient a therapeutically effective amount of a compound of any of formula (I), formula (10), formula (11), formula (12), formulas 1001-1256, or pharmaceutically acceptable salts, solvates, hydrates, cocrystals, or prodrugs thereof; a method of treating or preventing andropause-induced symptoms in a patient in need of said treatment, the method comprising administering to the patient a therapeutically effective amount of a compound of any of formula (I), formula (10), formula (11), formula (12), formulas 1001-1256, or pharmaceutically acceptable salts, solvates, hydrates, cocrystals, or prodrugs thereof; a method of treating or preventing orchiectomy-induced symptoms in a patient in need of said treatment, the method comprising administering to the patient a therapeutically effective amount of a compound of any of formula (I), formula (10), formula (11), formula (12), formulas 1001-1256, or pharmaceutically acceptable salts, solvates, hydrates, cocrystals, or prodrugs thereof.

Non-limiting examples of a depressive disorder include major depressive disorder, bipolar disorder, postpartum depression, drug withdrawal induced depression, treatment refractory depression, persistent depressive disorder (dysthymia), perinatal depression, seasonal affective disorder, seasonal depression, premenstrual dysphoric disorder, psychotic depression, suicidal ideation, suicidal actions, disruptive mood dysregulation disorder, premenstrual dysphoric disorder, substance/medication-induced depressive disorder, depressive disorder due to another medical condition, other specified depressive disorder, and an unspecified depressive disorder.

Non-limiting examples of anxiety disorders include general anxiety disorder, obsessive-compulsive disorder (OCD), post-traumatic stress disorder (PTSD), separation anxiety disorder, selective mutism, specific phobia, social anxiety disorder (social phobia), panic disorder, panic attack (specifier), agoraphobia, substance/medication-induced anxiety disorder, anxiety disorder due to another medical, other specified anxiety disorder, anhedonia, and unspecified anxiety disorder.

Non-limiting examples of drug addiction include nicotine addiction, alcohol addiction, cannabis addiction, cocaine addiction, and opioid addiction.

The disclosure includes a method of treating or preventing bipolar depression and major depressive disorder where an effective amount of the compound is an amount effective to decrease depressive symptoms, wherein a decrease in depressive symptoms is the achievement of a 50% or greater reduction of symptoms identified on a depression symptom rating scale, or a score less than or equal to 7 on the HRSD₁₇, or less than or equal to 5 on the QID-SR₁₆, or less than or equal to 10 on the MADRS.

The disclosure provides an amount effective to decrease painful symptoms; wherein a decrease in painful symptom is the achievement of a 50% or greater reduction of painful symptoms on a pain rating scale.

The disclosure provides a method of treating or preventing one or more of a depressive disorder, including major depressive disorder, bipolar disorder, postpartum depression, drug withdrawal induced depression, treatment refractory depression, persistent depressive disorder (dysthymia), perinatal depression, seasonal affective disorder, seasonal depression, premenstrual dysphoric disorder, psychotic depression, suicidal ideation, suicidal actions, disruptive mood dysregulation disorder, premenstrual dysphoric disorder, substance/medication-induced depressive disorder, depressive disorder due to another medical condition, other specified depressive disorder, and an unspecified depressive disorder, wherein a decrease in depressive symptoms is the achievement of a 50% or greater reduction of symptoms identified on a depression symptom rating scale, or a score less than or equal to 7 on the HRSD₁₇, or less than or equal to 5 on the QID-SR₁₆, or less than or equal to 10 on the MADRS.

The disclosure provides a method of treating or preventing one or more anxiety disorders, general anxiety disorder, obsessive-compulsive disorder (OCD), separation anxiety disorder, selective mutism, specific phobia, social anxiety disorder (social phobia), panic disorder, panic attack (specifier), agoraphobia, substance/medication-induced anxiety disorder, anxiety disorder due to another medical, other specified anxiety disorder, anhedonia, and unspecified anxiety disorder, wherein an effective amount is an amount effective to decrease anxiety symptoms; wherein a decrease in anxiety symptoms is the achievement of a 50% or greater reduction of anxiety symptoms on an anxiety symptom rating scale, or a score less than or equal to 39 on the STAT, or less than or equal to 9 on the BAT, or less than or equal to 7 on the HADS-A.

In some embodiments, the methods of the invention further includes administering to the patient psychotherapy, talk therapy, cognitive behavioral therapy, exposure therapy, systematic desensitization, mindfulness, dialectical behavior therapy, interpersonal therapy, eye movement desensitization and reprocessing, social rhythm therapy, acceptance and commitment therapy, family-focused therapy, psychodynamic therapy, light therapy, computer therapy, cognitive remediation, exercise, or other types of therapy.

In some aspects, the disclosure provides a method of treating or preventing opioid addiction in a patient in need thereof. In some embodiments, the method comprises administering to the patient a therapeutically effective amount of a compound of any of formula (I), formula (10), formula (11), formula (12), formulas 1001-1256, or a pharmaceutically acceptable salt, solvate, hydrate, cocrystal, or prodrug thereof. In some embodiments, the opioid addiction is further associated with a depressive disorder and/or an anxiety disorder. In some embodiments, the depressive disorder is selected from major depressive disorder, bipolar disorder, postpartum depression, drug withdrawal induced depression, treatment refractory depression, persistent depressive disorder (dysthymia), perinatal depression, seasonal affective disorder, seasonal depression, premenstrual dysphoric disorder, psychotic depression, suicidal ideation, suicidal actions, disruptive mood dysregulation disorder, premenstrual dysphoric disorder, substance/medication-induced depressive disorder, depressive disorder due to another medical condition, other specified depressive disorder, and an unspecified depressive disorder. In some embodiments, the anxiety disorder is selected from general anxiety disorder, obsessive-compulsive disorder (OCD), separation anxiety disorder, selective mutism, specific phobia, social anxiety disorder (social phobia), panic disorder, panic attack (specifier), agoraphobia, substance/medication-induced anxiety disorder, anxiety disorder due to another medical, other specified anxiety disorder, anhedonia, and unspecified anxiety disorder.

In one aspect, the disclosure provides a method of treating or preventing opioid withdrawal or one or more symptoms associated with opioid withdrawal. In some embodiments, the method comprises administering to the patient a therapeutically effective amount of a compound of any of formula (I), formula (10), formula (11), formula (12), formulas 1001-1256, or a pharmaceutically acceptable salt, solvate, hydrate, cocrystal, or prodrug thereof. In some embodiments, the one or more symptoms of opioid withdrawal is selected from dysphoria, anxiety, restlessness, irritability, insomnia, yawning, hallucinations, tremors, shaking, hot or cold flashes, goosebumps, sneezing, sweating, rapid breathing, elevated heart rate, elevated blood pressure, pupillary dilation, piloerection, headaches, body aches, muscle cramps, muscle aches, bone aches, joint aches, hyperalgesia, hyperkatifiteia, watery discharge from eyes and nose (lacrimation and rhinorrhea), nausea, vomiting, diarrhea, abdominal pain, anorexia and fever. In some embodiments, the opioid withdrawal is induced by administration of one or more opioid antagonists or partial agonists. In some embodiments, the opioid antagonist is naltrexone In some embodiments, the compound of any of formula (I), formula (10), formula (11), formula (12), formulas 1001-1256, or a pharmaceutically acceptable salt, solvate, hydrate, cocrystal, or prodrug is administered concurrently, before, or after administration of the opioid antagonist or partial agonist. In some embodiments, the opioid partial agonist is selected from morphine, methadone, fentanyl, sufentanil and heroin.

In one aspect, the disclosure provides a method of treating or preventing relapse of opioid addiction. In some embodiments, the method comprises administering to the patient a therapeutically effective amount of a compound of any of formula (I), formula (10), formula (11), formula (12), formulas 1001-1256, or a pharmaceutically acceptable salt, solvate, hydrate, cocrystal, or prodrug thereof. In some embodiments, the patient previously reduced or eliminated use of one or more opioids in response to treatment with an effective amount of an anti-addiction treatment, and is no longer exposed to an effective amount of the anti-addiction treatment. In some embodiments, the patient has undergone physical and/or physiological withdrawal from one or more opioids. In some embodiments, the relapse is stress-induced. In some embodiments, the patient has undergone physiological withdrawal from the one or more opioids during the period of abstinence from, or limited or reduced use of, the one or more opioids. In some embodiments, the patient is no longer exposed to an effective amount of the anti-addiction treatment because the patient has become conditioned to the anti-addiction treatment. In some embodiments, the patient is no longer exposed to an effective amount of the anti-addiction treatment because the patient has reduced or eliminated exposure to the anti-addiction treatment.

In some embodiments, the opioid addiction comprises and addiction of one or more opioids selected from oxycodone, morphine, buprenorphine, codeine, fentanyl, opium, methadone, heroin, hydrocodone, hydromorphone, oxymorphone, meperidine, tramadol, propoxyphene, diphenoxylate, loperamide, nalbuphine, butorphanol, pentazocine, carfentanil and other fentanyl analogues, and combinations thereof. In some embodiments, the compound of any of formula (I), formula (10), formula (11), formula (12), formulas 1001-1256, or a pharmaceutically acceptable salt, solvate, hydrate, cocrystal, or prodrug thereof is administered in a single dose.

In one aspect, the disclosure provides the use of the compound of any of formula (I), formula (10), formula (11), formula (12), formulas 1001-1256, or a pharmaceutically acceptable salt, solvate, hydrate, cocrystal, or prodrug thereof for treating or preventing opioid addiction in a patient in need thereof.

In one aspect, the disclosure provides the use of the compound of any of formula (I), formula (10), formula (11), formula (12), formulas 1001-1256, or a pharmaceutically acceptable salt, solvate, hydrate, cocrystal, or prodrug thereof for treating or preventing opioid withdrawal or one or more symptoms associated with opioid withdrawal.

In one aspect, the disclosure provides the use of the compound of any of formula (I), formula (10), formula (11), formula (12), formulas 1001-1256, or a pharmaceutically acceptable salt, solvate, hydrate, cocrystal, or prodrug thereof for treating or preventing relapse of opioid addiction.

In some embodiments, the methods of treatment include providing certain dosage amounts of a compound of any of formula (I), formula (10), formula (11), formula (12), formulas 1001-1256, or pharmaceutically acceptable salts, solvates, hydrates, cocrystals, or prodrugs thereof to a patient. In some embodiments, the dosage levels of each active agent of from about 0.1 mg to about 140 mg per kilogram of body weight per day are useful in the treatment of the above-indicated conditions (about 0.5 mg to about 7 g per patient per day). The amount of active ingredient that may be combined with the carrier materials to produce a single unit dosage form will vary depending upon the patient treated and the particular mode of administration.

In some embodiments a therapeutically effect amount is an amount that provide a plasma Cmax of a compound of a compound of any of formula (I), formula (10), formula (11), formula (12), formulas 1001-1256, or pharmaceutically acceptable salts, solvates, hydrates, cocrystals, or prodrugs thereof of about of 0.25 mcg/mL to about 125 mcg/mL, or about 1 mcg/mL to about 50 mcg/mL. For peripheral indications formulations and methods that provide a Cmax of about 0.25 mcg/mL to about 25 mcg/mL are preferred, while for CNS indications, formulations and methods that provide a plasma Cmax of about 0.25 mcg/mL to about 125 mcg/mL are preferred. The disclosure also includes IV pharmaceutical compositions that provide about 0.2 mg to about 500 mg per dose of a compound of any of formula (I), formula (10), formula (11), formula (12), formulas 1001-1256, or pharmaceutically acceptable salts, solvates, hydrates, cocrystals, or prodrugs thereof, In some embodiments, for peripheral indications, the pharmaceutical composition provides about 0.5 mg to about 500 mg/dose.

### Pharmaceutical Compositions

In an embodiment, the disclosure provides a pharmaceutical composition for use in the treatment of the diseases and conditions described herein.

The pharmaceutical compositions are typically formulated to provide a therapeutically effective amount of a compound of any of formula (I), formula (10), formula (11), formula (12), formulas 1001-1256, or pharmaceutically acceptable salts, solvates, hydrates, cocrystals, or prodrugs thereof, as described herein, as the active ingredient. Typically, the pharmaceutical compositions also comprise one or more pharmaceutically acceptable excipients, carriers, including inert solid diluents and fillers, diluents, including sterile aqueous solution and various organic solvents, permeation enhancers, solubilizers and adjuvants.

In one aspect, the pharmaceutical compositions described above are for use in the treatment or prevention of, without limitation, a disease or condition alleviated by activating and/or enhancing estrogen receptor-β (ERβ)-activity; a disease or condition selected from a depressive disorder, an anxiety disorder, post-traumatic stress disorder (PTSD), drug addiction, schizophrenia, Alzheimer's dementia, Parkinson's disease, stroke, traumatic brain injury (TBI), amyotrophic lateral sclerosis (ALS), complex regional pain syndrome (CRPS), chronic pain, neuropathic pain, anhedonia, fatigue, andropause-induced symptoms, and orchiectomy-induced symptoms, the pharmaceutical composition comprising one or more compounds, or pharmaceutically acceptable salts, solvates, hydrates, cocrystals, or prodrugs thereof, having any one of any of formula (I), formula (10), formula (11), formula (12), formulas 1001-1256, and a pharmaceutically acceptable carrier. In some embodiments, the concentration of a compound of formula (I), formula (10), formula (11), formula (12), formulas 1001-1256, or pharmaceutically acceptable salt thereof, provided in the pharmaceutical compositions of the disclosure is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of a compound of any of formula (I), formula (10), formula (11), formula (12), formulas 1001-1256, or pharmaceutically acceptable salt thereof, provided in the pharmaceutical compositions of the disclosure is independently greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of a compound of formula (I), formula (10), formula (11), formula (12), formulas 1001-1256, or pharmaceutically acceptable salt thereof, provided in the pharmaceutical compositions of the disclosure is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of a compound of formula (I), formula (10), formula (11), formula (12), formulas 1001-1256, or pharmaceutically acceptable salt thereof, provided in the pharmaceutical compositions of the disclosure is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of a compound of formula (I), formula (10), formula (11), formula (12), formulas 1001-1256, or pharmaceutically acceptable salt thereof, provided in the pharmaceutical compositions of the disclosure is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of a compound of formula (I), formula (10), formula (11), formula (12), formulas 1001-1256, or pharmaceutically acceptable salt thereof, provided in the pharmaceutical compositions of the disclosure is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5 g, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

Each of the compounds provided according to the disclosure is effective over a wide dosage range. For example, in the treatment of adult humans, dosages independently ranging from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the gender and age of the subject to be treated, the body weight of the subject to be treated, and the preference and experience of the attending physician.

Described below are non-limiting pharmaceutical compositions and methods for preparing the same.

### Pharmaceutical Compositions for Oral Administration

In preferred embodiments, the disclosure provides a pharmaceutical composition for oral administration containing: a compound of formula (I), formula (10), formula (11), formula (12), formulas 1001-1256, or pharmaceutically acceptable salt thereof, described herein, and a pharmaceutical excipient suitable for administration.

In preferred embodiments, the disclosure provides a solid pharmaceutical composition for oral administration containing: (i) an effective amount of: a compound of formula (I), formula (10), formula (11), formula (12), formulas 1001-1256, or pharmaceutically acceptable salt thereof, and (ii) a pharmaceutical excipient suitable for administration. In some embodiments, the composition further contains (iii) an effective amount of an additional active pharmaceutical ingredient. For example, additional active pharmaceutical ingredients, as used herein, may include one or more compounds that are useful in the treatment or prevention of a disease or condition, such as a disease or condition selected from a depressive disorder, an anxiety disorder, post-traumatic stress disorder (PTSD), drug addiction, schizophrenia, Alzheimer's dementia, Parkinson's disease, stroke, traumatic brain injury (TBI), amyotrophic lateral sclerosis (ALS), complex regional pain syndrome (CRPS), chronic pain, neuropathic pain, anhedonia, fatigue, andropause-induced symptoms, and orchiectomy-induced symptoms.

Non-limiting examples of additional active pharmaceutical ingredients or agents include:
Antidepressants: ketamine, (R)-ketamine, (S)-ketamine, (2R,6R)-hydroxynorketamine (HNK), escitalopram, fluoxetine, paroxetine, duloxetine, sertraline, citalopram, bupropion, venlafaxine, duloxetine, naltrexone, mirtazapine, venlafaxine, atomoxetine, bupropion, doxepin, amitriptyline, clomipramine, nortriptyline, buspirone, aripiprazole, clozapine, loxapine, olanzapine, quetiapine, risperidone, ziprasidone, carbamazepine, gabapentin, lamotrigine, phenytoin, pregabalin, donepezil, galantamine, memantine, rivastigmine, tramiprosate, or pharmaceutically active salts or prodrugs thereof, or a combination of the foregoing; Schizophrenia Medications: aripiprazole, lurasidone, asenapine, clozapine, ziprasidone, risperidone, quetiapine, stelazine, olanzapine, loxapine, flupentioxol, perphenazine, haloperidol, chlorpromazine, fluphenazine, prolixin, paliperidone;
Alzheimer's Dementia Medications: donepezil, rivastigmine, galantamine, memantine; ALS Medications: riluzole;
Pain Medications: acetaminophen, aspirin, NSAIDS, including Diclofenac, Diflunisal, Etodolac, Fenoprofen, Flurbiprofen, Ibuprofen, Indomethacin, Ketoprofen, Ketorolac, Meclofenamate, Mefenamic Acid, Meloxicam, Nabumetone, Naproxen, Oxaprozin, Phenylbutazone, Piroxicam, Sulindac, Tolmetinopiods, Cox-2 inhibitors such as celcoxib, and narcotic pain medications such as Buprenorphine, Butorphanol, Codeine, Hydrocodone, Hydromorphone, Levorphanol, Meperidine, Methadone, Morphine, Nalbuphine, Oxycodone, Oxymorphone, Pentazocine, Propoxyphene, the central analgesic tramadol;
Medications for treatment of drug addiction: benzodiazepines, antidepressants, buprenorphine, methadone, naltrexone, clonidine, and naloxone;
CNS active agents: d-cycloserine, dextromethorphan, escitalopram, fluoxetine, paroxetine, duloxetine, sertraline, citalopram, bupropion, venlafaxine, duloxetine, naltrexone, mirtazapine, venlafaxine, atomoxetine, bupropion, doxepin, amitriptyline, clomipramine, nortriptyline, vortioxetine, vilazadone, milnacipran, levomilacipran, pramipexole, buspirone, lithium, thyroid or other type of hormones (e.g., estrogen, progesterone, testosterone), aripiprazole, brexpiprazole, cariprazine, clozapine, loxapine, lurasidone, olanzapine, paliperidone, quetiapine, risperidone, ziprasidone, carbamazepine, oxcarbazepine, gabapentin, lamotrigine, phenytoin, pregabalin, donepezil, galantamine, memantine, minocycline, rivastigmine, riluzole, tramiprosate, ketamine, or pharmaceutically active salts or prodrugs thereof, or a combination of the foregoing;
Anti-anxiety and anti-psychotic drugs: hydroxyzine hydrochloride, lorazepam, buspirone hydrochloride, pazepam, chlordiazepoxide, meprobamate, oxazepam, trifluoperazine, clorazepate dipotassium, diazepam, clozapine, prochlorperazine, haloperidol, thioridazine, thiothixene, risperidone, trifluoperazine hydrochloride, chlorpromazine, and related substances.

In some embodiments, the pharmaceutical composition may be a liquid pharmaceutical composition suitable for oral consumption.

Pharmaceutical compositions of the disclosure suitable for oral administration can be presented as discrete dosage forms, such as capsules, sachets, or tablets, or liquids or aerosol sprays each containing a predetermined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms can be prepared by any of the methods of pharmacy, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The disclosure further encompasses anhydrous pharmaceutical compositions and dosage forms since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the disclosure can be prepared using anhydrous or low moisture containing ingredients and low moisture or low humidity conditions. Pharmaceutical compositions and dosage forms of the disclosure which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

Active pharmaceutical ingredients can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the disclosure to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the disclosure include, but are not limited to, calcium stearate, magnesium stearate, sodium stearyl fumarate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, silicified microcrystalline cellulose, or mixtures thereof. A lubricant can optionally be added in an amount of less than about 0.5% or less than about 1% (by weight) of the pharmaceutical composition.

When aqueous suspensions and/or elixirs are desired for oral administration, the active pharmaceutical ingredient(s) may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyllactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acyllactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof, polyoxyethylated vitamins and derivatives thereof, polyoxyethylene-polyoxypropylene block copolymers; and mixtures thereof, polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In an embodiment, the composition may include a solubilizer to ensure good solubilization and/or dissolution of the compound of the present disclosure and to minimize precipitation of the compound of the present disclosure. This can be especially important for compositions for non-oral use—e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon.-caprolactone and isomers thereof, S-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid.

### Pharmaceutical Compositions for Injection

In preferred embodiments, the disclosure provides a pharmaceutical composition for injection containing: a compound of formula (I), formula (10), formula (11), formula (12), formulas 1001-1256, or pharmaceutically acceptable salt thereof, described herein, and a pharmaceutical excipient suitable for injection. Components and amounts of compounds in the compositions are as described herein.

The forms in which the compositions of the disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, and thimerosal.

Sterile injectable solutions are prepared by incorporating a compound of formula (I), formula (10), formula (11), formula (12), formulas 1001-1256, or pharmaceutically acceptable salt thereof, described herein, in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Pharmaceutical Compositions for Topical Delivery

In preferred embodiments, the disclosure provides a pharmaceutical composition for transdermal delivery containing: a compound of formula (I), formula (10), formula (11), formula (12), formulas 1001-1256, or pharmaceutically acceptable salt thereof, described herein, and a pharmaceutical excipient suitable for transdermal delivery.

Compositions of the present disclosure can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present disclosure employs transdermal delivery devices (“patches”). Such transdermal patches may be used to provide continuous or discontinuous infusion of: a compound of formula (I), formula (10), formula (11), formula (12), formulas 1001-1256, or pharmaceutically acceptable salt thereof, described herein, in controlled amounts, either with or without another active pharmaceutical ingredient.

The construction and use of transdermal patches for the delivery of pharmaceutical agents is well known in the art. See, e.g., U.S. Pat. Nos. 5,023,252; 4,992,445 and 5,001,139. Such patches may be constructed for continuous, pulsatile, or on demand delivery of pharmaceutical agents.

### Pharmaceutical Compositions for Inhalation

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner. Dry powder inhalers may also be used to provide inhaled delivery of the compositions.

### Other Pharmaceutical Compositions

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, et al., eds., Handbook of Clinical Drug Data, Tenth Edition, McGraw-Hill, 2002; and Pratt and Taylor, eds., Principles of Drug Action, Third Edition, Churchill Livingston, N.Y., 1990, each of which is incorporated by reference herein in its entirety.

Administration of a compound of formula (I), formula (10), formula (11), formula (12), formulas 1001-1256, or pharmaceutically acceptable salt thereof, described herein, or a pharmaceutical composition of these compounds can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. In embodiments, the compound of formula (I), formula (10), formula (11), formula (12), formulas 1001-1256, or pharmaceutically acceptable salt thereof, described herein, can also be administered intraadiposally or intrathecally. In embodiments, the compound of formula (I), formula (10), formula (11), formula (12), formulas 1001-1256, or pharmaceutically acceptable salt thereof, described herein, is administered orally. In embodiments, the compound of formula (I), formula (10), formula (11), formula (12), formulas 1001-1256, or pharmaceutically acceptable salt thereof, described herein, is administered intravenously.

The compositions of the disclosure may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the disclosure may slow or inhibit the migration and proliferation of smooth muscle cells in the arterial wall which contribute to restenosis. A compound of the disclosure may be administered, for example, by local delivery from the struts of a stent, from a stent graft, from grafts, or from the cover or sheath of a stent. In some embodiments, a compound of the disclosure is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly(ether-ester) copolymers (e.g., PEO-PLLA); polydimethylsiloxane, poly(ethylene-vinylacetate), acrylate-based polymers or copolymers (e.g., polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. A compound of formula (I), formula (10), formula (11), formula (12), formulas 1001-1256, or pharmaceutically acceptable salt thereof, described herein, may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the disclosure in a suitable solvent, followed by evaporation of the solvent. Excess drug on the surface of the stent may be removed via an additional brief solvent wash. In yet other embodiments, compounds of the disclosure may be covalently linked to a stent or graft. A covalent linker may be used which degrades in vivo, leading to the release of the compound of the disclosure. Any bio-labile linkage may be used for such a purpose, such as ester, amide or anhydride linkages. A compound of formula (I), formula (10), formula (11), formula (12), formulas 1001-1256, or pharmaceutically acceptable salt thereof, described herein, may additionally be administered intravascularly from a balloon used during angioplasty. Extravascular administration of a compound of formula (I), formula (10), formula (11), formula (12), formulas 1001-1256, or pharmaceutically acceptable salt thereof, described herein, via the pericard or via advential application of formulations of the disclosure may also be performed to decrease restenosis.

Exemplary parenteral administration forms include solutions or suspensions of a compound of formula (I), formula (10), formula (11), formula (12), formulas 1001-1256, or pharmaceutically acceptable salt thereof, in sterile aqueous solutions, for example, aqueous propylene glycol or dextrose solutions. Such dosage forms can be suitably buffered, if desired.

The disclosure also provides kits. The kits include a compound of formula (I), formula (10), formula (11), formula (12), formulas 1001-1256, or pharmaceutically acceptable salt thereof, described herein, in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. In some embodiments, the compound of formula (I), formula (10), formula (11), formula (12), formulas 1001-1256, or pharmaceutically acceptable salt thereof, described herein, and another active pharmaceutical ingredient are provided as separate compositions in separate containers within the kit. In some embodiments, the compound of formula (I), formula (10), formula (11), formula (12), formulas 1001-1256, or pharmaceutically acceptable salt thereof, and the agent are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in some embodiments, be marketed directly to the consumer.

The kits described above are preferably for use in the treatment of the diseases and conditions described herein. In some embodiments, the kits described herein are for use in the treatment of a disease or condition selected from a depressive disorder, an anxiety disorder, drug addiction, schizophrenia, Alzheimer's dementia, amyotrophic lateral sclerosis, complex regional pain syndrome (CRPS), chronic pain, and neuropathic pain.

### Dosages and Dosing Regimens

The amounts of: a compound of formula (I), formula (10), formula (11), formula (12), formulas 1001-1256, or pharmaceutically acceptable salt thereof, described herein, administered will be dependent on the human or mammal being treated, the severity of the disorder or condition, the rate of administration, the disposition of the compounds and the discretion of the prescribing physician. However, an effective dosage of each is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day. The dosage of a compound of formula (I), formula (10), formula (11), formula (12), formulas 1001-1256, or pharmaceutically acceptable salt thereof, described herein, may be provided in units of mg/kg of body mass or in mg/m² of body surface area.

In some embodiments, a compound of formula (I), formula (10), formula (11), formula (12), formulas 1001-1256, or pharmaceutically acceptable salt thereof, described herein is administered in multiple doses. In a preferred embodiment, a compound of formula (I), formula (10), formula (11), formula (12), formulas 1001-1256, or pharmaceutically acceptable salt thereof, described herein is administered in multiple doses. Dosing may be once, twice, three times, four times, five times, six times, or more than six times per day. Dosing may be once a month, once every two weeks, once a week, or once every other day. In other embodiments, a compound of formula (I), formula (10), formula (11), formula (12), formulas 1001-1256, or pharmaceutically acceptable salt thereof, described herein, is administered about once per day to about 6 times per day. In some embodiments, a compound of formula (I), formula (10), formula (11), formula (12), formulas 1001-1256, or pharmaceutically acceptable salt thereof, described herein, is administered once daily, while in other embodiments, a compound of formula (I), formula (10), formula (11), formula (12), formulas 1001-1256, or pharmaceutically acceptable salt thereof, described herein is administered twice daily, and in other embodiments a compound of formula (I), formula (10), formula (11), formula (12), formulas 1001-1256, or pharmaceutically acceptable salt thereof, described herein, is administered three times daily.

Administration a compound of formula (I), formula (10), formula (11), formula (12), formulas 1001-1256, or pharmaceutically acceptable salt thereof, described herein, may continue as long as necessary. In some embodiments, a compound of formula (I), formula (10), formula (11), formula (12), formulas 1001-1256, or pharmaceutically acceptable salt thereof, described herein, is administered for more than 1, 2, 3, 4, 5, 6, 7, 14, or 28 days. In some embodiments, a compound of formula (I), formula (10), formula (11), formula (12), formulas 1001-1256, or pharmaceutically acceptable salt thereof, described herein is administered for less than 28, 14, 7, 6, 5, 4, 3, 2, or 1 day. In some embodiments, a compound of formula (I), formula (10), formula (11), formula (12), formulas 1001-1256, or pharmaceutically acceptable salt thereof, described herein is administered chronically on an ongoing basis—e.g., for the treatment of chronic effects. In another embodiment, the administration of a compound of formula (I), formula (10), formula (11), formula (12), formulas 1001-1256, or pharmaceutically acceptable salt thereof, described herein, continues for less than about 7 days. In yet another embodiment, the administration continues for more than about 6, 10, 14, 28 days, two months, six months, or one year. In some cases, continuous dosing is achieved and maintained as long as necessary.

In some embodiments, an effective dosage of a compound of formula (I), formula (10), formula (11), formula (12), formulas 1001-1256, or pharmaceutically acceptable salt thereof, described herein, is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg.

In some embodiments, an effective dosage of a compound of formula (I), formula (10), formula (11), formula (12), formulas 1001-1256, or pharmaceutically acceptable salt thereof, described herein, is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg.

In some instances, dosage levels below the lower limit of the aforesaid ranges may be more than adequate, while in other cases still larger doses may be employed without causing any harmful side effect—e.g., by dividing such larger doses into several small doses for administration throughout the day.

An effective amount of a compound of formula (I), formula (10), formula (11), formula (12), formulas 1001-1256, or pharmaceutically acceptable salt thereof, described herein, may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: Synthesis of Compounds of the Disclosure

General Synthetic Methods: All commercially available reagents and solvents were purchased and used without further purification. All microwave reactions were carried out in a sealed microwave vial equipped with a magnetic stir bar and heated in a Biotage Initiator Microwave Synthesizer. ¹H NMR spectra were recorded on Varian 400 MHz spectrometers in CD₃OD, CD₃CN, CDCl₃, or D₆-DMSO as indicated. For spectra recorded in CD₃OD, chemical shifts are reported in ppm with CD₃OD (3.31 ppm) as reference for ¹H NMR spectra. For spectra recorded in CDCl₃, chemical shifts are reported in ppm relative to dueterochloroform (7.26 ppm for ¹H NMR). For spectra recorded in CD₃CN, chemical shifts are reported in ppm relative to CD₃CN (1.93 ppm for ¹H NMR). For spectra in D₆-DMSO chemical shifts are reported in ppm relative to D₆-DMSO (2.50 ppm for ¹H NMR). The coupling constants (J value) are reported as Hertz (Hz). The splitting patterns of the peaks were described as: singlet (s); doublet (d); triplet (t); quartet (q); multiplet (m) and septet (septet).

Compounds were analyzed on an Agilent 1200 series LC/MS equipped with a Luna C18 (3 mm×75 mm, 3 μm) reversed-phase column with UV detection at λ=220 nm and λ=254 nm. The mobile phase consisted of water containing 0.05% trifluoroacetic acid as component A and acetonitrile containing 0.025% trifluoroacetic acid as component B. A linear gradient was run as follows: 0 min 4% B; 7 min 100% B; 8 min 100% B at a flow rate of 0.8 ml/min.

Reverse phase chromatography for purification purposes was performed on a Waters semipreparative HPLC equipment. The column used was a Phenomenex Luna C18 (5 μm, 30×75 mm) at a flow rate of 45 mL/min. The mobile phase consisted of acetonitrile and water (each containing 0.1% trifluoroacetic acid, 0.1% formic acid, or 0.1% ammonium hydroxide, as indicated). A gradient of 5%-100% acetonitrile in water was used during the purification. Fraction collection was triggered by UV detection (220 nm).

### General Synthetic Routes

In a non-limiting example, compounds of formula I can be prepared as illustrated in the general scheme I below:

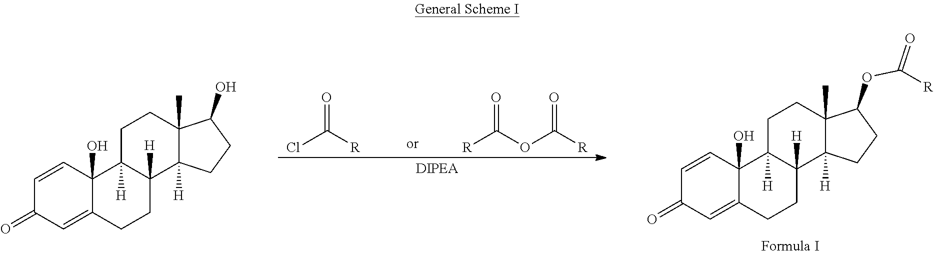

In a non-limiting example, compounds of formula II and formula III can also be prepared as per general scheme II:

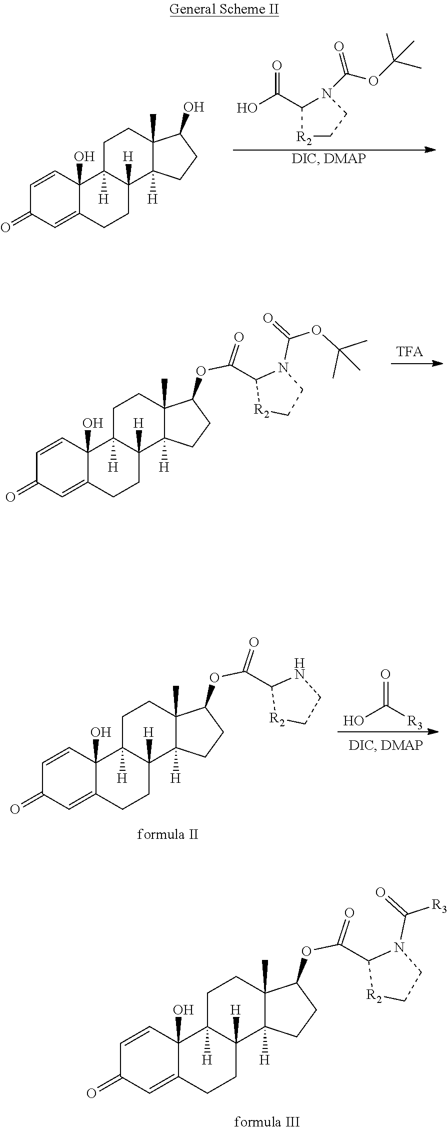

### Synthesis of Compounds

### Example 1.1

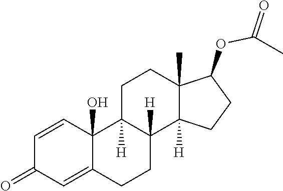

### (8S,9S,10S,13S,14S,17S)-10-hydroxy-13-methyl-3-oxo-6,7,8,9,10,11,12,13,14,15,16,17-dodecahydro-3H-cyclopenta[a]phenanthren-17-yl acetate

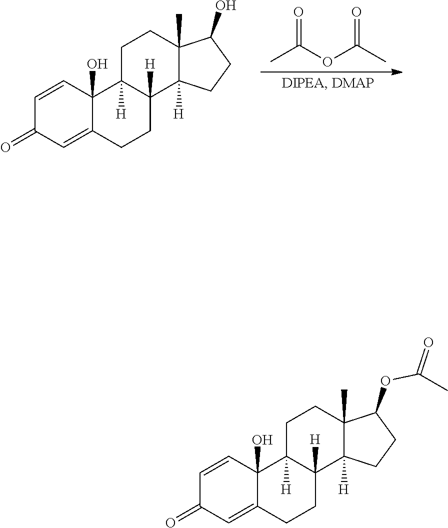

Step 1: A vial was charged with (8S,9S,10S,13S,14S,17S)-10,17-dihydroxy-13-methyl-6,7,8,9,10,11,12,13,14,15,16,17-dodecahydro-3H-cyclopenta[a]phenanthren-3-one (25.0 mg, 0.087 mmol, DHED). Then dichloromethane (2.0 ml) and tetrahydrofuran (2.0 ml) were added, followed by acetic anhydride (54.1 mg, 0.530 mmol, 50.0 μL), and diisopropylethyl amine (37.0 mg, 0.286 mmol, 50.0 μL). Then 4-dimethylaminopyridine (2.1 mg, 0.017 mmol) was added. The reaction was stirred at 23° C. for 2 hours. It was then quenched by being poured into an saturated aqueous solution of sodium bicarbonate, and was extracted with ethyl acetate. The organic phase was taken, and the solvent was removed by rotary evaporation. Purification by silica gel chromatography gave the title product. ¹H NMR (400 MHz, Chloroform-d) δ 7.06 (d, J=10.2 Hz, 1H), 6.15 (ddd, J=10.2, 2.0, 1.1 Hz, 1H), 5.97 (q, J=1.5 Hz, 1H), 4.58 (dd, J=9.2, 7.7 Hz, 1H), 2.83-2.67 (m, 1H), 2.32 (dt, J=12.9, 3.3 Hz, 1H), 2.16 (dtt, J=11.7, 8.4, 4.4 Hz, 1H), 2.03 (s, 3H), 2.03-1.88 (m, 4H), 1.80 (dt, J=12.9, 3.5 Hz, 1H), 1.73-1.60 (m, 2H), 1.57-1.46 (m, 1H), 1.38 (qd, J=12.0, 6.0 Hz, 1H), 1.23-0.98 (m, 3H), 0.89 (s, 3H). LC-MS [M+H+]: 331.2.

### Example 1.2

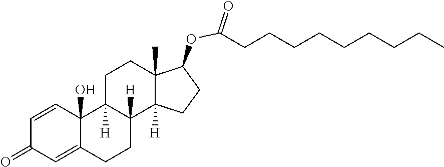

### (8S,9S,10S,13S,14S,17S)-10-hydroxy-13-methyl-3-oxo-6,7,8,9,10,11,12,13,14,15,16,17-dodecahydro-3H-cyclopenta[a]phenanthren-17-yl decanoate

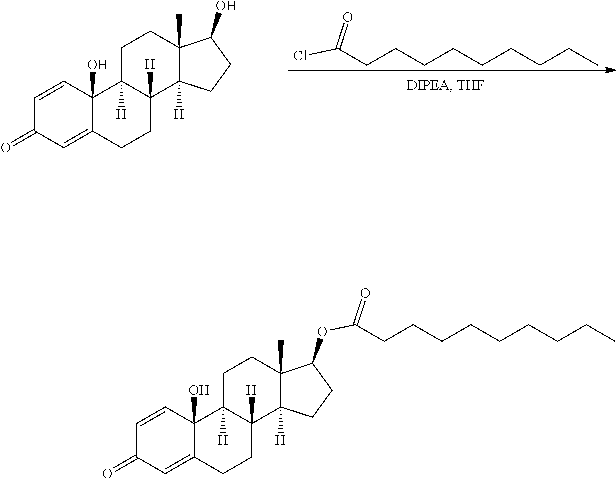

Step 1: A vial was charged with (8S,9S,10S,13S,14S,17S)-10,17-dihydroxy-13-methyl-6,7,8,9,10,11,12,13,14,15,16,17-dodecahydro-3H-cyclopenta[a]phenanthren-3-one (25.0 mg, 0.087 mmol, DHED). Then dichloromethane (2.0 ml) and tetrahydrofuran (2.0 ml) was added, followed by decanoyl chloride (20.0 mg, 0.105 mmol), then diisopropylethylamine (37.0 mg, 0.286 mmol, 50.0 μL). The reaction was stirred at 23° C. for 2 hours. It was then quenched by being poured into an saturated aqueous solution of sodium bicarbonate, and was extracted with ethyl acetate. The organic phase was taken, and the solvent was removed by rotary evaporation. Purification by silica gel chromatography gave the title product. ¹H NMR (400 MHz, Chloroform-d) δ 7.06 (dd, J=10.2, 0.5 Hz, 1H), 6.17 (dd, J=10.2, 2.0 Hz, 1H), 6.00 (t, J=1.7 Hz, 1H), 4.60 (dd, J=9.2, 7.7 Hz, 1H), 2.82-2.66 (m, 1H), 2.34 (ddd, J=12.9, 4.1, 2.3 Hz, 1H), 2.29 (dd, J=7.8, 7.2 Hz, 2H), 2.23-2.11 (m, 1H), 2.03-1.88 (m, 3H), 1.84-1.75 (m, 1H), 1.74-1.55 (m, 6H), 1.55-1.46 (m, 1H), 1.40 (td, J=12.0, 6.0 Hz, 1H), 1.36-1.21 (m, 11H), 1.21-1.00 (m, 3H), 0.89 (s, 3H), 0.88 (t, J=7.2 Hz, 3H) LC-MS [M+H+]: 443.3.

### Example 1.3

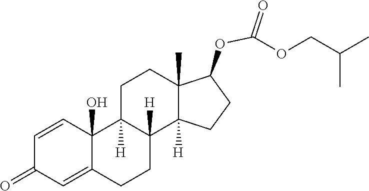

### (8S,9S,10S,13S,14S,17S)-10-hydroxy-13-methyl-3-oxo-6,7,8,9,10,11,12,13,14,15,16,17-dodecahydro-3H-cyclopenta[a]phenanthren-17-yl isobutyl carbonate

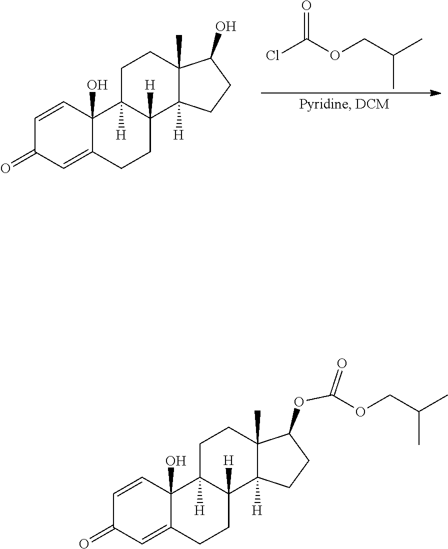

Step 1: A vial was charged with (8S,9S,10S,13S,14S,17S)-10,17-dihydroxy-13-methyl-6,7,8,9,10,11,12,13,14,15,16,17-dodecahydro-3H-cyclopenta[a]phenanthren-3-one (25.0 mg, 0.087 mmol, DHED). Then dichloromethane (1.0 ml) and pyridine (0.5 ml) was added, followed by isobutylchloroformate (12.0 mg, 0.087 mmol). The reaction was stirred at 23° C. for 0.5 hours. It was then quenched by being poured into a saturated aqueous solution of ammonium chloride, and was extracted with dichloromethane. The organic phase was taken, and the solvent was removed by rotary evaporation. Purification by silica gel chromatography gave the title product. LC-MS [M+H+]: 389.2.

### Example 1.4

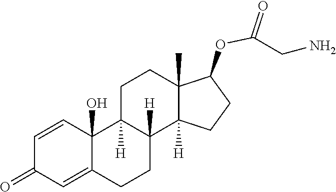

### (8S,9S,10S,13S,14S,17S)-10-hydroxy-13-methyl-3-oxo-6,7,8,9,10,11,12,13,14,15,16,17-dodecahydro-3H-cyclopenta[a]phenanthren-17-yl glycinate

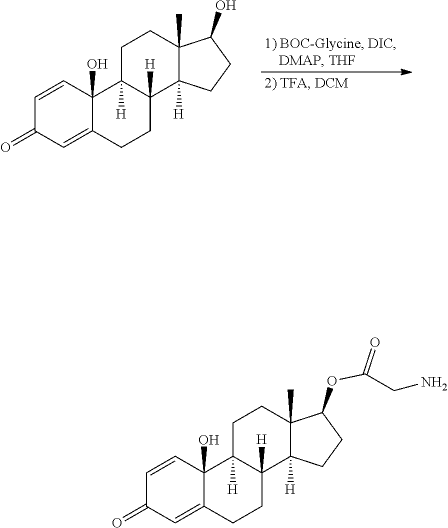

Step 1: A vial was charged with N-(tert-Butoxycarbonyl)glycine (61.0 mg, 0.347 mmol) followed by dichloromethane (2.0 ml), then N,N′-Diisopropylcarbodiimide (44.0 mg, 0.347 mmol). The mixture was stirred for 1 hour at 23° C. Then (8S,9S,10S,13S,14S,17S)-10,17-dihydroxy-13-methyl-6,7,8,9,10,11,12,13,14,15,16,17-dodecahydro-3H-cyclopenta[a]phenanthren-3-one (50.0 mg, 0.173 mmol, DHED) was added, followed by 4-dimethylaminopyridine (21.0 mg, 0.173 mmol). The reaction was stirred at 23° C. for 2 hours. It was then poured into a saturated solution of aqueous sodium bicarbonate and extracted into ethyl acetate. The organic phase was taken, and the solvent removed by rotary evaporation. Purification by silica gel chromatography gave the BOC-protected intermediate. ¹H NMR (400 MHz, Chloroform-d) δ 7.06 (dd, J=10.2, 0.5 Hz, 1H), 6.17 (dd, J=10.2, 2.0 Hz, 1H), 5.99 (t, J=1.8 Hz, 1H), 4.97 (s, 1H), 4.65 (dd, J=9.2, 7.7 Hz, 1H), 3.90 (d, J=5.6 Hz, 2H), 2.87-2.68 (m, 1H), 2.37-2.30 (m, 1H), 2.24-2.12 (m, 1H), 2.02-1.89 (m, 3H), 1.87-1.76 (m, 2H), 1.74-1.62 (m, 1H), 1.54 (ddd, J=11.6, 7.6, 2.8 Hz, 1H), 1.45 (s, 9H), 1.43-1.37 (m, 1H), 1.31-1.21 (m, 1H), 1.20-1.00 (m, 3H), 0.89 (d, J=0.6 Hz, 3H), 0.88-0.81 (m, 1H). LC-MS [M+H+]: 446.2.

Step 2: A vial was charged with the BOC-protected intermediate from step 1, (8S,9S,10S,13S,14S,17S)-10-hydroxy-13-methyl-3-oxo-6,7,8,9,10,11,12,13,14,15,16,17-dodecahydro-3H-cyclopenta[a]phenanthren-17-yl (tert-butoxycarbonyl)glycinate (0.030 g, 0.067 mmol). To this was added dichloromethane (1.0 ml), followed by trifluoroacetic acid (1.0 ml, 1.48 g, 12.98 mmol). The reaction was stirred at 23° C. for 0.33 hours. The solvent and trifluoroacetic acid were then removed by rotary evaporation. The residue was treated with aqueous sodium bicarbonate (saturated, ~5.0 ml), and extracted into ethyl acetate. The organic phase was taken, and the solvent was removed by rotary evaporation to give the title product.

LC-MS [M+H+]: 346.2.

### Example 1.5

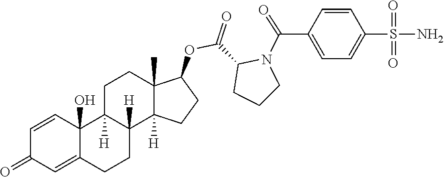

### (8S,9S,10S,13S,14S,17S)-10-hydroxy-13-methyl-3-oxo-6,7,8,9,10,11,12,13,14,15,16,17-dodecahydro-3H-cyclopenta[a]phenanthren-17-yl (4-sulfamoylbenzoyl)-D-prolinate

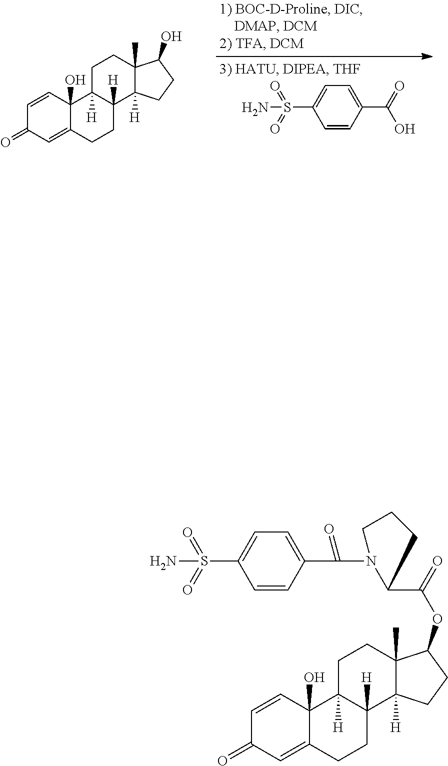

Step 1: A vial was charged with (tert-butoxycarbonyl)-D-proline (149 mg, 0.694 mmol) followed by dichloromethane (4.0 ml), then N,N′-Diisopropylcarbodiimide (88.0 mg, 0.694 mmol). The mixture was stirred for 1 hour at 23° C. Then (8S,9S,10S,13S,14S,17S)-10,17-dihydroxy-13-methyl-6,7,8,9,10,11,12,13,14,15,16, 17-dodecahydro-3H-cyclopenta[a]phenanthren-3-one (10.0 mg, 0.349 mmol, DHED) was added, followed by 4-dimethylaminopyridine (42.0 mg, 0.347 mmol). The reaction was stirred at 23° C. for 2 hours. It was then poured into a saturated solution of aqueous sodium bicarbonate and extracted into ethyl acetate. The organic phase was taken, and the solvent removed by rotary evaporation. Purification by silica gel chromatography gave the BOC-protected intermediate.

Step 2: A vial was charged with the BOC-protected intermediate from step 1, 1-(tert-butyl) 2-(8S,9S,10S,13S,14S,17S)-10-hydroxy-13-methyl-3-oxo-6,7,8,9,10,11,12,13,14,15,16,17-dodecahydro-3H-cyclopenta[a]phenanthren-17-yl) (R)-pyrrolidine-1,2-dicarboxylate (0.090 g, 0.185 mmol). To this was added dichloromethane (1.0 ml), followed by trifluoroacetic acid (1.0 ml, 1.48 g, 12.98 mmol). The reaction was stirred at 23° C. for 0.5 hours. The solvent and trifluoroacetic acid were removed by rotary evaporation. The residue was treated with aqueous sodium bicarbonate (saturated, ~5.0 ml), and extracted into ethyl acetate. The organic phase was taken and the solvent removed by rotary evaporation to give the intermediate.

Step 3: (8S,9S,10S,13S,14S,17S)-10-hydroxy-13-methyl-3-oxo-6,7,8,9,10,11,12,13,14,15,16,17-dodecahydro-3H-cyclopenta[a]phenanthren-17-yl D-prolinate (0.035 g, 0.091 mmol) was placed in a vial with a stirbar. To this was added tetrahydrofuran (4.0 ml). Then 4-sulfamoylbenzoic acid (0.040 g, 0.199 mmol) was added to the reaction mixture, followed by Hexafluorophosphate Azabenzotriazole Tetramethyl Uronium (0.060 g, 0.158 mmol, HATU), then triethyl amine (0.04 g, 0.395 mmol, 0.055 ml). The reaction was stirred at 23° C. for 2.0 hours. The reaction was then poured into aqueous sodium bicarbonate and extracted into ethyl acetate. The organic phase was taken and the solvent was removed by rotary evaporation. Purification by silica gel chromatography (hexanes-ethyl acetate) gave the title product. ¹H NMR (400 MHz, Methanol-d₄) δ 8.02-7.95 (m, 2H), 7.70 (d, J=7.9 Hz, 2H), 7.21 (d, J=9.2 Hz, 1H), 6.19-6.08 (m, 1H), 5.95 (d, J=6.6 Hz, 1H), 4.68 (t, J=8.4 Hz, 1H), 4.59 (dd, J=8.4, 4.4 Hz, 1H), 3.63-3.52 (m, 2H), 2.79 (dt, J=15.4, 7.7 Hz, 1H), 2.45-2.27 (m, 2H), 2.22-2.12 (m, 1H), 2.08-1.83 (m, 7H), 1.75-1.50 (m, 3H), 1.17-1.01 (m, 2H), 0.96 (s, 3H). LC-MS [M+H+]. 569.2.

### Example 1.6

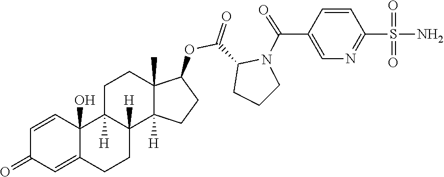

### (8S,9S,10S,13S,14S,17S)-10-hydroxy-13-methyl-3-oxo-6,7,8,9,10,11,12,13,14,15,16,17-dodecahydro-3H-cyclopenta[a]phenanthren-17-yl (6-sulfamoylnicotinoyl)-D-prolinate

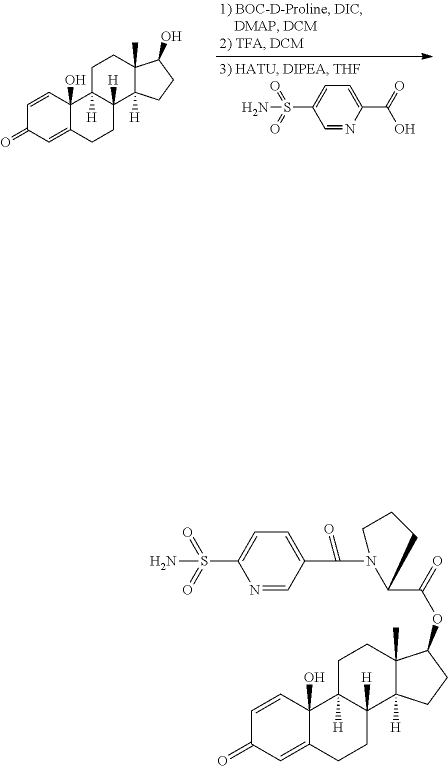

The compound of Example 1.6 was synthesized analogously to that of Example 1.5. ¹H NMR (400 MHz, Methanol-d₄) δ 8.85 (s, 1H), 8.20 (dd, J=8.1, 2.0 Hz, 1H), 8.09 (d, J=8.1 Hz, 1H), 7.20 (d, J=10.2 Hz, 1H), 6.12 (dd, J=10.2, 2.0 Hz, 1H), 6.04-5.93 (m, 1H), 4.69 (t, J=8.4 Hz, 1H), 4.60 (td, J=8.9, 3.8 Hz, 1H), 3.83-3.57 (m, 2H), 2.77 (tt, J=15.4, 7.8 Hz, 1H), 2.38 (dt, J=27.9, 8.8 Hz, 2H), 2.17 (dt, J=15.0, 8.5 Hz, 1H), 2.09-1.84 (m, 7H), 1.77-1.52 (m, 3H), 1.50-1.27 (m, 2H), 1.20-1.00 (m, 1H), 0.96 (s, 3H). LC-MS [M+H+]: 570.2.

### Example 2: Estradiol Prodrugs

At completion of the experiments described herein, an improved understanding of the role of estrogen signaling in depression, PTSD, and other stress-influenced neuropsychiatric conditions in males is attained and a novel treatment approach for both males and females with estrogen responsive disorders is defined. Additionally, data is generated that supports treatment for military personnel and veterans suffering from stress-induced neuropsychiatric conditions.

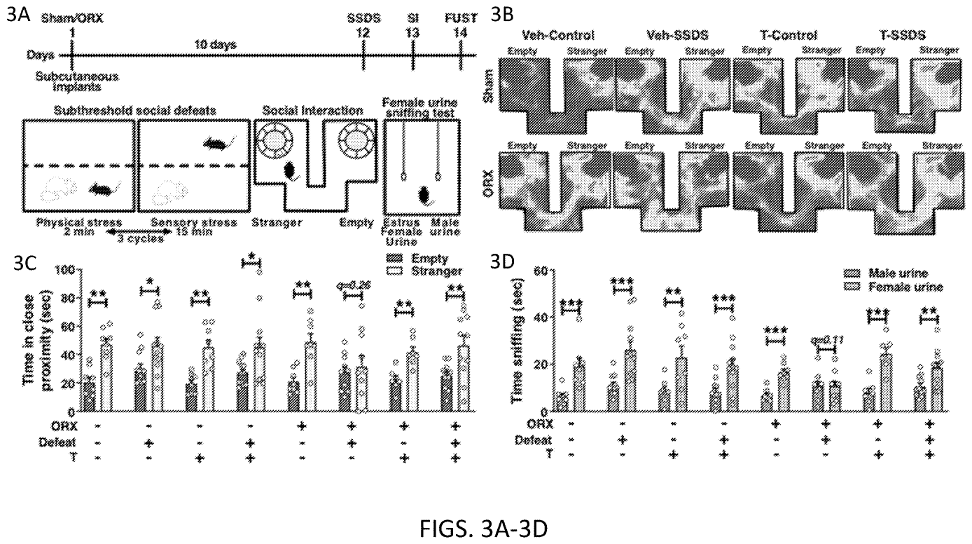

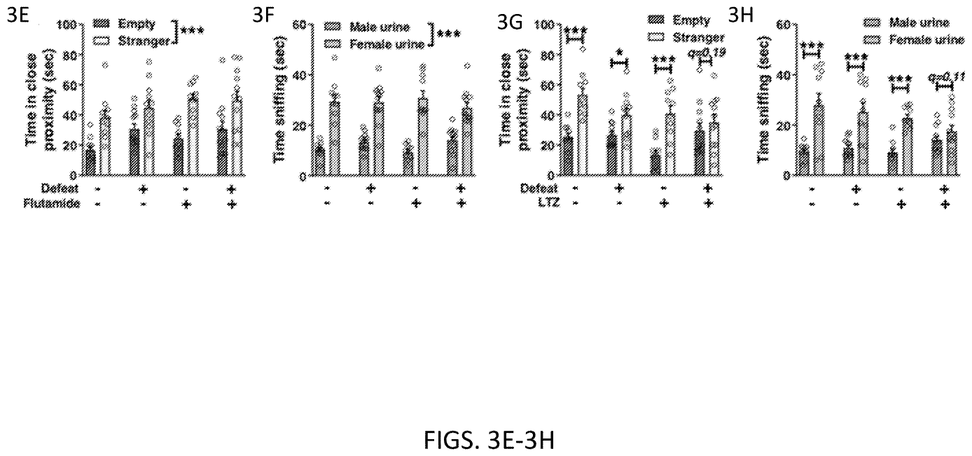

It is hypothesized that modifications to the DHED structure will have pronounced effects in extending its half-life and increasing oral bioavailability, resulting in an ideal drug candidate to treat estrogen-responsive neuropsychiatric disorders. This basic science discovery can be used for the benefits of veterans in need of care for stress-related psychiatric disorders. First, the potential novel roles of E2 signaling in the treatment of depression and PTSD in males is examined. Second, a prodrug strategy for DHED, similar to prior FDA-approved testosterone-based drugs (FIGS. 3A-3H) is examined, to boost DHED's oral bioavailability. It is proposed to use animal models to test innovative compounds, which are hypothesized to act as prodrugs to DHED (and bioprecursors to E2). The results these experiments can result in a profound breakthrough in the development of a new and safer, orally bioavailable, and brain selective E2 that could be utilized for human male proof of concept and treatment studies, and also provide a novel treatment for females for whom estrogen replacement therapy is not a viable treatment option.

Determine the broader efficacy of E2 in males using rodent models of psychiatric disease treatment effectiveness. The data is extended with rigorous experiments assessing effects of diverse stressors in both hypogonadal and gonadally intact, as well as aged mice, and the effectiveness of E2 and DHED treatment is defined. Rodent models of psychiatric disease treatment effectiveness can be used to further determine the role of E2 signaling in mediating stress susceptibility in males. Models tested, with relevance to neuropsychiatric disorders that disproportionately effect veterans, include hypogonadism- and age-induced stress susceptibility relevant to depression and anxiety, and stress-induced changes in startle response and fear retention and extinction relevant to PTSD. Responses in males are compared to those observed of females. These studies will address hypogonadism- and age-dependent mechanisms of enhanced susceptibility to stress and pathological fear, relevant to neuropsychiatric conditions that disproportionately affect those who have served in the military, and help identify which psychiatric disorders, in addition to depression, may be most amenable to E2-mediated treatment.

Optimize brain-selective estradiol for improved oral bioavailability properties. Candidate prodrugs for improved oral bioavailability are assessed, and the oral bioavailability of DHED prodrugs is determined by comparing levels of DHED and E2 in the brain following intravenous and oral administration in male and female rodents. Those prodrugs that exhibit favorable oral bioavailability and E2 production in the brain will be tested for in vivo response to E2-related biomarkers and therapeutic efficacy for stress-related phenotypes. A subset of DHED prodrug candidates are tested, and the oral bioavailability in male and female mice and rats obtained by comparing intravenous and oral administration by gavage in both male and female animals. Those prodrugs with sufficient oral availability, conversion to DHED and subsequent production of E2 are tested for in vivo response to E2 sensitive biomarkers and medication efficacy measures. Effects of compounds on progesterone receptor expression in the brain are assessed as a marker of central effects; galanin and C3 expression in the pituitary and uterus (in females), respectively, as markers of peripheral estrogen activity; models of stress susceptibility related to depression; and additional disease models previously identified.

At completion of these experiments, an improved understanding of the role of E2 signaling in depression as well as other stress-influenced neuropsychiatric diseases in males is determined, and a novel treatment approach for such disorders better defined. Preeclinical data is generated, enabling preclinical pharmacology and toxicology studies and a future treatment for veterans suffering from stress-induced psychiatric disorders.

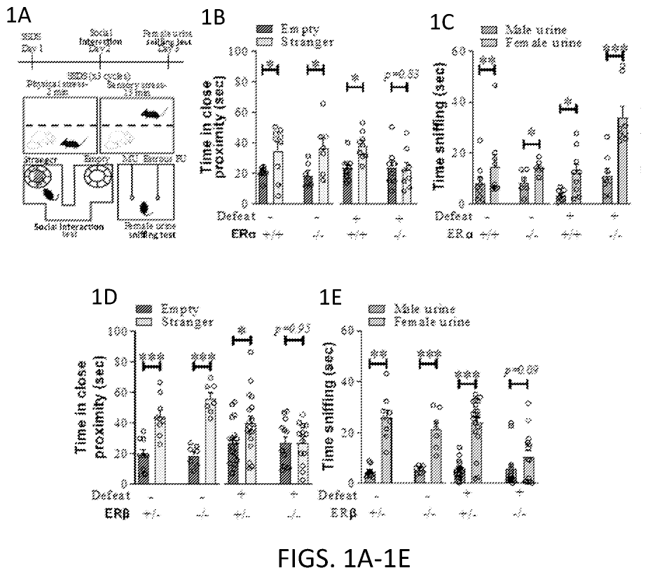

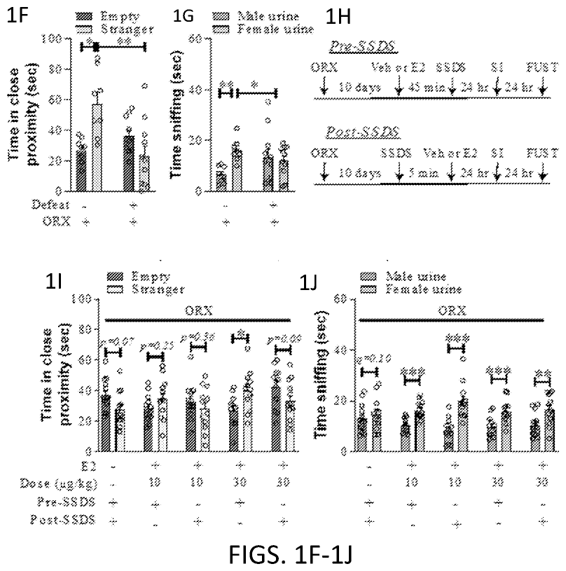

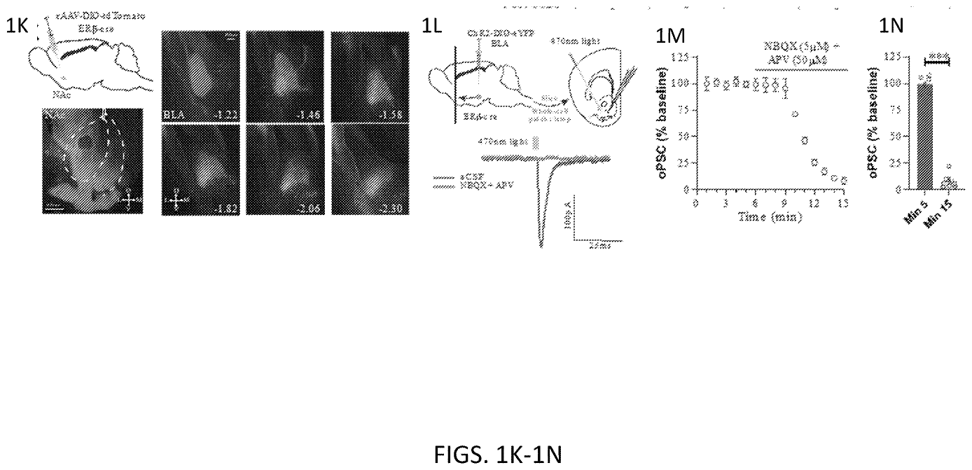

The basis of these studies was the hypothesis that testosterone acts as a precursor and that it is E2 that is responsible for testosterone's antidepressant effects in males through its action on estrogen receptors (ERs). It was assessed whether lack of ERα or ERβ in conventional knockout mice (ERKO or BERKO, respectively) results in maladaptive stress-sensitive behaviors. Following subthreshold social defeat stress, which is a single day stress paradigm that does not induce any depressive-related behavioral changes in control mice (FIGS. 1A-1D), it was found that male mice with a genetic deletion of ERα manifested susceptibility to develop social interaction deficits (FIG. 1), but not anhedonia as tested by preference for the smell of female versus male mouse urine (FIG. 1C). In contrast, genetic deletion of ERβ increased susceptibility to develop both social interaction deficits (FIG. 1D) and anhedonia (FIG. 1E) in male mice. While not wishing to be bound by any particular theory, these results suggested that ERβ might play a broader role in stress susceptibility than ERα. These maladaptive phenotypes were not observed in control ERKO or BERKO mice that were not subjected to stress (FIGS. 1D-1E), indicating that even a relatively mild stress can induce a depressive phenotype when ERβ-related signaling is reduced. Non-stressed BERKO male mice demonstrated the expected female urine preference, indicating that the lack of preference is due to stress and not due to other confounding factors. To test whether these behavioral effects were mediated by circulating gonadal hormones, male mice were orchiectomized to remove endogenous testosterone (and consequently E2) and subjected to subthreshold social defeat stress or control conditions. It was found that orchiectomized mice manifested the same stress-susceptible phenotype as observed in the BERKO mice (FIGS. 1F-1G). Without wishing to be bound by any particular theory, these results suggest that the testosterone likely promotes stress resilience under normal conditions. Importantly, ORX non-stress controls demonstrated social preference and preference to the female urine, demonstrating that these findings are due to stress and not due to the reduced chemosensory investigation of conspecifics by the loss of hormones as previously described. Moreover, acute administration of E2 prior to mild stress in orchiectomized male mice (for timeline see FIG. 1H) prevented the development of both sociability deficits (FIG. 1I) and anhedonia (FIG. 1J), whereas administration of E2 after stress reversed only the anhedonia phenotype (FIG. 1J). These data reveal that E2 levels and ERβ-related signaling mediate susceptibility to sub-threshold social stress in male mice.

Next, the neural circuit underlying the role of ERβ in male stress susceptibility was considered. There is ample evidence implicating the nucleus accumbens (NAc) in reward responses and stress resilience/susceptibility. Thus, it was investigated whether there are strong ERβ-expressing projections to the NAc by injecting a cre-sensitive retrograde adeno-associated virus into the NAc of mice expressing cre from the endogenous ERβ promoter (ERβ-icre). A novel ERβ circuit was identified that projects from the basolateral amygdala (BLA) to the NAc (FIG. 1K). Although known BLA-NAc projections are glutamatergic, ERβ expressing neurons in several amygdala subregions are considered exclusively GABAergic. Whole-cell patch-clamp electrophysiology was used to demonstrate that ERβ-expressing/NAc-projecting BLA neurons release glutamate, but not GABA (FIGS. 1I-1N).

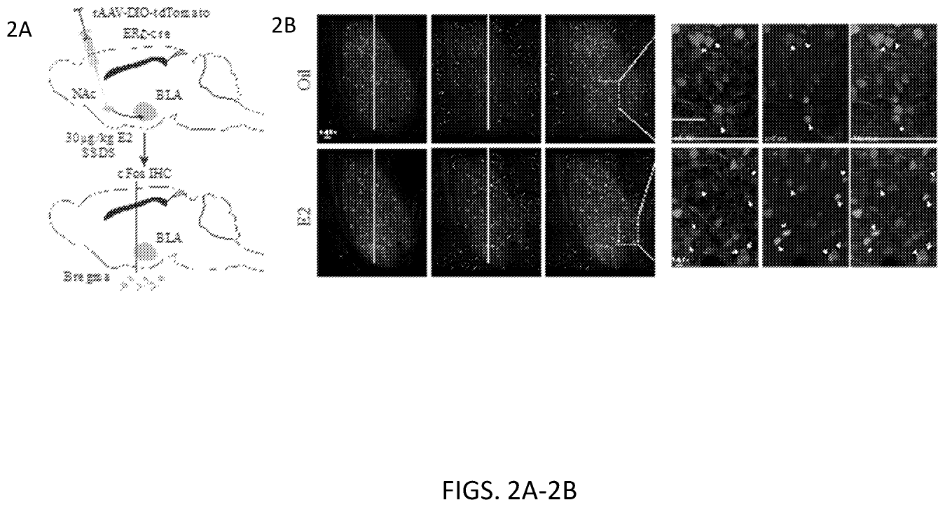

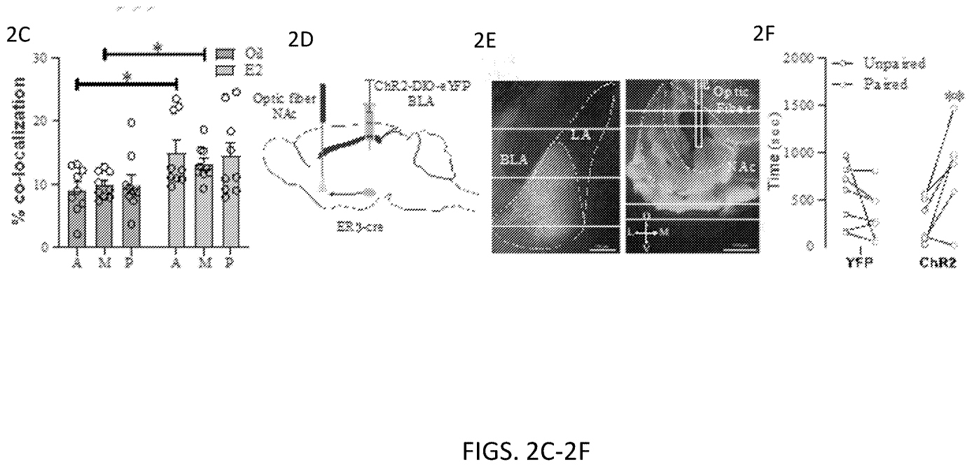

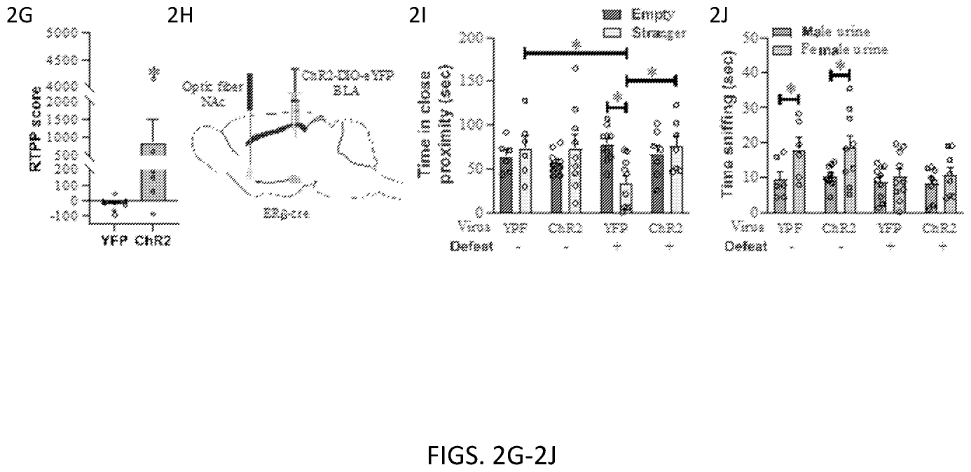

To test whether this projection is involved in stress-susceptibility, mice were orchiectomized, treated acutely with either E2 or vehicle (FIGS. 1H-1J), and underwent subthreshold social defeats, and c-Fos immunochemistry was used as a marker of BLA-NAc activation. Prior to E2 administration, mice received a NAc injection of Cre-sensitive rAAV, which is retrogradely transported to the BLA. The results demonstrate that following a brief stress exposure, ERβ-expressing BLA-NAc neurons are activated at a significantly higher degree in mice that received E2 (FIGS. 2A-2C). The possible involvement of the BLA-NAc ERβ-neuronal projection in reward was investigated by assessing place preference to optogenetic activation of this circuit. Optogenetic activation of the ERβ-expressing BLA terminals in NAc induced robust real-time place preference in male mice that received ChR2 in the BLA (FIGS. 2D-2G). To investigate whether manipulation of this projection might underlie the enhanced stress susceptibility observed, mice received an injection of ChR2 or YFP in the BLA (FIG. 2H) and were orchiectomized prior to subthreshold social defeats. Light activation of the ERβ-expressing BLA-to-NAc terminals prevented stress-induced social avoidance in orchiectomized mice, while no effect was observed in non-stressed controls, or in the FUST (FIGS. 2I-2J). These data indicate involvement of E2 mediated circuits in the development of maladaptive behaviours in males following exposure to mild stress.

The above circuit mechanisms have clear relevance to depression in veterans occurring as a consequence of stressful life events. The specific role of chronic circulating hormones in stress susceptibility was determined by assessing whether testosterone replacement would reverse stress-induced social interaction and hedonic deficits. Mice underwent orchiectomy or sham surgeries and were implanted with either testosterone-filled or empty/control silastic tube implants. Following 10 days of recovery and treatment, mice underwent subthreshold social defeat stress followed by social interaction and anhedonia tests (for timeline see FIG. 3A). Testosterone replacement reversed the observed maladaptive behaviors in orchiectomized mice (FIGS. 3B-3D), while no effect of testosterone was observed in gonadally intact or non-stressed mice regardless of gonadal status (FIGS. 3B-3D). Although testosterone replacement therapy is effective for the treatment of refractory-depression in men, potentially serious life-threatening side effects exist, such as polycythemia and cardiac dysfunction. Thus, potential options that lack testosterone's side effects and demonstrate specificity of testosterone's CNS action in mediating stress susceptibility are needed.

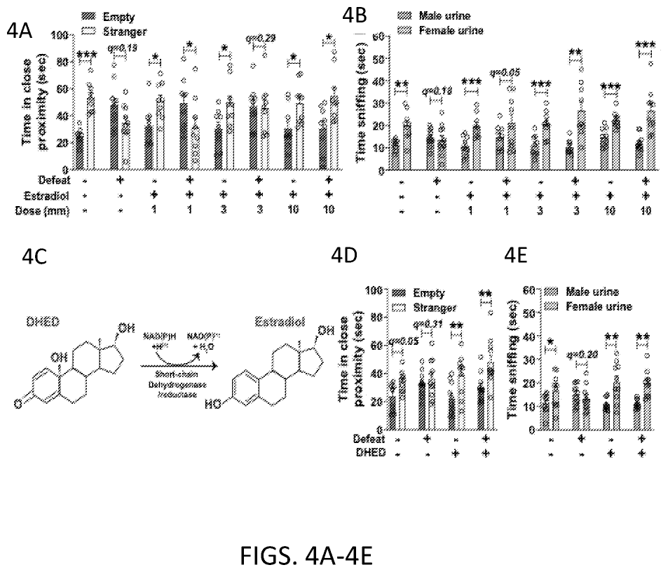

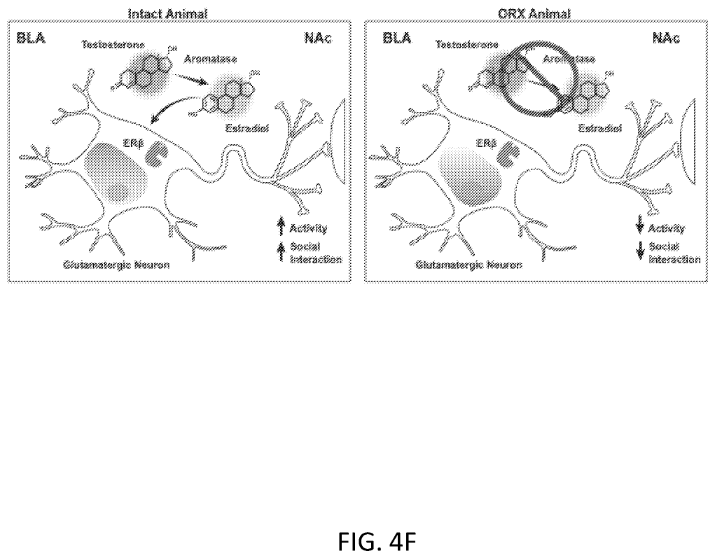

It was next demonstrated that blockade of the androgen receptor with flutamide, the only site of testosterone action, in intact mice induced neither social deficits (FIG. 3E) nor anhedonia (FIG. 3F) following stress, bringing into question the direct involvement of testosterone in stress susceptibility per se. Consistent with an E2-specific mechanism of testosterone action, it was demonstrated that treatment with letrozole, which blocks the aromatization of testosterone to E2, induced a stress-susceptible phenotype in intact male mice, as shown by deficits in social interaction (FIG. 3G) and anhedonia (FIG. 3H). These endophenotypes are consistent with what was observed with hypogonadism, indicating that the absence of E2 induces stress susceptibility independent of testosterone in males. In agreement with this conclusion, and earlier acute administration data (FIGS. 1H-1I), it was demonstrated that chronic treatment with E2 using silastic tube implants, reversed the stress-induced social avoidance (FIG. 4A) and anhedonia (FIG. 4B) in hypogonadal male mice. Moreover, acute administration data (FIGS. 1H-1I) suggests that E2 can act both as a prophylactic therapy, and, in part, as an antidepressant treatment following the development of the disease. Effectiveness of steroid administration and blockade was confirmed by changes in body, testis, and seminal vesicle weights. It is note that hormonal and other manipulations did not affect the aggressiveness of CD1 mice during stress as demonstrated by the similar number of attacks.

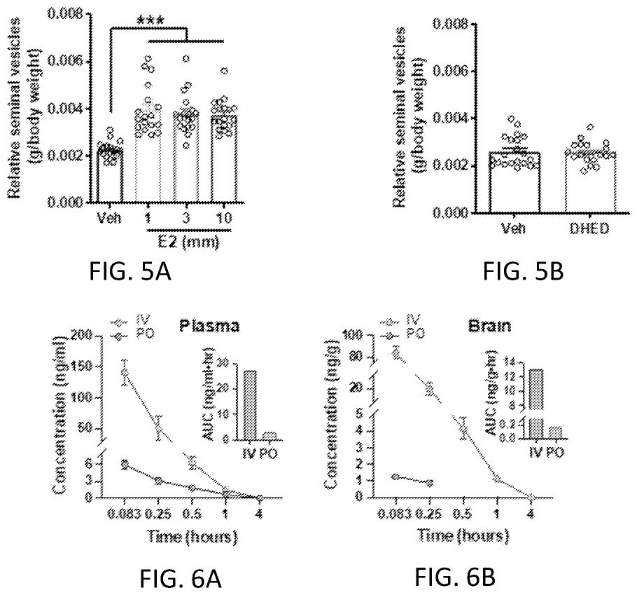

Although these findings support a critical role for E2, and not testosterone itself, as the biologically active hormone mediating stress susceptibility in hypogonadal male mice, E2 replacement therapy cannot be considered as a viable treatment in human male veteran populations due to its peripheral side effects, including gynecomastia and erectile dysfunction. However, isolating the effects of E2 to the central nervous system would provide a viable therapy. A previous study that revealed that 10β,17β-dihydroxyestra-1,4-dien-3-one (DHED) acts as a prodrug (FIG. 4C), that delivers E2 selectively in the brain via conversion of the prodrug by a NADPH-dependent reductase and hence avoiding E2's peripheral effects. Consistent with previous reports chronic treatment with DHED in mini osmotic pumps, in contrast with E2 treatment, did not increase body weight and seminal vesicle weight compared with vehicle-treated mice (FIG. 5) further supporting that DHED lacks E2 peripheral effects. More importantly, it was also found that chronic treatment with DHED via mini osmotic pumps reversed the social interaction and hedonic deficits induced by the combination of stress and hypogonadism (FIGS. 4D-4E). While E2 administration is not a viable option for the treatment of male depression due to the peripheral effects, these findings provide evidence for an exciting novel approach and suggest viability of future treatments for veterans via brain-selective E2 delivery. As a next step in advancing this treatment, the oral bioavailability of DHED was assessed, comparing oral versus intravenous delivery routes. It was found that only 10% oral bioavailability measured in plasma and less than 2% measured in brain (FIGS. 6A-6B).

Overall, it was demonstrated that E2 has a causal relationship and plays a pivotal role in male stress-susceptibility in an animal model relevant to depression. It was concluded that E2 but not testosterone levels per se mediates susceptibility to stress in hypogonadal males (FIG. 4F). These findings provide translationally relevant and paradigm-shifting evidence for the effectiveness of targeting utilizing the application of E2 as an antidepressant approach in males, as well as its studying underlying circuit-based mechanisms in response to stress. Beyond depression there other psychiatric disorders, in particular PTSD, that result in part from stress that may be amenable to this therapeutic approach. Thus, this basic science discovery is translated for the benefits of veterans in need of care for stress-related disorders. The proposed experiments advance the knowledge regarding a novel treatment mechanism for depression and PTSD, and further the early discovery and development of a brain selective and orally bioavailable prodrug of E2 based on the structure of this previously characterized molecule.

Sample sizes for the proposed experiments are estimated based on previous data, experience performing similar experiments, and power analyses. Animals are randomly assigned to treatment conditions and experimenters will remain blind during the collection and analysis of data. Drugs and chemicals are sourced from commercial supplier. All experiments include a control condition randomized within the study design. Statistical analyses are performed using STATISTICA software v6. D'Agostino-Pearson and Brown-Forsythe tests are used to assess equivalence of normality and homoscedasticity among groups, respectively. If these statistical parameters are met, parametric ANOVAs are used, followed by Holm-Šidák post-hoc comparisons. In some embodiments, these assumptions may not be met, and non-parametric equivalents are used (i.e., Kruskal-Wallis or Friedman repeated measures one-way ANOVA on ranks), followed by Dunn's correction for α error.

Biological variables: Experimental animals used within individual experiments are experimentally naïve (not used in previous experiments), and of the same age (see below age-range for each aim or experiment). For injection treatment studies, animals are weighed prior to each injection, and the amount of drug administered is based upon this weight. Silastic implant dose is not adjusted specifically to each animal's weight as the implants are prepared days prior to the surgical implantation. Cholesterol is not used as a control for E2 or testosterone as there is evidence of cholesterol's transformation to hormones in vivo. Male or female animals are utilized as described in the individual experiments.

Determine the broader efficacy of E2 in rodent models of psychiatric disease treatment effectiveness. Prior data indicate a critical role of E2 signaling in mediating maladaptive responses to stress following subthreshold social defeat stress. This finding has implications for the treatment of depression particularly associated with testosterone deficiency in males. However, there are a number of other stress-related psychiatric disorders in males which may similarly benefit from enhancing E2 signaling both as a consequence of low levels of circulating testosterone (and as a consequence low E2 in the brain) and in individuals with normal levels of gonadal hormones. Thus, the potential role of E2 signaling in the male brain is examined in additional rodent models of stress susceptibility relevant to the pathophysiology and treatment of depression and PTSD. The data is extended with rigorous experiments, and treatment effectiveness is defined in relevant disease models. The working hypothesis that in males E2 is involved in susceptibility to diverse stressors and is effective as a treatment for maladaptive behaviors that emerge as a consequence of those stressors is examined. The experimental approaches used are primarily behavioral, combined with neuroendocrine manipulations. Specific models tested, with relevance to neuropsychiatric disorders that disproportionately affect veterans include hypogonadism induced stress susceptibility relevant to depression, and stress-induced changes in startle response, and fear retention and extinction, relevant to PTSD. In select experiments, responses in males are compared to those of females. Completion of these experiments allows a better understanding of how E2 signaling interacts with stress, and in particular, its largely unexplored role as a susceptibility factor and treatment in the male brain. These findings provide models for studies to assess effects of oral DHED prodrug on reversal of psychiatric disease phenotypes identified.

Unless otherwise noted, consistent with prior data, all experiments in this aim use 8-12 week old C57BL/6J mice. Mice are gonadectomized 12 days prior to other experimental procedures. For the chronic administration paradigms, silastic tubes (1.02 mm inner diameter×2.16 mm outer diameter) are filled with testosterone or E2 (Sigma-Aldrich, St. Louis, MO, USA). Silastic implants are primed in 0.9% saline at 37° C. overnight prior to subcutaneous implantation to the mid scapular region of orchiectomized mice (e.g. FIGS. 3A-3H). This allows for stable release of hormone molecules over a long time-course. A standard dose of both testosterone and E2 is 10 mm (FIGS. 4A-4F), which is based upon previous literature. Unless otherwise noted, all experimental procedures utilize 12 mice per experimental group. Assuming the predicted medium to large effect sizes, the experiments described have all been sufficiently numbered to allow greater than 80% power to detect both main effects of each factor and, when relevant, interactions between factors.

Subthreshold social defeat stress: Retired CD1 breeders are singly housed and used as aggressors. Experimental mice are introduced to the home cage of an aggressor for 2 min to initiate the physical attack phase. Afterward, mice are transferred and housed on the opposite side of the aggressor, in the same cage separated by a perforated Plexiglass divider for 15 mins in order to maintain sensory contact. This process is repeated 3 times over a period of ~50 min.

Social Interaction: Mice are placed in a rectangular box (40 cm length×30 cm width×35 cm height; Stoelting, IL) divided into three compartments (two equal-sized end-chambers and a middle chamber) for five minute habituation phase (10-15 lux). After the habituation phase, two small wire cages are introduced into the two end chambers, one containing an unfamiliar, non-aggressive CD1 mouse and the other remaining empty. The amount of time mice spent sniffing each cage during the five-min test is assessed using CleverSys tracking software (CleverSys, Inc, Reston VA, USA).

Female Urine Preference Test: is performed 24 hours after the social interaction test as using previously described methods. The amount of time mice spent interacting with a cotton-tipped applicator soaked in either fresh male or female mouse urine during a total of 3 minutes is analyzed by an experimenter blind to the experimental groups. Focus is on these tests, as they have been found to be the most sensitive among distinct tests of stress susceptibility.

Assess sensitivity to social defeat stress in testosterone deficient male mice. These experiments thus far have largely modeled effects of the near absence of testosterone, due to complete removal of testicles. While this models a condition some veterans face (e.g. those who suffer from hormone-sensitive prostate cancer and therefore chemically or surgically castrated), many more veterans suffer from low testosterone conditions associated with advanced age or other conditions. It is proposed that low levels of testosterone are also be associated with susceptibility to subthreshold social defeat stress. The aromatase inhibitor experiment (FIG. 3G) likely caused a reduction, but not the complete elimination, of E2 production in the brain and resulted in mice susceptible to subthreshold social defeat. As such, the goal in this experiment is to substantially reduce, but not eliminate, circulating testosterone to better mimic human conditions. Testosterone levels associated with advanced age are first examined and a dose response experiment is conducted to determine a dose range that will model different circulating levels of testosterone. The following groups of mice (n=8/group) are compared: (1) 18 month old, aged mice obtained from the NIA aged rodent colony; (2) 24 month old, aged mice obtained from the NIA aged rodent colony; (3) Sham surgery; and (4-7) ORX+testosterone at doses of 0.3, 1, 3, and 10 mm testosterone packed into a silastic implant. Ten days later, plasma is collected, and samples sent for testosterone and E2 analysis. A testosterone dose is selected that results in circulating testosterone levels similar to that observed in 24 months old aged mice (low dose) and also a dose (middle dose) between this dose and the standard 10 mm dose. Susceptibility to sub-threshold social defeat stress are assessed in the following groups: (1) Sham Surgery+empty silastic tube; (2) ORX+empty silastic tube; (3) ORX+testosterone (Dose low) containing silastic tube as determined from the above experiment; (4) ORX+testosterone (Dose middle) containing silastic tube as determined from the above experiment; (5) ORX+testosterone (Dose 10 mm) containing silastic tube. Twenty-four hours after subthreshold social defeat stress, mice are tested for social interaction and female urine preference (as in FIGS. 3A-3H and FIGS. 4A-4F). It is anticipated that mice treated with the low dose show susceptibility similar to the empty silastic tube. If that proves to be the case, experiments combining low dose testosterone with the administration of E2 in orchiectomized mice are conducted, predicting that E2 administration will completely recover the phenotype induced by low testosterone.

Assess effects of E2 on advanced age-dependent social defeat stress susceptibility in males. It has previously been reported that aging increases susceptibility to standard social defeat paradigm. It is examined how low testosterone, associated with advanced age in mice, influences susceptibility to social stress and the role of E2 in those responses. It is hypothesize that stress-susceptibility will increase with age, inversely correlate with circulating levels of testosterone, and be prevented by E2 administration. Mice are obtained from the NIA Aged Rodent Colony. Mice are obtained at 12, 18, 24, and 30 months of age (though this may change somewhat due to colony availability) to compare to younger animals at 2 and 6 months. These experiments proceed in multiple cohorts over an extended period due to current NIA aged colony restrictions of a total of 10 animals delivered every other month. Different ages of mice are equally represented in each cohort. Twenty-four hours after subthreshold social defeat stress, mice are tested for social interaction and female urine preference (FIGS. 3A-3H and FIGS. 4A-4F). Following testing, mice are euthanized, plasma are collected, and samples analyzed for testosterone analysis. Pearson (parametric) or Spearman (non-parametric) correlation coefficient analysis are performed as appropriate to investigate whether there is a relationship between the levels of testosterone and development of maladaptive behaviors. It is anticipated that 1) susceptibility to subthreshold social defeat stress is significantly increased in mice aged 24 and 30 months compared to controls, and 2) circulating levels of testosterone are inversely correlated with susceptibility to subthreshold social defeat stress. If these predicted effects are observed, treatment continues with E2 and assessment for reversal. Gonadally intact aged mice (24-30 months of age) are implanted with either empty silastic tubes, silastic tubes containing testosterone, or silastic tubes containing E2 (both low and high dose). It is predict that both testosterone and E2 will protect against stress induced decreases in social interaction and female urine preference.

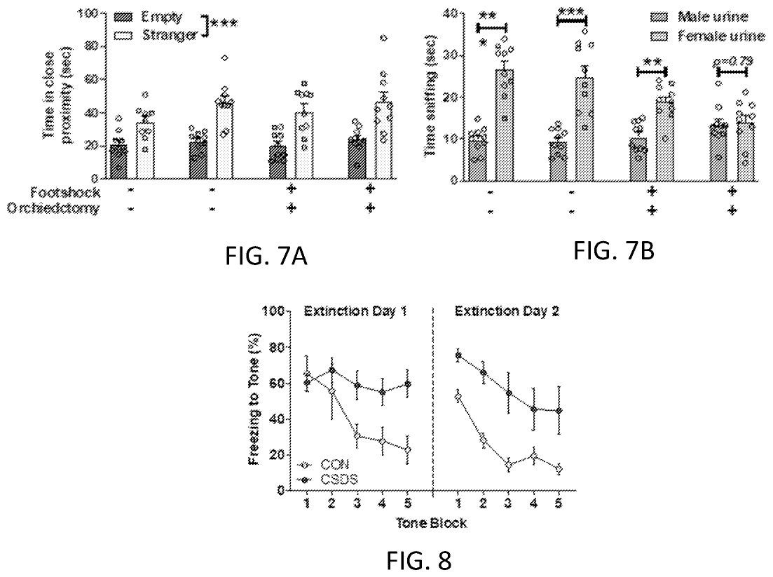

Determine effects of E2 on gonadectomy-induced susceptibility to inescapable traumatic shock stress in males and females. Most of these experiments assess the effects of social defeat stress. In this experiment the effects of an alternative type of stress-inescapable foot shocks are assessed—to determine whether the observed biological effects of E2 are stress specific. Prior experiments were conducted, finding no effect on social interaction behaviors, but orchiectomy+foot shock stress resulted in deficits in female urine preference (FIGS. 7A-7B). The effects of this stress on maladaptive behaviors and their possible reversal by testosterone and E2 are assessed. The following groups of mice are used for this experiment: (1) Sham Surgery+empty silastic tube; (2) ORX+empty silastic tube; (3) Sham Surgery+testosterone (10 mm) containing silastic tube; (4) ORX+testosterone (10 mm) containing silastic tube; (5) Sham Surgery+E2 (10 mm) containing silastic tube; (6) ORX+E2 (10 mm) containing silastic tube. Mice are exposed to inescapable foot shock (0.3 mA shocks; 180 shocks at 15 sec intervals) in a two-chambered shuttle box with a permanent divider in place. Controls are placed in the same chambers but will not receive shocks. Twenty-four hours later mice are subsequently tested for social interaction and female urine preference. As a comparison to male results, a similar experiment is conducted in females with the following groups: (1) Sham Surgery+empty silastic tube; (2) OVX+empty silastic tube; (3) Sham Surgery+E2 (10 mm) containing silastic tube; (4) OVX+E2 (10 mm) containing silastic tube. It is predicted that the effects of orchiectomy in males are reproduced to enhance stress-induced decreases in female urine preference deficits, which will be prevented by both testosterone and E2. It is anticipated that ovariectomy of females have similar effects, which are prevented by the replacement of E2.

Determine the role of E2 in the normal acquisition, extinction, and reinstatement of cued (Pavlovian) fear responses in males. The effect of orchiectomy on the acquisition, extinction, and reinstatement of fear in non-stressed male animals is determined. It has previously been reported that orchiectomy enhances tone conditioned freezing in male mice, which was prevented with systemic testosterone administration. It has also been reported that orchiectomy delayed extinction of fear responses in male rats. The role of E2 is known in mediating fear responses, extinction, and related cognitive functions in female rodents. Fear conditioning takes place in Coulbourn shuttle boxes. The floor of the chambers consists of parallel rods connected to a Coulbourn shock source that delivers foot shocks. A speaker mounted on the top of the chamber delivers an acoustic conditioned stimulus (pure tone). On Day 1, mice are placed in the chamber to acclimate for 3 minutes (Context A). After that time, a series of 5 auditory conditioned stimuli (CS; 3.5 KHz, 80 dB, 30 sec) are co-terminated by a scrambled foot shock (unconditioned stimulus, US; 0.7 mA, 2 sec) separated by 2 min inter-trial intervals. On Day 2, mice undergo extinction training in a novel context (Context B) that consists of a Coulbourn test chamber that is distinct from the conditioning chamber in terms of wall pattern, flooring texture, lighting, and scent. Mice are placed in the chamber for a 3 min habituation period, wherein baseline freezing to the novel context will be assessed. Following habituation, 15 auditory conditioned stimuli of the same tone duration, pressure, and frequency as with conditioning are delivered, with inter-trial intervals of 1 min. The percentage of time spent freezing during tone presentations issued to assess acquisition of the conditioned fear, which will also serve as an initial assessment of extinction. On Day 3, the animals are exposed to an extinction retention session in Context B, identical to the procedure carried out on Day 2. One week later, mice are assessed for reinstatement of the conditioned fear. This involves exposing mice to an unsignaled shock in Context A, and reassessing freezing to repeated presentations of the tone in Context B, as on Day 2 and 3. Importantly, the acquisition, extinction, and reinstatement phases of the experimental design recapitulate 1) the conditions that elicit pathological fear (acquisition), 2) the exposure therapy that is needed to attenuate the fear response (extinction), and 3) circumstances that often provoke relapse (reinstatement). This approach was based on the understanding that pharmacological treatment is often more effective when combined with exposure therapy, which together, optimizes resiliency against environmental triggers that remind the individual of the traumatic event. Time spent freezing across CS presentations during the extinction procedure (Days 2 and 3) is assessed with Coulbourn automated FreezeFrame software. Freezing is defined as the absence of movement aside from what is required for respiration. The experimental groups are: (1) Sham Surgery+empty silastic tube; (2) ORX+empty silastic tube; (3) Sham Surgery+testosterone (10 mm) containing silastic tube; (4) ORX+testosterone (10 mm) containing silastic tube; (5) Sham Surgery+E2 (10 mm) containing silastic tube; (6) ORX+E2 (10 mm) containing silastic tube. It is expected (consistent with earlier reports) that orchiectomy enhances fear conditioning and reduce the rate of extinction and that these effects are prevented by testosterone. It is predicted that E2 treatment will also reverse this effect, implicating E2, and not testosterone per se in exerting protective effects. If differences between the groups are detected, then whether these effects are due to altered shock sensitivity, are assessed. These results are compared to the extensive existing literature in females demonstrating that low E2 levels impairs fear extinction and administration of E2 can reverse these effects.

Determine the role of E2 in stress-moderated acquisition, extinction, and reinstatement of fear responses in males. Animal approaches to study PTSD should include an inescapable severe stressor. These experiments assess interactions between a stress and the effects of E2. The prior experiment may show that effects of testosterone on learned fear and its extinction are mediated directly by testosterone, or that they are mediated by E2. For either result, it is critical to understand if E2 mediates interactions with stress. This is because the acquisition of fear is an adaptive response that is not considered pathological in and of itself. However, veterans have often encountered stressful circumstances that lead to an exaggerated and pathological state of fear, which is both resistant to extinction and highly susceptible to relapse. Considering that social stress is an important risk factor contributing toward the onset of PTSD in response to a traumatic event, the social defeat stress paradigm is utilized in order to model the conditions under which veterans develop PTSD. Prior experiments with gonadally intact mice have revealed that mice exposed to a standard 10-day CSDS procedure display decreased extinction relative to controls (FIG. 8). It is anticipated that sub-threshold SDS (rather than 10 day) paradigm is sufficient to induce effects in orchiectomized mice. The following groups are tested: (1) Sham+empty silastic tube; (2) Sham+stress+empty silastic tube; (3) ORX+empty silastic tube; (4) ORX+stress+empty silastic tube; (5) ORX+testosterone (10 mm) containing silastic tube; (6) ORX+stress+testosterone (10 mm) containing silastic tube; (7) ORX+E2 (10 mm) containing silastic tube; (8) ORX+stress+E2 (10 mm) containing silastic tube. Mice are assessed for social interaction deficits 24 hours after the subthreshold defeat sessions to determine if they are resilient or susceptible. n=18/mice per group is used to account for only a subset of mice being susceptible to stress. It is predict orchiectomy-induced susceptibility to social defeat as in prior data (FIGS. 3A-3H). Subsequently, mice undergo conditioning, extinction, and reinstatement, as described above. It is predicted that stress attenuates the extinction of conditioned fear and reduce the propensity of relapse in the reinstatement procedure and that this is reversed with testosterone, as well as E2 treatment. It is also predicted that mice defined as “susceptible” in the social interaction outcomes show greater enhancement of learned fear and less efficient extinction.

Determine the role of E2 in traumatic stress-induced changes in startle response in males and females. Heightened startle reactivity is a DSM diagnostic criteria for PTSD. Individuals suffering from PTSD frequently report subjective elevations in startle response and objective studies have described increased startle reactivity in individuals with PTSD. Moreover, physiological startle response is correlated with the severity of self-reported hyperarousal symptom domains and attenuated by interventions that improve PTSD symptoms. In rodents, like humans, the startle response can be elicited by acoustic stimuli (acoustic startle response, ASR) and involves eyelid closure and skeletal muscle contractions. While ASR is frequently assessed via the eye blink reflex in humans, it can be accurately assayed via whole-body movements in rodents. Importantly, elevated ASR has been reported in rodents following traumatic stress, including electrical shock and social defeat stress. ASR is utilized as a measure of hyperarousal in mice following traumatic stress. Similar to previously described methods, ASR is assessed following acute foot shock stress. Baseline ASR is assessed over 10 startle stimuli (110 dB, pseudo-randomized average inter-stimulus interval of 30 sec). Mouse ASRs do not habituate to such a brief exposure, allowing retesting of mice to assess effects of a subsequent stress. 24 hours after a first assessment, mice are exposed to acute traumatic foot shock stress (10 shocks, 0.45 mA, 1 sec, mean randomized inter-shock interval of 150 sec). Changes in ASR are measured 24 hours after shock using the same startle protocol. Experimental groups are: (1) Sham+empty silastic tube; (2) Sham+testosterone (10 mm) containing silastic tube; (3) Sham+E2 (10 mm) containing silastic tube; (4) ORX+empty silastic tube; (5) ORX+testosterone (10 mm) containing silastic tube; (6) ORX+E2 (10 mm) containing silastic tube. It has previously been reported that orchiectomy has no effect on the acoustic startle response. It is also predict that stress+orchiectomy will result in increased startle compared to stress alone, recoverable by testosterone and also E2 treatment. These experiments are repeated with female mice, to compare with effects observed with males. The effects of ovariectomy are compared with or without replacement of E2.

At completion of these experiments the potential role of E2 in the male brain in rodent models of stress susceptibility in males relevant to the pathophysiology and treatment of depression and PTSD is determined. The role of low testosterone, in addition to the absence of testosterone, in mediating stress susceptibility, and the ability to reverse such effects by administration of E2, are defined. Similarly, it is predicted that aged mice will manifest increased susceptibility to stress, which will be recovered by the administration of E2. Orchiectomy will likely increase the development of cued fear conditioning, which is exaggerated by stress, and it is predicted this modulation by stress will be prevented by E2 treatment. It is expected that the protective effects of E2 extend to a distinct type of stress, inescapable foot shocks, as well as stress-potentiated startle associated with gonadal deficiency. As part of the experimental design (including intact animals), effects of E2 in modulating response to diverse stressors in gonadally intact mice are assessed in some experiments. The importance of these experiments is a determination of whether enhancement of E2 signaling is relevant to males with normal testosterone. Thus, a potential value of E2 is determined, and potentially of DHED prodrug in treating stress-related disorders not associated with hypogonadism. For some experiments, treatment with the aromatase inhibitor, letrozole, is adminstered (as in FIG. 3G) and it is expected the effects of orchiectomy will be phenocopied. However, if testosterone, but not E2, administration is effective in reversing orchiectomy-induced deficiencies, then the role of testosterone acting on the androgen receptor is confirmed by using the androgen receptor antagonist flutamide (as in FIG. 3E), which is expected to be sufficient to phenocopy the effects of orchiectomy.

In a non-limiting embodiment, if testing in the social interaction or female urine outcomes does not yield significant group differences, other behaviors, for example sucrose preference, operant tasks, or other measures of anhedonia, are tested for. It is anticipated that administration of low dose of testosterone will result in susceptibility similar to the empty silastic tube that may be evidenced by the development of maladaptive behaviors following subthreshold social defeat stress. In non-limiting embodiments, it may be that a greater level of stress is necessary to induce changes and if subthreshold defeat is not successful, different levels of social defeat stress are tested to better gauge susceptibility. In a non-limiting example, this may result in a low dose that does not confer susceptibility to the standard subthreshold social defeat stress paradigm, but perhaps to a 5-day social defeat paradigm, which is less than the typical 10 day paradigm typically employed. In a non-limiting example, if application of low dose testosterone is not successful, a chemical castration approach is considered (e.g. LHRH agonist or antagonist), which could be titrated to reduce, but not eliminate, testosterone production. In a non-limiting example, if there are difficulties eliciting maladaptive behavioral responses (in either male or female mice) using shock stress, a witness social defeat paradigm that will allow direct comparison of experimental results between male and female mice is used.

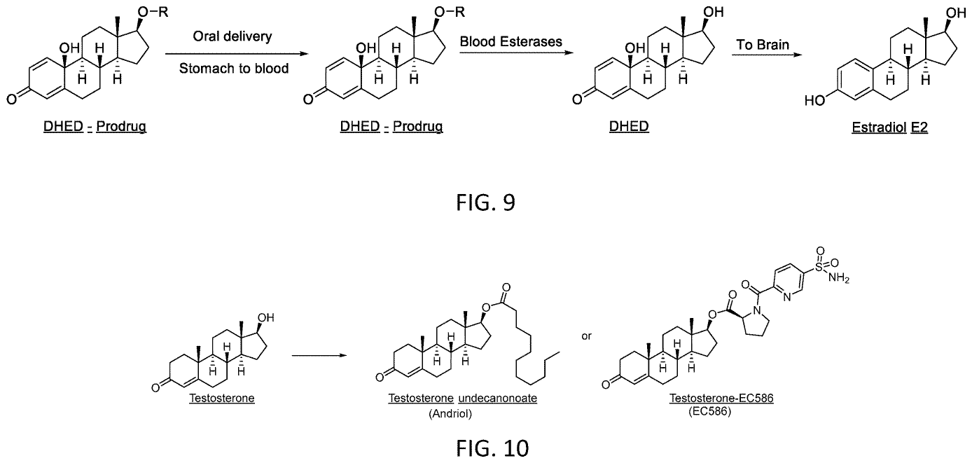

Optimize brain-selective estradiol for improved oral bioavailability properties. DHED is a previously characterized naturally-occurring precursor of E2 with the ability to selectively deliver E2 to the brain. However, it was found that DHED's potential clinical utility as a human therapy is limited due low oral bioavailability, and potentially its short half-life and (FIGS. 6A-6B). Modifications to the DHED structure could have pronounced effects in extending the half-life and increasing oral bioavailability resulting in an ideal drug candidate to treat estrogen-responsive neuropsychiatric disorders. Novel, orally bioavailable prodrugs of DHED as a therapy for estrogen-responsive neuropsychiatric disorders are examined (FIG. 9). Thus, the objective is to test novel synthetic prodrugs of DHED for improved pharmacokinetic properties, and to test efficacy of identified molecules in translatable neuropsychiatric disease models. A prodrug strategy for DHED to increase DHED's oral bioavailability is pursued (FIG. 10). The working hypothesis that addition of ester or carbonate modifications at the c17 position of DHED will improve oral bioavailability, as well as half-life, compared to the parent DHED molecule and provide similar efficacy in animal models of depression and PTSD symptoms compared to the s.c. administration of E2 or DHED (as positive controls) is tested. To test this hypothesis, synthetic chemistry, pharmacokinetics, in situ hybridization, qPCR, and rodent behavioral models are used. The oral bioavailability of DHED prodrugs in mice and rats are obtained by comparing intravenous and oral administration by gavage in both male and female animals. The prodrugs with sufficient oral availability and increased half-life of the DHED molecule are tested in vivo for response to E2 sensitive biomarkers and medication efficacy measures. The expectation is that at the completion a prodrug of DHED with improved oral bioavailability and other pharmacokinetic properties is identified, and will allow for the development of a new molecule to potentially treat stress-related disorders, including depression and PTSD, as well as potentially other psychiatric disorders.

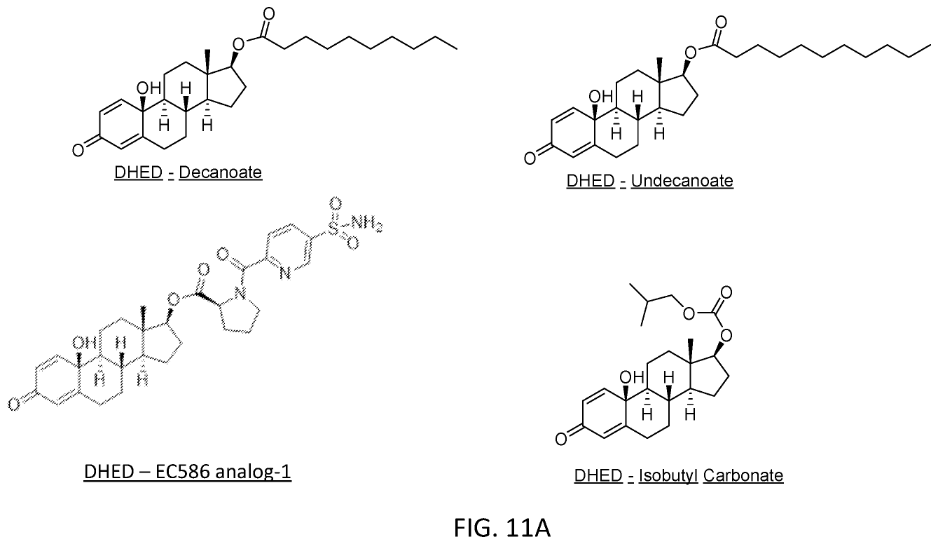

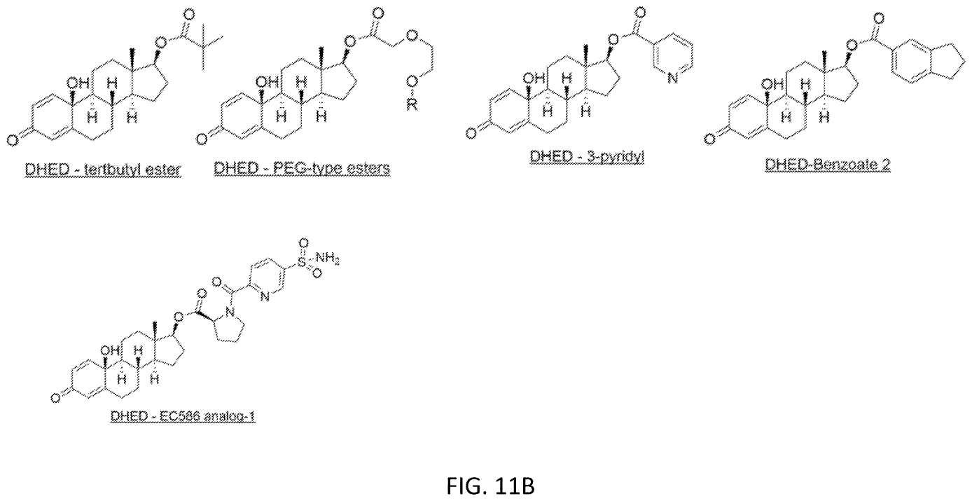

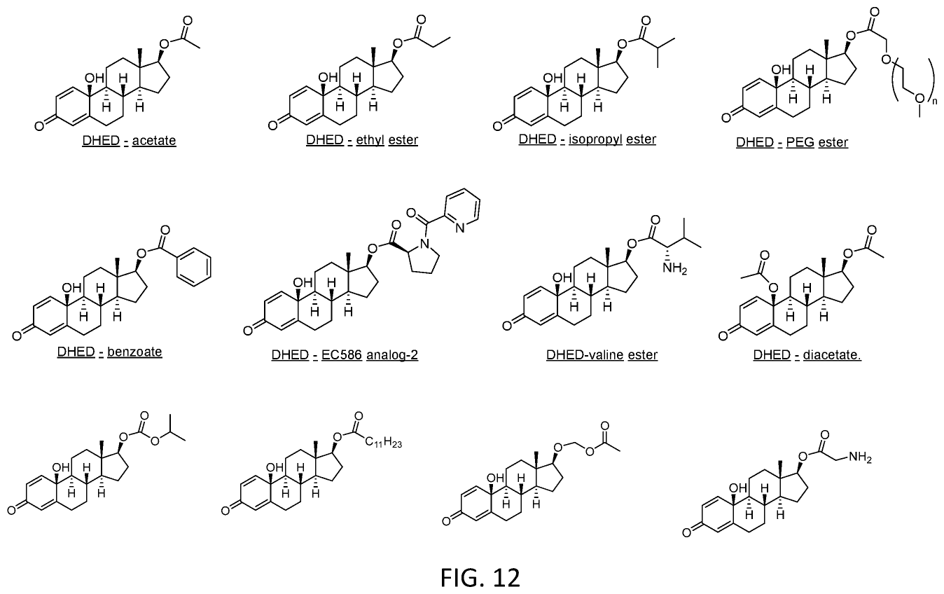

Synthesis of Candidate Prodrugs. To improve oral bioavailability, prodrugs are synthesized by introducing ester modifications onto the c17 position of DHED. It is hypothesized that a fatty acid ester may increase relative bioavailability. Once absorbed into the bloodstream, such a prodrug is typically efficiently converted to the parent drug by esterases circulating in blood. Thus, a number of ester prodrugs at the c17 position of DHED were designed, focusing on derivatives with high probability of improving oral bioavailability. Initial focus is on the DHED-decanoate and DHED-undecanoate (which differ by one carbon) due to hypothesized favorable ADME data. Non-limiting examples of proposed candidate prodrugs are shown in FIGS. 11A, 11B, and 12. The isobutyl carbonate prodrug was designed to hydrolyze more rapidly once the compound reached the plasma, providing a rapid release of DHED in vivo. In a non-limiting example, the compounds shown in FIG. 12 can be used comparators to very generally define how structure of the ester group imparts changes to DHED oral absorption and other ADME properties. Synthesis of the prodrugs is straight forward. Treatment of the commercially available DHED with the relevant appropriate acid chloride tetrahydrofuran, using diisopropyethylamine as a base gives moderate yields of the desired prodrugs on small scale, with only a single acylation event observed under these conditions. The DHED-decanoate and DHED-isobutyl carbonate compounds have been synthesized and are characterized by standard synthetic organic chemistry techniques (¹H NMR, ¹³C NMR, HRMS, LC-MS), and initial ADME studies (aqueous solubility, PAMPA, microsomal metabolic stability) for basic biophysical properties. In addition, plasma stability for the prodrugs is obtained. Low microsomal stability and low plasma stability is not necessarily a poor outcome for these prodrugs, as it is expected the prodrugs to convert in the plasma to the free DHED. The synthesis and these characterizations for all compounds is completed.

Pharmacokinetic (PK) assessment of prodrugs. As detailed in FIGS. 6A-6B, DHED has poor oral bioavailability, which limits its usefulness as a human drug treatment. Generally, this PK study and analysis proceeds similarly to previous studies aimed at assessing the oral bioavailability of (2R,6R)-HNK. As in previous data with DHED (FIGS. 6A-6B), male C57BL/6J mice (n=6/time point) receive 200 μg of prodrug in 0.9% (20% cyclodextrin) administered either IV or PO. Plasma and brain are collected at 5 min, 15 min, 30 min, 1 h, and 4 h after administration. PK is performed using tandem liquid chromatography-mass spectrometry (LC-MS/MS) for quantification. Pharmacokinetic analysis is performed using Phoenix WinNonLin. Non-compartmental analysis is performed to determine the following parameters: C0 (Back-extrapolated concentration at time 0); Cmax (Maximum observed concentration); Tmax (time of maximum observed concentration); AUC0-t (Area under the concentration-time curve from hour 0 to the last measurable concentration, estimated by the linear trapezoidal rule). Statistical significance is calculated using the unpaired t-test or Mann-Whitney U test for comparing AUC values. Importantly, pharmacokinetic measurements observe both the level of the prodrug and free DHED moiety in the plasma. A critical comparison is measuring the DHED levels after administration of the prodrug compared to after administration of DHED itself. The PK of top candidates is repeated in intact female C57BL/6J mice, gonadectomized male and female mice, and both male and female Sprague Dawley rats to confirm that enhanced bioavailability is not restricted to one species or hormone status. Top candidates include additional time points beyond the five time points initially studied. PK studies in non-rodent species are conducted to help predict generalizability of the finding to humans. Additional pharmacokinetic analysis is done to measure the levels of prodrug, DHED, and free E2 in both the plasma and the brain of the subject. E2 levels are measured with liquid chromatography tandem mass spectrometry (LC-MS/MS) using standard methods. When measuring brain levels animals are first briefly perfused with cold saline to flush any hormones from the circulation. If levels of E2 are too low to be quantified via this measure, and to distinguish naturally-produced from DHED prodrug-produced E2, E2 levels can be quantitated at the femtomole level utilizing deuterium-labelled E2 standards and triple quadrupole mass spectrometry.

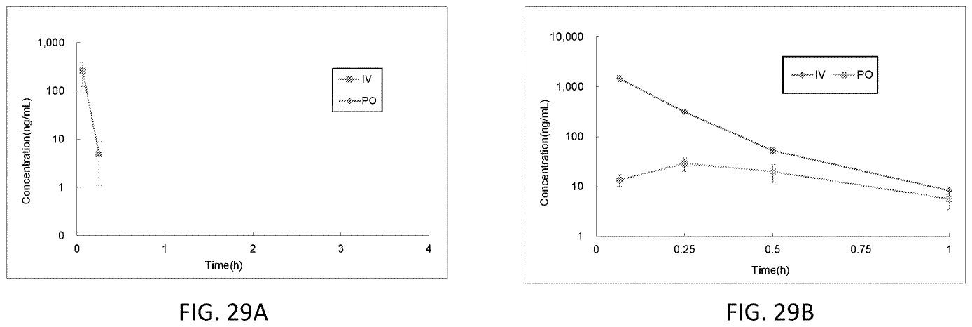

FIG. 29A shows that the DHED-acetate prodrug of Example 1.1 is very rapidly metabolized when administered via IV, and cannot be detected when administered via PO. FIG. 29B shows the PK of DHED after administration of the DHED-acetate prodrug of Example 1.1, demonstrating that the prodrug is hydrolyzed into DHED when administered either by IV or PO.

Determine dose-dependent functional effects on in vivo measures of brain versus peripheral actions of DHED prodrugs. These studies provide biological proof, in vivo, of the hypothesized brain-selective estrogen activities of prodrugs of the disclosure. It is anticipated that these studies are conducted with up to three DHED prodrugs identified in the prior experiments. Effects of compounds on progesterone receptor (PR) expression in the medial preoptic area of the hypothalamus is assessed as a marker of central estrogen effects, galanin expression in the pituitary, and C3 expression in the uterus (in females) as peripheral examples of estrogen targets. DHED increases medial preoptic area PR expression, but does not change pituitary galanin expression or uterus C3 expression. Experiments are conducted in male animals, as well as experiments in females, in separate experiments. All mice are gonadectomized as lack of gonadal hormones is necessary to see E2-mediated increase in target expression. Following 12 days, five groups of C57BL/6J gonadectomized mice (n=8/group) are treated daily by oral gavage: (1) vehicle; (2) ethynyl estradiol (EE, a water-soluble estrogen as a positive control; 200 μg/kg) as an internal positive control; (3-6) DHED prodrug at different doses determined by PK studies (e.g., 10.0, 30.0, 100.0, and 300.0 μg/kg). Mice are treated once a day for 10 days. Six hours after the final injection mice are euthanized, and their brains, pituitary glands, uteri (females only), and seminal vesicles and prostate gland (males only) are collected. Whole brains and other tissues are frozen on dry ice and later will be processed for expression of estrogen-regulated genes.

Effects on progesterone receptor (PR) expression in the brain. It has been previously shown that PR expression is upregulated by estrogens in the male as well as female medial preoptic area of the hypothalamus. The expression of progesterone receptor is measured with in situ hybridization histochemistry (ISHH) that allows precise evaluation of gene expression in well-defined nuclei/areas of the brain. Brains are cut with a cryostat and 20-am thick sections are placed on gelatin-coated slides. ISHH utilizes radioactive isotope-labeled riboprobe (cRNA) complementary to mouse PR. At the end of the hybridization, slides are placed over X-ray films and the optical density of the signal over the preoptic area of the hypothalamus is evaluated and quantified with C-Imaging (Pittsburgh, PA).

Effects on galanin expression in the pituitary. Following mRNA extraction from the pituitary with Trizol reagent (Invitrogen), total RNA is treated with DNase (Invitrogen). RNA (500 ng of total per sample) is reverse transcribed into cDNA in a 20-μl reaction using iScript cDNA Synthesis Kit (Bio-Rad). Real-time PCR is performed using the iQ SYBR Green Supermix (Bio-Rad) in a 25 μl reaction using primer pairs for galanin (F 5′-TCTCACCGCTGCTCAAGATG (SEQ ID NO: 1); R 5′-GCCATGCTTGTCGCTAAATG (SEQ ID NO: 2)) designed with the Accelrys Gene 2.0 software. Efficiency and consistency of cDNA synthesis is determined by amplification of the 18S gene, GAPDH and Actin-0, as controls. Fold changes are determined using the 2^{-ΔΔCt} method. It has been shown that galanin expression is upregulated by estrogens in the male as well as female pituitaries.

Additional assays of peripheral E2 function in females. In females, the weight of the uterus and C3 expression in the uterus is measured. The wet weight of the uterus is dramatically increased by peripheral estrogen exposure. E2 increases C3 expression in the uterus. These qPCR experiments proceed identical to the qPCR experiments, except that and a primer pair specific for C3 will be used.

Additional assays of peripheral E2 function in males. The seminal vesicles are an estrogen-responsive male reproductive tissue (FIGS. 5A-5B). Relative seminal vesicle weights are unaffected by s.c. DHED treatment, while s.c. E2 treatment produced significant increase compared to vehicle (saline) control (FIGS. 5A-5B). As such, seminal vesicles are collected and weighed. Recent studies have shown that in the prostate gland decreases in the expression of T-repressed prostatic message 2 (TRPM-2), ornithine decarboxylase (ODC), androgen receptor (AR), and ERα following E2 treatment. Thus, the expression of TRPM-2, ODC, AR and ERα in the prostate gland is evaluated.

Orally administered prodrug reversal of maladaptive behaviors induced by social defeat stress. A single prodrug from the prior experiments is selected to further test in behavioral assays. These experiments are conducted similar to prior experiments (FIGS. 3A-3H and FIGS. 4A-4F), where it was found that chronic administration of E2 (via a silastic tube) or DHED (via osmotic minipumps) resulted in the prevention of anhedonia deficits following subthreshold social defeat stress. The effects of orally administered DHED prodrug(s) to reverse maladaptive behaviors following orchiectomy and subthreshold social defeat stress in male mice are tested. Male C57BL/6J mice, 7 weeks of age, are orchiectomized, and 12 days later exposed to sub-threshold social defeat stress. Mice are then tested for social interaction and female urine preference deficits. Drugs are administered by oral gavage tube using the following groups (n=12/group): (1) Unstressed Vehicle; (2) Unstressed DHED prodrug low dose; (3) Unstressed DHED prodrug middle dose; (4) Unstressed DHED prodrug high dose; (5) Stressed Vehicle; (6) Stressed DHED prodrug low dose; (7) Stressed DHED prodrug middle dose; (8) Stressed DHED prodrug high dose. Specific doses are determined by prior PK and gene expression studies. The effects of chronic, oral gavage administration beginning immediately following orchiectomy for 10 days are first tested. The administration paradigm may depend upon PK of compounds but in a non-limiting embodiment is a single daily administration 1 hr prior to the subthreshold social defeat sessions. The chronic treatment paradigm is followed by an experiment assessing the effects of an acute, single administration 1 hr prior to initiation of stress and an acute, single administration 5 min after completion of stress.

Oral DHED prodrug reversal of additional psychiatric disease phenotypes. One goal of these experiments is to assess effects of newly identified DHED prodrug as in the previous experiments in neuropsychiatric disease models newly identified as E2 responsive. In a non-limiting embodiment, of the possible experiments that could be performed, focus is on PTSD considering the tremendous impact of PTSD on feterans. The specific experimental design will vary per the specific model. For example, if earlier experiments reveal effects of gonadectomy combined with stress on the development and/or extinction of conditioned fear, effects of orally administered DHED prodrug to reverse that phenotype using both acute and chronic administration paradigms are assessed. The selection of experimental groups and planned statistical analysis are identical as described above.

At completion of the above experiments, the discovery of a DHED prodrug with superior oral bioavailability to the parent compound is anticipated and that this prodrug molecule (or molecules) is rapidly converted to DHED in the blood, and then show the pharmacodynamic profile of DHED and EE in the brain as evidenced by increased expression of PR in the preoptic area, but unlike EE, it will not stimulate the expression of galanin in the anterior pituitary, expression of C3 and increase weight of the uterus, and increase weight of the seminal vesicle and gene expression of TRPM-2, ODC, AR and ERα in the prostate gland. This result can reveal superiority to the current standard of care. It is anticipated that the orally administered prodrug will reverse maladaptive behaviors induced by orchiectomy combined with subthreshold social defeat stress and also show efficacy in other E2-sensitive models.

In a non-limiting embodiment, the prodrugs comprise acetates, ethyl esters, benzoate, and isopropyl esters to improve permeability. In a non-limiting embodiment, the use of PEG-esters can help improve solubility for the compound, while simultaneously providing the same ease of hydrolysis as more standard esters. The EC586 analog is designed to have high oral bioavailability and minimal first-pass metabolism. Modifications of the EC586 structure may prove superior over the original structure. In a non-limiting embodiment, the ester moiety is placed at both of the free alcohols in the structure, forming a di-acetate. Such a structure may provide for differential properties. While reports assessing effects of E2 and DHED on PR, galanin, and C3 expression are in rats, in some embodiments experiments are conducted in mice; however rats can be used as well.

### Example 3: Estradiol Mediates Stress-Susceptibility in the Male Brain

Estradiol, often considered a female hormone, is distributed in the male brain via aromatization of testosterone. The role of estrogen receptors (ERs) in male depression is not well understood. It was found that the absence of ERβ is associated with stress susceptibility in male mice and that activity of ERβ-projecting neurons from the basolateral amygdala to nucleus accumbens is reduced in hypogonadal mice subjected to stress, while activation of this circuit reverses stress-induced maladaptive behaviors. It was identified that absence of estradiol, but not testosterone per se, underlies stress susceptibility and that brain-selective delivery of estradiol prevents the development of depression-related behaviors. These findings provide evidence for an estrogen-based mechanism underlying stress susceptibility and offer an unexpected therapeutic strategy for treating depression in males.

It was hypothesized that testosterone acts as a precursor and that it is E2 that is responsible for testosterone's antidepressant effects in males through its action on estrogen receptors (ERs).

### Materials and Methods

### Animals

Wild-type C57BL/6J mice used for behavioral pharmacology experiments were obtained from Jackson laboratories (Bar Harbor, ME, USA). CD1 retired breeders used for the subthreshold defeat experiments and regular non-aggressive CD1 mice used for strangers in the social interaction test were obtained from Charles River (Raleigh, NC, USA).

Esr1 and Esr2 breeding pairs were also obtained from Jackson laboratories. Esr2-icre knock-in (Esr2-icre) mice were used. Wild-type, heterozygous and homozygous Esr1 knockout mice were produced in-house by breeding heterozygous males and females, while heterozygous and homozygous Esr2 knockout mice were produced by breeding heterozygous females and homozygous males. Lastly, wild-type, heterozygous and homozygous Esr2-icre mice were produced in-house by breeding heterozygous or homozygous males and heterozygous females. All mouse lines were bred on a C57BL/6J background. At the time of behavioral testing, the age of the animals was in between 8 to 15 weeks. Mice were group housed and maintained under a 12 h light-dark cycle (lights on at 7:00 a.m.). Water and food were available ad libitum. All mice were housed in the same room in individually ventilated cages, with 3 to 5 mice per cage. All experimental procedures were approved by the University of Maryland Animal Care and Use Committee and conducted in full accordance with the National Institutes of Health Guide for the Care and Use of Laboratory Animals. Tail samples were obtained prior to weaning and genotyped by TransnetYX, Inc. (Cordova, TN, USA). All experiments and analyses were conducted in a randomized, and blind manner to avoid potential biases.

### Drugs

For the chronic administration paradigms, silastic tubes (1.02 mm inner diameter×2.16 mm outer diameter) were filled with testosterone (10 mm) or 17β-Estradiol (E2; 1,3 and 10 mm; Sigma-Aldrich, St. Louis, MO, USA). Silastic implants were primed in 0.9% saline at 37° C. overnight prior to their subcutaneous use. Letrozole (1 mg/kg/day; Sigma-Aldrich, St. Louis, MO, USA) was dissolved in propylene glycol (MP Biomedicals, Santa Ana, CA, USA) and administered through ALZET minipumps (Model 1002, 0.25 μl/hour, 14 days; Cupertino, CA, USA). DHED (synthesized using previously described methods) was dissolved in 0.9% saline and administered through ALZET minipumps (Model 1002, 0.25 μl/hour, 14 days; Cupertino, CA, USA) at a dose of 5 g/kg/day. Flutamide was administered through 1.5 mg slow-release subcutaneous pellets (Innovative Research of America, Sarasota, FL, USA). For acute administration, 17β-Estradiol (E2; 10 and 30 g/kg) was dissolved in sesame oil (4 ml/kg, s.c.; Sigma-Aldrich, St. Louis, MO, USA).

### Surgical Procedures

In all surgical procedures, mice were anesthetized with isoflurane at 3.5% and maintained at 2-2.5% throughout the surgery. Analgesia was provided to the mice in the form of carprofen (5 mg/kg, s.c.; Norbrook laboratories, Newry, UK) prior the start of surgery and after the surgery once per day for two days.

### Orchiectomy

Prior to incision, the scrotal area was thoroughly cleaned with Betadine solution (10% povidone-iodine surgical scrub) followed by ethanol (70%). A small cut was made in the midline of the scrotum using a pair of small sterile surgical scissors. Testicular arteries were crushed with a hemostat and cut below the crush point. Testes were separated from their blood supply, and subsequently removed. The skin was closed through suturing. Animals were allowed to recover for a minimum of ten days prior to any further experiments.

### Subcutaneous Implantation

Subcutaneous implantations were performed during the same time as the orchiectomies. The surgical area was thoroughly cleaned with Betadine solution (10% povidone-iodine surgical scrub) followed by 70% ethanol. A small, approximately 1-cm incision was made between the scapulae using a pair of small sterile surgical scissors. Hemostats were then inserted and spread laterally to the incision to create a cavity in the subcutaneous space in which ALZET mini-osmotic pumps or silastic implants were inserted for continuous drug/vehicle delivery. The skin was closed with suturing.

### Stereotaxic Surgery and Viral Delivery

Heterozygous Esr2-icre or wild-type mice were used for these experiments. Mice were positioned in a small animal stereotaxic frame (Kopf Instruments, Tujungam CA, USA) using head bars. Measurements were taken to ensure that the scalp was flat prior to recording the coordinates. All injections were performed at a rate of 20 nl/min using a 1 μl Hamilton Syringe (Knurled Hub Needle, 25 G).

For the tracing studies, wildtype or Esr2-icre mice received a 350 nl bilateral injection of retrograde AAV9 pCAG-FLEX-tdTomato-WPRE (Addgene, Watertown, MA, USA) or 1% retrograde conjugated cholera toxin labeled with Alexa Fluor 555 (ThermoFischer Scientific, Walthman, MA, USA) in the nucleus accumbens (AP: +1.6, ML: 1.5, DV −5.0 from the top of the skull). For the optogenetic (AAV5.EF1a-DIO-eYFP-WPRE-hGH or AAV5.EF1a-DIO-hChR2(H134R)-eYFP-WPRE-hGH; Penn Vector Core and Addgene) and fiber photometric (AAV5.Syn-Flex-GCaMP6S-WPRE-SV40; Addgene) experiments, mice received 400-nl bilateral injections in the basolateral amygdala (AP: −1.34, ML: 3.0, DV −5.5 from the top of the skull). Three weeks after viral injections, ceramic optic cannulas (400 μm, 0.39 numerical aperture (NA) for optogenetics and 200 mm, 0.37 NA for fiber photometry; Neurophotometrics Ltd, San Diego CA, USA) were implanted in the nucleus accumbens. The use of Esr2-icre-mice with cre-induced expression (DIO or FLEX) virus provide specificity to ERβ expressing cells for all optogenetic and fiber photometry experiments. To further increase the specificity of manipulations on ERβ-projections from the BLA to NAc, the optic cannulas was implanted to the NAc to stimulate/record from the neuronal terminals.

Following the completion of experiments, mice were perfused with 4% paraformaldehyde; their brains were extracted and post-fixed in the same solution for at least 24 hours prior to sectioning with a vibratome (50 μm thickness). Slices were then mounted, coverslipped with Vectashield Antifade Mounting Medium with DAPI (Vector Laboratories, Burlingame, CA, USA), and examined on a microscope (Leica Microsystems, DM6, Buffalo Grove, IL, USA). Fluorescent signal intensity in the GFP channel was used to confirm viral expression and the anatomical co-localization with DAPI staining was used to verify the position of optic fiber implantation.

### Behavioral Experiments

### Subthreshold Social Defeat Stress

Retired CD1 breeders were singly housed and used as aggressors. Two days prior to the start of the experiment, experimental mice were also singly housed. The stress procedure was performed using previously described methods. Briefly, experimental mice were introduced to the home cage of an aggressor for 2 min to initiate the physical attack phase. Afterward, mice were transferred and housed on the opposite side of the aggressor, in the same cage separated by a perforated Plexiglass divider for 15 mins in order to maintain sensory contact. This process was repeated 3 times over a period of ~50 min. Following the completion of the third cycle, mice were placed back in their home cages.

Acute E2 was administered 45 min prior to the initiation of stress or 5 min after the completion of stress. For the optogenetic stimulation, ChR2 and YFP-injected mice received a 4 Hz, 5 ms pulse width, 10-15 mW 470 nm light stimulation for 45 min prior to the initiation of stress. This stimulation paradigm was shown previously to increase synaptic transmission. The optogenetic set up consisted of an LED driver paired with a fiber-coupled LED light source attached to a bifurcated optogenetic path cable (400 μm, 0.49 NA; ThorLabs, Newton NJ, USA).

### Inescapable Foot-Shock Stress

Experimental mice were placed in one side of two-chambered shuttle boxes (Coulbourn Instruments, Whitehall, PA, USA), with the door between the chambers closed. Following a 5-min adaptation period, mice received foot shocks (0.3 mA, 2-sec shock duration, 15-sec inter-trial interval) for 51 min which is equivalent to the duration of the subthreshold social defeat stress.

### Social Interaction

Social interaction was performed 24 hours following subthreshold social defeat or inescapable foot-shock stress. For the habituation phase, mice were placed in a rectangular box (40 cm length×30 cm width×35 cm height; Stoelting, IL) divided into three compartments (two equal-sized end-chambers and a middle chamber) for five minutes (10-15 lux). After the habituation phase, two small wire cages were introduced into the two end chambers, one containing an unfamiliar, non-aggressive CD1 mouse and the other remaining empty. The amount of time mice spent sniffing each cage during the five-min test was assessed using CleverSys tracking software (CleverSys, Inc, Reston VA, USA).

### Female Urine Sniffing Test (FUST)

FUST procedure was performed 24 hours after the social interaction test and 48 hours after stress. FUST was performed using previously described methods. The amount of time mice spent interacting with a cotton-tipped applicator soaked in either fresh male or female mouse urine during a total of 3 minutes was analyzed by an experimenter blind to the experimental groups.

### Open-Field Test (OFT)

OFT was performed under 300-Lux white light. Mice were individually placed into open-field arenas (100×100×38 cm; San Diego Instruments, San Diego, CA) for a 10-min period. The sessions were recorded using an overhead, digital video camera. Distance traveled and time spent in the center of the arena was analyzed using TopScan v2.0 (CleverSys, Inc., Reston VA).

### Novel-Object Recognition (NOR)

Short-term recognition memory was assessed using the novel object recognition task protocol using previously described methods. NOR was performed in dim white lighting conditions (~10-15 lux). The apparatus and objects used here have been previously described. The test was conducted over two days. On the first day, the habituation phase, the animals explored an empty NOR apparatus (40×9×23 cm) for 30 min and then returned to their home cages. On the second day, the mice were re-introduced into the same apparatus, but this time containing two identical objects fixed onto the floor, which they explored for 30 min. After this familiarization phase, mice were immediately returned to their home cages for another 30 min. The mice were then placed back into the NOR apparatus, in which one of the “familiar” objects was replaced by a “novel” object for a 4 min test phase. All three phases of NOR were recorded via an overhead video camera and analyzed using TopScan v2.0 automated scoring software (CleverSys, Inc., Reston VA). The time spent interacting with the familiar and novel objects during the retention phase was measured. A discrimination ratio was calculated by dividing the time spent interacting with the novel object by the total time spent interacting with both objects during the retention phase.

### Sucrose Preference Test

Mice were singly housed and presented with two identical bottles containing either tap water or 1% sucrose solution. The sucrose consumption was measured over 48 hours. The location of the sucrose and tap water bottles was changed every day to avoid the development of side preference.

### Elevated-Plus Maze (EPM)

EPM was carried out in dim white lighting conditions (~5 lux). The EPM apparatus consisted of 2 closed arms and 2 open arms (39×5 cm each) and was elevated 50 cm above the floor (Stoelting, Woodale, IL). The experiment was carried out using previously described methods. The time spent in the open and closed arms of EPM during the 5-min test was recorded by an overhead digital video camera and scored using TopScan v2.0 (CleverSys, Inc., Reston VA). Amount of time spent in the open arms was used as the primary outcome for the anxiety behavioral assessment.

### Forced-Swim Test (FST)

FST was performed in normal white light conditions (~300 lux) using previously described methods. Briefly, mice were subjected to a 6-min swim session in clear Plexiglass cylinders (30-cm height×20-cm diameter) filled with 15 cm of water (23±1° C.). Sessions were recorded using a digital video camera. Immobility time, defined as passive floating with no additional activity other than that necessary to keep the animal's head above water, was scored for the last 4 min of the 6-min test by a trained experimenter blind to the genotypes.

### Real-Time Place Preference

The same three-chambered box or the social interaction test was used. However, for this test, one compartment was altered with white vertical stripes 4 cm apart on each wall and a smooth grey floor, while the other with white horizontal stripes 4 cm apart on the walls and a smooth grey floor. Allocation of the light-paired compartment was counterbalanced to avoid any side preferences. ChR2 and YFP-injected mice were connected to optogenetic fiber cables (ThorLabs, Newton NJ, USA) and placed in the middle compartment, and tracked by an overhead camera. During the 30 min testing period, mice who crossed into the allocated light-paired compartment received a 20 Hz continuous 470 nm ~10-15 mW stimulation. Mouse crossings into each compartment were detected by a Bonsai script which communicated to the LED through connected Arduinos for light delivery.

### Whole-Cell Voltage-Clamp Electrophysiology

Mice (n=3 males) that were previously injected with ChR2 in the BLA were deeply anesthetized with isoflurane before decapitation and brain extraction. Coronal 250 μm thick sections were collected in ice cold 95% oxygen, 5% carbon dioxide-bubbled modified artificial cerebrospinal fluid (aCSF, 194 mM sucrose, 30 mM NaCl, 4.5 mM KCl, 1 mM MgCl₂, 26 mM NaHCO₃, 1.2 mM NaH₂PO₄, and 10 mM D-glucose) before incubating at 32° C. for 30 min in aCSF (124 mM NaCl, 4.5 mM KCl, 2 mM CaCl₂), 1 mM MgCl₂, 26 mM NaHCO₃, 1.2 mM NaH₂PO₄, and 10 mM D-glucose). Slices were then stored at room temperature until recording.

Slices were hemisected, placed into a recording chamber, and perfused with temperature controlled aCSF (29-31° C.). NAc medium spiny neurons (MSNs) were visualized using infrared differential interference contrast light microscopy, Q-capture camera, and associated Pro 7 software. MSNs were voltage clamped at −60 mV using a MultiClamp 700B Amplifier (Molecular Devices, San Jose, CA, USA). Optically evoked post synaptic currents (oPSC) were elicited by an optical fiber placed in the recording chamber that delivered 4 ms pulses of light (473 nm) every 20s for a total recording duration of 15 min. oPSCs were recorded using borosilicate glass pipettes (2-4 MΩ resistance) filled with Cesium Methanesulfonate internal solution (135 mM cesium methanesulfonate, 3 mM NaCl, 10 mM HEPES, 0.6 mM EGTA, 4 mM MgATP, 0.3 mM NaGTP, 5 mM QX-314C₁, pH 7.2, 310 mOsm). Signals were filtered at 2 kHz, digitized at 10 kHz, and acquired using the Clampex 10.4.1.4 software (Molecular Devices). oPSC amplitudes were averaged per minute and expressed as a percentage change from baseline measurements (5 min baseline recording). Following the baseline recording, the AMPAR blocker NBQX (5 M) and NMDAR blocker APV (50 M) were drip perfused into the recording chamber for a 10-min recording period.

### In Vivo Fiber Photometry

Heterozygous Esr2-icre mice received bilateral infusions of AAV-FLEX-GCaMP6s in the BLA. Three to four weeks after the virus infusion, mice underwent orchiectomy or sham surgeries and were bilaterally implanted with fiber optic cannulae (200 μm, 0.37 NA) in the NAc. Following a minimum of 10-day recovery, mice went through subthreshold social defeat stress. Twenty-four hours after the exposure to stress, mice went through the social interaction test. On the day of the social interaction test, mice were connected to the fiber photometry system (Neurophotometrics, San Diego, CA, USA) to record calcium transients in the BLA to NAc afferent terminals using a low-autofluorescence bifurcated optic patch cable (Doric Lenses Inc., Quebec, Canada). Independence of signals was tested prior to the start of the experiment. Excitation wavelengths used were 470 nm for the calcium-dependent GCaMP6s signal, temporally interleaved with 410 nm light at 40 Hz for the calcium-independent (isosbestic) signal. Mice were placed and habituated to the three-chamber arena for measuring social interaction for 5 min. During this period, the photometer's excitation LEDs were on to minimize any rapid initial photobleaching during the test. For the test, a stranger (novel CD1 mouse) was placed in one of the two chambers before reintroducing the test mouse. During a test period of 5 minutes, photometry data was recorded, and the initiation and termination of interactions with either the occupied (stranger) or empty chambers were manually timestamped by the experimenter.

The fiber photometry data was analyzed offline using custom-created MATLAB scripts. Briefly, photometry signals were deinterleaved, and then corrected for baseline drift due to photobleaching using the AirPLS algorithm. A smoothing filter was applied to minimize the high-frequency noise. After z-scoring both signals, the 410 nm signal was scaled and fitted to the 470 nm one before subtraction to correct for any motion artifacts. The resulting z-scored, motion-corrected 470 nm fluorescent signal was used for all subsequent analyses. Using the manually encoded timestamps, periods of interaction with either the stranger or the empty chambers were defined and used to further analyze the signal. For each animal, a mean z-scored fluorescence response to each type of interaction was generated by averaging the segments of the signal ranging temporally from 120 frames (5.76s) prior to the initiation of interaction to 120 frames (5.76s) after the interaction. Any interactions of a type that occurred within 5 s of each other were treated as a single interaction for this analysis, and interactions that occurred within 120 frames of the start or the end of the recording were excluded. For the time-locked analysis, a baseline correction was applied to each response prior to averaging, to account for variation in the fluorescence preceding the interaction. This involved zeroing the mean z-scored fluorescence value corresponding to the period of 90 to 40 frames before the interaction. This baseline period of 2.4 s was chosen as it did not appear to overlap with any observed “anticipatory” activity related to the interactions. Individually, the resultant mean z-scored fluorescence responses were used for the final analysis, which involved comparing the group mean AUC before and after social interaction for each condition.

### Pharmacokinetic Measurements of Distribution of DHED-Derived Estradiol

The experimental design (including the synthesis of a deuterium-labelled DHED) was carried out using previously described methods. Briefly, mice (n=3-4/timepoint) received d₃-DHED (200 μg/kg, s.c.,) and euthanized 0.5, 1, 2, 4 and 8 hours after the injection. Blood was collected by cardiac puncture and the animals were then perfused with cold saline through the left ventricle, and brain tissues were harvested immediately after finishing the perfusion. The brain (cortical) tissues were homogenized in pH 7.4 phosphate buffer to obtain 20% w/v homogenates. Serum samples were obtained from the collected blood by centrifugation (1,500 g at 4° C. for 10 min). Sera and brain homogenates were processed by liquid-liquid extraction using ¹³C-labeled 17β-estradiol (¹³C₆-E2 added at 100 pg/ml serum and 1.7 ng/g wet tissue) as internal standard followed by quantitative isotope-dilution liquid chromatography-tandem mass spectrometry (LC-MS/MS) analyses after dansylation using previously described methods.

cFos Immunofluorescence

Esr2-icre mice received an injection of rAAV9 pCAG-FLEX-tdTomato-WPRE virus. Following 4 weeks, mice were either treated with E2 or Veh and underwent subthreshold social defeat stress. Two hours following the treatment injections mice were perfused with 4% paraformaldehyde. Brains were extracted, post-fixed via immersion in 4% paraformadehyde for 24 hours at 4° C., and cryoprotected in 30% sucrose solution. Consecutive 30 μm-thick free-floating sections were collected in a cryostat (Leica CM3050S) and stored in cryoprotectant at −20° C. until further processing for immunofluorescence.

Six representative brain sections (each 180 μm apart) containing the BLA were selected for each animal. Sections were washed in 1×tris buffered saline (TBS) three times for 15 minutes each, blocked in 5% normal horse serum in 0.4% in Triton X-100 in 1×TBS for an hour at room temperature (RT), and incubated in monoclonal primary antibody against cFos (Cell Signaling Technologies 2250S, 1:3000) for an hour at RT, then 48 hours at 4° C. Sections were protected from light at all washes and incubations to prevent potential photobleaching. Sections were then washed in 1×TBS 3 times for 15 minutes each, and incubated in Alexa fluor 488 conjugated secondary antibody (Cell Signaling Technologies 4412S, 1:2500). After secondary antibody incubation, the sections were washed in 1×TBS two times for 15 minutes each, and in 1×PBS for an additional 15 min. Finally, sections were nuclear stained in DAPI, mounted, coverslipped using Vectashield Vibrance antifade mounting medium (Vector Laboratories), and allowed to set overnight prior to imaging.

Multichannel tiled z-stacked fluorescent images were acquired using a Nikon W1 spinning disk confocal microscope with the 40× objective and following filter cubes: DAPI (405 nm), GFP (488 nm), and RFP (561 nm). All images corresponding to a single immunofluorescence experiment were taken with constant excitation laser intensity, exposure, and display adjustment. Image stitching and projection were completed using NIS-elements software (Nikon, Melville, NY, USA).

For the quantitative assessment of cFos and rAAV colocalization in the BLA, images were imported into Bitplane software Imaris9.6 (Oxford instruments, Concord, MA, USA). A set of 3D reconstructed surface objects were created for the GFP and RFP channel by mapping and thresholding the fluorescent signals from cFos immunohistochemistry and endogenous tdTomato tag expressed by the virus. Next, objects were segmented into individual cells using the built-in watershed algorithm. Colocalization was then determined by setting the shortest distance between the two distance-transformed classes of objects to zero. All images corresponding to a single experiment were batch-analyzed together, with the program automatically generated the count for total number of rAAV labeled cells and the number cFos-rAAV colocalized cells. After which, a scorer blind to group assignments screened through the automatic detection results and manually confirmed its accuracy.

### RNAscope

RNAscope Fluorescent Multiplex Detection Reagent Kit v2 (ACDBio, Newark, CA, USA) was used to assess the colocalization of Esr2 and iCre in the heterozygous Esr2-iCre line and the colocalization of Esr2 to the CTb retrograde tracer targeting the BLA to NAc projections. Four heterozygous Esr2-iCre mice and two wildtype mice with CTb retrograde tracer injected in the NAc were briefly (~1 min) anesthetized with 3.5% isoflurane. Animals were then decapitated, their brains extracted and rapidly frozen in isopentane. 20 μm thick fresh frozen brain sections were collected with a cryostat, directly mounted, and fixed in 4% paraformaldehyde in 1× phosphate buffered saline (15 min), and dehydrated in graded ethanol solutions (50% 70% 100% 100%, 5 min each). Sections were air dried and hydrophobic barrier pen outlines were drawn prior to treatment with hydrogen peroxide (10 min) and Protease plus (5 mins), with two 1×PBS washes (2 min each) in between. All of the remaining incubation steps were completed in the RNAscope hybridization oven at 40° C. Sections were hybridized with probes for Esr2 and iCre (50:1 dilution), or Esr2 alone for the retrograde tracer animals (2 hrs). After 2 washes in RNAscope Washing Buffer (2 mins each), sections were incubated with AMP1 (30 min), AMP2 (30 min), AMP3 (15 min) with two Washing Buffer washes (2 min each) in between each step. Esr2 signals were then developed with successive incubation in HRP-C1 (15 min), TSA plus fluorescein (30 min, 1:1000 in TSA diluent), and HRP blocker (20 min) with 2 intervening washes in RNAscope Washing Buffer (2 min each). For the heterozygous ER_{β}-iCre mice, iCre signals were developed by successive incubation in HRP-C2 (15 min), TSA plus Cy3 (30 min, 1:1000 in TSA diluent), and HRP blocker (20 min) with 2 intervening washes with RNAscope Washing Buffer between each step. Upon completion of signal development, all sections were stained with DAPI for 5 min, coverslipped with FluorSave mounting medium, and set overnight at 4° C. prior to imaging.

BLA and NAc sections were imaged at 20× for all animals. Nuclei expressing ≥2 foci of both Esr2 and iCre were counted as colocalization. These colocalized cells were contrasted to the total number of cells expressing ≥2 foci of iCre to determine the percentage of colocalization of Esr2 to iCre.

The colocalization of Esr2 to a retrograde tracer targeting the projections from the BLA to NAc was similarly determined through counting the total number of nuclei expressing retrograde tracer and ≥2 foci of Esr2. The total colocalized Esr2+tracer nuclei were contrasted to the total number of nuclei expressing tracer (both alone and when colocalized) to determine the percentage of colocalization of Esr2 to tracer. All the images obtained from the RNAscope were manually analyzed by an experienced experimenter blind to the experimental groups.

### Statistical Analysis

Required samples sizes were estimated based upon on past experience performing similar experiments. All studies were performed using simple and stratified randomization methods. Studies were performed using distinct animal groups with the exception of the baseline behaviors where a battery of behavioral experiments was performed using the same mice. Experimentation and analyses were performed by experimenters' blind to the group assignments. All statistical tests were two-tailed, and significance was set at p<0.05. Data were tested for equal variance and Gaussian distribution using the Brown-Forsythe test and Shapiro-Wilk test, respectively using SigmaPlot v14.5. When normality and equal variance was achieved, parametric statistical tests were performed. When normality of samples failed non-parametric tests were used and when equal variance failed Brown Forsythe ANOVA test was used. In the cases were there were more than 2 levels of repeated or matched variables with ε<1.00, Geisser Greenhouse correction was used. Planned comparisons were performed in the cases that there was a priori hypothesis. Correction for multiple comparisons following ANOVAs, Kruskal-Wallis, Brown Forsythe ANOVA test, was performed using either Holm-Sidak post-hoc test or the two-stage linear step up procedure of Benjamini, Krieger and Yekutieli. Spearman correlation analysis was used to test relationships between fiber photometry signals and behavioral outcomes. Statistical analyses were performed using GraphPad Prism v9.0 and SigmaPlot v14.5.

Required samples sizes were estimated based upon on past experience performing similar experiments. All studies were performed using simple and stratified randomization methods. Studies were performed using distinct animal groups with the exception of the baseline behaviors where a battery of behavioral experiments was performed using the same mice. Experimentation and analyses were performed by experimenters blind to the group assignments. All statistical tests were two-tailed, and significance was set at p<0.05. Data were tested for equal variance and Gaussian distribution using the Brown-Forsythe test and Shapiro-Wilk test, respectively using SigmaPlot v14.5. When normality and equal variance was achieved, parametric statistical tests were performed. When normality of samples failed non-parametric tests were used and when equal variance failed Brown Forsythe ANOVA test was used. In the cases were there were more than 2 levels of repeated or matched variables with ε<1.00, Geisser Greenhouse correction was used. Planned comparisons were performed in the cases that there was a priori hypothesis. Correction for multiple comparisons following ANOVAs, Kruskal-Wallis, Brown Forsythe ANOVA test, was performed using either Holm-Sidak post-hoc test or the two-stage linear step up procedure of Benjamini, Krieger and Yekutieli. Spearman correlation analysis was used to test relationships between fiber photometry signals and behavioral outcomes. Statistical analyses were performed using GraphPad Prism v9.0 and SigmaPlot v14.5.

### ERβ is Involved in the Development of Maladaptive Behaviors in Males Following Exposure to Mild Stress

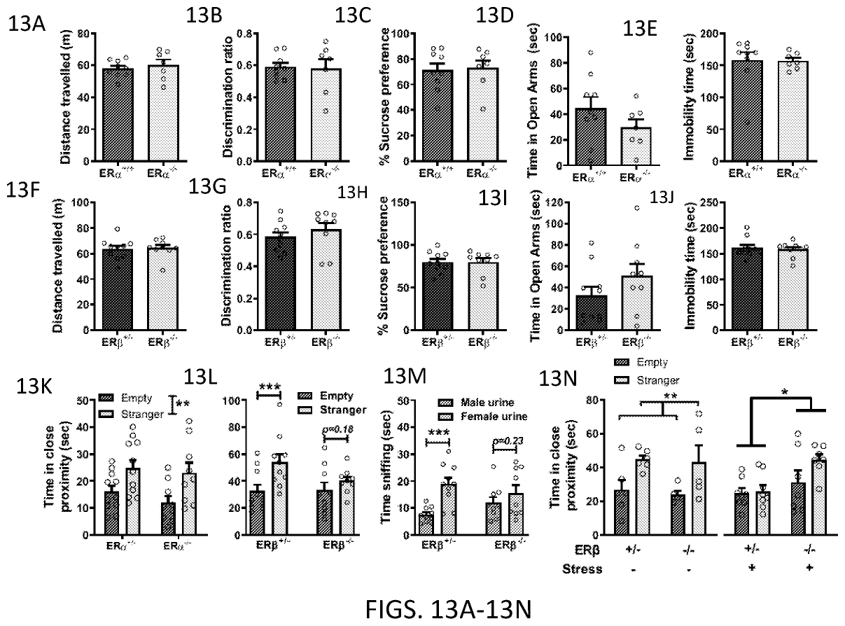

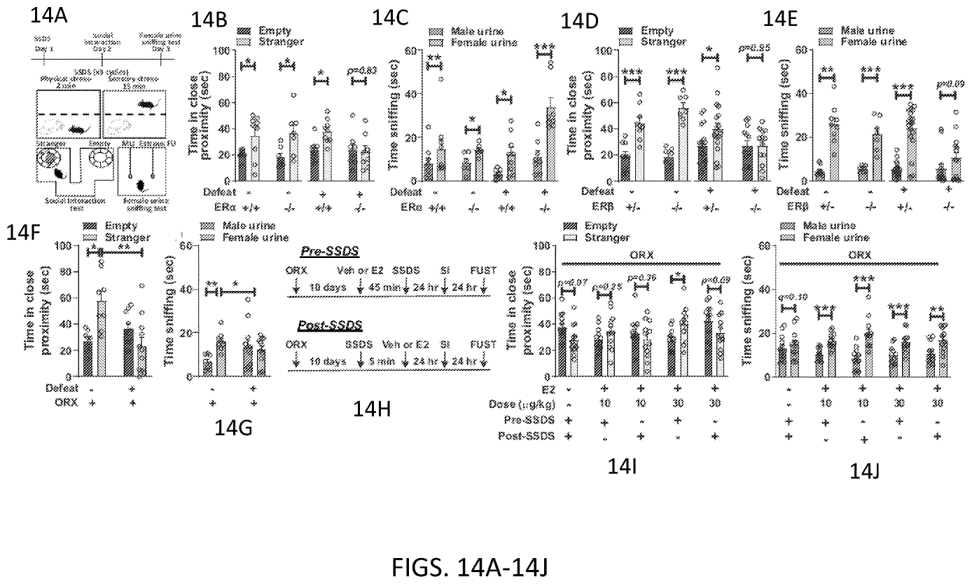

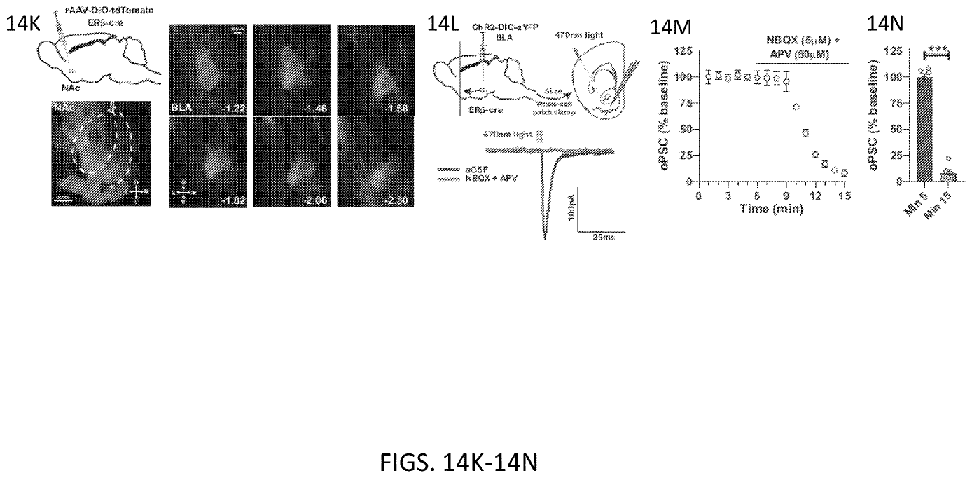

It was assessed whether lack of ERα or ERβ in conventional knockout mice (ERKO or BERKO, respectively) results in maladaptive stress-sensitive behaviors. Under baseline conditions it was found that neither a lack of ERα (FIGS. 13A-13E) nor ERβ (FIGS. 13F-13J) induce robust behavioral changes in gonadally-intact male mice. However, following subthreshold social defeat stress, which is a single day stress paradigm that does not induce any depressive-related behavioral changes in control mice (FIGS. 14A-14D), male mice with a genetic deletion of ERα manifested susceptibility to develop social interaction deficits (FIG. 14B), but not anhedonia as tested by preference for the smell of female versus male mouse urine (FIG. 14C). In agreement with this finding, it was previously demonstrated that ERKO mice did not manifest any social interaction deficits (FIG. 13K) or sucrose preference deficits following inescapable footshock stress. In contrast, genetic deletion of ERβ increased susceptibility to develop both social interaction deficits (FIG. 14D) and anhedonia (FIG. 14E) in male mice. While not wishing to be bound by theory, this result suggests that ERβ might play a greater role in stress susceptibility than ERα. These maladaptive phenotypes were not observed in control ERKO or BERKO mice that were not subjected to stress (FIGS. 14D-14E), indicating that a stress episode, albeit relatively mild, is essential to reveal a susceptible depressive phenotype induced by the lack of ERβ. Social interaction deficits and anhedonia were also induced by foot shock stress (FIGS. 13L-13M), indicating that the ERβ mediated stress susceptibility observed is not dependent on a specific type of stress. It was previously shown that BERKO, but not ERKO mice, demonstrate decrease in sucrose preference following foot shock stress. While not wishing to be bound by theory, this result suggests that the observed effects are not due to the effects of ERβ on chemo-investigation. In support of this conclusion, non-stress BERKO male mice demonstrated the expected female urine preference, suggesting that the lack of preference is due to stress and not due to other confounding factors. While it was demonstrated that ERβ contributes to susceptibility in males, it was previously identified that lack of ERβ in female mice imparts stress resilience following inescapable foot shock induced escape deficits and inescapable foot shock induced social interaction deficits (FIG. 13N), suggesting a sex-dependent differential phenotype and warrants further investigation.

To test whether these behavioral effects were similarly mediated by circulating gonadal hormones, male mice were orchiectomized to remove endogenous testosterone and consequently E2, and subjected to subthreshold social defeat stress or control conditions. It was found that orchiectomized mice manifested the same stress-susceptible phenotype as it was observed in the BERKO mice (FIGS. 14F-14G). Importantly, ORX non-stress controls demonstrated social preference and preference to the female urine, demonstrating that these findings are due to stress and not due to the reduced chemosensory investigation of conspecifics by the loss of hormones using previously described methods. Moreover, acute administration of E2 prior to mild stress in orchiectomized male mice (for timeline see FIG. 14H) prevented the development of both sociability deficits (FIG. 14I) and anhedonia (FIG. 14J), though administration of E2 after stress reversed only the anhedonia phenotype (FIG. 14J). These data reveal that E2 levels and ERβ mediate susceptibility to sub-threshold social stress in male mice.

### Activation of ERβ Expressing Neurons in the BLA to NAc is Rewarding

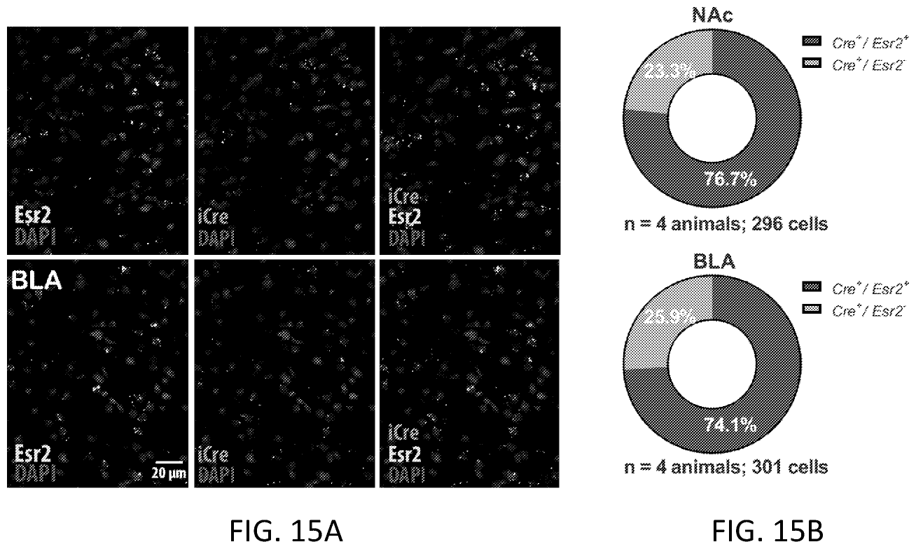

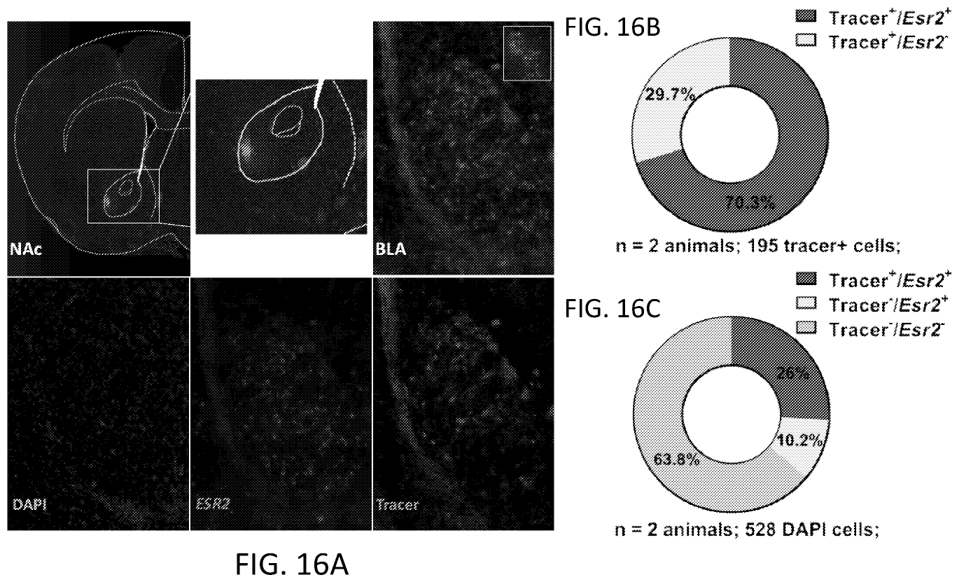

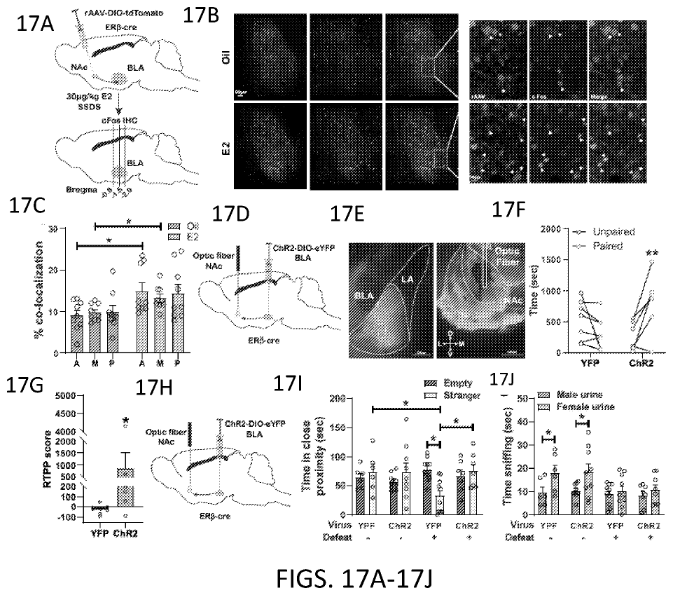

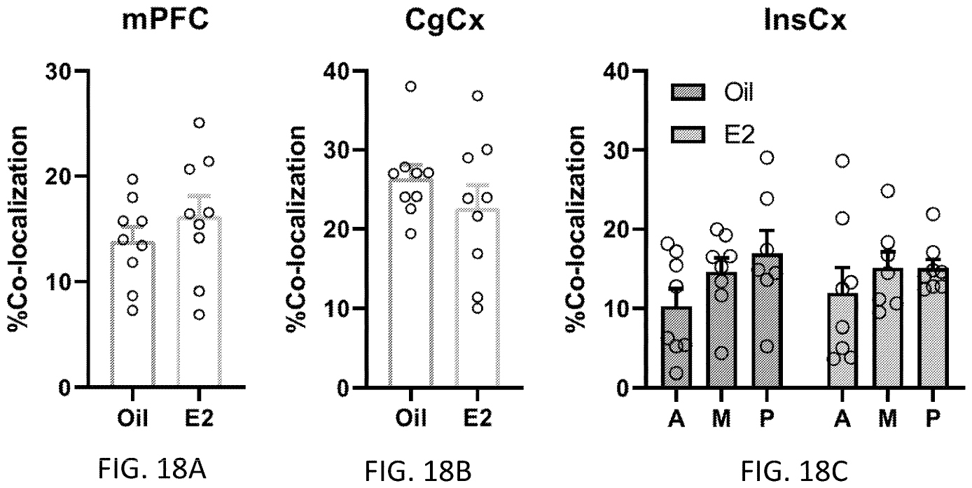

Next, he neural circuit underlying the above-identified role of ERβ in male stress susceptibility was identified. There is ample evidence implicating the nucleus accumbens (NAc) in reward responses and stress resilience/susceptibility. Thus, it was investigated whether there are strong ERβ-expressing projections to the NAc by injecting a cre-sensitive retrograde adeno-associated virus into the NAc of mice expressing cre from the endogenous ERβ promoter (ERβ-icre) (see Extended data FIG. 2 for confirmation of expected cre expression). Neurons strongly expressing ERβ that project from the basolateral amygdala (BLA) to the NAc (FIG. 1K) were identified, which was confirmed by injecting a retrograde conjugate cholera toxin B subunit neuronal tract tracer in the NAc of wild-type mice followed by assessing Esr2 expression co-labelling in the BLA (FIGS. 15A-15B) demonstrating that ~70% of the tracer positive cells also express Esr2. Importantly, the Esr2 expression seems to be enriched specifically in the BLA to NAc projection compared with the whole BLA as Esr2 is expressed at ~10% of the total BLA cells (DAPI labelled) compare with ~26% expression in the BLA to NAc projecting cells (FIGS. 16A-16C). Although the known projections from the BLA to NAc are characterized as glutamatergic, ERβ expressing neurons in the several amygdala subregions are reported to be exclusively GABAergic. In contrast with this prior knowledge but in line with recent evidence, it was demonstrated using whole-cell electrophysiology recordings that the ERβ-expressing BLA to NAc neurons release glutamate but not GABA (FIGS. 13I-13N). To test whether this projection is involved in stress-susceptibility, mice were orchiectomized, treated acutely with either E2 or vehicle (as in FIGS. 14H-14J), and underwent subthreshold social defeats followed by assessment for activation of the BLA-to-NAc projection using c-Fos immunochemistry. Prior to E2 administration, mice received an injection of Cre-sensitive rAAV administered to the NAc, which is retrogradely transported to the BLA. These results demonstrated that following a brief stress exposure, ERβ-expressing BLA-to-NAc neurons are activated at a significantly higher degree in mice that received E2 (FIGS. 17A-17C). Importantly, activation of any of the other strong ERβ-projections to the NAc in response to E2 was observed, suggesting that these effects are possibly specific to the BLA to NAc projections (FIGS. 18A-18C).

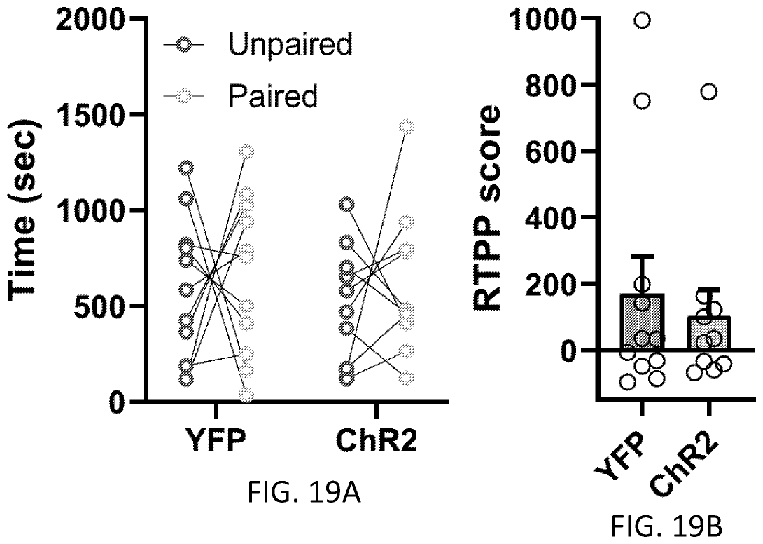

The possible involvement of the ERβ-neuronal projection from the BLA to NAc was then investigated in reward-related behaviors by assessing place preference induced by optogenetic activation of this circuit. Optogenetic activation of the ERβ-expressing BLA-to-NAc terminals induced robust real-time place preference in male (FIGS. 17D-17G) but not female (FIGS. 19A-19B) mice that received ChR2 in the BLA, while control, YFP-injected control mice did not develop real-time place preference (FIGS. 17D-17G). To investigate whether manipulation of this projection might underlie observed stress susceptibility behavioral effects, mice received an injection of ChR2 or YPF in the BLA (FIG. 17H) and were orchiectomized prior to subthreshold social defeats. Light activation of the ERβ-expressing BLA-to-NAc terminals prevented stress-induced social avoidance in orchiectomized mice, while no effect was observed in non-stressed controls, or in the FUST (FIGS. 17I-17J). Although a role of ERα in stress susceptibility cannot be precluded, findings suggest that genetic deletion of ERβ exerts a stronger stress susceptibility phenotype (FIGS. 14B-14E and FIGS. 13K13M). Moreover, in contrast to a role of accumbal ERα in the NAc, these manipulations were focused on the ERβ-expressing cells in BLA to NAc circuit. Interestingly, ERα does not act as a pro-resilient factor in the PFC, thus suggesting a region-specific role of ERα in stress resilience. It is likely ERα exerts its effects by changing local NAc activity, rather than controlling NAc excitability through other projecting regions, such as the PFC or BLA.

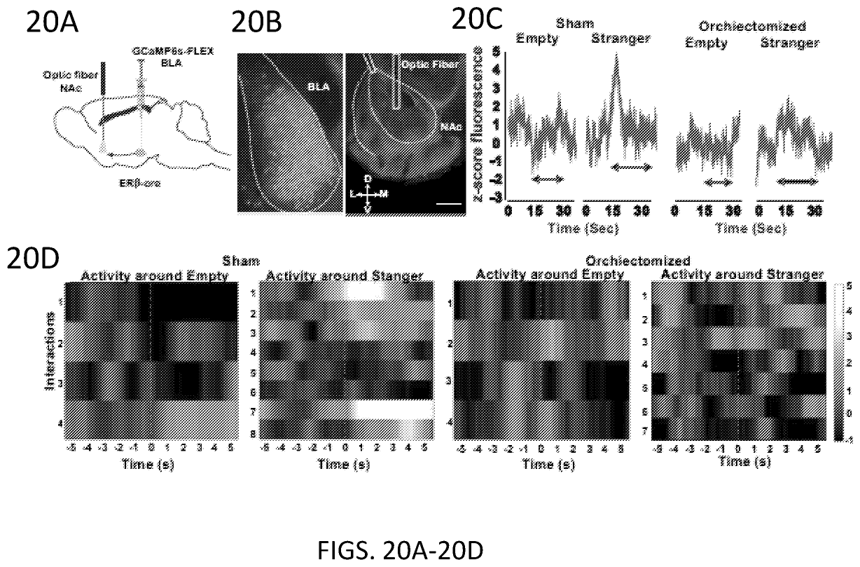

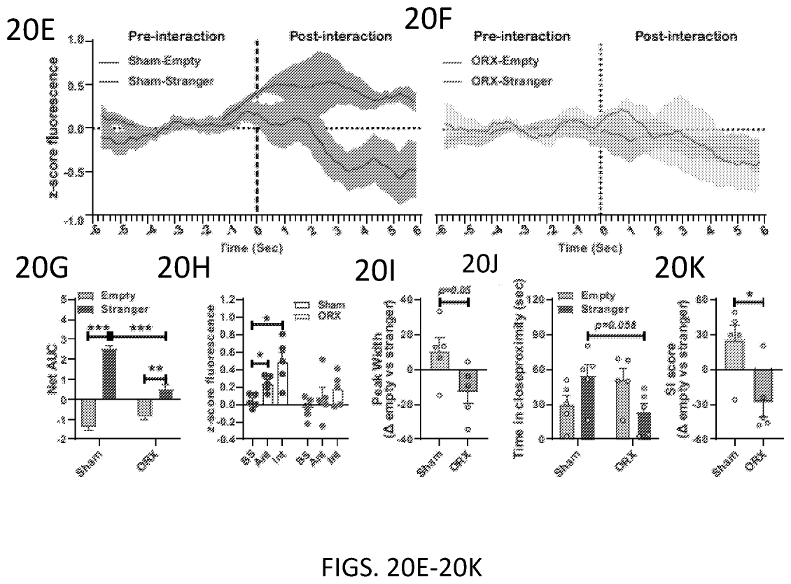

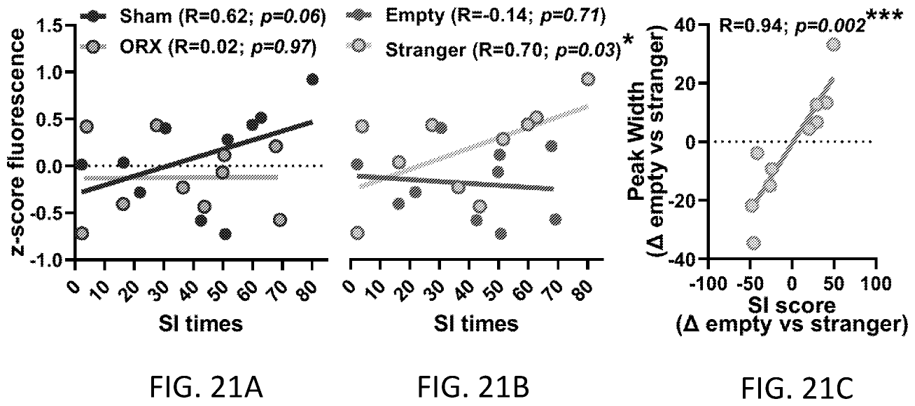

It was hypothesized that in the absence of E2 and following stress exposure, terminal activity of ERβ neurons projecting from BLA to NAc would be decreased during social interaction. To test this hypothesis, in vivo fiber photometry measurements were performed in awake-behaving orchiectomized mice following subthreshold social defeat stress in the social interaction test. This was accomplished by injecting Cre-dependent virus expressing GCaMP6s into the BLA of ERβ-Cre mice and recording calcium transients in the NAc terminals thus providing specificity in recording from only the ERβ-projecting BLA neurons (FIGS. 20A-20B). It was determined that during interaction with a different male mouse, axon terminal calcium activity is decreased in orchiectomized mice which received prior stress, compared to sham controls (FIGS. 20C-20I). Interestingly, these data demonstrate that ERβ BLA to NAc neurons remain partially responsive to the stranger mouse, albeit at lesser extent compared with intact mice (FIG. 20G). While not wishing to be bound by theory, this result suggests that these neurons did not lose their ability to respond but rather that the reward value of the stranger mouse interaction decreased. In gonadally intact mice an increase in calcium transients was observed prior to initiation of social interaction. While not wishing to be bound by theory, this result suggests that this neuronal response might be due to the anticipation of the rewarding stimulus; however, this response was absent in orchiectomized mice (FIGS. 17E, 17F, 17I). These circuit-specific changes were concomitant with social avoidance in orchiectomized mice (FIGS. 20J-20K). Importantly, social interaction scores were positively correlated with the calcium signals evoked during social interactions (FIGS. 21A-21C). Altogether, these data suggest that the ERβ projection from BLA to NAc plays a specific role in E2-related stress susceptibility and development of social impairments.

### Absence of Estradiol Induces Stress Susceptibility Independent of Testosterone in Males

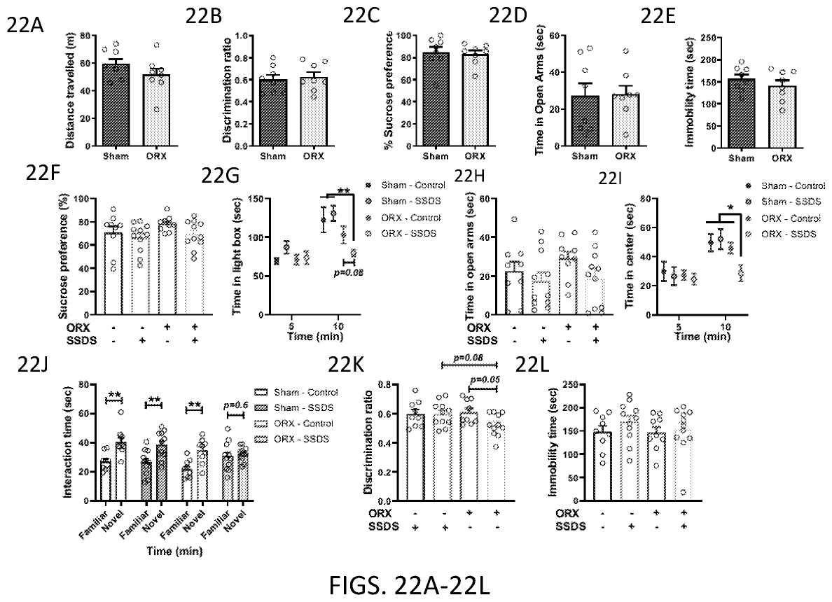

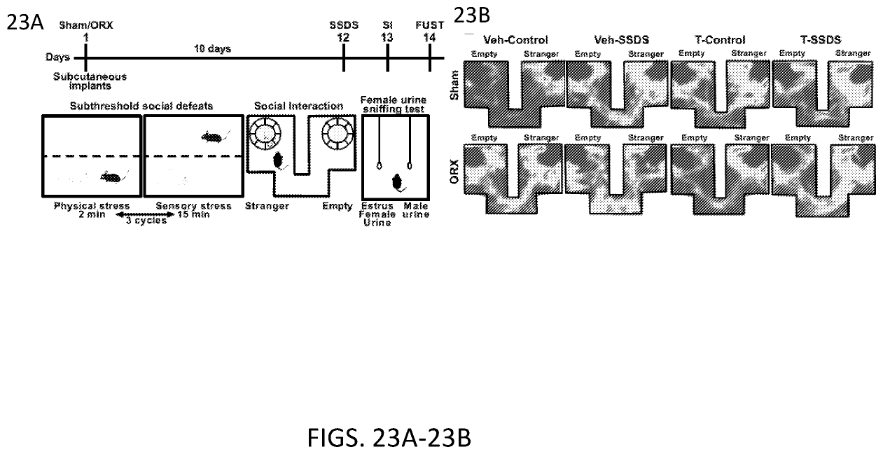

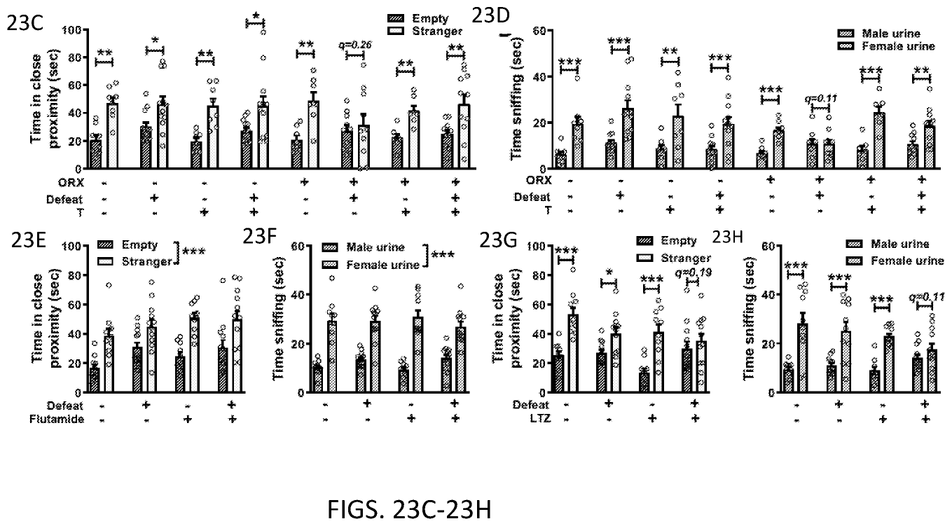

The above findings have clear relevance to human depression occurring as a consequence of stressful life events. The specific role of chronic circulating hormones in stress susceptibility was investigated. First, it was demonstrated that orchiectomy on its own, i.e. elimination of gonadal hormones, did not induce any maladaptive behaviors (FIGS. 22A-22E), suggesting that similar to humans a second factor like stress is needed to reveal a maladaptive phenotype. In support of this, it was demonstrated that orchiectomized mice demonstrate deficits following an acute stress in social behaviors such as social interaction (FIG. 14F) and female urine sniffing (FIG. 14G) and deficits in non-social behaviors such as anxiety (FIGS. 22G-22I), and short-term memory (FIGS. 22G-22K), but no deficits in sucrose preference and forced swim test (FIGS. 22F and 22L). Whether testosterone replacement would reverse stress-induced social interaction and hedonic deficits was then assessed. Mice underwent orchiectomy or sham surgeries and were implanted with either testosterone-filled or empty/control silastic tube implants. Following 10 days of recovery and treatment, mice underwent subthreshold social defeat stress followed by social interaction and anhedonia tests (for timeline see FIG. 23A). Testosterone replacement reversed the observed maladaptive behaviors in orchiectomized mice (FIGS. 23B-23D), while no effect of testosterone was observed in gonadally intact or non-stressed mice regardless of gonadal status (FIGS. 23B-23D). Although testosterone replacement therapy is effective for the treatment of refractory-depression in men, potentially serious life-threatening side effects exist, such as polycythemia and cardiac dysfunction. Thus, potential options that lack testosterone's side effects and demonstrate specificity of testosterone's CNS action in mediating stress susceptibility are desirable.

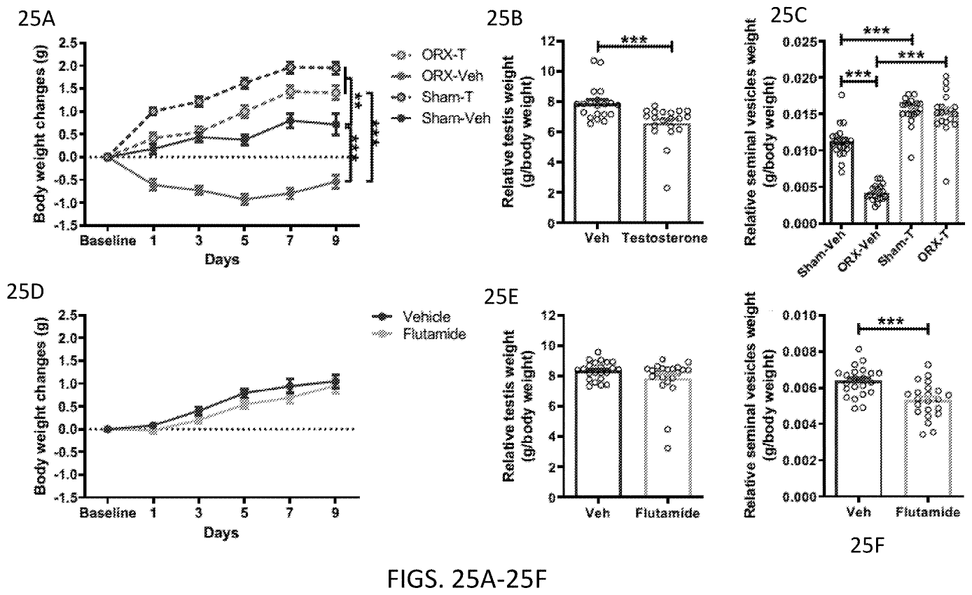

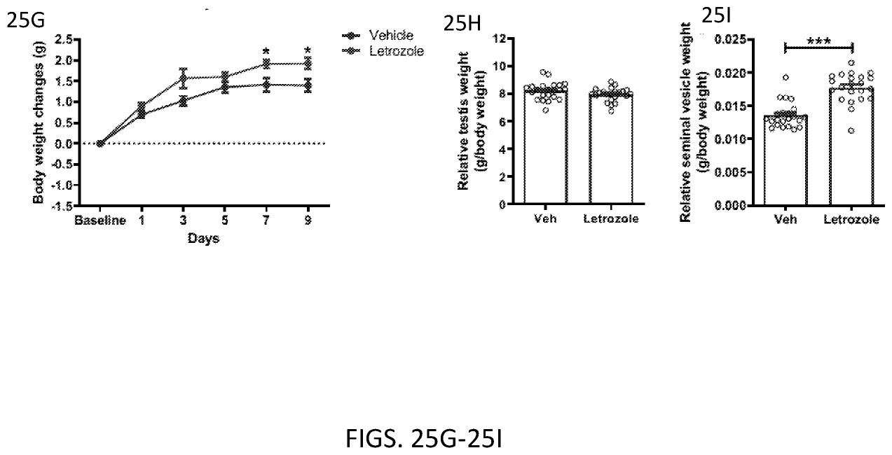

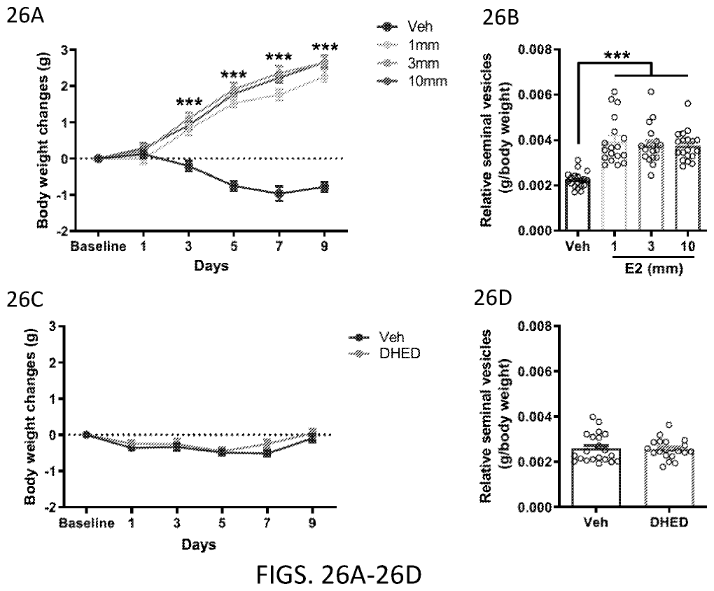

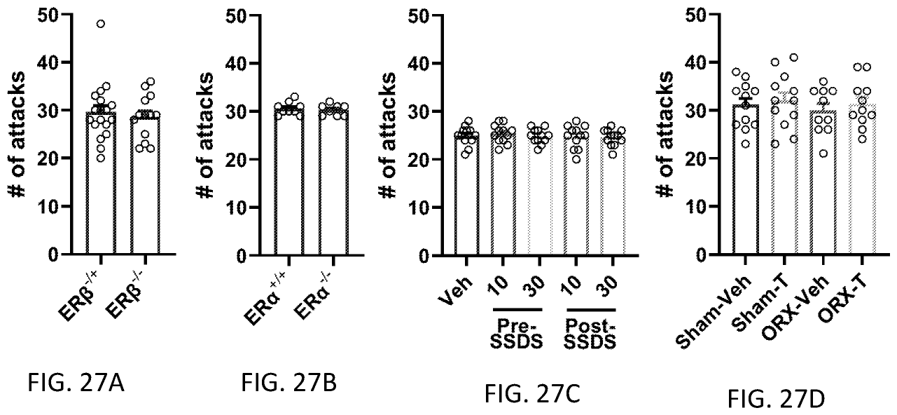

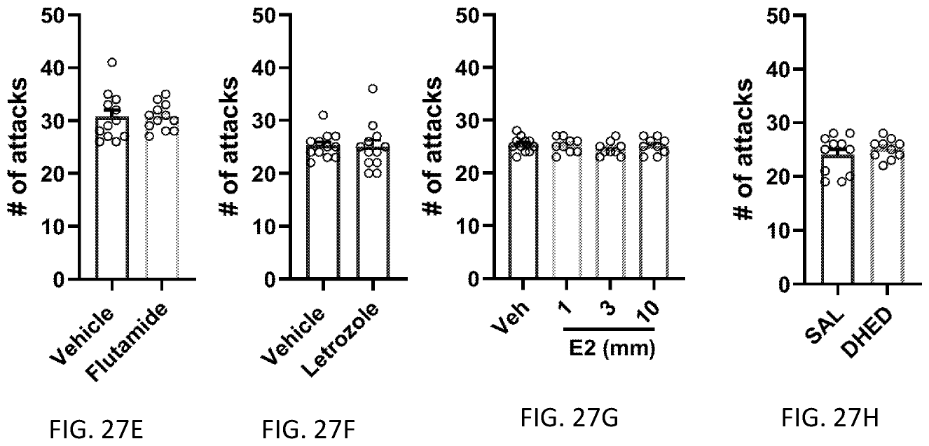

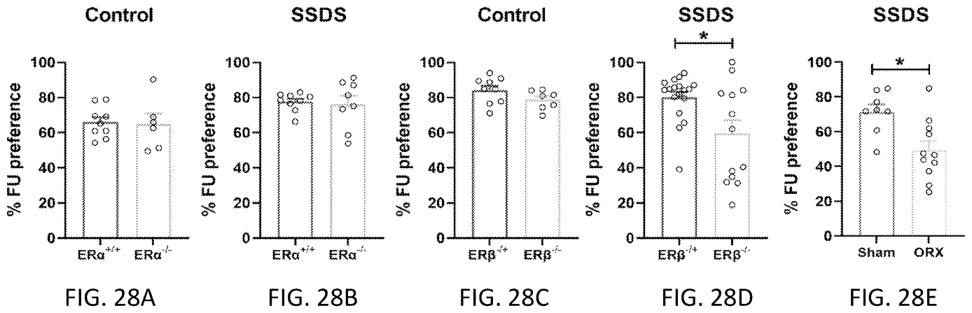

It was next demonstrated that blockade of the androgen receptor, which is the only site of testosterone action, with flutamide in intact mice induced neither social deficits (FIG. 23E) nor anhedonia (FIG. 23F) following stress, bringing into question the direct involvement of testosterone per se. Consistent with an E2-specific mechanism of testosterone action, it was demonstrated that treatment with letrozole, which blocks the aromatization of testosterone to E2, induced a stress-susceptible phenotype in intact male mice, as shown by deficits in social interaction (FIG. 23G) and anhedonia (FIG. 23H). These endophenotypes are consistent with what was observed with hypogonadism. While not wishing to be bound by any particular theory, these results suggest a direct involvement of E2 in regulating the development of depressive-related behaviors following mild stress in males. In agreement with this conclusion, and earlier acute administration data (FIGS. 14H-14I), it was demonstrated that chronic treatment with E2 through the use of silastic tube implants, reversed the stress-induced social avoidance (FIG. 23A) and anhedonia (FIG. 23B) in hypogonadal male mice. Moreover, acute administration data (FIGS. 14H-14I) suggests that E2 can act both as a prophylactic therapy, and, in part, as an antidepressant treatment following the development of the disease. Effectiveness of steroid administration and blockade was confirmed by changes in body, testis and seminal vesicle weights (FIGS. 25A-25I and 26A-26B). Importantly, hormonal and other manipulations did not affect the aggressiveness of CD1 nice during stress as demonstrated by the similar number of attacks (FIGS. 27A-27H).

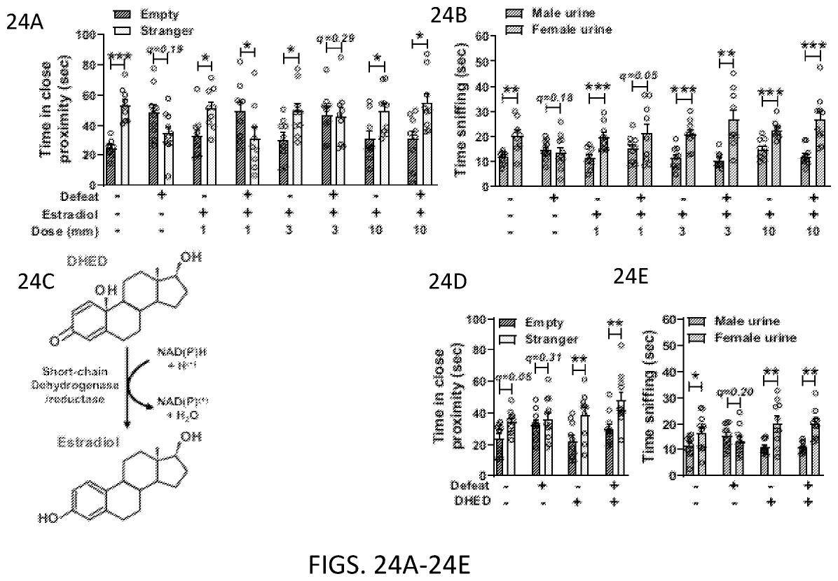

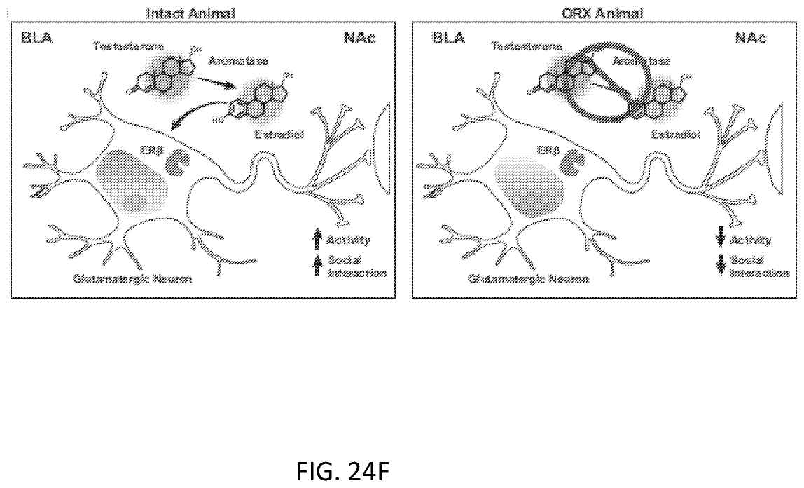

Although these findings support a critical role for E2, and not testosterone itself, as the biologically active hormone mediating stress susceptibility in hypogonadal male mice, E2 replacement therapy cannot be considered as a viable treatment in human male populations due to its peripheral side effects, including gynecomastia and erectile dysfunction. However, isolating the effects of E2 to the central nervous system would provide a viable therapy. It was previously shown that 10β,17β-dihydroxyestra-1,4-dien-3-one (DHED) acts as a prodrug (FIG. 24C), that delivers E2 selectively in the brain via conversion of the prodrug by a NADPH-dependent reductase and hence avoiding E2's peripheral effects. Chronic treatment with DHED, in contrast with E2 treatment (FIGS. 26A-26B), did not increase body weight and seminal vesicle weight compared with vehicle-treated mice (FIGS. 26C-26D) further supporting that DHED lacks E2 peripheral effects. More importantly, it was also found that chronic treatment with DHED reversed the social interaction and hedonic deficits induced by the combination of stress and hypogonadism (FIGS. 24D-24E). While E2 administration is not a viable option for the treatment of male depression due to the peripheral effects, these findings provide evidence for an exciting novel approach and suggest viability of future treatments via brain-selective E2 delivery.

Overall, it was demonstrated that E2 has a causal relationship and plays a pivotal role in male stress-susceptibility in an animal model relevant to depression. It was found that decreased E2 but not testosterone levels per se mediates susceptibility to stress in hypogonadal males, through its action on the ERβ. It was further demonstrated that ERβ is highly expressed in neurons projecting from the BLA to NAc and that neuronal calcium activity in the NAc terminals of ERβ BLA projection neurons is decreased during social interaction after stress in the absence of E2, and that such changes are correlated with social interaction scores. Stimulation of this circuit was found to rescue social avoidance observed following the combination of stress and hypogonadism (i.e. lack of E2), demonstrating that activity of this circuit is causally related with stress susceptibility (FIG. 24F). These findings provide translationally relevant evidence for the effectiveness of targeting ERs, and in particular ERβ, in the male brain as an effective antidepressant approach. In addition, these studies have elucidated a circuit likely involved in the antidepressant actions of testosterone in hypogonadal males. Altogether, these results provide novel evidence for the development of new potential paradigm-shifting and specific therapeutic approaches for the effective treatment of male depression.

### REFERENCES

1. Blow F C, Bohnert A S, Ilgen M A et al. Suicide mortality among patients treated by the Veterans Health Administration from 2000 to 2007. Am J Public Health 2012; 102 Suppl 1: S98-104.
2. Shores M M, Matsumoto A M, Sloan K L et al. Low serum testosterone and mortality in male veterans. Arch Intern Med 2006; 166: 1660-5.
3. Shores M M, Moceri V M, Sloan K L et al. Low testosterone levels predict incident depressive illness in older men: effects of age and medical morbidity. J Clin Psychiatry 2005; 66: 7-14.
4. Liu Y, Sayam S, Shao X et al. Prevalence of and Trends in Diabetes Among Veterans, United States, 2005-2014. Prev Chronic Dis 2017; 14: E135.
5. Olenick M, Flowers M, Diaz V J. U S veterans and their unique issues: enhancing health care professional awareness. Adv Med Educ Pract 2015; 6: 635-9.
6. Wharton W, Gleason C E, Olson S R et al. Neurobiological Underpinnings of the Estrogen Mood Relationship. Curr Psychiatry Rev 2012; 8: 247-56.
7. de Novaes Soares C, Almeida O P, Joffe H et al. Efficacy of Estradiol for the Treatment of Depressive Disorders in Perimenopausal Women: A Double-blind, Randomized, Placebo-Controlled Trial. Archives of General Psychiatry 2001; 58: 529-34.
8. Whooley M A, Grady D, Cauley J A. Postmenopausal estrogen therapy and depressive symptoms in older women. J Gen Intern Med 2000; 15: 535-41.
9. Schmidt P J, Nieman L, Danaceau M A et al. Estrogen replacement in perimenopause-related depression: a preliminary report. Am J Obstet Gynecol 2000; 183: 414-20.
10. Callard G V, Petro Z, Ryan K J. Conversion of Androgen to Estrogen and Other Steroids in the Vertebrate Brain. American Zoologist 2015; 18: 511-23.
11. Perez-Palacios G, Larsson K, Beyer C. Biological significance of the metabolism of androgens in the central nervous system. J Steroid Biochem 1975; 6: 999-1006.
12. Danielson P B. The cytochrome P450 superfamily: biochemistry, evolution and drug metabolism in humans. Curr Drug Metab 2002; 3: 561-97.
13. Carrier N, Kabbaj M. Testosterone and imipramine have antidepressant effects in socially isolated male but not female rats. Horm Behav 2012; 61: 678-85.
14. Carrier N, Kabbaj M. Extracellular signal-regulated kinase 2 signaling in the hippocampal dentate gyrus mediates the antidepressant effects of testosterone. Biol Psychiatry 2012; 71: 642-51.
15. Seidman S N, Rabkin J G. Testosterone replacement therapy for hypogonadal men with SSRI-refractory depression. J Affect Disord 1998; 48: 157-61.
16. Shores M M, Kivlahan D R, Sadak T I et al. A randomized, double-blind, placebo-controlled study of testosterone treatment in hypogonadal older men with subthreshold depression (dysthymia or minor depression). J Clin Psychiatry 2009; 70: 1009-16.
17. Walther A, Wasielewska J M, Leiter O. The antidepressant effect of testosterone: An effect of neuroplasticity?Neurology, Psychiatry and Brain Research 2019; 32: 104-10.
18. Turvin J C, Messer W S, Jr., Kritzer M F. On again, off again effects of gonadectomy on the acoustic startle reflex in adult male rats. Physiol Behav 2007; 90: 473-82.
19. Gogos A, Martin S, Jones M E et al. Oestrogen modulation of the effect of 8-O H-DPAT on prepulse inhibition: effects of aromatase deficiency and castration in mice. Psychopharmacology (Berl) 2006; 188: 100-10.
20. Martin S, Jones M, Simpson E et al. Impaired spatial reference memory in aromatase-deficient (ArKO) mice. Neuroreport 2003; 14: 1979-82.
21. Frye C A, Paris J J, Rhodes M E. Estrogen is necessary for 5alpha-pregnan-3alpha-ol-20-one (3alpha,5alpha-THP) infusion to the ventral tegmental area to facilitate social and sexual, but neither exploratory nor affective behavior of ovariectomized rats. Pharmacology, biochemistry, and behavior 2008; 91: 261-70.
22. Frye C A, Koonce C J, Edinger K L et al. Androgens with activity at estrogen receptor beta have anxiolytic and cognitive-enhancing effects in male rats and mice. Horm Behav 2008; 54: 726-34.
23. Chrysant S G, Chrysant G S. Cardiovascular benefits and risks of testosterone replacement therapy in older men with low testosterone. Hosp Pract (1995) 2018; 46: 47-55.
24. Basaria S, Coviello A D, Travison T G et al. Adverse events associated with testosterone administration. N Engl J Med 2010; 363: 109-22.
25. Ohlander S J, Varghese B, Pastuszak A W. Erythrocytosis Following Testosterone Therapy. Sex Med Rev 2018; 6: 77-85.
26. Narula H S, Carlson H E. Gynaecomastia—pathophysiology, diagnosis and treatment. Nat Rev Endocrinol 2014; 10: 684-98.
27. Schulster M, Bernie A M, Ramasamy R. The role of estradiol in male reproductive function. Asian J Androl 2016; 18: 435-40.
28. Wibowo E, Schellhammer P, Wassersug R J. Role of estrogen in normal male function: clinical implications for patients with prostate cancer on androgen deprivation therapy. Journal of Urology 2011; 185: 17-23.
29. Prokai L, Nguyen V, Szarka S et al. The prodrug DHED selectively delivers 17beta-estradiol to the brain for treating estrogen-responsive disorders. Sci Transl Med 2015; 7: 297ra113.
30. Merchenthaler I, Lane M, Sabnis G et al. Treatment with an orally bioavailable prodrug of 17beta-estradiol alleviates hot flushes without hormonal effects in the periphery. Sci Rep 2016; 6: 30721.
31. Merchenthaler I, Lane M, Stennett C et al. Brain-Selective Estrogen Therapy Prevents Androgen Deprivation-Associated Hot Flushes in a Rat Model. Pharmaceuticals (Basel) 2020; 13.
32. Nieschlag E, Nieschlag S. Testosterone deficiency: a historical perspective. Asian J Androl 2014; 16: 161-8.
33. Ahmed G, Elger W, Meece F et al. A prodrug design for improved oral absorption and reduced hepatic interaction. Bioorg Med Chem 2017; 25: 5569-75.
34. Golden S A, Covington H E, 3rd, Berton O et al. A standardized protocol for repeated social defeat stress in mice. Nat Protoc 2011; 6: 1183-91.
35. Malkesman O, Scattoni M L, Paredes D et al. The female urine sniffing test: a novel approach for assessing reward-seeking behavior in rodents. Biol Psychiatry 2010; 67: 864-71.
36. Kudwa A E, Bodo C, Gustafsson J A et al. A previously uncharacterized role for estrogen receptor beta: defeminization of male brain and behavior. Proc Natl Acad Sci USA 2005; 102: 4608-12.
37. Nestler E J. Role of the Brain's Reward Circuitry in Depression: Transcriptional Mechanisms. Int Rev Neurobiol 2015; 124: 151-70.
38. Bagot R C, Parise E M, Pena C J et al. Ventral hippocampal afferents to the nucleus accumbens regulate susceptibility to depression. Nat Commun 2015; 6: 7062.
39. Heshmati M, Christoffel D J, LeClair K et al. Depression and Social Defeat Stress Are Associated with Inhibitory Synaptic Changes in the Nucleus Accumbens. J Neurosci 2020; 40: 6228-33.
40. Golden S A, Christoffel D J, Heshmati M et al. Epigenetic regulation of RAC1 induces synaptic remodeling in stress disorders and depression. Nat Med 2013; 19: 337-44.
41. Francis T C, Chandra R, Friend D M et al. Nucleus accumbens medium spiny neuron subtypes mediate depression-related outcomes to social defeat stress. Biol Psychiatry 2015; 77: 212-22.
42. Brog J S, Salyapongse A, Deutch A Y et al. The patterns of afferent innervation of the core and shell in the “accumbens” part of the rat ventral striatum: immunohistochemical detection of retrogradely transported fluoro-gold. J Comp Neurol 1993; 338: 255-78.
43. Christie M J, Summers R J, Stephenson J A et al. Excitatory amino acid projections to the nucleus accumbens septi in the rat: a retrograde transport study utilizing D[3H]aspartate and [3H]GABA. Neuroscience 1987; 22: 425-39.
44. O'Donnell P, Grace A. Synaptic interactions among excitatory afferents to nucleus accumbens neurons: hippocampal gating of prefrontal cortical input. The Journal of Neuroscience 1995; 15: 3622-39.
45. Blurton-Jones M, Tuszynski M H. Estrogen receptor-beta colocalizes extensively with parvalbumin-labeled inhibitory neurons in the cortex, amygdala, basal forebrain, and hippocampal formation of intact and ovariectomized adult rats. J Comp Neurol 2002; 452: 276-87.
46. O'Leary T P, Sullivan K E, Wang L et al. Extensive and spatially variable within-cell-type heterogeneity across the basolateral amygdala. Elife 2020; 9.
47. de Ronde W, de Jong F H. Aromatase inhibitors in men: effects and therapeutic options. Reprod Biol Endocrinol 2011; 9: 93.
48. Mosquera L, Shepherd L, Torrado A I et al. Comparison of Two Methods of Estradiol Replacement: their Physiological and Behavioral Outcomes. J Vet Sci Technol 2015; 6: 276.
49. Vellers H L, Letsinger A C, Walker N R et al. High Fat High Sugar Diet Reduces Voluntary Wheel Running in Mice Independent of Sex Hormone Involvement. Front Physiol 2017; 8: 628.
50. Bowen R S, Ferguson D P, Lightfoot J T. Effects of Aromatase Inhibition on the Physical Activity Levels of Male Mice. J Steroids Horm Sci 2011; 1: 1-7.
51. Walsh T J, Shores M M, Fox A E et al. Recent trends in testosterone testing, low testosterone levels, and testosterone treatment among Veterans. Andrology 2015; 3: 287-92.
52. Oizumi H, Kuriyama N, Imamura S et al. Influence of aging on the behavioral phenotypes of C57BL/6J mice after social defeat. PLoS One 2019; 14: e0222076.
53. Chen L S, Tzeng W Y, Chuang J Y et al. Roles of testosterone and amygdaloid LTP induction in determining sex differences in fear memory magnitude. Horm Behav 2014; 66: 498-508.
54. Perry C J, Ganella D E, Nguyen L D et al. Assessment of conditioned fear extinction in male and female adolescent rats. Psychoneuroendocrinology 2020; 116: 104670.
55. Kovacsics C E, Gould T D. Shock-induced aggression in mice is modified by lithium. Pharmacol Biochem Behav 2010; 94: 380-6.
56. Graham B M, Milad M R. Blockade of estrogen by hormonal contraceptives impairs fear extinction in female rats and women. Biol Psychiatry 2013; 73: 371-8.
57. Graham B M, Milad M R. Inhibition of estradiol synthesis impairs fear extinction in male rats. Learn Mem 2014; 21: 347-50.
58. Deslauriers J, Toth M, Der-Avakian A et al. Current Status of Animal Models of Posttraumatic Stress Disorder: Behavioral and Biological Phenotypes, and Future Challenges in Improving Translation. Biol Psychiatry 2018; 83: 895-907.
59. Ozbay F, Johnson D C, Dimoulas E et al. Social support and resilience to stress: from neurobiology to clinical practice. Psychiatry (Edgmont) 2007; 4: 35-40.
60. Hoge C W, Riviere L A, Wilk J E et al. The prevalence of post-traumatic stress disorder (PTSD) in U S combat soldiers: a head-to-head comparison of DSM-5 versus DSM-IV-TR symptom criteria with the PTSD checklist. Lancet Psychiatry 2014; 1: 269-77.
61. Shalev A Y, Peri T, Orr S P et al. Auditory startle responses in help-seeking trauma survivors. Psychiatry Res 1997; 69: 1-7.
62. Morgan C A, 3rd, Grillon C, Southwick S M et al. Fear-potentiated startle in posttraumatic stress disorder. Biol Psychiatry 1995; 38: 378-85.
63. Morgan C A, 3rd, Grillon C, Southwick S M et al. Exaggerated acoustic startle reflex in Gulf War veterans with posttraumatic stress disorder. Am J Psychiatry 1996; 153: 64-8.
64. Butler R W, Braff D L, Rausch J L et al. Physiological evidence of exaggerated startle response in a subgroup of Vietnam veterans with combat-related PTSD. Am J Psychiatry 1990; 147: 1308-12.
65. Orr S P, Lasko N B, Metzger L J et al. Physiologic responses to non-startling tones in Vietnam veterans with post-traumatic stress disorder. Psychiatry Res 1997; 73: 103-7.
66. Seppala E M, Nitschke J B, Tudorascu D L et al. Breathing-based meditation decreases posttraumatic stress disorder symptoms in U.S. military veterans: a randomized controlled longitudinal study. J Trauma Stress 2014; 27: 397-405.
67. Rothbaum B O, Price M, Jovanovic T et al. A randomized, double-blind evaluation of D-cycloserine or alprazolam combined with virtual reality exposure therapy for posttraumatic stress disorder in Iraq and Afghanistan War veterans. Am J Psychiatry 2014; 171: 640-8.
68. Griffin M G, Resick P A, Galovski T E. Does physiologic response to loud tones change following cognitive-behavioral treatment for posttraumatic stress disorder? J Trauma Stress 2012; 25: 25-32.
69. Koch M. The neurobiology of startle. Prog Neurobiol 1999; 59: 107-28.
70. Pynoos R S, Ritzmann R F, Steinberg A M et al. A behavioral animal model of posttraumatic stress disorder featuring repeated exposure to situational reminders. Biol Psychiatry 1996; 39: 129-34.
71. Polta S A, Fenzl T, Jakubcakova V et al. Prognostic and symptomatic aspects of rapid eye movement sleep in a mouse model of posttraumatic stress disorder. Front Behav Neurosci 2013; 7: 60.
72. Dirks A, de Jongh R, Groenink L et al. Footshock-induced sensitization of the acoustic startle response in two strains of mice. Behav Brain Res 2001; 123: 17-21.
73. Pulliam J V, Dawaghreh A M, Alema-Mensah E et al. Social defeat stress produces prolonged alterations in acoustic startle and body weight gain in male Long Evans rats. J Psychiatr Res 2010; 44: 106-11.
74. Bonasera S J, Schenk A K, Luxenberg E J et al. Mice Lacking Serotonin 2C Receptors Have increased Affective Responses to Aversive Stimuli. PLoS One 2015; 10: e0142906.
75. Hebb A L, Zacharko R M, Gauthier M et al. Exposure of mice to a predator odor increases acoustic startle but does not disrupt the rewarding properties of VTA intracranial self-stimulation. Brain Res 2003; 982: 195-210.
76. van den Buuse M, Low J K, Kwek P et al. Selective enhancement of NMDA receptor-mediated locomotor hyperactivity by male sex hormones in mice. Psychopharmacology (Berl) 2017; 234: 2727-35.
77. Tolkach Y, Joniau S, Van Poppel H. Luteinizing hormone-releasing hormone (LHRH) receptor agonists vs antagonists: a matter of the receptors? BJU Int 2013; 111: 1021-30.
78. Gao Y, Gesenberg, C., ad Zheng, W. Oral Formulations for Preclinical Studies: Principle, Design and Development Considerations. In: Yihong Qiu Y C, Geoff G. Z. Zhang, Lawrence Yu, Rao V. Mantri (ed). Developing Solid Oral Dosage Forms, 2 edn. Elsevier Inc.2017, pp 455-95.
79. Zawilska J B, Wojcieszak J, Olejniczak A B. Prodrugs: a challenge for the drug development. Pharmacol Rep 2013; 65: 1-14.
80. Hamada Y. Recent progress in prodrug design strategies based on generally applicable modifications. Bioorg Med Chem Lett 2017; 27: 1627-32.
81. Nieschlag E, Mauss J, Coert A et al. Plasma androgen levels in men after oral administration of testosterone or testosterone undecanoate. Acta Endocrinol (Copenh) 1975; 79: 366-74.
82. Highland J N, Morris P J, Zanos P et al. Mouse, rat, and dog bioavailability and mouse oral antidepressant efficacy of (2R,6R)-hydroxynorketamine. J Psychopharmacol 2019; 33: 12-24.
83. Merchenthaler I, Lennard D E, Lopez F J et al. Neonatal imprinting predetermines the sexually dimorphic, estrogen-dependent expression of galanin in luteinizing hormone-releasing hormone neurons. Proc Natl Acad Sci USA 1993; 90: 10479-83.
84. Vrontakis M E, Yamamoto T, Schroedter I C et al. Estrogen induction of galanin synthesis in the rat anterior pituitary gland demonstrated by in situ hybridization and immunohistochemistry. Neurosci Lett 1989; 100: 59-64.
85. Shughrue P J, Stumpf W E, Sar M. The distribution of progesterone receptor in the 20-day-old fetal mouse: an autoradiographic study with [125I]progestin. Endocrinology 1988; 123: 2382-9.
86. Shughrue P J, Lubahn D B, Negro-Vilar A et al. Responses in the brain of estrogen receptor alpha-disrupted mice. Proc Natl Acad Sci USA 1997; 94: 11008-12.
87. Leiva M C, Lyttle C R, Jellinck P H. Complement C3 synthesis, peroxidase activity and eosinophil chemotaxis in the rat uterus: effect of estradiol and testosterone. Mol Cell Endocrinol 1991; 81: 105-11.
88. Diel P, Schulz T, Smolnikar K et al. Ability of xeno- and phytoestrogens to modulate expression of estrogen-sensitive genes in rat uterus: estrogenicity profiles and uterotropic activity. J Steroid Biochem Mol Biol 2000; 73: 1-10.
89. Shughrue P J, Lane M V, Merchenthaler I. Regulation of progesterone receptor messenger ribonucleic acid in the rat medial preoptic nucleus by estrogenic and antiestrogenic compounds: an in situ hybridization study. Endocrinology 1997; 138: 5476-84.
90. Schmittgen T D, Livak K J. Analyzing real-time PCR data by the comparative C(T) method. Nat Protoc 2008; 3: 1101-8.
91. Leibl H, Bieglmayer C, Spona J. Effects of 17 beta-estradiol, synthetic estrogens, and antiestrogens on prolactin secretion, nuclear translocation of pituitary estrogen receptor and on progesterone receptor levels in rat pituitary, hypothalamus and uterus. Endocrinol Exp 1981; 15: 35-44.
92. Sundstrom S A, Komm B S, Ponce-de-Leon H et al. Estrogen regulation of tissue-specific expression of complement C₃. J Biol Chem 1989; 264: 16941-7.
93. Nellemann C, Dalgaard M, Holst B et al. Gene expression changes in rat prostate after activation or blocking of the androgen and estrogen receptor. Mol Cell Endocrinol 2005; 237: 25-35.
94. J. A. McHenry et al., Hormonal gain control of a medial preoptic area social reward circuit. Nat Neurosci 20, 449-458 (2017).
95. K. E. Yoest, J. A. Cummings, J. B. Becker, Estradiol, dopamine and motivation. Cent Nerv Syst Agents Med Chem 14, 83-89 (2014).
96. S. S. Kokane, L. I. Perrotti, Sex Differences and the Role of Estradiol in Mesolimbic Reward Circuits and Vulnerability to Cocaine and Opiate Addiction. Front Behav Neurosci 14, 74 (2020).
97. P. Georgiou, P. Zanos, C. E. Jenne, T. D. Gould, Sex-Specific Involvement of Estrogen Receptors in Behavioral Responses to Stress and Psychomotor Activation. Front Psychiatry 10, 81 (2019).
98. A. Sansone, F. Romanelli, M. Sansone, A. Lenzi, L. Di Luigi, Gynecomastia and hormones. Endocrine 55, 37-44 (2017).
99. T. Chen et al., Different levels of estradiol are correlated with sexual dysfunction in adult men. Sci Rep 10, 12660 (2020).
100. C. E. Terrillion, T. C. Francis, A. C. Puche, M. K. Lobo, T. D. Gould, Decreased Nucleus Accumbens Expression of Psychiatric Disorder Risk Gene Cacna1c Promotes Susceptibility to Social Stress. Int J Neuropsychopharmacol 20, 428-433 (2017).
101. P. Zanos et al., NMDAR inhibition-independent antidepressant actions of ketamine metabolites. Nature 533, 481-486 (2016).
102. P. Zanos et al., Sex-dependent modulation of age-related cognitive decline by the L-type calcium channel gene Cacna1c (Cav 1.2). Eur J Neurosci 42, 2499-2507 (2015).
103. A. Can et al., The mouse forced swim test. J Vis Exp, e3638 (2012).
104. E. Martianova, S. Aronson, C. D. Proulx, Multi-Fiber Photometry to Record Neural Activity in Freely-Moving Animals. J Vis Exp, (2019).
105. Z. M. Zhang, S. Chen, Y. Z. Liang, Baseline correction using adaptive iteratively reweighted penalized least squares. Analyst 135, 1138-1146 (2010).

A number of patent and non-patent publications are cited herein in order to describe the state of the art to which this disclosure pertains. The entire disclosure of each of these publications is incorporated by reference herein.

While certain embodiments of the present disclosure have been described and/or exemplified above, various other embodiments will be apparent to those skilled in the art from the foregoing disclosure. The present disclosure is, therefore, not limited to the particular embodiments described and/or exemplified, but is capable of considerable variation and modification without departure from the scope and spirit of the appended claims.

1. A compound of formula (I), or a pharmaceutically acceptable salt, solvate, hydrate, cocrystal, or prodrug thereof: 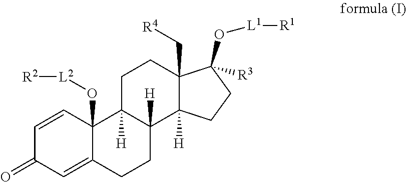
wherein in formula (I):
L¹ and L² are each a linker comprising independently one or more of a bond, —C(O)—, —O—, —S—, —NR^{a}—, —CR^{a}₂—, —C(O)O—, —C(O)S—, —C(O)NR^{a}—, —C(O)NR^{a}SO₂—, disubstituted alkyl, disubstituted heteroalkyl, disubstituted alkenyl, disubstituted alkynyl, disubstituted cycloalkyl, disubstituted heterocycloalkyl, disubstituted aryl, disubstituted arylalkyl, disubstituted heteroaryl, and disubstituted heteroarylalkyl;
R^{a} is independently selected at each occurrence from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl;
R¹ is selected from optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl;
R² is selected from hydrogen, optionally substituted alkyl, optionally substituted fluoroalkyl, optionally substituted carbocyclyl, optionally substituted carbocyclylalkyl, optionally substituted aryl, optionally substituted aralkyl, optionally substituted heterocycloalkyl, optionally substituted heterocycloalkylalkyl, optionally substituted heteroaryl, and optionally substituted heteroarylalkyl;
R³ is selected from hydrogen and optionally substituted alkynyl; and
R⁴ is selected from hydrogen and optionally substituted alkyl.

2. The compound of claim 1, wherein L¹ comprises one or more of a bond, disubstituted alkyl, —C(O)—, —C(O)O—, and —(CH₂)ₚO—wherein p is an integer from 1 to 5.

3. The compound of claim 1 or 2, wherein the compound of formula (I) is a compound of formula (10), formula (11), or formula (12), or a pharmaceutically acceptable salt, solvate, hydrate, cocrystal, or prodrug thereof:

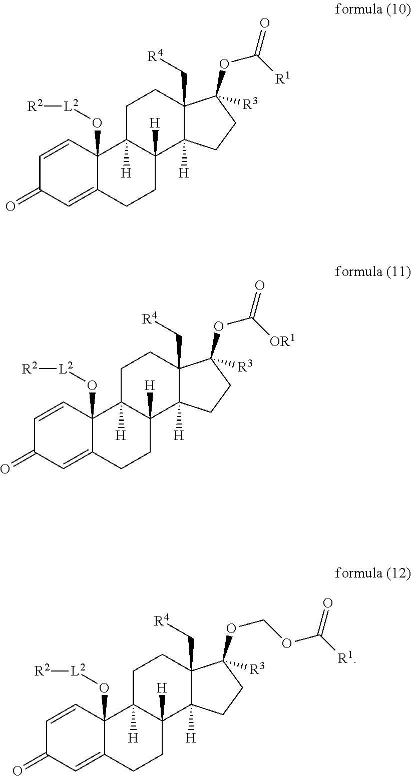

4. The compound of any one of claims 1-3, wherein R¹ is selected from optionally substituted alkyl, optionally substituted aryl, optionally substituted heterocycloalkyl, and optionally substituted heteroaryl.

5. The compound of any one of claims 1-4, wherein R¹ is selected from methyl, ethyl, i-propyl, -n-butyl, n-pentyl, n-hexyl, n-heptyl, n-octyl, -i-butyl, -t-butyl, —C₉H₁₉, —C₁₀H₂₁, —C₁₁H₂₃, 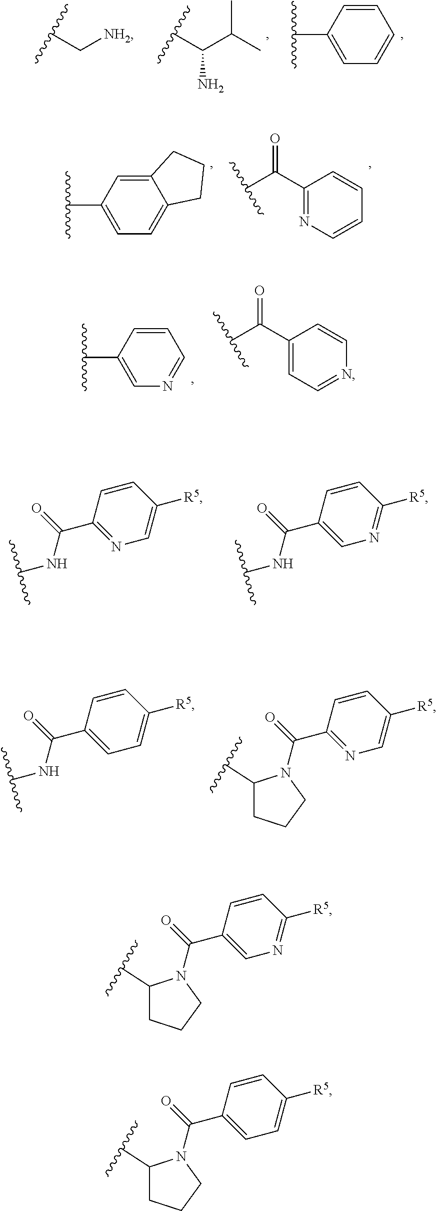
wherein R⁵ is selected from hydrogen, optionally substituted alkyl, —C(O)R⁶, —C(O)N(R⁶)₂, —S(O)R⁶, —S(O)₂R⁶, —S(O)N(R⁶)₂, —S(O)₂N(R⁶)₂, 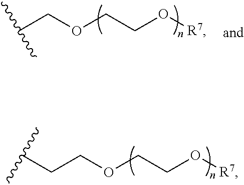
optionally R⁵ is selected from hydrogen and —S(O)₂NH₂, optionally R⁵ is —S(O)₂NH₂, wherein R⁶ is selected from hydrogen and optionally substituted alkyl; n is an integer from 1 to 5, and R⁷ is optionally substituted alkyl, optionally R⁷ is methyl.

6. The compound of any one of claims 1-5, wherein R¹-L¹- is selected from 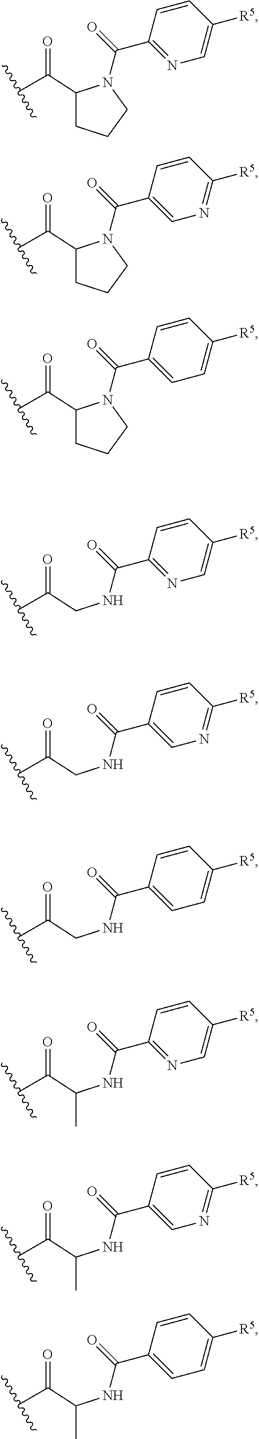
wherein R⁵ is selected from hydrogen, optionally substituted alkyl, —C(O)R⁶, —C(O)N(R⁶)₂, —S(O)R⁶, —S(O)₂R⁶, —S(O)N(R⁶)₂, —S(O)₂N(R⁶)₂, 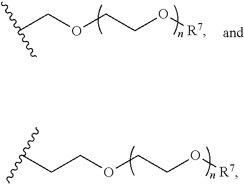
optionally R⁵ is selected from hydrogen and —S(O)₂NH₂, optionally R⁵ is —S(O)₂NH₂, wherein R⁶ is selected from hydrogen and optionally substituted alkyl; n is an integer from 1 to 5, and R⁷ is optionally substituted alkyl, optionally R⁷ is methyl.

7. The compound of any one of claims 1-5, wherein R¹-L¹- is selected from

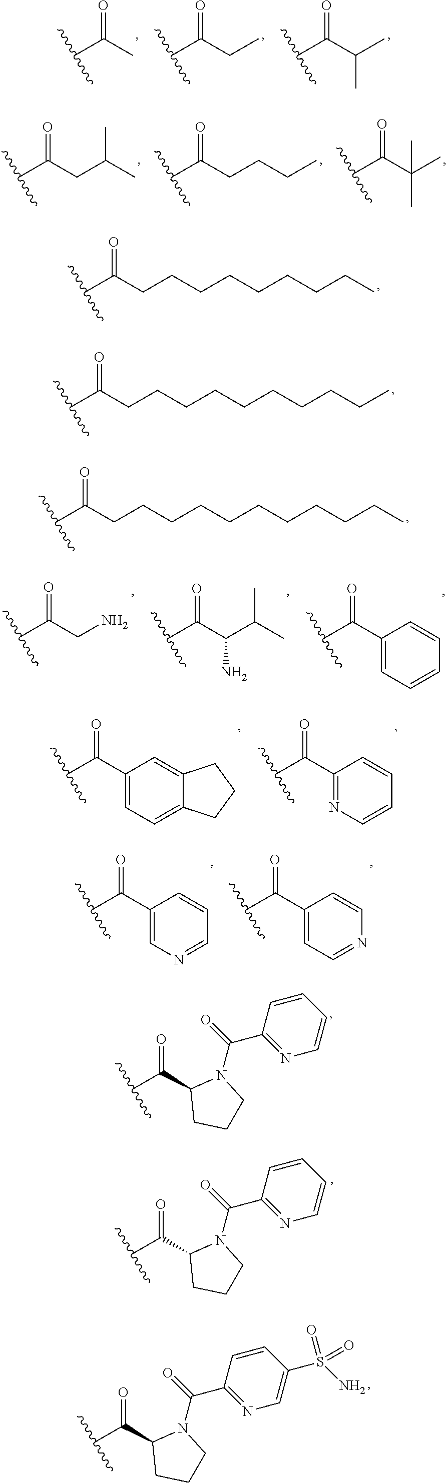

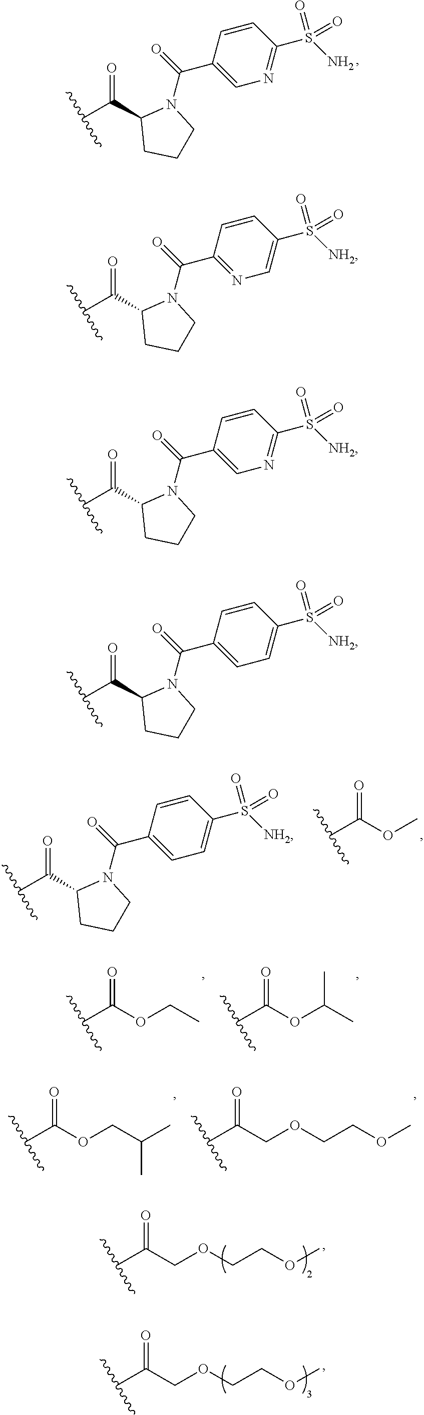

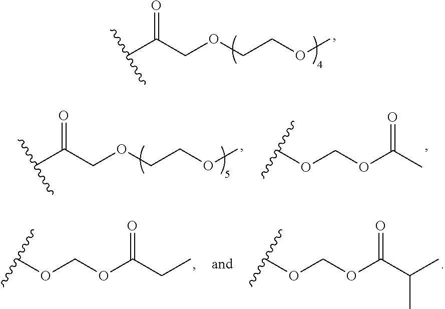

8. The compound of any one of claims 1-7, wherein L² comprises one or more of a bond, —C(O)—, —C(O)O—, and —(CH₂)ₚO—wherein p is an integer from 1 to 5.

9. The compound of any one of claims 1-8, wherein R² is selected from hydrogen, optionally substituted alkyl, optionally substituted aryl, optionally substituted heterocycloalkyl, and optionally substituted heteroaryl.

10. The compound of any one of claims 1-8, wherein R² is selected from methyl, ethyl, i-propyl, 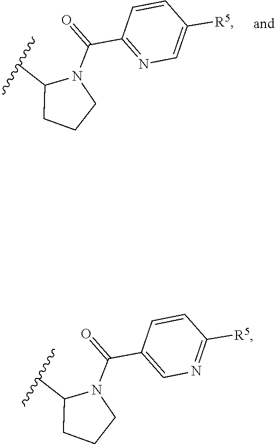
wherein R⁵ is selected from hydrogen, optionally substituted alkyl, —C(O)R⁶, —C(O)N(R⁶)₂, —S(O)R⁶, —S(O)₂R⁶, —S(O)N(R⁶)₂, —S(O)₂N(R⁶)₂, 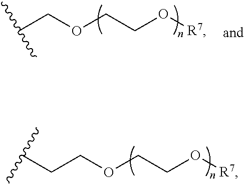
optionally R⁵ is selected from hydrogen and —S(O)₂NH₂, optionally R⁵ is —S(O)₂NH₂, wherein R⁶ is selected from hydrogen and optionally substituted alkyl; n is an integer from 1 to 5, and R⁷ is optionally substituted alkyl, optionally R⁷ is methyl.

11. The compound of any one of claims 1-10, wherein R³ is selected from hydrogen and

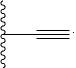 [SMILES: *C#C]

12. The compound of any one of claims 1-11, wherein R⁴ is selected from hydrogen and methyl.

13. The compound of claim 1, wherein the compound of formula (I) is a compound of any one of formulas 1001-1256, or a pharmaceutically acceptable salt, solvate, hydrate, cocrystal, or prodrug thereof:

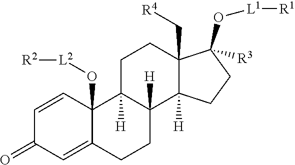

| Formula | | | | |
|---|---|---|---|---|
| No. | —L¹—R¹ | R²—L²— | R³ | R⁴ |
| 1001 | 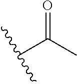 [SMILES: *C(C)=O] | H | H | H |
| 1002 | 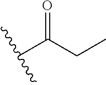 [SMILES: *C(=O)CC] | H | H | H |
| 1003 | 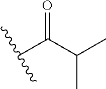 [SMILES: *C(=O)C(C)C] | H | H | H |
| 1004 | 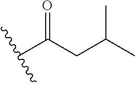 [SMILES: *C(=O)CC(C)C] | H | H | H |
| 1005 | 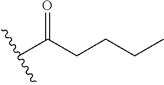 [SMILES: *C(=O)CCCC] | H | H | H |
| 1006 | 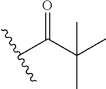 [SMILES: *C(=O)C(C)(C)C] | H | H | H |
| 1007 | 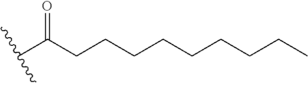 [SMILES: *C(=O)CCCCCCCCC] | H | H | H |
| 1008 | 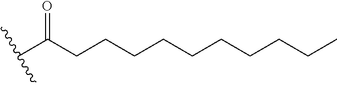 [SMILES: *C(=O)CCCCCCCCCC] | H | H | H |
| 1009 | 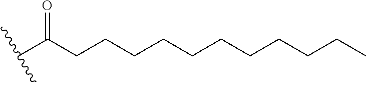 [SMILES: *C(=O)CCCCCCCCCCC] | H | H | H |
| 1010 | 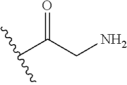 [SMILES: *C(=O)CN] | H | H | H |
| 1011 | 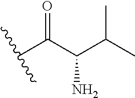 [SMILES: *C(=O)[C@@H](N)C(C)C] | H | H | H |
| 1012 | 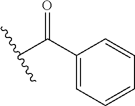 [SMILES: *C(=O)c1ccccc1] | H | H | H |
| 1013 | 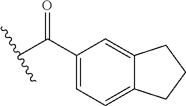 [SMILES: *C(=O)c1ccc2c(c1)CCC2] | H | H | H |
| 1014 | 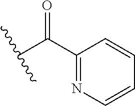 [SMILES: *C(=O)c1ccccn1] | H | H | H |
| 1015 | 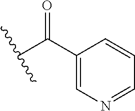 [SMILES: *C(=O)c1cccnc1] | H | H | H |
| 1016 | 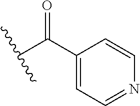 [SMILES: *C(=O)c1ccncc1] | H | H | H |
| 1017 | 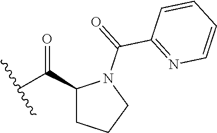 [SMILES: *C(=O)[C@@H]1CCCN1C(=O)c1ccccn1] | H | H | H |
| 1018 | 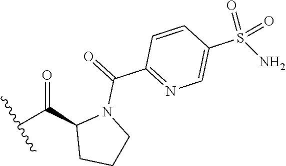 [SMILES: *C(=O)[C@@H]1CCCN1C(=O)c1ccc(S(N)(=O)=O)cn1] | H | H | H |
| 1019 | 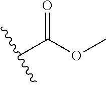 [SMILES: *C(=O)OC] | H | H | H |
| 1020 | 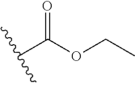 [SMILES: *C(=O)OCC] | H | H | H |
| 1021 | 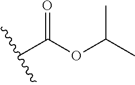 [SMILES: *C(=O)OC(C)C] | H | H | H |
| 1022 | 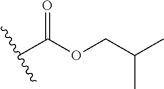 [SMILES: *C(=O)OCC(C)C] | H | H | H |
| 1023 | 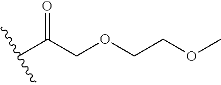 [SMILES: *C(=O)COCCOC] | H | H | H |
| 1024 | 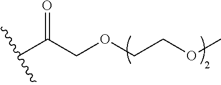 | H | H | H |
| 1025 | 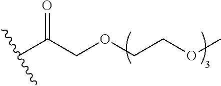 | H | H | H |
| 1026 | 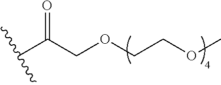 | H | H | H |
| 1027 | 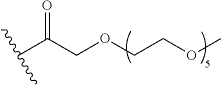 | H | H | H |
| 1028 | 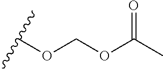 [SMILES: *OCOC(C)=O] | H | H | H |
| 1029 | 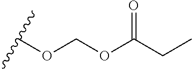 [SMILES: *OCOC(=O)CC] | H | H | H |
| 1030 | 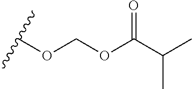 [SMILES: *OCOC(=O)C(C)C] | H | H | H |
| 1031 | 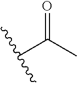 [SMILES: *C(C)=O] | 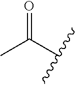 [SMILES: *C(C)=O] | H | H |
| 1032 | 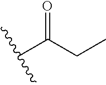 [SMILES: *C(=O)CC] | 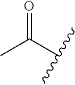 [SMILES: *C(C)=O] | H | H |
| 1033 | 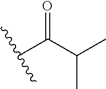 [SMILES: *C(=O)C(C)C] | 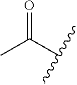 [SMILES: *C(C)=O] | H | H |
| 1034 | 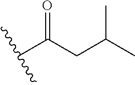 [SMILES: *C(=O)CC(C)C] | 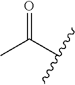 [SMILES: *C(C)=O] | H | H |
| 1035 | 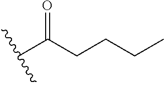 [SMILES: *C(=O)CCCC] | 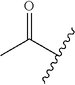 [SMILES: *C(C)=O] | H | H |
| 1036 | 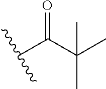 [SMILES: *C(=O)C(C)(C)C] | 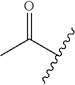 [SMILES: *C(C)=O] | H | H |
| 1037 | 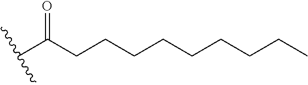 [SMILES: *C(=O)CCCCCCCCC] | 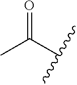 [SMILES: *C(C)=O] | H | H |
| 1038 | 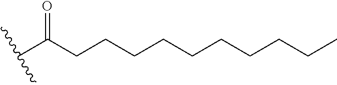 [SMILES: *C(=O)CCCCCCCCCC] | 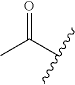 [SMILES: *C(C)=O] | H | H |
| 1039 | 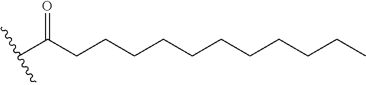 [SMILES: *C(=O)CCCCCCCCCCC] | 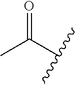 [SMILES: *C(C)=O] | H | H |
| 1040 | 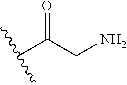 [SMILES: *C(=O)CN] | 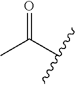 [SMILES: *C(C)=O] | H | H |
| 1041 | 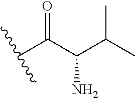 [SMILES: *C(=O)[C@@H](N)C(C)C] | 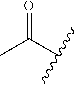 [SMILES: *C(C)=O] | H | H |
| 1042 | 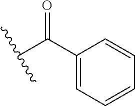 [SMILES: *C(=O)c1ccccc1] | 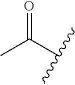 [SMILES: *C(C)=O] | H | H |
| 1043 | 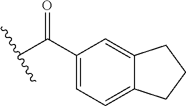 [SMILES: *C(=O)c1ccc2c(c1)CCC2] | 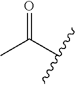 [SMILES: *C(C)=O] | H | H |
| 1044 | 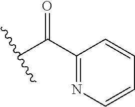 [SMILES: *C(=O)c1ccccn1] | 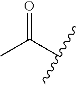 [SMILES: *C(C)=O] | H | H |
| 1045 | 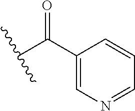 [SMILES: *C(=O)c1cccnc1] | 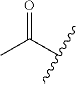 [SMILES: *C(C)=O] | H | H |
| 1046 | 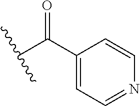 [SMILES: *C(=O)c1ccncc1] | 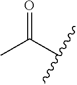 [SMILES: *C(C)=O] | H | H |
| 1047 | 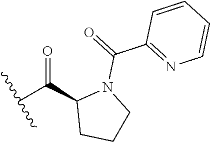 [SMILES: *C(=O)[C@@H]1CCCN1C(=O)c1ccccn1] | 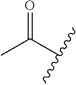 [SMILES: *C(C)=O] | H | H |
| 1048 | 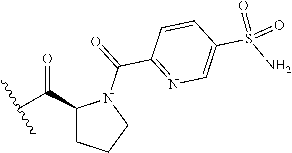 [SMILES: *C(=O)[C@@H]1CCCN1C(=O)c1ccc(S(N)(=O)=O)cn1] | 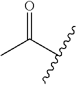 [SMILES: *C(C)=O] | H | H |
| 1049 | 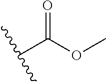 [SMILES: *C(=O)OC] | 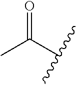 [SMILES: *C(C)=O] | H | H |
| 1050 | 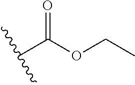 [SMILES: *C(=O)OCC] | 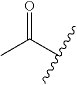 [SMILES: *C(C)=O] | H | H |
| 1051 | 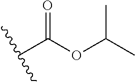 [SMILES: *C(=O)OC(C)C] | 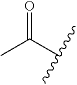 [SMILES: *C(C)=O] | H | H |
| 1052 | 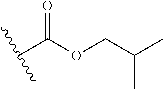 [SMILES: *C(=O)OCC(C)C] | 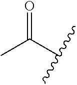 [SMILES: *C(C)=O] | H | H |
| 1053 | 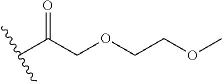 [SMILES: *C(=O)COCCOC] | 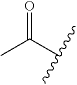 [SMILES: *C(C)=O] | H | H |
| 1054 | 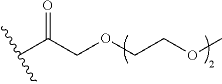 | 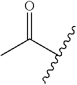 [SMILES: *C(C)=O] | H | H |
| 1055 | 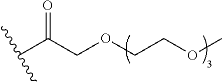 | 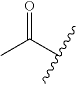 [SMILES: *C(C)=O] | H | H |
| 1056 | 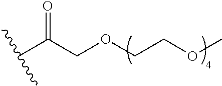 | 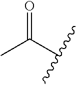 [SMILES: *C(C)=O] | H | H |
| 1057 | 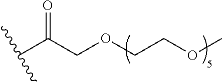 | 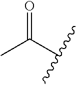 [SMILES: *C(C)=O] | H | H |
| 1058 | 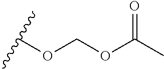 [SMILES: *OCOC(C)=O] | 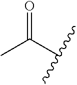 [SMILES: *C(C)=O] | H | H |
| 1059 | 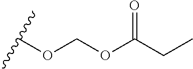 [SMILES: *OCOC(=O)CC] | 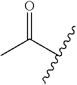 [SMILES: *C(C)=O] | H | H |
| 1060 | 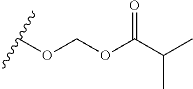 [SMILES: *OCOC(=O)C(C)C] | 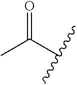 [SMILES: *C(C)=O] | H | H |
| 1061 | 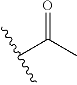 [SMILES: *C(C)=O] | H | 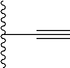 [SMILES: *C#C] | H |
| 1062 | 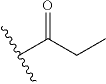 [SMILES: *C(=O)CC] | H | 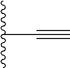 [SMILES: *C#C] | H |
| 1063 | 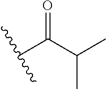 [SMILES: *C(=O)C(C)C] | H | 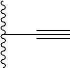 [SMILES: *C#C] | H |
| 1064 | 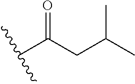 [SMILES: *C(=O)CC(C)C] | H | 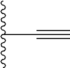 [SMILES: *C#C] | H |
| 1065 | 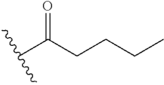 [SMILES: *C(=O)CCCC] | H | 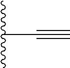 [SMILES: *C#C] | H |
| 1066 | 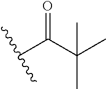 [SMILES: *C(=O)C(C)(C)C] | H | 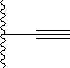 [SMILES: *C#C] | H |
| 1067 | 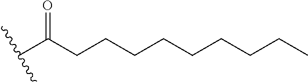 [SMILES: *C(=O)CCCCCCCCC] | H | 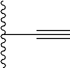 [SMILES: *C#C] | H |
| 1068 | 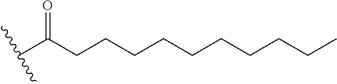 [SMILES: *C(=O)CCCCCCCCCC] | H | 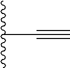 [SMILES: *C#C] | H |
| 1069 | 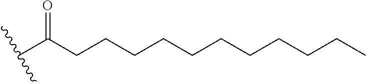 [SMILES: *C(=O)CCCCCCCCCCC] | H | 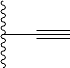 [SMILES: *C#C] | H |
| 1070 | 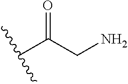 [SMILES: *C(=O)CN] | H | 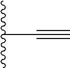 [SMILES: *C#C] | H |
| 1071 | 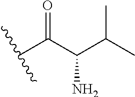 [SMILES: *C(=O)[C@@H](N)C(C)C] | H | 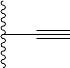 [SMILES: *C#C] | H |
| 1072 | 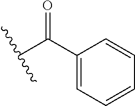 [SMILES: *C(=O)c1ccccc1] | H | 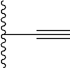 [SMILES: *C#C] | H |
| 1073 | 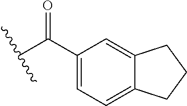 [SMILES: *C(=O)c1ccc2c(c1)CCC2] | H | 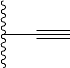 [SMILES: *C#C] | H |
| 1074 | 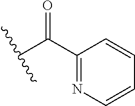 [SMILES: *C(=O)c1ccccn1] | H | 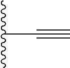 [SMILES: *C#C] | H |
| 1075 | 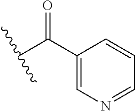 [SMILES: *C(=O)c1cccnc1] | H | 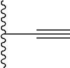 [SMILES: *C#C] | H |
| 1076 | 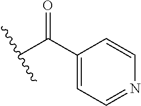 [SMILES: *C(=O)c1ccncc1] | H | 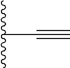 [SMILES: *C#C] | H |
| 1077 | 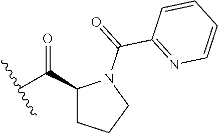 [SMILES: *C(=O)[C@@H]1CCCN1C(=O)c1ccccn1] | H | 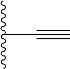 [SMILES: *C#C] | H |
| 1078 | 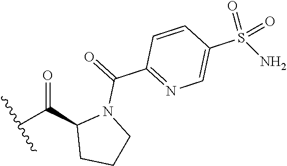 [SMILES: *C(=O)[C@@H]1CCCN1C(=O)c1ccc(S(N)(=O)=O)cn1] | H | 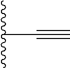 [SMILES: *C#C] | H |
| 1079 | 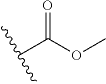 [SMILES: *C(=O)OC] | H | 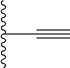 [SMILES: *C#C] | H |
| 1080 | 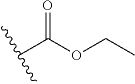 [SMILES: *C(=O)OCC] | H | 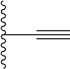 [SMILES: *C#C] | H |
| 1081 | 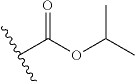 [SMILES: *C(=O)OC(C)C] | H | 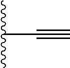 [SMILES: *C#C] | H |
| 1082 | 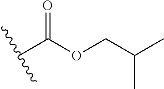 [SMILES: *C(=O)OCC(C)C] | H | 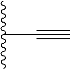 [SMILES: *C#C] | H |
| 1083 | 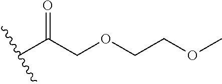 [SMILES: *C(=O)COCCOC] | H | 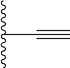 [SMILES: *C#C] | H |
| 1084 | 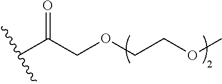 | H | 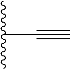 [SMILES: *C#C] | H |
| 1085 | 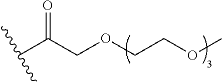 | H | 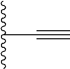 [SMILES: *C#C] | H |
| 1086 | 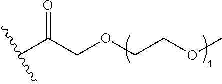 | H | 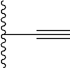 [SMILES: *C#C] | H |
| 1087 | 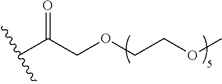 | H | 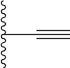 [SMILES: *C#C] | H |
| 1088 | 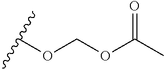 [SMILES: *OCOC(C)=O] | H | 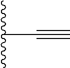 [SMILES: *C#C] | H |
| 1089 | 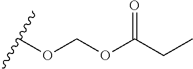 [SMILES: *OCOC(=O)CC] | H | 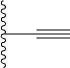 [SMILES: *C#C] | H |
| 1090 | 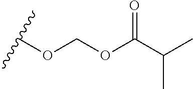 [SMILES: *OCOC(=O)C(C)C] | H | 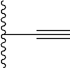 [SMILES: *C#C] | H |
| 1091 | 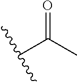 [SMILES: *C(C)=O] | 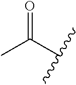 [SMILES: *C(C)=O] | 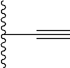 [SMILES: *C#C] | H |
| 1092 | 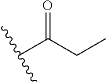 [SMILES: *C(=O)CC] | 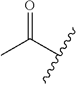 [SMILES: *C(C)=O] | 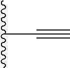 [SMILES: *C#C] | H |
| 1093 | 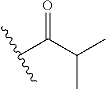 [SMILES: *C(=O)C(C)C] | 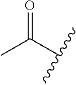 [SMILES: *C(C)=O] | 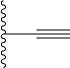 [SMILES: *C#C] | H |
| 1094 | 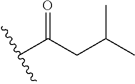 [SMILES: *C(=O)CC(C)C] | 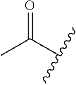 [SMILES: *C(C)=O] | 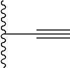 [SMILES: *C#C] | H |
| 1095 | 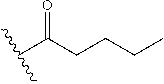 [SMILES: *C(=O)CCCC] | 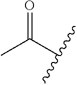 [SMILES: *C(C)=O] | 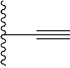 [SMILES: *C#C] | H |
| 1096 | 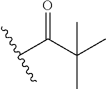 [SMILES: *C(=O)C(C)(C)C] | 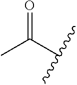 [SMILES: *C(C)=O] | 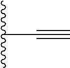 [SMILES: *C#C] | H |
| 1097 | 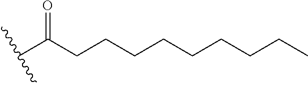 [SMILES: *C(=O)CCCCCCCCC] | 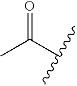 [SMILES: *C(C)=O] | 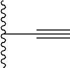 [SMILES: *C#C] | H |
| 1098 | 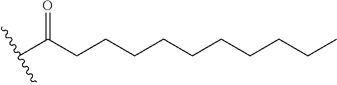 [SMILES: *C(=O)CCCCCCCCCC] | 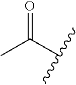 [SMILES: *C(C)=O] | 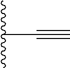 [SMILES: *C#C] | H |
| 1099 | 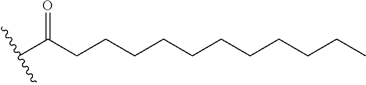 [SMILES: *C(=O)CCCCCCCCCCC] | 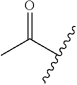 [SMILES: *C(C)=O] | 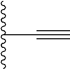 [SMILES: *C#C] | H |
| 1100 | 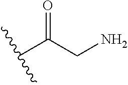 [SMILES: *C(=O)CN] | 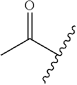 [SMILES: *C(C)=O] | 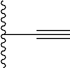 [SMILES: *C#C] | H |
| 1101 | 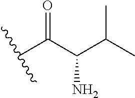 [SMILES: *C(=O)[C@@H](N)C(C)C] | 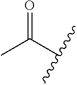 [SMILES: *C(C)=O] | 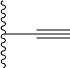 [SMILES: *C#C] | H |
| 1102 | 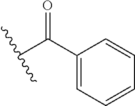 [SMILES: *C(=O)c1ccccc1] | 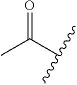 [SMILES: *C(C)=O] | 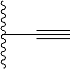 [SMILES: *C#C] | H |
| 1103 | 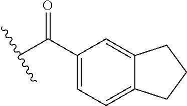 [SMILES: *C(=O)c1ccc2c(c1)CCC2] | 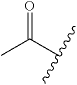 [SMILES: *C(C)=O] | 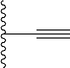 [SMILES: *C#C] | H |
| 1104 | 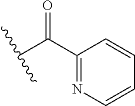 [SMILES: *C(=O)c1ccccn1] | 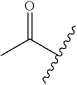 [SMILES: *C(C)=O] | 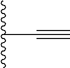 [SMILES: *C#C] | H |
| 1105 | 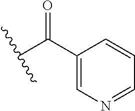 [SMILES: *C(=O)c1cccnc1] | 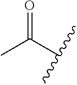 [SMILES: *C(C)=O] | 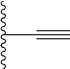 [SMILES: *C#C] | H |
| 1106 | 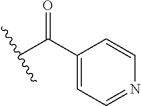 [SMILES: *C(=O)c1ccncc1] | 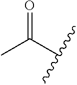 [SMILES: *C(C)=O] | 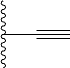 [SMILES: *C#C] | H |
| 1107 | 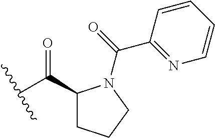 [SMILES: *C(=O)[C@@H]1CCCN1C(=O)c1ccccn1] | 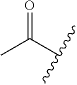 [SMILES: *C(C)=O] | 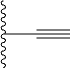 [SMILES: *C#C] | H |
| 1108 | 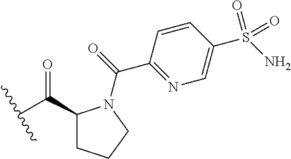 [SMILES: *C(=O)[C@@H]1CCCN1C(=O)c1ccc(S(N)(=O)=O)cn1] | 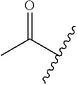 [SMILES: *C(C)=O] | 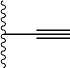 [SMILES: *C#C] | H |
| 1109 | 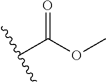 [SMILES: *C(=O)OC] | 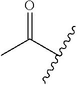 [SMILES: *C(C)=O] | 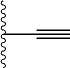 [SMILES: *C#C] | H |
| 1110 | 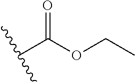 [SMILES: *C(=O)OCC] | 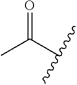 [SMILES: *C(C)=O] | 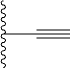 [SMILES: *C#C] | H |
| 1111 | 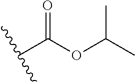 [SMILES: *C(=O)OC(C)C] | 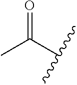 [SMILES: *C(C)=O] | 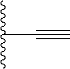 [SMILES: *C#C] | H |
| 1112 | 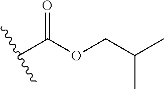 [SMILES: *C(=O)OCC(C)C] | 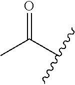 [SMILES: *C(C)=O] | 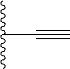 [SMILES: *C#C] | H |
| 1113 | 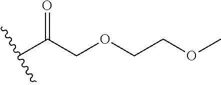 [SMILES: *C(=O)COCCOC] | 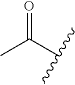 [SMILES: *C(C)=O] | 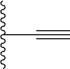 [SMILES: *C#C] | H |
| 1114 | 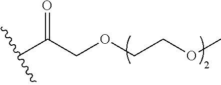 | 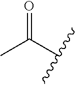 [SMILES: *C(C)=O] | 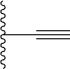 [SMILES: *C#C] | H |
| 1115 | 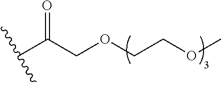 | 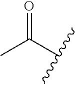 [SMILES: *C(C)=O] | 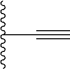 [SMILES: *C#C] | H |
| 1116 | 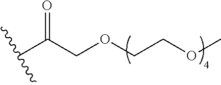 | 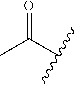 [SMILES: *C(C)=O] | 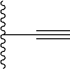 [SMILES: *C#C] | H |
| 1117 | 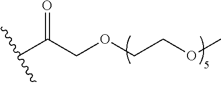 | 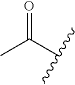 [SMILES: *C(C)=O] | 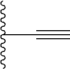 [SMILES: *C#C] | H |
| 1118 | 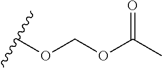 [SMILES: *OCOC(C)=O] | 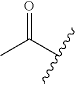 [SMILES: *C(C)=O] | 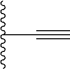 [SMILES: *C#C] | H |
| 1119 | 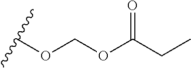 [SMILES: *OCOC(=O)CC] | 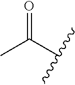 [SMILES: *C(C)=O] | 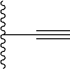 [SMILES: *C#C] | H |
| 1120 | 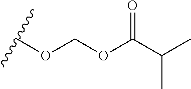 [SMILES: *OCOC(=O)C(C)C] | 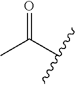 [SMILES: *C(C)=O] | 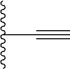 [SMILES: *C#C] | H |
| 1121 | 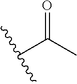 [SMILES: *C(C)=O] | H | H | Me |
| 1122 | 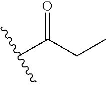 [SMILES: *C(=O)CC] | H | H | Me |
| 1123 | 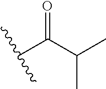 [SMILES: *C(=O)C(C)C] | H | H | Me |
| 1124 | 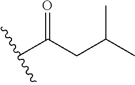 [SMILES: *C(=O)CC(C)C] | H | H | Me |
| 1125 | 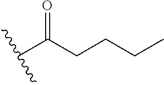 [SMILES: *C(=O)CCCC] | H | H | Me |
| 1126 | 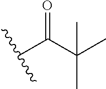 [SMILES: *C(=O)C(C)(C)C] | H | H | Me |
| 1127 | 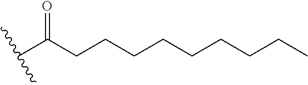 [SMILES: *C(=O)CCCCCCCCC] | H | H | Me |
| 1128 | 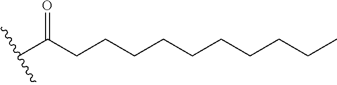 [SMILES: *C(=O)CCCCCCCCCC] | H | H | Me |
| 1129 | 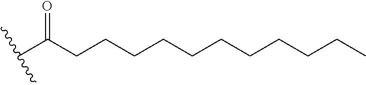 [SMILES: *C(=O)CCCCCCCCCCC] | H | H | Me |
| 1130 | 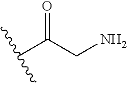 [SMILES: *C(=O)CN] | H | H | Me |
| 1131 | 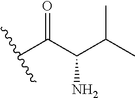 [SMILES: *C(=O)[C@@H](N)C(C)C] | H | H | Me |
| 1132 | 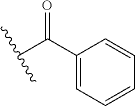 [SMILES: *C(=O)c1ccccc1] | H | H | Me |
| 1133 | 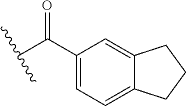 [SMILES: *C(=O)c1ccc2c(c1)CCC2] | H | H | Me |
| 1134 | 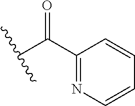 [SMILES: *C(=O)c1ccccn1] | H | H | Me |
| 1135 | 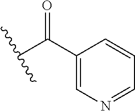 [SMILES: *C(=O)c1cccnc1] | H | H | Me |
| 1136 | 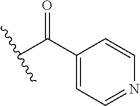 [SMILES: *C(=O)c1ccncc1] | H | H | Me |
| 1137 | 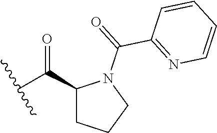 [SMILES: *C(=O)[C@@H]1CCCN1C(=O)c1ccccn1] | H | H | Me |
| 1138 | 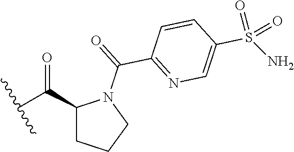 [SMILES: *C(=O)[C@@H]1CCCN1C(=O)c1ccc(S(N)(=O)=O)cn1] | H | H | Me |
| 1139 | 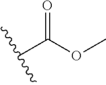 [SMILES: *C(=O)OC] | H | H | Me |
| 1140 | 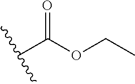 [SMILES: *C(=O)OCC] | H | H | Me |
| 1141 | 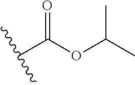 [SMILES: *C(=O)OC(C)C] | H | H | Me |
| 1142 | 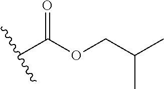 [SMILES: *C(=O)OCC(C)C] | H | H | Me |
| 1143 | 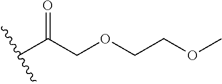 [SMILES: *C(=O)COCCOC] | H | H | Me |
| 1144 | 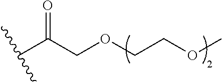 | H | H | Me |
| 1145 | 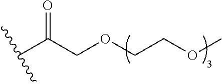 | H | H | Me |
| 1146 | 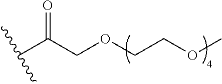 | H | H | Me |
| 1147 | 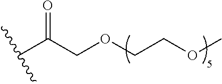 | H | H | Me |
| 1148 | 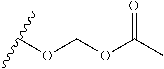 [SMILES: *OCOC(C)=O] | H | H | Me |
| 1149 | 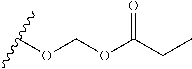 [SMILES: *OCOC(=O)CC] | H | H | Me |
| 1150 | 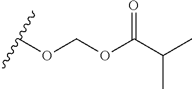 [SMILES: *OCOC(=O)C(C)C] | H | H | Me |
| 1151 | 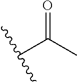 [SMILES: *C(C)=O] | 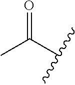 [SMILES: *C(C)=O] | H | Me |
| 1152 | 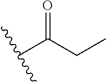 [SMILES: *C(=O)CC] | 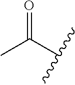 [SMILES: *C(C)=O] | H | Me |
| 1153 | 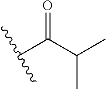 [SMILES: *C(=O)C(C)C] | 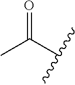 [SMILES: *C(C)=O] | H | Me |
| 1154 | 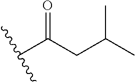 [SMILES: *C(=O)CC(C)C] | 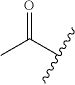 [SMILES: *C(C)=O] | H | Me |
| 1155 | 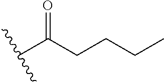 [SMILES: *C(=O)CCCC] | 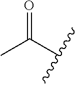 [SMILES: *C(C)=O] | H | Me |
| 1156 | 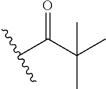 [SMILES: *C(=O)C(C)(C)C] | 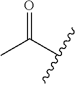 [SMILES: *C(C)=O] | H | Me |
| 1157 | 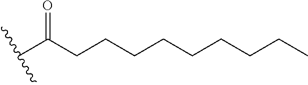 [SMILES: *C(=O)CCCCCCCCC] | 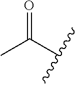 [SMILES: *C(C)=O] | H | Me |
| 1158 | 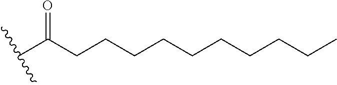 [SMILES: *C(=O)CCCCCCCCCC] | 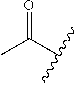 [SMILES: *C(C)=O] | H | Me |
| 1159 | 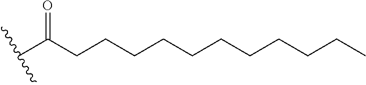 [SMILES: *C(=O)CCCCCCCCCCC] | 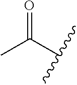 [SMILES: *C(C)=O] | H | Me |
| 1160 | 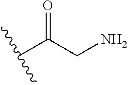 [SMILES: *C(=O)CN] | 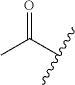 [SMILES: *C(C)=O] | H | Me |
| 1161 | 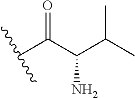 [SMILES: *C(=O)[C@@H](N)C(C)C] | 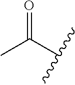 [SMILES: *C(C)=O] | H | Me |
| 1162 | 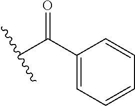 [SMILES: *C(=O)c1ccccc1] | 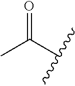 [SMILES: *C(C)=O] | H | Me |
| 1163 | 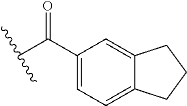 [SMILES: *C(=O)c1ccc2c(c1)CCC2] | 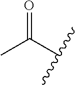 [SMILES: *C(C)=O] | H | Me |
| 1164 | 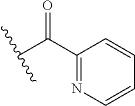 [SMILES: *C(=O)c1ccccn1] | 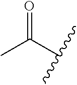 [SMILES: *C(C)=O] | H | Me |
| 1165 | 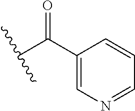 [SMILES: *C(=O)c1cccnc1] | 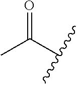 [SMILES: *C(C)=O] | H | Me |
| 1166 | 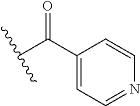 [SMILES: *C(=O)c1ccncc1] | 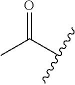 [SMILES: *C(C)=O] | H | Me |
| 1167 | 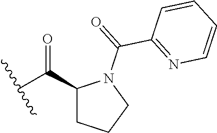 [SMILES: *C(=O)[C@@H]1CCCN1C(=O)c1ccccn1] | 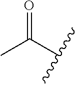 [SMILES: *C(C)=O] | H | Me |
| 1168 | 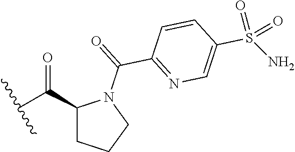 [SMILES: *C(=O)[C@@H]1CCCN1C(=O)c1ccc(S(N)(=O)=O)cn1] | 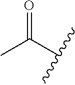 [SMILES: *C(C)=O] | H | Me |
| 1169 | 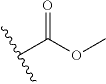 [SMILES: *C(=O)OC] | 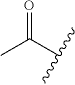 [SMILES: *C(C)=O] | H | Me |
| 1170 | 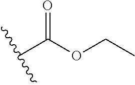 [SMILES: *C(=O)OCC] | 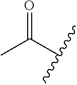 [SMILES: *C(C)=O] | H | Me |
| 1171 | 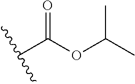 [SMILES: *C(=O)OC(C)C] | 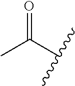 [SMILES: *C(C)=O] | H | Me |
| 1172 | 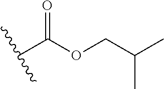 [SMILES: *C(=O)OCC(C)C] | 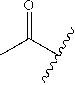 [SMILES: *C(C)=O] | H | Me |
| 1173 | 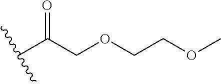 [SMILES: *C(=O)COCCOC] | 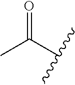 [SMILES: *C(C)=O] | H | Me |
| 1174 | 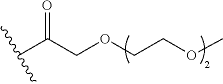 | 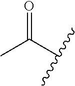 [SMILES: *C(C)=O] | H | Me |
| 1175 | 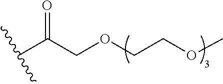 | 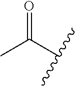 [SMILES: *C(C)=O] | H | Me |
| 1176 | 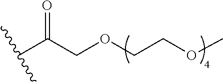 | 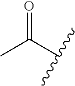 [SMILES: *C(C)=O] | H | Me |
| 1177 | 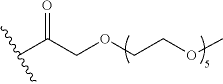 | 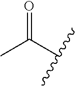 [SMILES: *C(C)=O] | H | Me |
| 1178 | 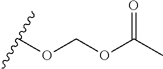 [SMILES: *OCOC(C)=O] | 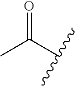 [SMILES: *C(C)=O] | H | Me |
| 1179 | 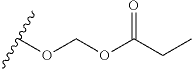 [SMILES: *OCOC(=O)CC] | 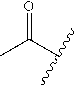 [SMILES: *C(C)=O] | H | Me |
| 1180 | 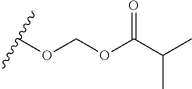 [SMILES: *OCOC(=O)C(C)C] | 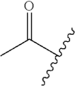 [SMILES: *C(C)=O] | H | Me |
| 1181 | 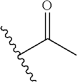 [SMILES: *C(C)=O] | H | 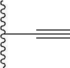 [SMILES: *C#C] | Me |
| 1182 | 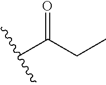 [SMILES: *C(=O)CC] | H | 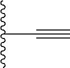 [SMILES: *C#C] | Me |
| 1183 | 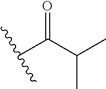 [SMILES: *C(=O)C(C)C] | H | 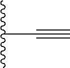 [SMILES: *C#C] | Me |
| 1184 | 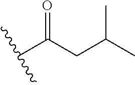 [SMILES: *C(=O)CC(C)C] | H | 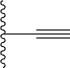 [SMILES: *C#C] | Me |
| 1185 | 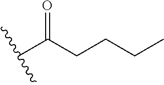 [SMILES: *C(=O)CCCC] | H | 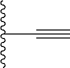 [SMILES: *C#C] | Me |
| 1186 | 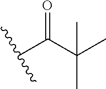 [SMILES: *C(=O)C(C)(C)C] | H | 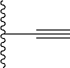 [SMILES: *C#C] | Me |
| 1187 | 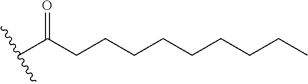 [SMILES: *C(=O)CCCCCCCCC] | H | 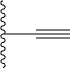 [SMILES: *C#C] | Me |
| 1188 | 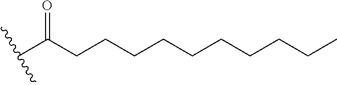 [SMILES: *C(=O)CCCCCCCCCC] | H | 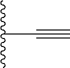 [SMILES: *C#C] | Me |
| 1189 | 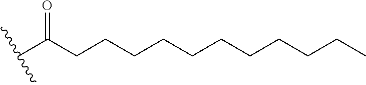 [SMILES: *C(=O)CCCCCCCCCCC] | H | 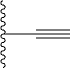 [SMILES: *C#C] | Me |
| 1190 | 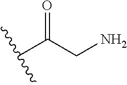 [SMILES: *C(=O)CN] | H | 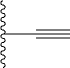 [SMILES: *C#C] | Me |
| 1191 | 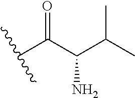 [SMILES: *C(=O)[C@@H](N)C(C)C] | H | 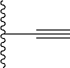 [SMILES: *C#C] | Me |
| 1192 | 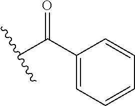 [SMILES: *C(=O)c1ccccc1] | H | 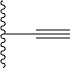 [SMILES: *C#C] | Me |
| 1193 | 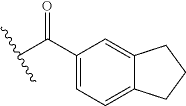 [SMILES: *C(=O)c1ccc2c(c1)CCC2] | H | 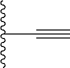 [SMILES: *C#C] | Me |
| 1194 | 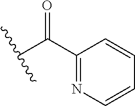 [SMILES: *C(=O)c1ccccn1] | H | 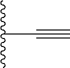 [SMILES: *C#C] | Me |
| 1195 | 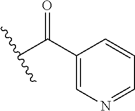 [SMILES: *C(=O)c1cccnc1] | H | 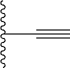 [SMILES: *C#C] | Me |
| 1196 | 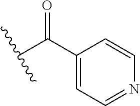 [SMILES: *C(=O)c1ccncc1] | H | 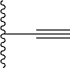 [SMILES: *C#C] | Me |
| 1197 | 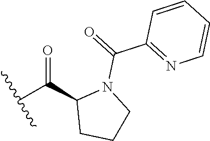 [SMILES: *C(=O)[C@@H]1CCCN1C(=O)c1ccccn1] | H | 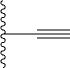 [SMILES: *C#C] | Me |
| 1198 | 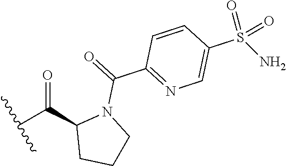 [SMILES: *C(=O)[C@@H]1CCCN1C(=O)c1ccc(S(N)(=O)=O)cn1] | H | 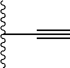 [SMILES: *C#C] | Me |
| 1199 | 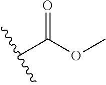 [SMILES: *C(=O)OC] | H | 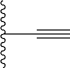 [SMILES: *C#C] | Me |
| 1200 | 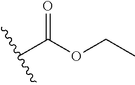 [SMILES: *C(=O)OCC] | H | 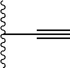 [SMILES: *C#C] | Me |
| 1201 | 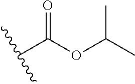 [SMILES: *C(=O)OC(C)C] | H | 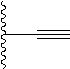 [SMILES: *C#C] | Me |
| 1202 | 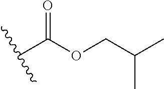 [SMILES: *C(=O)OCC(C)C] | H | 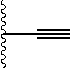 [SMILES: *C#C] | Me |
| 1203 | 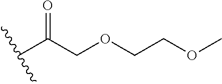 [SMILES: *C(=O)COCCOC] | H | 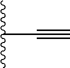 [SMILES: *C#C] | Me |
| 1204 | 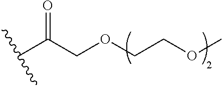 | H | 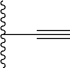 [SMILES: *C#C] | Me |
| 1205 | 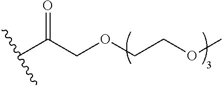 | H | 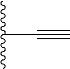 [SMILES: *C#C] | Me |
| 1206 | 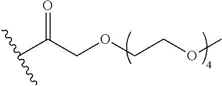 | H | 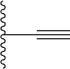 [SMILES: *C#C] | Me |
| 1207 | 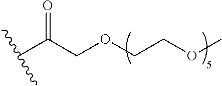 | H | 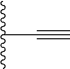 [SMILES: *C#C] | Me |
| 1208 | 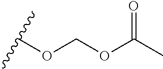 [SMILES: *OCOC(C)=O] | H | 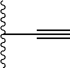 [SMILES: *C#C] | Me |
| 1209 | 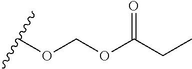 [SMILES: *OCOC(=O)CC] | H | 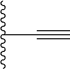 [SMILES: *C#C] | Me |
| 1210 | 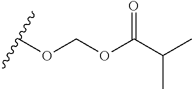 [SMILES: *OCOC(=O)C(C)C] | H | 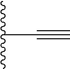 [SMILES: *C#C] | Me |
| 1211 | 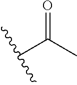 [SMILES: *C(C)=O] | 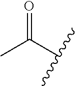 [SMILES: *C(C)=O] | 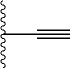 [SMILES: *C#C] | Me |
| 1212 | 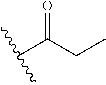 [SMILES: *C(=O)CC] | 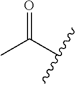 [SMILES: *C(C)=O] | 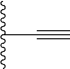 [SMILES: *C#C] | Me |
| 1213 | 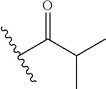 [SMILES: *C(=O)C(C)C] | 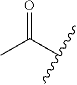 [SMILES: *C(C)=O] | 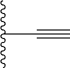 [SMILES: *C#C] | Me |
| 1214 | 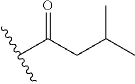 [SMILES: *C(=O)CC(C)C] | 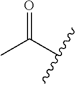 [SMILES: *C(C)=O] | 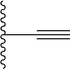 [SMILES: *C#C] | Me |
| 1215 | 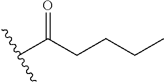 [SMILES: *C(=O)CCCC] | 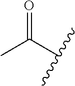 [SMILES: *C(C)=O] | 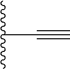 [SMILES: *C#C] | Me |
| 1216 | 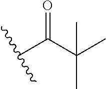 [SMILES: *C(=O)C(C)(C)C] | 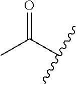 [SMILES: *C(C)=O] | 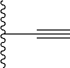 [SMILES: *C#C] | Me |
| 1217 | 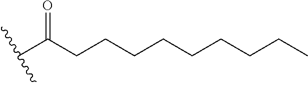 [SMILES: *C(=O)CCCCCCCCC] | 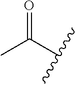 [SMILES: *C(C)=O] | 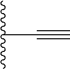 [SMILES: *C#C] | Me |
| 1218 | 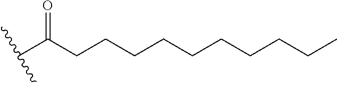 [SMILES: *C(=O)CCCCCCCCCC] | 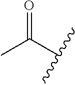 [SMILES: *C(C)=O] | 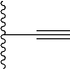 [SMILES: *C#C] | Me |
| 1219 | 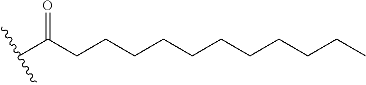 [SMILES: *C(=O)CCCCCCCCCCC] | 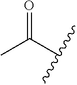 [SMILES: *C(C)=O] | 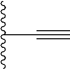 [SMILES: *C#C] | Me |
| 1220 | 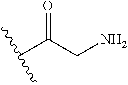 [SMILES: *C(=O)CN] | 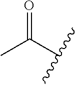 [SMILES: *C(C)=O] | 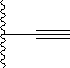 [SMILES: *C#C] | Me |
| 1221 | 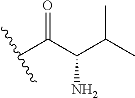 [SMILES: *C(=O)[C@@H](N)C(C)C] | 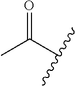 [SMILES: *C(C)=O] | 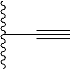 [SMILES: *C#C] | Me |
| 1222 | 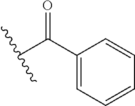 [SMILES: *C(=O)c1ccccc1] | 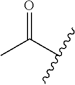 [SMILES: *C(C)=O] | 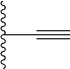 [SMILES: *C#C] | Me |
| 1223 | 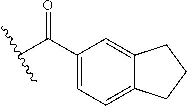 [SMILES: *C(=O)c1ccc2c(c1)CCC2] | 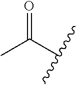 [SMILES: *C(C)=O] | 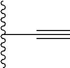 [SMILES: *C#C] | Me |
| 1224 | 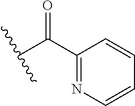 [SMILES: *C(=O)c1ccccn1] | 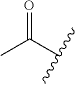 [SMILES: *C(C)=O] | 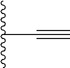 [SMILES: *C#C] | Me |
| 1225 | 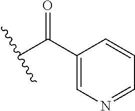 [SMILES: *C(=O)c1cccnc1] | 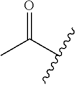 [SMILES: *C(C)=O] | 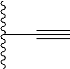 [SMILES: *C#C] | Me |
| 1226 | 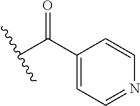 [SMILES: *C(=O)c1ccncc1] | 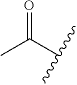 [SMILES: *C(C)=O] | 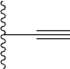 [SMILES: *C#C] | Me |
| 1227 | 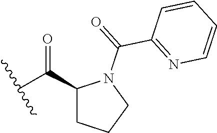 [SMILES: *C(=O)[C@@H]1CCCN1C(=O)c1ccccn1] | 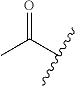 [SMILES: *C(C)=O] | 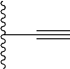 [SMILES: *C#C] | Me |
| 1228 | 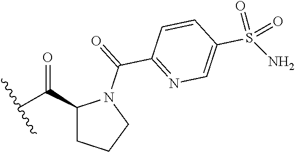 [SMILES: *C(=O)[C@@H]1CCCN1C(=O)c1ccc(S(N)(=O)=O)cn1] | 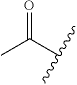 [SMILES: *C(C)=O] | 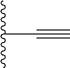 [SMILES: *C#C] | Me |
| 1229 | 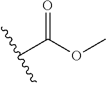 [SMILES: *C(=O)OC] | 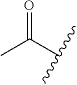 [SMILES: *C(C)=O] | 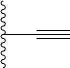 [SMILES: *C#C] | Me |
| 1230 | 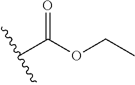 [SMILES: *C(=O)OCC] | 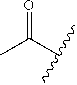 [SMILES: *C(C)=O] | 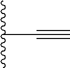 [SMILES: *C#C] | Me |
| 1231 | 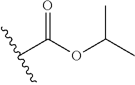 [SMILES: *C(=O)OC(C)C] | 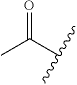 [SMILES: *C(C)=O] | 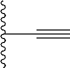 [SMILES: *C#C] | Me |
| 1232 | 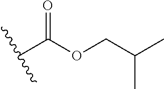 [SMILES: *C(=O)OCC(C)C] | 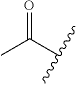 [SMILES: *C(C)=O] | 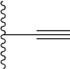 [SMILES: *C#C] | Me |
| 1233 | 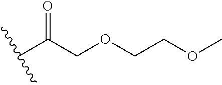 [SMILES: *C(=O)COCCOC] | 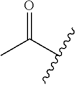 [SMILES: *C(C)=O] | 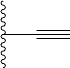 [SMILES: *C#C] | Me |
| 1234 | 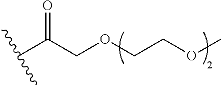 | 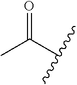 [SMILES: *C(C)=O] | 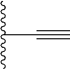 [SMILES: *C#C] | Me |
| 1235 | 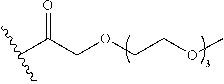 | 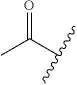 [SMILES: *C(C)=O] | 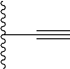 [SMILES: *C#C] | Me |
| 1236 | 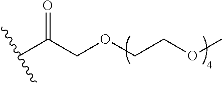 | 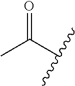 [SMILES: *C(C)=O] | 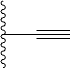 [SMILES: *C#C] | Me |
| 1237 | 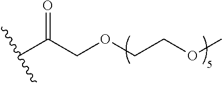 | 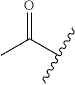 [SMILES: *C(C)=O] | 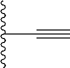 [SMILES: *C#C] | Me |
| 1238 | 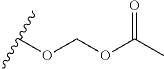 [SMILES: *OCOC(C)=O] | 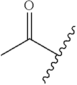 [SMILES: *C(C)=O] | 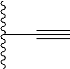 [SMILES: *C#C] | Me |
| 1239 | 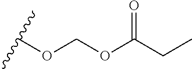 [SMILES: *OCOC(=O)CC] | 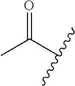 [SMILES: *C(C)=O] | 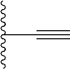 [SMILES: *C#C] | Me |
| 1240 | 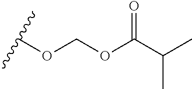 [SMILES: *OCOC(=O)C(C)C] | 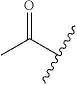 [SMILES: *C(C)=O] | 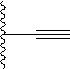 [SMILES: *C#C] | Me |
| 1241 | 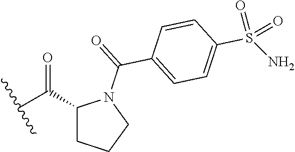 [SMILES: *C(=O)[C@H]1CCCN1C(=O)c1ccc(S(N)(=O)=O)cc1] | H | H | H |
| 1242 | 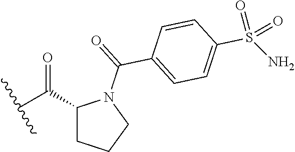 [SMILES: *C(=O)[C@H]1CCCN1C(=O)c1ccc(S(N)(=O)=O)cc1] | 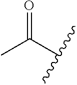 [SMILES: *C(C)=O] | H | H |
| 1243 | 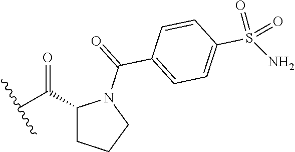 [SMILES: *C(=O)[C@H]1CCCN1C(=O)c1ccc(S(N)(=O)=O)cc1] | H | 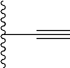 [SMILES: *C#C] | H |
| 1244 | 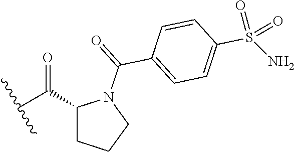 [SMILES: *C(=O)[C@H]1CCCN1C(=O)c1ccc(S(N)(=O)=O)cc1] | 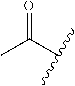 [SMILES: *C(C)=O] | 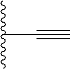 [SMILES: *C#C] | H |
| 1245 | 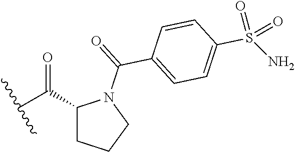 [SMILES: *C(=O)[C@H]1CCCN1C(=O)c1ccc(S(N)(=O)=O)cc1] | H | H | Me |
| 1246 | 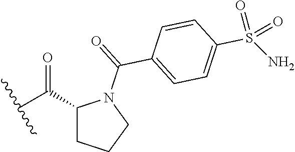 [SMILES: *C(=O)[C@H]1CCCN1C(=O)c1ccc(S(N)(=O)=O)cc1] | 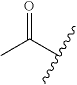 [SMILES: *C(C)=O] | H | Me |
| 1247 | 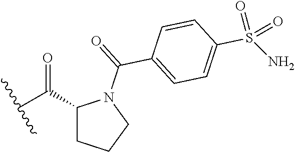 [SMILES: *C(=O)[C@H]1CCCN1C(=O)c1ccc(S(N)(=O)=O)cc1] | H | 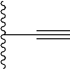 [SMILES: *C#C] | Me |
| 1248 | 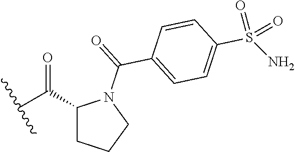 [SMILES: *C(=O)[C@H]1CCCN1C(=O)c1ccc(S(N)(=O)=O)cc1] | 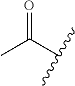 [SMILES: *C(C)=O] | 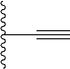 [SMILES: *C#C] | Me |
| 1249 | 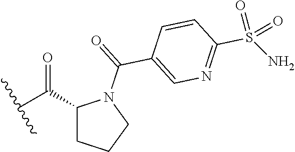 [SMILES: *C(=O)[C@H]1CCCN1C(=O)c1ccc(S(N)(=O)=O)nc1] | H | H | H |
| 1250 | 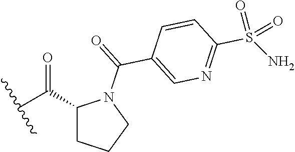 [SMILES: *C(=O)[C@H]1CCCN1C(=O)c1ccc(S(N)(=O)=O)nc1] | 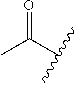 [SMILES: *C(C)=O] | H | H |
| 1251 | 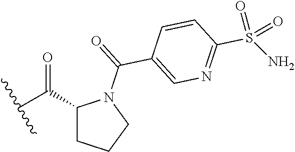 [SMILES: *C(=O)[C@H]1CCCN1C(=O)c1ccc(S(N)(=O)=O)nc1] | H | 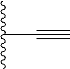 [SMILES: *C#C] | H |
| 1252 | 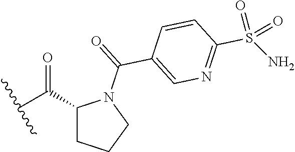 [SMILES: *C(=O)[C@H]1CCCN1C(=O)c1ccc(S(N)(=O)=O)nc1] | 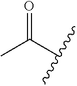 [SMILES: *C(C)=O] | 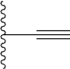 [SMILES: *C#C] | H |
| 1253 | 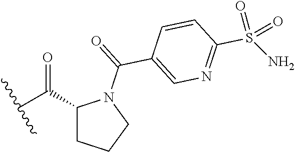 [SMILES: *C(=O)[C@H]1CCCN1C(=O)c1ccc(S(N)(=O)=O)nc1] | H | H | Me |
| 1254 | 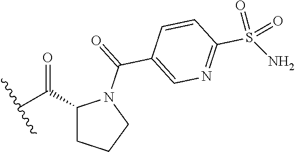 [SMILES: *C(=O)[C@H]1CCCN1C(=O)c1ccc(S(N)(=O)=O)nc1] | 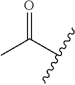 [SMILES: *C(C)=O] | H | Me |
| 1255 | 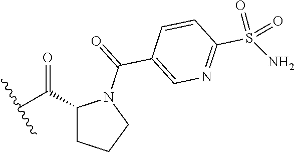 [SMILES: *C(=O)[C@H]1CCCN1C(=O)c1ccc(S(N)(=O)=O)nc1] | H | 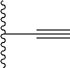 [SMILES: *C#C] | Me |
| 1256 | 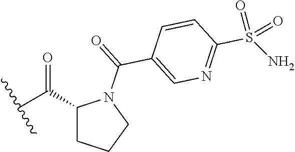 [SMILES: *C(=O)[C@H]1CCCN1C(=O)c1ccc(S(N)(=O)=O)nc1] | 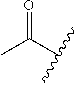 [SMILES: *C(C)=O] | 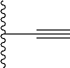 [SMILES: *C#C] | Me |

14. A pharmaceutical composition comprising one or more compounds according to any one of claims 1-13, or a pharmaceutically acceptable salt, solvate, hydrate, cocrystal, or prodrug thereof, and a pharmaceutically acceptable carrier.

15. A pharmaceutical composition for treating or preventing a disease or condition alleviated by activating and/or enhancing estrogen receptor-β (ERβ)-activity, the pharmaceutical composition comprising one or more compounds according to any one of claims 1-13, or a pharmaceutically acceptable salt thereof, and a physiologically compatible carrier.

16. The pharmaceutical composition of claim 15, wherein the disease or condition is selected from a depressive disorder, an anxiety disorder, post-traumatic stress disorder (PTSD), drug addiction, schizophrenia, Alzheimer's dementia, Parkinson's disease, stroke, traumatic brain injury (TBI), amyotrophic lateral sclerosis (ALS), complex regional pain syndrome (CRPS), chronic pain, neuropathic pain, anhedonia, fatigue, andropause-induced symptoms, and orchiectomy-induced symptoms.

17. A pharmaceutical composition for treating a disease or condition selected from a depressive disorder, an anxiety disorder, post-traumatic stress disorder (PTSD), drug addiction, schizophrenia, Alzheimer's dementia, Parkinson's disease, stroke, traumatic brain injury (TBI), amyotrophic lateral sclerosis (ALS), complex regional pain syndrome (CRPS), chronic pain, neuropathic pain, anhedonia, fatigue, andropause-induced symptoms, and orchiectomy-induced symptoms, the pharmaceutical composition comprising one or more compounds according to any of claims 1 to 11, or a pharmaceutically acceptable salt, solvate, hydrate, cocrystal, or prodrug thereof, and a pharmaceutically acceptable carrier.

18. The pharmaceutical composition of claim 16 or 17, wherein the depressive disorder is selected from major depressive disorder, bipolar disorder, postpartum depression, drug withdrawal induced depression, treatment refractory depression, persistent depressive disorder (dysthymia), perinatal depression, seasonal affective disorder, seasonal depression, premenstrual dysphoric disorder, psychotic depression, suicidal ideation, suicidal actions, disruptive mood dysregulation disorder, premenstrual dysphoric disorder, substance/medication-induced depressive disorder, depressive disorder due to another medical condition, other specified depressive disorder, and an unspecified depressive disorder.

19. The pharmaceutical composition of claim 16 or 17, wherein the anxiety disorder is selected from general anxiety disorder, obsessive-compulsive disorder (OCD), separation anxiety disorder, selective mutism, specific phobia, social anxiety disorder (social phobia), panic disorder, panic attack (specifier), agoraphobia, substance/medication-induced anxiety disorder, anxiety disorder due to another medical, other specified anxiety disorder, anhedonia, and unspecified anxiety disorder.

20. The pharmaceutical composition of claim 16 or 17, wherein the drug addiction is selected from nicotine addiction, alcohol addiction, cannabis addiction, cocaine addiction, and opioid addiction.

21. The pharmaceutical composition of any one of claims 14-20, wherein the pharmaceutical composition is formulated for oral administration.

22. A method of treating or preventing a disease or condition alleviated by activating and/or enhancing estrogen receptor-β (ERβ)-activity in a patient in need thereof, the method comprising administering a therapeutically effective amount of one or more compounds according to any one of claims 1-13, or a pharmaceutically acceptable salt, solvate, hydrate, cocrystal, or prodrug thereof.

23. The method of claim 22, wherein the disease or condition is selected from a depressive disorder, an anxiety disorder, post-traumatic stress disorder (PTSD), drug addiction, schizophrenia, Alzheimer's dementia, Parkinson's disease, stroke, traumatic brain injury (TBI), amyotrophic lateral sclerosis (ALS), complex regional pain syndrome (CRPS), chronic pain, neuropathic pain, anhedonia, fatigue, andropause-induced symptoms, and orchiectomy-induced symptoms.

24. A method of treating or preventing a disease or condition selected from a depressive disorder, an anxiety disorder, post-traumatic stress disorder (PTSD), drug addiction, schizophrenia, Alzheimer's dementia, Parkinson's disease, stroke, traumatic brain injury (TBI), amyotrophic lateral sclerosis (ALS), complex regional pain syndrome (CRPS), chronic pain, neuropathic pain, anhedonia, fatigue, andropause-induced symptoms, and orchiectomy-induced symptoms in a patient in need thereof, the method comprising administering a therapeutically effective amount of one or more compounds according to any of claims 1 to 11, or a pharmaceutically acceptable salt, solvate, hydrate, cocrystal, or prodrug thereof.

25. The method of claim 23 or 24, wherein the depressive disorder is selected from major depressive disorder, bipolar disorder, postpartum depression, drug withdrawal induced depression, treatment refractory depression, persistent depressive disorder (dysthymia), perinatal depression, seasonal affective disorder, seasonal depression, premenstrual dysphoric disorder, psychotic depression, suicidal ideation, suicidal actions, disruptive mood dysregulation disorder, premenstrual dysphoric disorder, substance/medication-induced depressive disorder, depressive disorder due to another medical condition, other specified depressive disorder, and an unspecified depressive disorder.

26. The method of claim 23 or 24, wherein the anxiety disorder is selected from general anxiety disorder, obsessive-compulsive disorder (OCD), separation anxiety disorder, selective mutism, specific phobia, social anxiety disorder (social phobia), panic disorder, panic attack (specifier), agoraphobia, substance/medication-induced anxiety disorder, anxiety disorder due to another medical, other specified anxiety disorder, anhedonia, and unspecified anxiety disorder.

27. The method of claim 23 or 24, wherein the drug addiction is selected from nicotine addiction, alcohol addiction, cannabis addiction, cocaine addiction, and opioid addiction.

28. The method of any one of claims 22-27, wherein the patient is male.
